(19) United States
(12) Patent Application Publication
(10) Pub. No.: US 2024/0390485 A1
(43) Pub. Date: Nov. 28, 2024

(54) HYPOALLERGENIC PEANUT ALLERGENS, PRODUCTION AND USE THEREOF

(71) Applicant: Ukko Inc., San Francisco, CA (US)

(72) Inventors: Yanay OFRAN, Tel Aviv (IL); Moshe BEN DAVID, Rehovot (IL); Orly MARCU GARBER, Modiin (IL); Si NAFTALY KIROS, Kiryat Bialik (IL); Gil DIAMANT, Tel Aviv (IL); Almog BREGMAN COHEN, Rehovot (IL); Maayan KORMAN, Har Adar (IL); Anna CHUPRIN, Tel Aviv (IL); Zohar BIRON SOREK, Tel Aviv (IL); Lior ROSENFELD SHLAMKOVICH, Modi’in (IL); Etai ROTEM, Rehovot (IL); Yair GAT, Rehovot (IL)

(73) Assignee: Ukko Inc., San Francisco, CA (US)

(21) Appl. No.: 18/292,959

(22) PCT Filed: Aug. 2, 2022
(86) PCT No.: PCT/IB2022/057144

§ 371 Date: Jan. 29, 2024

Related U.S. Application Data
(60) Provisional application No. 63/228,604, filed on Aug. 3, 2021.
(60) Provisional application No. 63/228,606, filed on Aug. 3, 2021.
(60) Provisional application No. 63/284,108, filed on Nov. 30, 2021.
(60) Provisional application No. 63/292,441, filed on Dec. 22, 2021.
(60) Provisional application No. 63/311,117, filed on Feb. 17, 2022.
(60) Provisional application No. 63/319,393, filed on Mar. 14, 2022.
(60) Provisional application No. 63/319,394, filed on Mar. 14, 2022.

(51) Int. Cl. A61K 39/35; A61P 37/08; C07K 14/415

(52) U.S. Cl. CPC A61K 39/35; A61P 37/08; C07K 14/415

(57) ABSTRACT

In one embodiment, the present disclosure provides hypoallergenic peanut allergens Ara h 1 or Ara h 2 variants lacking at least one epitope recognized by anti-Ara h 1 antibodies or anti-Ara h 2 antibodies, thereby having reduced or abolished antibody binding to the peanut allergen variants. These hypoallergenic peanut allergen variants may be used in methods of inducing desensitization to peanuts in a subject allergic to peanuts.

### SEQUENCE LISTING STATEMENT

The instant application contains a Sequence Listing which has been submitted electronically in .xml format and is hereby incorporated by reference in its entirety. Said .xml copy, created on Jul. 29, 2022, is named P-605376-PC_SL.xml and is 421.4 Kilo bytes in size.

### FIELD OF INVENTION

The disclosure relates in general to recombinant hypoallergenic peanut allergens Ara h 1 and Ara h 2, methods of producing same, and uses thereof.

### BACKGROUND

One of the most severe food allergies known today is peanut allergy, where allergic individuals respond to exposure to peanuts, even at low concentrations, with symptoms ranging from mild, local effects, to severe, life-threatening effects. Peanuts are the leading cause for food induced anaphylactic shock in the United States (Finkelman, (2010) Current Opinion in Immunology, 22(6):783-788) and some form of allergic reaction to peanuts is reported in around 1% of the US population (Sicherer S H, et al., (2010). J Allergy Clin Immunol. 125(6):1322-6).

So far, 16 peanut proteins have been identified to be those leading to the IgE mediated allergic reaction (Palladino, C., & Breiteneder, H. (2018). Molecular immunology, 100:58-70). Of these proteins, the seed storage proteins Ara h 1, Ara h 2, Ara h 3 and Ara h 6 are considered major allergens, those whose recognition by an IgE antibody mediated response is correlated with more severe symptoms (Palladino, et al., 2018; ibid) (Bernard, et al., (2007) J Agric Food Chem. 55(23):9663-9). Out of the 16 peanut allergens, Ara h 2 is considered to be the most important, as it is recognized by around 75-80% of sera IgE from American children of ages 3-6 (Valcour, et. al., Ann Allergy Asthma Immunol 119 (2017)) (Koppelman et al., (2004). Clin Exp Allergy. 34(4):583-90) Ara h 2 is a 17 kD monomeric polypeptide that is a member of the 2S albumin family, belonging to the prolamine protein superfamily (Lehmann K, Schweimer K, Reese G, Randow S, Suhr M, Becker W M, et al. (2006) Biochem J. 395(3):463-72.). It comprises 6-10% of the total protein in peanut extract (Koppelman, S. J., et al. (2001) Allergy 56:2). Ara h 2 causes sensitization directly through the gastrointestinal tract. Its core structure is highly resistant to proteolysis due to the high stability structure generated from well-conserved Cystines forming disulfide bonds. A comparison between the folded and unfolded versions of Ara h 2 revealed that IgE antibodies recognize both linear epitopes and conformational epitopes, which are bound by sera only when tested against the folded protein (Bernard et al., (2015) J Allergy Clin Immunol. 135(5):1267-74.el-8.).

Ara h 1 is 63 kDa peanut seed protein comprises 12-16% of the total protein in peanut extracts (Koppelman, S. J., et al. ibid). Ara h 1 possesses a heat-stable 7S vicilin-like globulin with a stable homotrimeric form. (Pomés et al. (2003) The Journal of Allergy and Clinical Immunology. 111 (3): 640-5) Ara h 1 is initially a pre-pro-protein which, following two endoproteolytic cleavages, becomes the mature form found in peanuts. The mature form has flexible regions and a core region. The crystal structure of the Ara h 1 core (residues 170-586) (3S7I.pdb; 3SMH.pdb) shows that the central part of the allergen has a bicupin fold. Previously, linear IgE binding epitopes have been mapped in Ara h 1 and substitutions of only one amino acid per epitope led to the loss of IgE binding. (Burks et al. (1997). Eur J Biochem 1997; 245(2):334-9). However, conformational epitopes to the thermostable trimer surface are less studied.

Other than complete avoidance of exposure to the allergen patients have been offered treatment of controlled exposure to increasing doses of the respective allergens (i.e. immunotherapy (IT)). The focus of IT treatment to increase the amount of allergen that does not trigger an allergic reaction, effectively reducing the chance for allergenicity while re-educating the immune system to deal with the allergen, thus potentially preventing allergic response upon accidental ingestion of the allergen. Immunotherapy treatment is currently provided in clinics. In recent years companies have developed products that standardize the peanut extract, in order to offer a treatment regimen that is safer and applicable for at home use.

There remains an unmet need for hypoallergenic peanut proteins and methods of use thereof for standardized immunotherapeutic treatment, in subjects allergic to peanut allergens.

### SUMMARY

Described herein are several epitope mapping approaches for designing hypoallergenic peanut allergens that maintain biophysical and functional characteristics, for example, for the generation of Ara h 1 and Ara h 2 allergen variants. In one aspect, disclosed herein are hypoallergenic peanut allergens Ara h 1 or Ara h 2 variants lacking at least one epitope recognized by an anti-Ara h 1 antibody or anti-Ara h 2 antibody, thereby reducing or abolishing antibody binding to the peanut allergen variants. In one aspect, these hypoallergenic peanut allergen variants may be used in methods of inducing desensitization to peanuts in a subject allergic to peanuts.

In one aspect, provided herein is a recombinant Ara h 2 variant polypeptide comprising an amino acid sequence that is at least 50% identical to the sequence set forth in SEQ ID NO: 3, wherein the variant comprises one or more amino acid substitutions, deletions, insertions, or any combination thereof that are located within a single epitope recognized by an anti-Ara h 2 antibody. In another embodiment, the Ara h 2 variant comprises one or more amino acid substitutions, deletions, insertions, or any combination thereof that are located within at least two epitopes recognized by anti-Ara h 2 antibodies.

In one embodiment, the recombinant Ara h 2 variant polypeptide comprises the amino acid sequence set forth in SEQ ID NO: 4, and the substitutions, deletions, insertions, or any combination thereof at one or more of positions 12, 15, 16, 22, 24, 46, 53, 65, 80, 83, 86, 87, 90, 104, 115, 123, 127, or 140 of SEQ ID NO: 4, as compared with the amino acid residues at those same positions in SEQ ID NO: 3. In one embodiment, the recombinant Ara h 2 variant comprises
one or more of the following substitution mutation(s):
(a) N, Q, E, D, T, S, G, P, C, K, H, Y, W, M, I, L, V, or A at position 12;
(b) R, E, K, Y, W, F, M, I, V, C, D, G, or A at position 15;
(c) R, K, D, Q, T, M, P, C, E, or W at position 16;
(d) F, Y, W, Q, E, T, S, A, M, I, L, C, R, or H at position 22;
(e) D, E, H, K, S, T, N, Q, L, I, M, W, Y, F, P, A, or G at position 24;
(f) T, V, E, H, S, A, G, Q, N, D, R, P, M, I, L, or C at position 46;
(g) T, S, Q, V, A, G, C, P, M, L, I, E, H, R, K, N, or D at position 53;
(h) T, A, N, D, Q, R, K, H, I, L, M, V, W, P, G, C, or E at position 65;
(i) N, S, T, V, A, I, L, M, F, Y, W, C, E, K, R, or G at position 80;
(j) D, A, C, F, I, P, T, V, W, Y, or Q at position 83;
(k) Y, F, H, R, E, C, G, I, L, M, V, T, S, or Q at position 86;
(l) F, Y, I, L, M, V, A, S, Q, R, K, D, or P at position 87;
(m) S, P, or R at position 90;
(n) L, M, K, R, H, E, D, A, Y, N, S, or W at position 104;
(o) V, D, E, I, L, K, M, N, S, T, A, I, W, F, Y, or H at position 115;
(p) I, Q, or A at position 123;
(q) H, A, D, E, F, G, L, N, P, S, T, W, Y, Q, or V at position 127; or
(r) G, A, C, E, Y, F, H, K, L, M, N, P, Q, S, or V at position 140.

In one embodiment, the recombinant Ara h 2 variant further comprises additional substitutions, deletions, insertions, or any combination thereof at one or more of positions, 28, 44, 48, 51, 55, 63, 67, 107, 108, 109, 124, 125, or 142 of SEQ ID NO: 4, as compared with the amino acid residues at those same positions in SEQ ID NO: 3. In one embodiment, the recombinant Ara h 2 variant comprises one or more of the following substitution mutation(s):
(a) S, T, V, N, A, P, I, L, F, Y, H, R, K, E, or D at position 28;
(b) I, A, C, G, H, L, F, Y, N, P, Q, K, E, S, T, V, M, or R at position 44;
(c) V, G, C, E, H, Q, F, K, L, I, W, Y, N, R, S, T, V, A, or D at position 48;
(d) S, G, Y, F, W, M, N, Q, E, R, K, H, T, or V at position 51;
(e) G, A, D, E, F, Y, H, Q, V, I, L, M, R, K, S, T, C, or W at position 55;
(f) P, C, F, V, I, L, M, W, Y, N, S, T, Q, G, H, K, or R at position 63;
(g) E, Q, N, R, H, Y, F, W, M, L, V, T, S, A, or G at position 67;
(h) A, C, F, G, H, I, K, L, M, Q, P, R, S, T, V, W, or Y at position 107;
(i) T, V, D, E, R, H, Y, W, I, G, A, Q, or K at position 108;
(j) K, C, S, R, G, P, Y, W, or I at position 109;
(k) D, A, C, F, G, H, I, N, S, T, V, Y, L, or Q at position 124;
(l) M, I, L, W, Y, G, K, N, T, V, or A at position 125; or
(m) M, A, C, E, F, G, H, I, K, L, N, P, Q, R, S, T, V, W, or Y at position 142.

In one embodiment, the recombinant Ara h 2 variant comprises the amino acid sequence as set forth in any one of SEQ ID NOs:10-63, 168, 170, 195-201, 204-210, 247-249, or comprises an amino acid sequence having at least 80% identity with the amino acid sequences set forth in any of SEQ ID NOs: 10-63, 168, 170, 195-201, 204-210, 247-249.

Also provided herein are a nucleotide sequence encoding any one of the above recombinant Ara h 2 variants, an expression vector comprising the nucleotide sequence, as well as a cell comprising the expression vector. There is also provided a method of using the expression vector to produce the recombinant Ara h 2 variants disclosed herein.

In another aspect, provided herein is a recombinant Ara h 1 variant polypeptide comprising an amino acid sequence that is at least 50% identical to the sequence set forth in SEQ ID NO: 65, wherein the Ara h 1 variant comprises one or more substitutions, deletions, insertions, or any combination thereof that are located within a single epitope recognized by an anti-Ara h 1 antibody. In another embodiment, the Ara h 1 variant comprises one or more amino acid substitutions, deletions, insertions, or any combination thereof that are located within at least two epitopes recognized by anti-Ara h 1 antibodies.

In one embodiment, the recombinant Ara h 1 variant polypeptide comprises the amino acid sequence set forth in SEQ ID NO: 67, and the substitutions, deletions, insertions, or any combination thereof at one or more of positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65. In one embodiment, the recombinant Ara h 1 variant comprises one or more of the following substitution mutation(s):
(a) D at position 194;
(b) A at position 195;
(c) H at position 213;
(d) R, D, L, I, F, or A at position 215;
(e) A at position 231;
(f) E at position 234;
(g) R at position 245;
(h) E at position 267;
(i) D at position 287;
(j) E at position 294;
(k) A or H at position 312;
(l) H at position 331;
(m) E, V, or A at position 419;
(n) R or A at position 422;
(o) A at position 443;
(p) A at position 455;
(q) A, K, or T at position 462;
(r) S at position 463;
(s) A or S at position 464;
(t) Q at position 480;
(u) A, E, or N at position 494; and
(v) K at position 500

In one embodiment, the recombinant Ara h 1 variant further comprises additional substitutions, deletions, insertions, or any combination thereof at one or more of positions 12, 24, 27, 30, 42, 57, 58, 73, or 523 of SEQ ID NO: 67, as compared with the amino acid residues at those same positions in SEQ ID NO: 65. In one embodiment, the recombinant Ara h 1 variant comprises one or more of the following substitution mutation(s): K or A at position 12; V or E at position 24; A or H at position 27; E or A at position 30; L or K at position 42; D or L at position 57; S or R at position 58; A or M at position 73; and A or K at position 523.

In one embodiment, the recombinant Ara h 1 variant further comprises additional substitutions, deletions, insertions, or any combination thereof at one or more of positions 87, 88, 96, 99, 196, 197, 200, 209, 238, 249, 260, 261, 263, 265, 266, 278, 283, 288, 290, 295, 318, 322, 334, 336, 378, 417, 421, 441, 445, 481, 484, 485, 487, 488, or 491 of SEQ ID NO: 67, as compared with the amino acid residues at those same positions in SEQ ID NO: 65. In one embodiment, the recombinant Ara h 1 variant comprises one or more of the following substitution mutation(s):
(a) A at position 87;
(b) A at position 88;
(c) A at position 96;
(d) A at position 99;
(e) H at position 196;
(f) A at position 197;
(g) V, A or Q at position 200;
(h) S at position 209;
(i) Q at position 238;
(j) N at position 249;
(k) K at position 260;
(l) R at position 261;
(m) K or L at position 263;
(n) S at position 265;
(o) R or L at position 266;
(p) R at position 278;
(q) E at position 283
(r) Q at position 288;
(s) R at position 290;
(t) A at position 295;
(u) H at position 318;
(v) A or K at position 322;
(w) D, A or N at position 334;
(x) R or S at position 336;
(y) K or E at position 378;
(z) R at position 417;
(aa) E or S at position 421;
(bb) N at position 441;
(cc) A at position 443;
(dd) A or S at position 481;
(ee) R, S, A, or M at position 484;
(ff) A at position 485;
(gg) S or K at position 487;
(hh) A at position 488; or
(ii) A, S or E at position 491.

In one embodiment, the recombinant Ara h 1 variant further comprises a substitution mutation of A at position 84 of SEQ ID NO:67.

In one embodiment, the recombinant Ara h 1 variant comprises the amino acid sequence as set forth in any one of SEQ ID NOs: 68-161. 174, 176, 178, 180, 182, 184, 193, 194, 211-246, or comprises an amino acid sequence having at least 80% identity with the amino acid sequences set forth in any of SEQ ID NOs: 68-161, 174, 176, 178, 180, 182, 184, 193, 194, 211-246.

Also provided herein are nucleotide sequences encoding any one of the above recombinant Ara h 1 variants, an expression vector comprising the nucleotide sequence, as well as a cell comprising the expression vector. There is also provided a method of using the expression vector to produce the recombinant Ara h 1 variants disclosed herein.

In another aspect, the present disclosure also provides a method of inducing desensitization to peanuts in subject allergic to peanuts, the method comprises administering to the subject a composition comprising the hypo-allergenic Ara h 1 variants or the hypo-allergenic Ara h 2 variants disclosed herein, or a combination thereof, thereby inducing desensitization to peanuts in the subject.

In another aspect, the present disclosure also provides a method of inducing desensitization to peanuts in subject allergic to peanuts, the method comprises administering to the subject a composition comprising nucleotide or modified nucleotide sequences encoding the hypo-allergenic Ara h 1 variants or the hypo-allergenic Ara h 2 variants disclosed herein, or a combination thereof, thereby inducing desensitization to peanuts in the subject.

In another aspect, the present disclosure also provides a genetically modified peanut plant expressing the hypo-allergenic Ara h 1 variants or the hypo-allergenic Ara h 2 variants disclosed herein, or a combination thereof.

In another aspect, the present disclosure also provides a processed food product comprising the hypo-allergenic Ara h 1 variants or the hypo-allergenic Ara h 2 variants disclosed herein, or a combination thereof.

### BRIEF DESCRIPTION OF THE DRAWINGS

The subject matter regarded the hypoallergenic polypeptide variants described herein having reduced allergenicity while maintaining immunogenicity, and methods of making the same is particularly pointed out and distinctly claimed in the concluding portion of the specification. The engineered Ara h 1 and Ara h 2 polypeptide variants and methods of making the same, however, both as to organization and method of operation, together with objects, features, and advantages thereof, may best be understood by reference to the following detailed description when read with the accompanying drawings in which:

FIG. 1. Ara h specific monoclonal antibodies (mAbs) discovery pipeline. FIG. 1 presents a flow schematic of the epitope mapping procedure based on the discovery of monoclonal antibodies (mAbs) from peanut allergic patients, from patient sample to residue-level mapping of epitopes. The steps shown are from collection of patient PMBCs to purification of the mAbs by single cell sorting (upper panel) or phage display panning (lower panel).

FIG. 2: Three approaches for epitope mapping of Ara h purified mAbs. In Approach 1, for each isolated Ara h specific mAb, an Ara h yeast display single site saturation mutations library is sorted for binding. Sorted high and low binding populations are sent to deep sequencing and variant enrichments are analyzed to identify the mAb bound Ara h region. In Approach 2, peptide arrays are utilized for the analysis of identified binding sites. Two types of Celluspot™ peptide microarray-based immunoassays are carried out for each mAb, a peptide array with wild-type (WT) Ara h sequences and an additional one with point mutations, to determine the binding of linear epitopes. In Approach 3, mutational patch analysis is performed. Structure-based in-silico design of surface-exposed patches mutagenesis was followed by an indirect ELISA screen of Ara h variants to the specific Ara h mAb. Reduction or elimination of mAb binding to the mutated variant confirms the epitope location in the sequence.

FIGS. 3A and 3B. Point mutants of Ara h 2 exhibit lower binding to serum-derived anti-Ara h 2 monoclonal antibody (mAb) B701. FIG. 3A presents FACS sorting of Ara h 2 saturation library based on expression (x-axis) and binding (y-axis) of Ara h 2 variants to mAb B701 a. FIG. 3B presents enrichment ratio of library point mutants in the S2 low-B701 binding population, expressed as log 2(fB701_S2_low/fs1), where fB701_S2_low is the fraction of a given mutation in the sorted library and fs1 is its fraction in the S1 library. Coloring is from white (depletion) to blue (enrichment, indicating the point mutation leads to reduction in mAb binding). Position numbering (X-axis) is based on SEQ ID No 1.

FIGS. 4A and 4B. Ara h 1 and 2 variants show reduced binding to anti-Ara h 1 and anti-Ara h 2 mAbs, B536 and B843 respectively. Indirect ELISA titration with increasing concentrations of the anti Ara h mAb was used to test binding to wild-type (WT) Ara h 2 or modified Ara h 2 variants (FIG. 4A) and WT Ara h 1 or modified Ara h 1 variants (FIG. 4B). The data presented demonstrates that modified Ara h 1 and Ara h 2 variants show dramatically reduced binding to serum-derived anti-Ara h 2 mAb B536 (FIG. 4A) or anti-Ara h 1 mAb B843 (FIG. 4B). An ELISA assay analysis was used for measuring binding of WT Ara h 2 polypeptide (SEQ ID NO: 2) or modified Ara h 2 variant polypeptides to increasing concentrations of the anti-Ara h 2 B536 mAb (FIG. 4A) or anti-Ara h 1 B843 (FIG. 4B). Bovine serum albumin (BSA) was used as a negative control.

FIGS. 5A and 5B. Linear epitope mapping and de-epitoping reveals mutations that abolish binding to Ara h 2 epitopes. FIG. 5A: Linear epitope mapping of patient P70 reveals IgE binding to Ara h 1, Ara h 2, Ara h 3 and Ara h 6. Black box highlights an Ara h 2 mapped epitope L3 (a peptide derived from positions 42-56 of SEQ ID No 3). FIG. 5B: Linear de-epitoping of patient P70 Ara h 2 epitopes. Black box highlights the same peptide as in FIG. 5A. The box highlights a spot where a point mutation dramatically reduced binding to L3.

FIGS. 6A and 6B. Modified Ara h 2 and Ara h 1 variants exhibit reduced activation potential. FIGS. 6A and 6B present data showing that modified Ara h 2 variants (FIG. 6A) and Ara h 1 variants (FIG. 6B) exhibit reduced activation of basophils. Representative results of a rat basophilic leukemia (RBL) SX-38 cell degranulation assay, testing serum IgE-mediated cellular response to either WT (black) or modified (grey colored) Ara h 2 variants. Results are shown for eight patient sera, denoted S70, S129, A182, B192, W11, S95, S101, and E282.

FIGS. 7A and 7B. Modified Ara h 2 variants exhibit dramatically reduced activation potential of human basophils compared to natural Ara h 2. FIGS. 7A and 7B present data from two different peanut allergy patient blood samples showing that modified Ara h 2 variants exhibit dramatically reduced activation of basophils. Representative results of a basophil activation test (BAT), testing sera IgE-mediated cellular response to either WT (nArah2 and rArah2) or modified (B764 (SEQ ID NO:11) and B1001 (SEQ ID NO:10) recombinant Ara h 2 variants. Ara h 2—natural Ara h 2, extracted from peanuts, rAra h 2—recombinant Ara h 2. EC50 values noted below were derived with a 3-parameter function (where not noted, reactivity was too low to derive a value).

FIGS. 8A and 8B. Activation of allergy-patient derived peripheral blood T helper cells by recombinant WT and modified Ara h 2 variants. FIGS. 8A and 8B present activation of allergy-patient derived peripheral blood T helper cells (FIG. 8A—patient SH409 & FIG. 8B—patient B293) by WT and representative modified Ara h 2 variants (B764 and B1001). Representative results are shown. Cells were stained by “Celltrace” proliferation dye, activated with various allergens (WT or variants) or left un-activated (untreated), and incubated for 7 days. Cells were harvested, stained for viability and T helper cell markers and Live, proliferating T helper cells were isolated (CD3+, CD4+, Viability dye-, proliferation dye—dim). Graphs present mean and SE of % proliferating T helper cells per treatment.

FIGS. 9A-9F. Modified Ara h 2 variants maintain high thermal stability. Circular dichroism (CD) analysis of recombinant Ara h 2 WT (Ara h 2_B123) and mutated variants (Ara h 2_B764 and Ara h 2_B1001) is presented. FIG. 9A (WT), FIG. 9B (Ara h 2_B764), and FIG. 9C (Ara h 2_B1001) show the CD spectra of the WT and the variants at 25° C., exhibiting similar secondary structure composition of the variants relative to the WT. FIG. 9D (WT), FIG. 9E (Ara h 2_B764), and FIG. 9F (Ara h 2_B1001) shows the stability of Ara h 2 WT and variants at temperature ranges of 20-90° C., displaying a high Thermal melting temperature (TM)>90° C., suggesting no significant deviation from the natural fold, as expected for at least the WT (Lehmann, K., et al., (2006). Structure and stability of 2S albumin-type peanut allergens: implications for the severity of peanut allergic reactions. The Biochemical journal, 395(3), 463-472).

FIG. 10. Expression and secretion of allergen from transfected cells. Mammalian cells were transfected with vectors encoding for wild-type or de-epitoped variants of the peanut allergens Ara h 2 and Ara h 1. The secreted allergen protein was purified and characterized by SDS-PAGE analysis. (panel a) wild-type Ara h 2, (panel b) wild-type Ara h 1, (panel c) de-epitoped Ara h 2, (panel d) two de-epitoped variants of Ara h 1.

FIG. 11. Binding to IgE in allergic patients' sera. Ara h 1 was expressed and secreted from HEK293 cells, purified and assayed for binding of IgE following binding to allergic patient sera or control non-allergic serum. Binding was compared to natural Ara h 1 (nArah1), recombinant E. coli-derived wild-type Ara h 1 (rAra h 1), and recombinant HEK293 cell-derived wild-type Ara h 1 (HEK Ara h 1).

FIG. 12. Binding to anti-Ara h 2 monoclonal antibodies. Peanut allergen Ara h 2 was expressed, secreted and purified from HEK293 cells. Binding to well-characterized anti-Ara h 2 monoclonal IgG antibodies was assayed and compared between recombinant Ara h 2 (rAra h 2) and the HEK-derived Ara h 2 (HEK Ara h 2 wild-type). Binding characteristics are shown using six IgGs (mAb 1-6) and medium from HEK293 cell medium was used as negative control.

FIG. 13 shows a HPLC size exclusion chromatogram trace of purified Ara h 1 expressed from transfected mammalian cells, demonstrating a correct trimetric state.

FIG. 14 presents a total-mass measurement of Ara h 2 expressed from transfected mammalian cells, showing a mass of 18966.8 Da, the expected mass of the sequence—8 Da, corresponding to the four disulfide bonds of oxidatively folded Ara h 2.

FIG. 15 presents a general outline of patient sample-based pipeline for allergen de-epitoping.

FIGS. 16A-16C presents biochemical characterization of the Ara h 2 variant B1001. FIG. 16A: Identification of Ara h 2 B1001 by western blot. Proteins were separated on stain-free SDS-PAGE and imaged by UV (left pane) as loading control. Proteins were then transferred to a PVDF membrane and detected using a commercial polyclonal antibody anti Ara h 2 (right pane). Lanes: 1, Natural Ara h 2; 2, recombinant WT Ara h 2; 3, B1001 Ara h 2 variant; 4, recombinant Ara h 1 (peanut negative control); 5, BSA (general negative control). FIG. 16B: Size Exclusion Chromatography (SEC)-HPLC analysis for molecule size and oligomeric state estimation. Purified natural Ara h 2 (top pane), recombinant WT Ara h 2 (middle pane) and Ara h 2 variant B1001 (bottom pane) were analyzed by SEC-HPLC, chromatograms are shown. Retention times (RT) and estimated Mw are indicated inside panes. FIG. 16C: Circular dichroism (CD) analysis of WT Ara h 2 and B1001 variant. Left panels show the CD spectra of WT Ara h 2 and B1001 at 25° C. Right panes show the CD spectra across a temperature range of 20-90° C., indicating stability of secondary structures (curve ° C. marked by color noted in legend).

FIGS. 17A-17B show reduced patient plasma binding to B1001 is differential for IgE and IgG. ELISA assays were carried out on plates coated with Ara h 2 or B1001. Plasma samples from 24 peanut allergy patients were serially diluted and incubated on plates to detect patient IgE or IgG binding to each allergen. Titration curves were derived and used to calculate area under the curve (AUC) values. FIG. 17A: Relative binding of patient IgE or IgG to Ara h 2 or B1001. Figure shows AUC medians and ranges. Wilcoxon matched-pairs signed rank test p-values are noted. FIG. 17B: B1001/Ara h 2 AUC ratios were calculated to express reduced binding of variant. Figure shows individual AUC ratios with IgE and IgG ratios pairing by patient marked with thin lines and group medians marked with thick lines. Wilcoxon matched-pairs signed rank test p-values are noted.

FIGS. 18A-18B show allergenic potential of B1001 is markedly reduced compared to natural Ara h 2. FIG. 18A: RBL SX-38 assay. Cells were incubated overnight with patient plasma, washed and incubated with noted proteins at increasing concentrations in Tyrode's buffer. Buffer was then moved to a separate plate and incubated with a colorimetric substrate of the granular enzyme Beta-hexosaminidase. OD was measured at 450 nm and net-degranulation was calculated by subtracting OD of untreated wells and dividing by OD of lysed wells. Reactions were carried out in duplicates. Plot shows means±S.E for 28 patients. FIG. 18B: BAT assay. Fresh patient blood was induced with varying allergen concentrations according to available volume of blood, but with at least 6 concentrations covering the 1-10,000 ng/ml range. Samples were then incubated for 30 minutes, stained, washed, fixed and analyzed by flow cytometry. Plot shows means±S.E for each concentration with baseline subtracted, representing 18-44 patients. EC50 values were derived from the resulting curves by fitting to a 4-parameter logistic regression model.

FIGS. 19A-19B show B1001 retains partial immunogenicity for peanut allergy patient peripheral blood T cells. PBMC were isolated from peanut allergy patient blood, stained with Celltrace violet proliferation dye and incubated for 7 days with DMSO alone or with DMSO-dissolved peptide pools covering the entire sequence of Ara h 2 or B1001 (4-8 replicates/treatment, 2-2.5×10⁵ cells/well). Media was then removed and stored for cytokine secretion analysis while cells were stained for Th identification and analyzed by flow cytometry to detect proliferation. Media was analyzed by sandwich ELISA to detect IL-5, IL-13 and IFNγ secretion. Data was collected only from tests with a WT Ara h 2 response that was [S.I>2+M.W p-value<0.1]. FIG. 19A: Charts show means±S.E, sample number at the top left and p-values for pairwise comparisons by Wilcoxon rank-sum test above bars. FIG. 19B: Estimated overall B1001 reactivity. A sample was considered B1001-reactive if showed a response with a M.W p-value <0.1 in least one test. A sample was estimated as comparably reactive to B1001 and Ara h 2 if found reactive in at least 3 of the 4 tests and with majority of the tests having B1001 vs. Ara h 2 M.W p-value of >0.2.

FIGS. 20A-20B show B1001 has markedly improved safety over Ara h 2 and comparable immunotherapeutic efficacy to peanut extract in murine allergy model. C3H/HeJ mice were orally sensitized using peanut extract (PE) and cholera toxin. FIG. 20A: Mice were i.p-challenged with 30 μg natural Ara h 2 or B1001. On subsequent days, the B1001-challenged mice from the previous day were randomized into two sub-groups and re-challenged with a 2-fold higher dose og Ara h 2 or B1001, up to 240 μg. Top pane: anaphylactic scores taken 120 minutes after challenges. Chart shows individual values and mean±S.E with Mann-Whitney significance noted above. Bottom pane: body temperature was taken at noted times following challenge. Means±S.E are shown. FIG. 20B: Sensitized mice were administered oral immunotherapy with peanut flour extract (PE), B1001 or PBS (Sham). Top pane: anaphylactic scores taken 120 minutes after challenge with 35 μg natural Ara h 2. Chart shows individual values and means±S.E with Mann-Whitney p-values noted above. Bottom pane: Mesenteric lymph node cells were isolated from each mouse, seeded in 96-well and incubated for 72 hours with 200 μg/ml natural Ara h 2. Media cytokine levels were measured using the ProcartaPlex Luminex panel assay. Means±S.E are shown (n: 3 control, 4 sham, 6 PE, 5 B1001) with Mann-Whitney significance noted above.

FIG. 21 shows SEC-HPLC analysis of Ara h 1 WT and PLP595 (C159) (SEQ ID NO:156). Shown are chromatograms of Ara h 1 natural, recombinant WT Arah1, and Ara h 1 PLP595. The retention times and the estimated M.W. are presented. All proteins have a similar retention time and a similar estimated M.W. about 200 kDa, which fits a trimer fold.

FIGS. 22A-22B present secondary structure evaluation using Circular Dichroism (CD) of Ara h 1 and PLP595 (C159) variant. FIG. 22A: Normalized CD spectra of WT Ara h 1 (dashed line) and PLP595 Arah1 (solid line), both present similar CD signature at 2° C. FIG. 22B: CD signal normalized ellipticity at 205 nm at 20-90° C. of recombinant WT (circles) and PLP595 (triangles). Both Ara h 1 variants show secondary structure stability over 85° C.

FIG. 23 shows molecular weight of Ara h 1 and PLP595 (C159) variant analysis using mass photometry. An overlay histogram of normalized counts measurements recombinant Ara h 1 WT and PLP595 (C159) proteins. The result supports trimer fold formation (each monomer expected mass is 63 kDa).

FIGS. 24A-24B present allergenic potential comparison of three different Ara h 1 variants using RBL SX-38 degranulation assay. RBL SX-38 cells were sensitized with allergic patients' plasma or serum for 18 hours. Cells were then treated with wild type (WT) Ara h 1, Combo 57(PLP 243), combo 68(B1305) or combo 159(PLP595) for 1 hour at concentrations ranging from 5 ug/ml to 0.5 ng/ml. Degranulation was measured using β-Hexosaminidase activity assay. Area under the curve (AUC) values were extracted for each individual patient (each represented as a dot). FIG. 24A: Reactivity comparison of 13 Ara h 1 reactive patients to Combo 57 and combo 68. FIG. 24B: Reactivity comparison of 47 Ara h 1 reactive patients to combo 57 and combo 159. Combo 159 exhibits reduced allergenicity superiority over combo 57 and 68 with more than 70% of patients exhibiting no reactivity. Combo 159 median AUC (1.7) is reduced by 97% compared to WT Ara h 1 (56.3).

FIGS. 25A-25B present allergenic potential evaluation of different Ara h 1 variants using RBL SX-38 degranulation assay. RBL SX-38 cells were sensitized with allergic patients' plasma or serum for 18 hours. Cells were then treated with wild type (WT) Ara h 1, KLH (as negative control), Combo 51 (B1291), 52 (B1292), 74 (B1309), 75 (B1304), or 116 (PLP499) for 1 hour at concentrations ranging from 5 ug/ml to 0.05 ng/ml or 0.5 ng/ml. Degranulation was measured using β-Hexosaminidase activity assay. FIG. 25A: Example of two sera tested with Combo 51 and 52. FIG. 25B: Example of two sera tested with Combo 74.

FIGS. 26A-26B present allergenic potential evaluation of different Ara h 1 variants using RBL SX-38 degranulation assay. RBL SX-38 cells were sensitized with allergic patients' plasma or serum for 18 hours. Cells were then treated with wild type (WT) Ara h 1, KLH (as negative control), Combo 51, 52, 74, 75, or 116 for 1 hour at concentrations ranging from 5 ug/ml to 0.05 ng/ml or 0.5 ng/ml. Degranulation was measured using β-Hexosaminidase activity assay. FIG. 26A: Example of two sera tested with Combo 75. FIG. 26B: Example of two sera tested with Combo 116.

FIG. 27 shows allergenic potential of C159 (PLP595) is markedly reduced compared to natural Ara h 1. Results were from BAT assay. Fresh patient blood was induced with 11 allergen concentrations in the range pf 6,600-0.06 ng/ml (log 3 stepwise dilutions). Samples were then incubated of for 30 minutes, stained, washed, fixed and analyzed by flow cytometry. Plot shows averages and S.E for each concentration with baseline subtracted, representing 19 patients. EC50 values derived from the resulting curves by fitting to a 4-parameter logistic regression model suggest C159 has >1000-fold reduced reactivity at the population level.

FIG. 28 shows an example of back-to-consensus variants of DE Ara h 2 1001 expressed in HEK293 cells. Variants 1-23 were transfected in duplicates. The medium supernatant was analyzed by either reducing or non-reducing SDS PAGE (left or right panels respectively). Black arrows denote the Ara h 2 double band. The expression levels are compared to the poorly expressing DE Ara h 2 1001 (rightmost lane in all gels). Highly expressing variants were analyzed for allergenicity and selected taken for the next optimization round accordingly, in this case the variants denoted as numbers 2 and 4 (SEQ ID NOs: 208 and 209).

FIG. 29 demonstrates a summary of RBL activation assays (n=18) showing the area under the curve of each individual assay. Assays measured reactivity towards the various proteins at 0.05 ng/ml-5 μg/ml, except for the Fc fusion that was measured at 0.1 ng/ml-10 μg/ml to account for the dimer presenting two allergens per molecule. Results comparing natural Arah 2 (nArah2), de-epitoped Ara h 2 1001 [SEQ ID NO: 168], Arah 2_conbo31 [SEQ ID NO:247] and 1001-Fc IgG4 fusion [SEQ ID NO:207] (labeled DE Ara h 2 1001, DE Ara h 2 var. 31, DE Ara h 2-Fc IgG4). The left panel showing the same results scaled up to emphasize the subtle differences between the engineered Ara h 2 constructs. All de-epitoped versions had a slight reaction towards one serum tested (R567), though markedly reduced compared to nAra h 2.

FIG. 30 shows a comparison of HEK293 expression levels between de-epitoped Ara h 2 1001 [SEQ ID NO: 168] and de-epitoped Ara h 2—Fc fusion [SEQ ID NO:202]. The constructs were transfected to HEK293 cells in duplicate and the medium supernatant analyzed by SDS PAGE (left) and Western blot, detected by anti-DE Ara h 2 antibodies (right). Each sample was run non-reduced or reduced with β-mercaptoethanol (β-ME). Fusion to the Fc dramatically increased the secretion levels of de-epitoped Ara h 2 1001. Reduction of the sample interferes with detection by Western blot.

FIG. 31 shows the expression of transmembrane fusion of de-epitoped Ara h 2 compared to secreted de-epitoped Ara h 2 1001. HEK293 cells were transfected with either secreted de-epitoped 1001-TM Ara h 2 [SEQ ID NO: 248] or a glycosylation deficient mutant of the 1001 construct (GM1001-TM) [SEQ ID NO: 249], both fused to the TM domain of HLA-A. Cells were lysed and separated to soluble and membrane fractions by centrifugation. The separated fractions were analyzed by SDS-PAGE (left panel), either in non-reducing or reducing conditions with the addition of β-mercaptoethanol (β-ME). A Western blot of the same gel (right panel) was used to detect the presence and cellular location of de-epitoped Ara h 2. The membrane fraction signal is roughly four-fold higher than the soluble fraction. The signal corresponding to de-epitoped Ara h 2 is denoted by the black arrows. Partial glycosylation of 1001 (the band at ˜30 kDa, denoted by the top arrow) is observed in the secreted version of this protein and is expected to occur with the antigen oriented to the extracellular space. The presence of the de-epitoped antigen and its orientation being on the extracellular surface was confirmed by immunohistochemistry (not shown).

FIG. 32 shows the B cell response against various constructs as monitored following DNA delivery. Naïve mice were injected with expression plasmids encoding for various potential mRNA therapy constructs. Mice were injected 3 times weekly and bled on day 21 following the initial delivery (for each group n=5). Allergen specific antibodies were detected by ELISA. Values shown here were subtracted from a KLH negative control. No antibodies were detected at time 0 (prior to the initial DNA injection). Top panel: average IgG titers for wild type and de-epitoped Ara h 1 constructs. Bottom panel: average IgG titers for wild type and de-epitoped Ara h 2. Ara h 1 and Ara h 1 derived constructs are markedly more immunogenic than de-epitoped Ara h 2 constructs. No antibodies were detected in response to wildtype Ara h 2 and DE Ara h 2 1001 encoding constructs, where 1001 was not fused to an additional protein domain (not show).

FIG. 33 shows peanut extract separation on Q Sepharose column by salt gradient. Chromatogram shows the elution pattern of different peanut proteins represented as the absorbance units (AU) at 280 nm (left axis) against mobile phase volume (mL). The linear line represents the percentage of the salt reservoir that was used for separation. Areas on the chromatogram divided by vertical black lines represent the fractions in which the Ara h protein were eluted (depicted above each area).

FIG. 34 shows the SDS-PAGE analysis using Coomassie staining of the eluted fractions from FIG. 33. The different Ara h proteins are indicated by arrows and the molecular masses indicated at the left. The dotted line stretched between the chromatogram on FIG. 33 and the gel on FIG. 34 represent the areas where each of the four major peanut allergens were eluted (Ara h 1, Ara h 2, Ara h 3 and Ara h 6).

FIG. 35 shows a typical elution pattern of nAra h 2 on Superdex 75 SEC column.

FIG. 36 shows the SDS-PAGE pattern of nAra h 2 on Superdex 75 SEC column. Ara h 2 was eluted as duplet.

FIG. 37 shows hypersensitivity reactions as measured by body temperature drop in mice treated sublingually with 5 μg peanut protein (SLIT 5 μg) or 50 μg peanut protein (SLIT 50 μg), or treated orally with 500 μg peanut protein (OIT 500 μg). No SL/OIT treatment denotes mice not treated with peanut protein sublingually or orally.

FIGS. 38A-38B show that Ara h 1 variant C159 (SEQ ID NO: 156) retains partial immunogenicity towards peripheral blood T cells from peanut allergy patients, as detected by T cell activation assay, PBMC were isolated from peanut allergy patient blood, stained with Celltrace proliferation dye and incubated for 7 days with either PBS, recombinant WT Ara h 1 or Ara h 1 variant C159 (4-8 replicates/treatment, 2-2.5×10⁵ cells/well). Media was removed and stored for later analysis and cells were stained for Th identification and analyzed by flow cytometry to detect proliferation. Media was analyzed by sandwich ELISA to detect secretion of IL-5, IL-13 and IFNγ. Data was collected only from tests with a WT Ara h 1 response that was [S.I>2+Mann-Whitney p-value<0.1]. FIG. 38A: Charts show means±S.E, sample number at the top left and p-values for pairwise comparisons by Wilcoxon rank-sum test above bars. FIG. 38B: Estimated overall C159 reactivity. A sample was considered C159-reactive if showed a response with a M.W p-value<0.1 in least one test. A sample was estimated as comparably reactive to C159 and Ara h 1 if found reactive in at least 3 of the 4 tests and with majority of the tests having C159 vs. Ara h 1 M.W p-value of >0.2.

FIGS. 39A-39B show that reduced patient plasma binding to C159 is differential for IgE and IgG. ELISA assays were carried out on plates coated with Ara h 1 or Ara h 1 variant C159. Plasma samples from 24 peanut allergy patients were serially diluted and incubated on plates to detect patient IgE or IgG binding to each allergen. Titration curves were derived and used to calculate area under the curve (AUC) values. FIG. 39 A: Relative binding of patient IgE or IgG to Ara h 1 or C159. Figure shows AUC medians and ranges. Wilcoxon matched-pairs signed rank test p-values are noted above bars, ratio of Arah1/C159 medians ratio is noted below each chart.

FIG. 39 B: C159/Ara h 1 AUC ratios were calculated to express reduced binding of variant. Figure shows individual AUC ratios with IgE and IgG ratios pairing by patient marked with thin lines and group medians marked with thick lines. Wilcoxon matched-pairs signed rank test p-values are noted.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the recombinant Ara h 1 and Ara h 2 allergen variants disclosed, and uses thereof. However, it will be understood by those skilled in the art that the recombinant Ara h 1 and Ara h 2 allergen variants described and uses thereof, may be practiced without these specific details. In other instances, well-known methods, procedures, and components have not been described in detail so as not to obscure the present recombinant Ara h 1 and Ara h 2 variants described and uses thereof.

In some embodiments, recombinant Ara h 1 and Ara h 2 variants were mutated based on data collected during the epitope mapping process. Mutation sites were selected based on the likelihood of a mutation, alone or in combination with additional mutations, altering or destroying one or more epitopes recognized by anti-Ara h 1 or anti-Ara h 2 antibodies. The allergenicity of Ara h 1 and Ara h 2 variants was assessed by rat basophil leukemia (RBL) or Basophil Activation Tests (BAT) cell-based immunological assay with peanut-allergic patient samples. The desired immunogenicity, i.e., the ability of the engineered Ara h 1 and or Ara h 2 to trigger a response of the immune system without triggering mast cells/basophils mediated allergic reaction, was measured by T cell activation assays.

A skilled artisan would appreciate that the term “epitope” may be used interchangeably with the term “antigenic determinant” having all the same meanings and qualities, and may encompass a site on an antigen to which an immunoglobulin or antibody (or antigen binding fragment thereof) specifically binds. Epitopes can be formed both from contiguous amino acids and noncontiguous amino acids juxtaposed by tertiary folding of a protein. Epitopes formed from contiguous amino acids (linear epitopes) are typically retained on exposure to denaturing solvents, whereas epitopes formed by tertiary folding (conformational epitopes) are typically lost upon treatment with denaturing solvents. An epitope typically includes at least 3, 4, 5, 6, 7, S, 9, 10, 11, 12, 13, 14 or 15 amino acids in a unique spatial conformation. In some embodiments, the epitope is as small as possible while still maintaining immunogenicity. Immunogenicity is indicated by the ability to elicit an immune response, as described herein, for example, by the ability to bind an MHC class II molecule and to induce a T cell response, e.g., by measuring T cell cytokine production.

As used herein, “de-epitoped polypeptide X” refers to a modified polypeptide X that has reduced or abolished binding with anti-polypeptide X antibodies (as compared to antibody binding to its wild-type counterpart) due to mutation(s) at one or more epitopes recognized by the anti-polypeptide X antibodies.

As used herein, “de-epitoped Ara h 1 allergen” refers to a modified Ara h 1 allergen that has reduced or abolished binding with anti-Ara h 1 antibodies (as compared to antibody binding to the wild-type Ara h 1) due to mutation(s) at one or more epitopes recognized by the anti-Ara h 1 antibodies. In one embodiment, the de-epitoped Ara h 1 allergen has reduced allergenicity as compared to its wild-type counterpart.

As used herein, “de-epitoped Ara h 2 allergen” refers to a modified Ara h 2 allergen that has reduced or abolished binding with anti-Ara h 2 antibodies (as compared to antibody binding to the wild-type Ara h 2) due to mutation(s) at one or more epitopes recognized by the anti-Ara h 2 antibodies. In one embodiment, the de-epitoped Ara h 2 allergen has reduced allergenicity as compared to its wild-type counterpart.

As used herein, an “epitope” refers to the part of a macromolecule (e.g., Ara h 1, or Ara h 2 allergen) that is bound by an antibody or an antigen-binding fragment thereof. Within a protein sequence, there are continuous epitopes, which are linear sequences of amino acids bound by the antibody, or discontinuous epitopes, which exist only when the protein is folded into a particular conformation.

As used herein, an “allergen” refers to a substance, protein, or non-protein, capable of inducing allergy or specific hypersensitivity.

As used herein, “allergenicity” or “allergenic” refers to the ability of an antigen or allergen to induce an abnormal immune response, which is an overreaction and different from a normal immune response in that it does not result in a protective/prophylaxis effect but instead causes physiological function disorder or tissue damage.

As used herein, “hypoallergenic” refers to a substance having little or reduced likelihood of causing an allergic response.

In some embodiments, the present disclosure provides peanut allergen (e.g., Ara h 1, Ara h 2) variants that were mutated to diminish or abolish one or more epitopes bound by anti-peanut allergen antibodies. In one embodiment, the mutation does not affect the biophysical and/or functional characteristics of the peanut allergen. The mutation in one aspect may be substitution, deletion, or insertion, or any combination thereof. A deletion, for example, may comprise the removal of a single amino acid that is crucial for antibody binding, or of a whole mapped epitope region.

Six general classes of amino acid side chains have been categorized and include: Class I (Cys); Class II (Ser, Thr, Ala, Gly); Class III (Asn, Asp, Gln, Glu); Class IV (His, Arg, Lys); Class V (Ile, Leu, Val, Met); and Class VI (Phe, Tyr, Trp). In addition, a Pro may be substituted in the variant structures. Conservative amino acid substitution refers to substitution of an amino acid in one class by an amino acid of the same class. For example, substitution of an Asp for another class III residue such as Asn, Gln, or Glu, is a conservative substitution. Non-conservative amino acid substitution refers to substitution of an amino acid in one class with an amino acid from another class; for example, substitution of an Ala, a class II residue, with a class III residue such as Asp, Asn, Glu, or Gln. Methods of substitution mutations at the nucleotide or amino acid sequence level are well-known in the art.

The term “modifying,” or “modification,” as used herein, refers to changing one or more amino acids in an antibody or antigen-binding portion thereof. The change can be produced by adding, substituting, or deleting an amino acid at one or more positions. The change can be produced using known techniques, such as PCR mutagenesis. For example, in some embodiments, an antibody or an antigen-binding portion thereof identified using the methods provided herein can be modified, to thereby modify the binding affinity of the antibody or antigen-binding portion thereof to the peanut allergen.

### Ara h 1 Variants

In one embodiment, the present disclosure provides a recombinant Ara h 1 variant polypeptide comprising an amino acid sequence that is at least 50% identical to the sequence set forth in SEQ ID NO: 65, wherein the Ara h 1 variant comprises one or more substitutions, deletions, insertions, or any combination thereof, that are located within a single epitope recognized by an anti-Ara h 1 antibody. In another embodiment, the Ara h 1 variant comprises one or more amino acid substitutions, deletions, insertions, or any combination thereof, that are located within at least two epitopes recognized by anti-Ara h 1 antibodies.

In one embodiment, the recombinant Ara h 1 variant polypeptide comprising the amino acid sequence set forth in SEQ ID NO: 67, wherein the variant comprises substitutions, deletions, insertions, or any combination thereof, at one or more of positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65. In one embodiment, the substitution mutation is D at position 194. In one embodiment, the substitution mutation is A at position 195. In one embodiment, the substitution mutation is H at position 213. In one embodiment, the substitution mutation is R, D, L, I, F, or A at position 215. In one embodiment, the substitution mutation is A at position 231. In one embodiment, the substitution mutation is E at position 234. In one embodiment, the substitution mutation is R at position 245. In one embodiment, the substitution mutation is E at position 267. In one embodiment, the substitution mutation is D at position 287. In one embodiment, the substitution mutation is E at position 294. In one embodiment, the substitution mutation is A or H at position 312. In one embodiment, the substitution mutation is H at position 331. In one embodiment, the substitution mutation is E, V, or A at position 419. In one embodiment, the substitution mutation is R or A at position 422. In one embodiment, the substitution mutation is A at position 443. In one embodiment, the substitution mutation is A at position 455. In one embodiment, the substitution mutation is A or K, or T at position 462. In one embodiment, the substitution mutation is S at position 463. In one embodiment, the substitution mutation is A or S at position 464. In one embodiment, the substitution mutation is Q at position 480. In one embodiment, the substitution mutation is A or E, or N at position 494. In one embodiment, the substitution mutation is K at position 500.

A skilled artisan would appreciate that percent identity (% identity) provides a number that describes how similar the query sequence is to the target sequence (i.e., how many amino acids in each sequence are identical). The higher the percent identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

In some embodiments, the variant Ara h 1 polypeptides comprises an amino acid sequence that is at least 50%, at least 55%, at least 60%, at least 65%, at least 70%, at least 75%, at least 80%, at least 85%, or at least 90% identical to a polypeptide or a portion thereof disclosed herein, as determined using BlastP software of the National Center of Biotechnology Information (NCBI) using default parameters.

In some embodiments, the Ara h 1 variants may encompass deletion, insertion, or amino acid substitution mutations. In one embodiment, the variant polypeptide comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In some embodiments of the above recombinant Ara h 1 variants, the Ara h 1 variants comprise at least 2, 3, 4, 5, 6, 7, 8, 9, 10, 11, 12, 13, 14, 15, 16, 17, 18, 19, 20, 21 or 22 substitution mutations at positions selected from positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65.

In some embodiments of the above recombinant Ara h 1 variants, the Ara h 1 variants further comprise additional substitutions, deletions, insertions, or any combination thereof at one or more of positions 12, 24, 27, 30, 42, 57, 58, 73, or 523 of SEQ ID NO: 67, as compared with the amino acid residues at those same positions in SEQ ID NO: 65. In one embodiment, the substitution mutation is K or A at position 12. In one embodiment, the substitution mutation is V or E at position 24. In one embodiment, the substitution mutation is A or H at position 27. In one embodiment, the substitution mutation is E or A at position 30. In one embodiment, the substitution mutation is L or K at position 42. In one embodiment, the substitution mutation is D or L at position 57. In one embodiment, the substitution mutation is S or R at position 58. In one embodiment, the substitution mutation is A or M at position 73. In one embodiment, the substitution mutation is A or K at position 523. In some embodiments of the above recombinant Ara h 1 variants, the Ara h 1 variants further comprise additional substitutions, deletions, insertions, or any combination thereof at one or more of positions 87, 88, 96, 99, 196, 197, 200, 209, 238, 249, 260, 261, 263, 265, 266, 278, 283, 288, 290, 295, 318, 322, 334, 336, 378, 417, 421, 441, 445, 481, 484, 485, 487, 488, or 491 of SEQ ID NO: 67, as compared with the amino acid residues at those same positions in SEQ ID NO: 65. In one embodiment, the substitution mutation is A at position 87. In one embodiment, the substitution mutation is A at position 88. In one embodiment, the substitution mutation is A at position 96. In one embodiment, the substitution mutation is A at position 99. In one embodiment, the substitution mutation is H at position 196. In one embodiment, the substitution mutation is A at position 197. In one embodiment, the substitution mutation is V, A or Q at position 200 In one embodiment, the substitution mutation is S at position 209. In one embodiment, the substitution mutation is Q at position 238. In one embodiment, the substitution mutation is N at position 249. In one embodiment, the substitution mutation is K at position 260. In one embodiment, the substitution mutation is R at position 261. In one embodiment, the substitution mutation is K or L at position 263. In one embodiment, the substitution mutation is K at position 263. In one embodiment, the substitution mutation is S at position 265. In one embodiment, the substitution mutation is R or L at position 266. In one embodiment, the substitution mutation is R at position 278. In one embodiment, the substitution mutation is E at position 283. In one embodiment, the substitution mutation is Q at position 288. In one embodiment, the substitution mutation is R at position 290. In one embodiment, the substitution mutation is A at position 295. In one embodiment, the substitution mutation is H at position 318. In one embodiment, the substitution mutation is A or K at position 322. In one embodiment, the substitution mutation is D, A or N at position 334. In one embodiment, the substitution mutation is R or S at position 336. In one embodiment, the substitution mutation is K or E at position 378. In one embodiment, the substitution mutation is R at position 417. In one embodiment, the substitution mutation is E or S at position 421. In one embodiment, the substitution mutation is N at position 441. In one embodiment, the substitution mutation is A at position 443. In one embodiment, the substitution mutation is A or S at position 481. In one embodiment, the substitution mutation is R, S, A, or M at position 484. In one embodiment, the substitution mutation is A at position 485. In one embodiment, the substitution mutation is S or K at position 487. In one embodiment, the substitution mutation is A at position 488. In one embodiment, the substitution mutation is A, S or E at position 491.

In some embodiments of the above recombinant Ara h 1 variants, the Ara h 1 variants further comprise substitution mutation at position 84 of SEQ ID NO: 67, as compared with the amino acid residues at those same positions in SEQ ID NO: 65. In one embodiment, the substitution mutation is A at position 84.

In some embodiments of the above recombinant Ara h 1 variants, the Ara h 1 variants comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 substitution mutations at positions selected from positions 12, 24, 27, 30, 42, 52, 57, 58, 73, 84, 87, 88, 96, 99, 194, 195, 196, 197, 200, 209, 213, 215, 231, 234, 238, 245, 249, 260, 261, 263, 265, 266, 267, 278, 283, 287, 288, 290, 294, 295, 312, 318, 322, 331, 334, 336, 378, 417, 419, 421, 422, 441, 443, 445, 455, 462, 463, 464, 480, 481, 484, 485, 487, 488, 491, 494, 500, or 523 of SEQ ID NO: 67, as compared with the amino acid residues at those same positions in SEQ ID NO: 65.

In some embodiments of the above recombinant Ara h 1 variants, the Ara h 1 variant comprises an amino acid sequence that is at least 70%, 75%, 80%, 85%, or 90% identical to the sequence set forth in SEQ ID NO: 65.

In some embodiments of the above recombinant Ara h 1 variants, the Ara h 1 variant comprises one or more substitutions, deletions, insertions, or any combination thereof at one or more positions of 12, 24, 27, 30, 42, 52, 57, 58, 73, 84, 87, 88, 96, 99, 194-197, 200, 209, 213, 215, 231, 234, 238, 245, 249, 260, 261, 263, 265, 266, 267, 278, 283, 287, 288, 290, 294, 295, 312, 318, 322, 331, 334, 336, 378, 417, 419, 421, 422, 441, 443, 445, 455, 462, 463, 464, 480, 481, 484, 485, 487, 488, 491, 494, 500, and 523 of SEQ ID NO: 67, as compared with the amino acid residues at those same positions in SEQ ID NO: 65.

In some embodiments of the above recombinant Ara h 1 variants, the Ara h 1 variants comprise the amino acid sequence set forth in any of SEQ ID NOs: 68-161, 174, 176, 178, 180, 182, 184, 193, 194, 211-246, or comprises an amino acid sequence having at least 80% identity with the amino acid sequences set forth in any of SEQ ID NOs: 68-161, 174, 176, 178, 180, 182, 184, 193, 194, 211-246.

In some embodiments of the above recombinant Ara h 1 variants, basophile degranulation release induced by the variants is at least 3-fold lower compared with that induced by an Ara h 1 wild-type polypeptide.

In some embodiments of the above recombinant Ara h 1 variants, the variant has a binding EC50 or KD that is reduced 50% or more as compared with that of an Ara h 1 wild-type polypeptide.

### Ara h 2 Variants

In one embodiment, provided herein is a recombinant Ara h 2 variant polypeptide comprising an amino acid sequence that is at least 50% identical to the sequence set forth in SEQ ID NO: 3, wherein the variant comprises one or more amino acid substitutions, deletions, insertions, or any combination thereof, that are located within a single epitope recognized by an anti-Ara h 2 antibody. In another embodiment, the Ara h 2 variant comprises one or more amino acid substitutions, deletions, insertions, or any combination thereof, that are located within at least two epitopes recognized by anti-Ara h 2 antibodies.

In one embodiment, the recombinant Ara h 2 variant polypeptide comprising the amino acid sequence set forth in SEQ ID NO: 4, wherein the variant comprises substitution mutation(s) at one or more of positions 12, 15, 16, 22, 24, 46, 53, 65, 80, 83, 86, 87, 90, 104, 115, 123, 127, or 140 of SEQ ID NO: 4, as compared with the amino acid residues at those same positions in SEQ ID NO: 3. In one embodiment, the substitution mutation is N, Q, E, D, T, S, G, P, C, K, H, Y, W, M, I, L, V, or A at position 12. In one embodiment, the substitution mutation is R, E, K, Y, W, F, M, I, V, C, D, G, or A at position 15. In one embodiment, the substitution mutation is R, K, D, Q, T, M, P, C, E, or W at position 16. In one embodiment, the substitution mutation is F, Y, W, Q, E, T, S, A, M, I, L, C, R, or H at position 22. In one embodiment, the substitution mutation is D, E, H, K, S, T, N, Q, L, I, M, W, Y, F, P, A, or G at position 24. In one embodiment, the substitution mutation is T, V, E, H, S, A, G, Q, N, D, R, P, M, I, L, or C at position 46. In one embodiment, the substitution mutation is T, S, Q, V, A, G, C, P, M, L, I, E, H, R, K, N, or D at position 53. In one embodiment, the substitution mutation is T, A, N, D, Q, R, K, H, I, L, M, V, W, P, G, C, or E at position 65. In one embodiment, the substitution mutation is N, S, T, V, A, I, L, M, F, Y, W, C, E, K, R, or G at position 80. In one embodiment, the substitution mutation is D, A, C, F, I, P, T, V, W, Y, or Q at position 83. In one embodiment, the substitution mutation is Y, F, H, R, E, C, G, I, L, M, V, T, S, or Q at position 86. In one embodiment, the substitution mutation is F, Y, I, L, M, V, A, S, Q, R, K, D, N, E, or P at position 87. In one embodiment, the substitution mutation is S, P, Q or R at position 90. In one embodiment, the substitution mutation is L, M, K, R, H, E, D, A, Y, N, S, or W at position 104. In one embodiment, the substitution mutation is V, D, E, I, L, K, M, N, S, T, A, I, W, F, Y, or H at position 115. In one embodiment, the substitution mutation is I, Q, or A at position 123. In one embodiment, the substitution mutation is H, A, D, E, F, G, L, N, P, S, T, W, Y, Q, or V at position 127. In one embodiment, the substitution mutation is G, A, C, E, Y, F, H, K, L, M, N, P, Q, S, or V at position 140.

In some embodiments, the variant Ara h 2 polypeptides comprises an amino acid sequence that is at least 50%, at least 55%, at least 60%, at least 65%, at least 70%, at least 75%, at least 80%, at least 85%, or at least 90% identical to a polypeptide or a portion thereof disclosed herein, as determined using BlastP software of the National Center of Biotechnology Information (NCBI) using default parameters.

In some embodiments, the Ara h 2 variants may encompass deletion, insertion, or amino acid substitution mutations. In one embodiment, the Ara h 2 variant polypeptide comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In some embodiments of the above Ara h 2 variants, the variants comprise at least 2, 3, 4, 5, 6, 7, 8, 9, 10, 11, 12, 13, 14, 15, 16, 17 or 18 substitution mutations at positions selected from positions 12, 15, 16, 22, 24, 46, 53, 65, 80, 83, 86, 87, 90, 104, 115, 123, 127, and 140 of SEQ ID NO: 4 as compared with the amino acid residues at those same positions in SEQ ID NO: 3.

In some embodiments of the above Ara h 2 variants, the amino acids at positions 12-16 of SEQ ID NO: 4 comprise the sequence set forth in SEQ ID NO: 5.

In some embodiments of the above Ara h 2 variants, the amino acids at positions 44-65 of SEQ ID NO: 4 comprise the sequence set forth in SEQ ID NO: 6.

In some embodiments of the above Ara h 2 variants, the amino acids at positions 44-67 of SEQ ID NO: 4 comprise the sequence set forth in SEQ ID NO: 9.

In some embodiments of the above Ara h 2 variants, the amino acids at positions 11-90 of SEQ ID NO: 4 comprise the sequence set forth in SEQ ID NO: 7 or SEQ ID NO: 8.

In some embodiments of the above Ara h 2 variants, the variants further comprise additional substitutions, deletions, insertions, or any combination thereof, at one or more of positions 28, 44, 48, 51, 55, 63, 67, 107, 108, 109, 124, 125, or 142 of SEQ ID NO: 4, as compared with the amino acid residues at those same positions in SEQ ID NO: 3. In one embodiment, the substitution mutation is S, T, V, N, A, P, I, L, F, Y, H, R, K, E, or D at position 28. In one embodiment, the substitution mutation is I, A, C, G, H, L, F, Y, N, P, Q, K, E, S, T, V, M, or R at position 44. In one embodiment, the substitution mutation is V, G, C, E, H, Q, F, K, L, I, W, Y, N, R, S, T, V, A, or D at position 48. In one embodiment, the substitution mutation is S, G, Y, F, W, M, N, Q, E, R, K, H, T, D, or V at position 51. In one embodiment, the substitution mutation is G, A, D, E, F, Y, H, Q, V, I, L, M, R, K, S, T, C, or W at position 55. In one embodiment, the substitution mutation is P, C, F, V, I, L, M, W, Y, N, S, T, Q, G, H, K, or R at position 63. In one embodiment, the substitution mutation is E, Q, N, R, H, Y, F, W, M, L, V, T, S, A, P, or G at position 67. In one embodiment, the substitution mutation is A, C, F, G, H, I, K, L, M, Q, P, R, S, T, V, W, or Y at position 107. In one embodiment, the substitution mutation is T, V, D, E, R, H, Y, W, I, G, A, Q, or K at position 108. In one embodiment, the substitution mutation is K, C, S, R, G, P, Y, W, L, or I at position 109. In one embodiment, the substitution mutation is D, A, C, F, G, H, I, N, S, T, V, Y, L, E, or Q at position 124. In one embodiment, the substitution mutation is M, I, L, W, Y, G, K, N, T, V, or A at position 125. In one embodiment, the substitution mutation is M, A, C, E, F, G, H, I, K, L, N, P, Q, R, S, T, V, W, or Y at position 142.

In some embodiments of the above Ara h 2 variants, the variants comprise 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 substitution mutations at positions selected from positions 12, 15, 16, 22, 24, 28, 44, 46, 48, 51, 53, 55, 63, 65, 67, 80, 83, 86, 87, 90, 104, 107, 108, 109, 115, 123, 124, 125, 127, 140, or 142 of SEQ ID NO: 4, as compared with the amino acid residues at those same positions in SEQ ID NO: 3.

In one embodiment of the above Ara h 2 variants, the variant comprises substitution mutations at positions 44, 48, 51, 55, 63, and 67 of SEQ ID NO: 4, as compared with the amino acid residues at those same positions in SEQ ID NO: 3.

In some embodiments of the above Ara h 2 variants, the variant comprises an amino acid sequence that is at least 70%, 75%, 80%, 85%, or 90% identical to the sequence set forth in SEQ ID NO: 3.

In some embodiments of the above Ara h 2 variants, the variant comprises one of more substitutions, deletions, insertions, or any combination thereof at one of more positions of 6, 11-28, 32, 39, 44-56, 58, 60, 63, 69, 80-87, 89-90, 92, 96-97, 99, 100, 102-105, 107-119, 123, 125, 127-131, 133, 134, 136-144, 146, or 148-153 of SEQ ID NO: 3.

In some embodiments of the above recombinant Ara h 2 variants, the variant comprises the amino acid sequence as set forth in any one of SEQ ID NOs: 10-63, 168, 170, 195-201, 204-210, 247-249, or comprises an amino acid sequence having at least 80% identity with the amino acid sequences set forth in any of SEQ ID NOs:10-63, 168, 170, 195-201, 204-210, 247-249.

In some embodiments of the above recombinant Ara h 2 variants, basophile degranulation release induced by the variants is at least 10-fold lower compared with that induced by an Ara h 2 wild-type polypeptide.

In some embodiments of the above recombinant Ara h 2 variants, the variant has a binding EC50 or KD that is reduced 50% or more as compared with that of an Ara h 2 wild-type polypeptide.

### Nucleotides, Vectors, and Host Cells

The term “nucleotide”, “nucleotide sequence” or “nucleic acid molecule” as used herein is intended to include DNA molecules and RNA molecules or modified RNA molecules. A nucleic acid molecule may be single-stranded or double-stranded. In some embodiments, a nucleotide comprises a modified nucleotide. In some embodiments, a nucleotide comprises an mRNA. In some embodiments, a nucleotide comprises a modified mRNA. In some embodiments, a nucleotide comprises a modified mRNA, wherein the modified mRNA comprises a 5′-capped mRNA. In some embodiments, a modified mRNA comprises a molecule in which some of the nucleosides have been replaced by either naturally modified or synthetic nucleosides. In some embodiments, a modified nucleotide comprises a modified mRNA comprising a 5′-capped mRNA and wherein some of the nucleosides have been replaced by either naturally modified or synthetic nucleosides.

The term “isolated nucleotide” or “isolated nucleic acid molecule” as used herein refers to nucleic acids encoding the peanut allergen variants disclosed herein (e.g., Ara h 1 variants, Ara h 2 variants) in which the nucleotide sequences are essentially free of other genomic nucleotide sequences that naturally flank the nucleic acid in genomic DNA.

Disclosed herein, in one aspect is a nucleotide or nucleic acid sequence encoding the peanut allergen variants disclosed herein (e.g., Ara h 1 variants, Ara h 2 variants).

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of affecting expression of such nucleic acids. Thus, an expression vector may refer to a DNA or RNA construct, such as a plasmid, a phage, recombinant virus, or other vector that, upon introduction into an appropriate host cell, results in expression of the nucleic acids. Appropriate expression vectors are well known to those of skill in the art and include those that are replicable in prokaryotic cells and/or eukaryotic cells, and those that remain episomal or those which integrate into the host cell genome.

Disclosed herein, in one aspect is an expression vector comprising the nucleic acid construct encoding the peanut allergen variants disclosed herein (e.g., Ara h 1 variants, Ara h 2 variants).

The term “recombinant host cell” (or simply “host cell”) as used herein refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Disclosed herein, in one aspect is a host cell comprising an expression vector carrying the nucleic acid construct encoding the peanut allergen variants disclosed herein (e.g., Ara h 1 variants, Ara h 2 variants). In one embodiment, the cell or host cell is a prokaryotic cell or a eukaryotic cell. In one embodiment, the eukaryotic cell is a yeast cell, a fungi cell, an algae cell, a plant cell, or a mammalian cell. In some embodiments, the peanut allergen variants may be produced in bacteria, such as E. Coli. In some other embodiments, the peanut allergen variants may be produced in yeast or fungi, such as Saccharomyces cerevisiae Aspergillus, Trichoderma or Pichia pastoris.

### Nucleic Acid Encoding Ara h 1 Variants

In one embodiment, provided herein are nucleic acid or modified nucleic acid molecules encoding a recombinant Ara h 1 variant polypeptide comprising an amino acid sequence that is at least 50% identical to the sequence set forth in SEQ ID NO:65, wherein the Ara h 1 variant comprises one or more substitutions, deletions, insertions, or any combination thereof that are located within a single epitope recognized by an anti-Ara h 1 antibodies.

In another embodiment, the nucleic acid or modified nucleic acid molecules encode a recombinant Ara h 1 variant comprising an amino acid sequence that is at least 50% identical to the sequence set forth in SEQ ID NO: 65, wherein the Ara h 1 variant comprises one or more amino acid substitutions, deletions, insertions, or any combination thereof that are located within at least two epitopes recognized by anti-Ara h 1 antibodies.

A skilled artisan would appreciate that percent identity (% identity) provides a number that describes how similar the query sequence is to the target sequence (i.e., how many amino acids in each sequence are identical). The higher the percent identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

In some embodiments, the variant Ara h 1 polypeptides comprises an amino acid sequence that is at least 50%, at least 55%, at least 60%, at least 65%, at least 70%, at least 75%, at least 80%, at least 85%, or at least 90% identical to the amino acid sequence SEQ ID NO:65 or a portion thereof disclosed herein, as determined using BlastP software of the National Center of Biotechnology Information (NCBI) using default parameters.

In some embodiments, the Ara h 1 variants described herein may encompass deletion, insertion, or amino acid substitution mutations. In one embodiment, the variant polypeptide comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In one embodiment, the nucleic acid or modified nucleic acid is DNA or mRNA. In one embodiment, the mRNA comprises a UTR, or the mRNA comprises a leader sequence, or the mRNA comprises a UTR and a leader sequence. In one embodiment, the UTR comprises a chimeric or novel sequence that may outperform a natural UTR sequence, promoting overall higher protein expression.

In one embodiment, the mRNA comprises (i) a UTR having the sequence of SEQ ID NO:162 or 163, and (ii) a leader sequence having the sequence of SEQ ID NO:185, 187, 189, or 191.

In one embodiment, the mRNA comprises an optimized sequence. As used herein, an “optimized sequence” encompasses an mRNA sequence comprising a computationally altered nucleotide sequence that facilitates higher expression levels in human cells, compared with the non-altered sequence, while maintaining characteristics that are favorable for in vitro transcription (IVT) and enzymatic capping.

In one embodiment, the nucleic acid or modified nucleic acid molecules disclosed herein encode an Ara h 1 variant comprising the amino acid sequence set forth in any one of SEQ ID NOs:68-161, 174, 176, 178, 180, 182, 184, 193, 194, 211-246, or comprises an amino acid sequence having at least 80% identity with the amino acid sequences set forth in any of SEQ ID NOs: 68-161, 174, 176, 178, 180, 182, 184, 193, 194, 211-246.

In one embodiment, the nucleic acid or modified nucleic acid comprises the nucleotide sequence of SEQ ID NO:173. In one embodiment, the nucleic acid or modified nucleic acid comprises the nucleotide sequence of SEQ ID NO:175. In one embodiment, the nucleic acid or modified nucleic acid comprises the nucleotide sequence of SEQ ID NO:177. In one embodiment, the nucleic acid or modified nucleic acid comprises the nucleotide sequence of SEQ ID NO:179. In one embodiment, the nucleic acid or modified nucleic acid comprises the nucleotide sequence of SEQ ID NO:181. In one embodiment, the nucleic acid or modified nucleic acid comprises the nucleotide sequence of SEQ ID NO:183.

In one embodiment, the nucleic acid or modified nucleic acid molecules disclosed herein encode a recombinant Ara h 1 variant polypeptide comprising the amino acid sequence set forth in SEQ ID NO: 67, wherein the variant comprises substitutions, deletions, insertions, or any combination thereof, at one or more of positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65. In one embodiment, the substitution mutation is D at position 194. In one embodiment, the substitution mutation is A at position 195. In one embodiment, the substitution mutation is H at position 213. In one embodiment, the substitution mutation is R, D, L, I, F, or A at position 215. In one embodiment, the substitution mutation is A at position 231. In one embodiment, the substitution mutation is E at position 234. In one embodiment, the substitution mutation is R at position 245. In one embodiment, the substitution mutation is E at position 267. In one embodiment, the substitution mutation is D at position 287. In one embodiment, the substitution mutation is E at position 294. In one embodiment, the substitution mutation is A or H at position 312. In one embodiment, the substitution mutation is H at position 331. In one embodiment, the substitution mutation is E, V, or A at position 419. In one embodiment, the substitution mutation is R or A at position 422. In one embodiment, the substitution mutation is A at position 443. In one embodiment, the substitution mutation is A at position 455. In one embodiment, the substitution mutation is A or K, or T at position 462. In one embodiment, the substitution mutation is S at position 463. In one embodiment, the substitution mutation is A or S at position 464. In one embodiment, the substitution mutation is Q at position 480. In one embodiment, the substitution mutation is A or E, or N at position 494. In one embodiment, the substitution mutation is K at position 500.

In one embodiment, the nucleic acid or modified nucleic acid molecules disclosed herein encode a recombinant Ara h 1 variant having the amino acid sequence of SEQ ID NO:67, wherein the Ara h 1 variant comprises at least 2, 3, 4, 5, 6, 7, 8, 9, 10, 11, 12, 13, 14, 15, 16, 17, 18, 19, 20, 21 or 22 substitution mutations at positions selected from positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65.

In one embodiment, the nucleic acid or modified nucleic acid molecules disclosed herein encode a recombinant Ara h 1 variant having the amino acid sequence of SEQ ID NO:67, wherein the Ara h 1 variant further comprises, in addition to the substitution mutations described above, substitution mutation(s) at one or more of positions 24, 27 or 30 of SEQ ID NO: 67, as compared with the amino acid residues at those same positions in SEQ ID NO: 65. In one embodiment, the substitution mutation is V at position 24. In one embodiment, the substitution mutation is A at position 27. In one embodiment, the substitution mutation is E at position 30.

In one embodiment, the nucleic acid or modified nucleic acid molecules disclosed herein encode a recombinant Ara h 1 variant having the amino acid sequence of SEQ ID NO:67, wherein the Ara h 1 variant further comprises, in addition to the substitution mutations described above, substitution mutation(s) at one or more of positions 87, 88, 96, 99, 196, 197, 209, 288, 290, 295, 322, 334, 336, 481, 484, 485, 487, 488, or 491 of SEQ ID NO: 67, as compared with the amino acid residues at those same positions in SEQ ID NO: 65. In one embodiment, the substitution mutation is A at position 87. In one embodiment, the substitution mutation is A at position 88. In one embodiment, the substitution mutation is A at position 96. In one embodiment, the substitution mutation is A at position 99. In one embodiment, the substitution mutation is H at position 196. In one embodiment, the substitution mutation is A at position 197. In one embodiment, the substitution mutation is S at position 209. In one embodiment, the substitution mutation is Q at position 288. In one embodiment, the substitution mutation is R at position 290. In one embodiment, the substitution mutation is A at position 295. In one embodiment, the substitution mutation is A or K at position 322. In one embodiment, the substitution mutation is D or N at position 334. In one embodiment, the substitution mutation is R at position 336. In one embodiment, the substitution mutation is A or S at position 481. In one embodiment, the substitution mutation is R, S, A, or M at position 484. In one embodiment, the substitution mutation is A at position 485. In one embodiment, the substitution mutation is S or K at position 487. In one embodiment, the substitution mutation is A at position 488. In one embodiment, the substitution mutation is A or E at position 491.

In one embodiment, the nucleic acid or modified nucleic acid molecules disclosed herein encode a recombinant Ara h 1 variant having the amino acid sequence of SEQ ID NO:67, wherein the Ara h 1 variant further comprises, in addition to the substitution mutations described above, substitution mutation at position 84 of SEQ ID NO: 67, as compared with the amino acid residues at those same positions in SEQ ID NO: 65. In one embodiment, the substitution mutation is A at position 84.

In one embodiment, the nucleic acid or modified nucleic acid molecules disclosed herein encode a recombinant Ara h 1 variant having the amino acid sequence of SEQ ID NO:67, wherein there are 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 substitution mutations at positions selected from positions 24, 27, 30, 84, 87, 88, 96, 99, 194, 195, 196, 197, 209, 213, 215, 287, 288, 290, 294, 295, 322, 331, 334, 336, 419, 422, 455, 462, 464, 480, 481, 484, 485, 487, 488, 491, or 494 of SEQ ID NO: 67, as compared with the amino acid residues at those same positions in SEQ ID NO: 65.

In one embodiment, the nucleic acid or modified nucleic acid molecules disclosed herein encode a Ara h 1 variant comprising an amino acid sequence that is at least 70%, 75%, or 80% identical to the sequence set forth in SEQ ID NO: 65.

In one embodiment, the nucleic acid or modified nucleic acid molecules disclosed herein encode a Ara h 1 variant having the amino acid sequence of SEQ ID NO: 67, wherein the Ara h 1 variant comprises one or more substitution mutations at one or more positions of 24, 27, 30, 84, 87, 88, 96, 99, 194-197, 200, 209, 213, 215, 263, 267, 271, 287, 288, 290, 294, 295, 322, 331, 334, 336, 378, 417, 419, 421, 422, 439, 455, 462-464, 468, 480, 481, 484, 485, 487, 488, 491, 494, 500, and 502 of SEQ ID NO: 67, as compared with the amino acid residues at those same positions in SEQ ID NO: 65.

### Nucleic Acid Encoding Ara h 2 Variants

In one embodiment, provided herein are nucleic acid or modified nucleic acid molecules encoding a recombinant Ara h 2 variant polypeptide comprising an amino acid sequence that is at least 50% identical to the sequence set forth in SEQ ID NO:3, wherein the Ara h 2 variant comprises one or more substitutions, deletions, insertions, or any combination thereof, that are located within a single epitope recognized by an anti-Ara h 2 antibody.

In another embodiment, the nucleic acid or modified nucleic acid molecules encode a recombinant Ara h 2 variant comprising an amino acid sequence that is at least 50% identical to the sequence set forth in SEQ ID NO:3, wherein the Ara h 2 variant comprises one or more amino acid substitutions, deletions, insertions, or any combination thereof, that are located within at least two epitopes recognized by anti-Ara h 2 antibodies.

A skilled artisan would readily appreciate percent identity (% identity) as described above. In some embodiments, the variant Ara h 2 polypeptides comprises an amino acid sequence that is at least 50%, at least 55%, at least 60%, at least 65%, at least 70%, at least 75%, or at least 80%, identical to the amino acid sequence SEQ ID NO:3 or a portion thereof disclosed herein, as determined using BlastP software of the National Center of Biotechnology Information (NCBI) using default parameters.

In some embodiments, the Ara h 2 variants described herein may encompass deletion, insertion, or amino acid substitution mutations. In one embodiment, the variant polypeptide comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In one embodiment, the nucleic acid or modified nucleic acid is DNA or mRNA. In one embodiment, the mRNA comprises a UTR, or the mRNA comprises a leader sequence, or the mRNA comprises a UTR and a leader sequence. In one embodiment, the UTR comprises a chimeric or novel sequence that may outperform a natural UTR sequence, promoting overall higher protein expression.

In one embodiment, the mRNA comprises (i) a UTR having the sequence of SEQ ID NO:162 or 163, and (ii) a leader sequence having the sequence of SEQ ID NO:185, 187, 189, or 191.

In one embodiment, the mRNA comprises an optimized sequence. As used herein, an “optimized sequence” encompasses an mRNA sequence comprising a computationally altered nucleotide sequence that facilitates higher expression level in human cells, compared with the non-altered sequence, while maintaining characteristics that are favorable for in vitro transcription (IVT) and enzymatic capping.

In one embodiment, the nucleic acid or modified nucleic acid disclosed herein encode a recombinant Ara h 2 variant polypeptide comprising the amino acid sequence as set forth in any one of SEQ ID NOs:10-63, 168, 170, 195-201, 204-210, 247-249, or comprises an amino acid sequence having at least 80% identity with the amino acid sequences set forth in any of SEQ ID NOs: 10-63, 168, 170, 195-201, 204-210, 247-249.

In one embodiment, the nucleic acid or modified nucleic acid comprises the nucleotide sequence of SEQ ID NO:167. In one embodiment, the nucleic acid or modified nucleic acid comprises the nucleotide sequence of SEQ ID NO:169.

In one embodiment, the nucleic acid or modified nucleic acid molecules disclosed herein encode a recombinant Ara h 2 variant polypeptide comprising the amino acid sequence set forth in SEQ ID NO: 4, wherein the Ara h 2 variant comprises substitution mutation(s) at one or more of positions 12, 15, 16, 22, 24, 46, 53, 65, 80, 83, 86, 87, 90, 104, 115, 123, 127, or 140 of SEQ ID NO: 4, as compared with the amino acid residues at those same positions in SEQ ID NO: 3. In one embodiment, the substitution mutation is N, Q, E, D, T, S, G, P, C, K, H, Y, W, M, I, L, V, or A at position 12. In one embodiment, the substitution mutation is R, E, K, Y, W, F, M, I, V, C, D, G, or A at position 15. In one embodiment, the substitution mutation is R, K, D, Q, T, M, P, C, E, or W at position 16. In one embodiment, the substitution mutation is F, Y, W, Q, E, T, S, A, M, I, L, C, R, or H at position 22. In one embodiment, the substitution mutation is D, E, H, K, S, T, N, Q, L, I, M, W, Y, F, P, A, or G at position 24. In one embodiment, the substitution mutation is T, V, E, H, S, A, G, Q, N, D, R, P, M, I, L, or C at position 46. In one embodiment, the substitution mutation is T, S, Q, V, A, G, C, P, M, L, I, E, H, R, K, N, or D at position 53. In one embodiment, the substitution mutation is T, A, N, D, Q, R, K, H, I, L, M, V, W, P, G, C, or E at position 65. In one embodiment, the substitution mutation is N, S, T, V, A, I, L, M, F, Y, W, C, E, K, R, or G at position 80. In one embodiment, the substitution mutation is D, A, C, F, I, P, T, V, W, Y, or Q at position 83. In one embodiment, the substitution mutation is Y, F, H, R, E, C, G, I, L, M, V, T, S, or Q at position 86. In one embodiment, the substitution mutation is F, Y, I, L, M, V, A, S, Q, R, K, D, N, E, or P at position 87. In one embodiment, the substitution mutation is S, P, Q or R at position 90. In one embodiment, the substitution mutation is L, M, K, R, H, E, D, A, Y, N, S, or W at position 104. In one embodiment, the substitution mutation is V, D, E, I, L, K, M, N, S, T, A, I, W, F, Y, or H at position 115. In one embodiment, the substitution mutation is I, Q, or A at position 123. In one embodiment, the substitution mutation is H, A, D, E, F, G, L, N, P, S, T, W, Y, Q, or V at position 127. In one embodiment, the substitution mutation is G, A, C, E, Y, F, H, K, L, M, N, P, Q, S, or V at position 140.

In one embodiment, the nucleic acid or modified nucleic acid molecules disclosed herein encode a recombinant Ara h 2 variant polypeptide comprising the amino acid sequence set forth in SEQ ID NO: 4, wherein there are at least 2, 3, 4, 5, 6, 7, 8, 9, 10, 11, 12, 13, 14, 15, 16, 17 or 18 substitution mutations at positions selected from positions 12, 15, 16, 22, 24, 46, 53, 65, 80, 83, 86, 87, 90, 104, 115, 123, 127, and 140 of SEQ ID NO: 4 as compared with the amino acid residues at those same positions in SEQ ID NO: 3.

In one embodiment, the nucleic acid or modified nucleic acid molecules disclosed herein encode a recombinant Ara h 2 variant having the amino acid sequence of SEQ ID NO:4, wherein amino acids at positions 12-16 of SEQ ID NO:4 comprise the sequence set forth in SEQ ID NO: 5.

In one embodiment, the nucleic acid or modified nucleic acid molecules disclosed herein encode a recombinant Ara h 2 variant having the amino acid sequence of SEQ ID NO:4, wherein amino acids at positions 44-65 of SEQ ID NO: 4 comprise the sequence set forth in SEQ ID NO: 6.

In one embodiment, the nucleic acid or modified nucleic acid molecules disclosed herein encode a recombinant Ara h 2 variant having the amino acid sequence of SEQ ID NO:4, wherein amino acids at positions 44-67 of SEQ ID NO: 4 comprise the sequence set forth in SEQ ID NO: 9.

In one embodiment, the nucleic acid or modified nucleic acid molecules disclosed herein encode a recombinant Ara h 2 variant having the amino acid sequence of SEQ ID NO:4, wherein amino acids at positions 11-90 of SEQ ID NO: 4 comprise the sequence set forth in SEQ ID NO: 7 or SEQ ID NO: 8.

In one embodiment, the nucleic acid or modified nucleic acid molecules disclosed herein encode a recombinant Ara h 2 variant having the amino acid sequence of SEQ ID NO: 4, wherein the Ara h 2 variant further comprises, in addition to the substitution mutations described above, additional substitutions, deletions, insertions, or any combination thereof, at one or more of positions 28, 44, 48, 51, 55, 63, 67, 107, 108, 109, 124, 125, or 142 of SEQ ID NO: 4, as compared with the amino acid residues at those same positions in SEQ ID NO: 3. In one embodiment, the substitution mutation is S, T, V, N, A, P, I, L, F, Y, H, R, K, E, or D at position 28. In one embodiment, the substitution mutation is I, A, C, G, H, L, F, Y, N, P, Q, K, E, S, T, V, M, or R at position 44. In one embodiment, the substitution mutation is V, G, C, E, H, Q, F, K, L, I, W, Y, N, R, S, T, V, A, or D at position 48. In one embodiment, the substitution mutation is S, G, Y, F, W, M, N, Q, E, R, K, H, T, D, or V at position 51. In one embodiment, the substitution mutation is G, A, D, E, F, Y, H, Q, V, I, L, M, R, K, S, T, C, or W at position 55. In one embodiment, the substitution mutation is P, C, F, V, I, L, M, W, Y, N, S, T, Q, G, H, K, or R at position 63. In one embodiment, the substitution mutation is E, Q, N, R, H, Y, F, W, M, L, V, T, S, A, P, or G at position 67. In one embodiment, the substitution mutation is A, C, F, G, H, I, K, L, M, Q, P, R, S, T, V, W, or Y at position 107. In one embodiment, the substitution mutation is T, V, D, E, R, H, Y, W, I, G, A, Q, or K at position 108. In one embodiment, the substitution mutation is K, C, S, R, G, P, Y, W, L, or I at position 109. In one embodiment, the substitution mutation is D, A, C, F, G, H, I, N, S, T, V, Y, L, E, or Q at position 124. In one embodiment, the substitution mutation is M, I, L, W, Y, G, K, N, T, V, or A at position 125. In one embodiment, the substitution mutation is M, A, C, E, F, G, H, I, K, L, N, P, Q, R, S, T, V, W, or Y at position 142.

In one embodiment, the nucleic acid or modified nucleic acid molecules disclosed herein encode a recombinant Ara h 2 variant polypeptide comprising the amino acid sequence set forth in SEQ ID NO: 4, wherein there are at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 substitution mutations at positions selected from positions 12, 15, 16, 22, 24, 28, 44, 46, 48, 51, 53, 55, 63, 65, 67, 80, 83, 86, 87, 90, 104, 107, 108, 109, 115, 123, 124, 125, 127, 140, or 142 of SEQ ID NO: 4, as compared with the amino acid residues at those same positions in SEQ ID NO: 3.

In one embodiment, the nucleic acid or modified nucleic acid molecules disclosed herein encode a recombinant Ara h 2 variant having the amino acid sequence of SEQ ID NO:4, wherein there are substitution mutations at positions 44, 48, 51, 55, 63, and 67 of SEQ ID NO: 4, as compared with the amino acid residues at those same positions in SEQ ID NO: 3.

In one embodiment, the nucleic acid or modified nucleic acid molecules disclosed herein encode an Ara h 2 variant comprising an amino acid sequence that is at least 70%, 75%, 80%, 85% or 90% identical to the sequence set forth in SEQ ID NO:3.

In one embodiment, the nucleic acid or modified nucleic acid molecules disclosed herein encode a recombinant Ara h 2 variant polypeptide having the amino acid sequence of SEQ ID NO:4, wherein the Ara h 2 variant comprises one of more substitution mutations at one of more positions of 6, 11-28, 32, 39, 44-56, 58, 60, 63, 69, 80-87, 89-90, 92, 96-97, 99, 100, 102-105, 107-119, 123, 125, 127-131, 133, 134, 136-144, 146, or 148-153 of SEQ ID NO:4, as compared with the amino acid residues at those same positions in SEQ ID NO: 3.

### Methods of Production

In some embodiments, variant polypeptides disclosed herein can be produced using a cell free in-vitro translation system, as is well known in the art for example but not limited to methods reviewed in Dondapati et al. (2020) BioDrugs 34(3):327-348. In one embodiment, the present disclosure provides a method of producing a hypo-allergenic peanut allergen comprising Ara h 1 variants disclosed herein, the method comprising culturing cells comprising the expression vector described above under conditions to express the Ara h 1 variant. In one embodiment, the cell is a prokaryotic cell or a eukaryotic cell. In one embodiment, the eukaryotic cell is a yeast cell, a fungi cell, a plant cell, or a mammalian cell.

In one embodiment, the present disclosure provides a method of producing a hypo-allergenic peanut allergen comprising Ara h 2 variants disclosed herein, the method comprising culturing cells comprising the expression vector described above under conditions to express the Ara h 2 variant. In one embodiment, the cell is a prokaryotic cell or a eukaryotic cell. In one embodiment, the eukaryotic cell is a yeast cell, a fungi cell, a plant cell, or a mammalian cell.

In some embodiments, the nucleic acid or modified nucleic acid molecules disclosed herein, is transcribed in an in vitro transcription system (IVT), wherein the transcribed nucleic acid or modified nucleic acid may then be used for immunotherapy by gene delivery, wherein administration of the mRNA results in the in vivo production of a peanut allergen or peanut allergen variants.

In some embodiments, the nucleic acid molecule encodes a wild-type (WT) peanut allergen. In some embodiments, the nucleic acid molecule encodes a variant peanut allergen comprising one or more amino acid substitutions, deletions, insertions, or any combination thereof that are located within a single epitope recognized by an antibody to the allergen.

In some embodiments of a method of production, the nucleic acid molecule encodes a WT Ara h 1 polypeptide. In some embodiments of a method of production, the nucleic acid molecule encoding a WT Ara h 1 polypeptide is selected from the sequence set forth in any of SEQ ID NO:171 and 172. In some embodiments of a method of production, the nucleic acid molecule encodes a WT Ara h 2 polypeptide. In some embodiments of a method of production, the nucleic acid molecule encoding a WT Ara h 2 polypeptide is set forth in any of SEQ ID NO: 164, and 165,

In some embodiments of a method of production, the nucleic acid molecule encodes a variant Ara h 1 polypeptide comprising one or more amino acid substitutions, deletions, insertions, or any combination thereof that are located within a single epitope recognized by an anti-Ara h 1 antibody. In some embodiments, the nucleic acid comprises a modified nucleic acid encoding a variant Ara h 1 polypeptide comprising one or more amino acid mutations that are located within a single epitope recognized by an anti-Ara h 1 antibody. In some embodiments of a method of production, the nucleic acid molecule encoding a variant Ara h 1 polypeptide comprises the sequence set forth in any of SEQ ID NOs: 173, 175, 177, 179, 181, and 183. In some embodiments of a method of production, the nucleic acid molecule encoding a variant Ara h 1 polypeptide having the amino acid sequence set forth in any of SEQ ID NOs: 68-161, 174, 176, 178, 180, 182, 184, 193, 194, 211-246.

In some embodiments of a method of production, the nucleic acid molecule encodes a variant Ara h 2 polypeptide comprising one or more amino acid substitution mutations that are located within a single epitope recognized by an anti-Ara h 2 antibody. In some embodiments, the nucleic acid comprises a modified nucleic acid encoding a variant Ara h 2 polypeptide comprising one or more amino acid mutations that are located within a single epitope recognized by an anti-Ara h 2 antibody. In some embodiments of a method of production, the nucleic acid molecule encoding a variant Ara h 2 polypeptide comprises the sequence set forth in any of SEQ ID NOs: 167 and 169. In some embodiments of a method of production, the nucleic acid molecule encoding a variant Ara h 2 polypeptide having the amino acid sequence set forth in any of SEQ ID NOs:10-63,168,170, 195-201, 204-210, 247-249.

Synthesis and capping of RNA molecules, either by chemical synthesis or by enzymatic processes such as bacteriophage RNA polymerases are well established methods in the art for mRNA production as described by Elain T. Schenborn Methods in Molecular Biology, Vol. 37: In Vitro Transcript/on and Translation Protocols pages 1-12 DOI: 10.1385/0-89603-288-4:1.

One skilled in the art would appreciate that other known IVT systems may be used to transcribe the nucleic acid or modified nucleic acid molecules described herein. In some embodiments, an mRNA molecule is transcribed in vitro using an IVT system.

Production of peanut allergen variants, Ara h 1 variants and Ara 2 variants, may comprise in vivo translation, wherein a transcribed mRNA is administered to a subject (in vivo translation).

In some embodiments, the nucleic acid or modified nucleic acid molecules disclosed herein, can be used to produce peanut allergen variant polypeptides in vivo, comprising administration of a nucleic acid or modified nucleic acid molecule by viral, nonviral or physical means such as liposome, cationic lipid, cationic polymer or hybrid lipid polymer systems, retroviral or DNA viral delivery e.g. lentiviral, foamyviral, adenoviral etc. sonoporation, electroporation, hydrodynamic delivery to a subject. In some embodiments, the nucleic acid molecules disclosed herein can be used to produce peanut allergen WT polypeptides in vivo, comprising administration of a nucleic acid molecule by viral, nonviral or physical means such as liposome, cationic lipid, cationic polymer or hybrid lipid polymer systems, retroviral or DNA viral delivery e.g. lentiviral, foamyviral, adenoviral etc. sonoporation, electroporation, hydrodynamic delivery to a subject. In vivo methods of administration of nucleic acid molecules, for example the mRNA molecules described herein encoding Ara h 1 or Ara h 2 variants, are well known in the art for example but not limited to methods reviewed in Jones et al., Overcoming Nonviral Gene Delivery Barriers: Perspective and Future. Mol. Pharmaceutics 2013, 10, 11, 4082-4098; Kamimura et al. Advances in Gene Delivery Systems. Pharmaceut Med. 25(5):293-306; and Nayerossadat et al., Viral and nonviral delivery systems for gene delivery. Adv Biomed Res 2012; 1:27, which are incorporated herein in full.

In some embodiments, a subject comprises a human subject. In certain embodiments, a subject comprises a baby, a child, an adolescent, a young adult, or a mature adult human. In some embodiments, a subject comprises a baby.

In some embodiments, a subject comprises one in need of inducing desensitization to peanuts. In some embodiments, a subject is allergic to peanuts. In some embodiments, a subject suffers from other food allergies. In some embodiments, a subject may be prone to develop peanut allergy.

### Method of Use

In one embodiment, the present disclosure provides a method of inducing desensitization to peanuts in subject allergic to peanuts, the method comprising administering to the subject a composition comprising the hypo-allergenic Ara h 1 variants disclosed herein, thereby inducing desensitization to peanuts in the subject.

In one embodiment, the present disclosure provides a method of inducing desensitization to peanuts in subject allergic to peanuts, the method comprising administering to the subject a composition comprising the hypo-allergenic Ara h 2 variants disclosed herein, thereby inducing desensitization to peanuts in the subject.

In one embodiment, the present disclosure provides a method of inducing desensitization to peanuts in subject allergic to peanuts, the method comprising administering to the subject a composition comprising a combination of hypo-allergenic Ara h 1 and Ara h 2 variants disclosed herein, thereby inducing desensitization to peanuts in the subject.

In some embodiments, the methods described herein comprise the use of adjuvant. “Adjuvant”, according to the present invention, refers to a compound or mixture that enhances the immune response to an antigen. An adjuvant may also serve as a tissue depot that slowly releases the antigen. Examples of adjuvants include, but are not limited to, monophosphoryl lipid A (MPL-A), MicroCrystalline Tyrosine (MCT), Calcium phosphate, complete Freund's adjuvant, incomplete Freund's adjuvant, saponin, mineral gels such as aluminum hydroxide, surface active substances such as lysolecithin, pluronic polyols, polyanions, peptides, Levamisol, CpG-DNA, oil or hydrocarbon emulsions, and potentially useful adjuvants such as BCG (bacille Calmette-Guerin) and Corynebacterium parvum. In some embodiments, Arah1 and Arah 2 variants are adsorbed to the MCT and administered with or without MPL-A. Both MCT and MPL-A should improve the efficacy of allergy immunotherapy and may have a synergistic effect when combined. Specifically, the adjuvants' administration may decrease the number of injections needed, decrease the dose and result in enhanced production of protective IgG antibodies. In addition, MCT adsorption may improve the safety of the product due to depot effect and gradual release of the proteins.

In one embodiment, the present disclosure provides a method of inducing desensitization to peanuts in subject allergic to peanuts, the method comprising administering to the subject a composition comprising nucleotide or modified nucleotide sequences encoding the recombinant hypo-allergenic Ara h 1 variants disclosed herein, thereby inducing desensitization to peanuts in the subject. In one embodiment, the present disclosure provides a method of inducing desensitization to peanuts in subject allergic to peanuts, the method comprising administering to the subject a composition comprising nucleotide or modified nucleotide sequences encoding the recombinant hypo-allergenic Ara h 2 variants disclosed herein, thereby inducing desensitization to peanuts in the subject. In one embodiment, the present disclosure provides a method of inducing desensitization to peanuts in subject allergic to peanuts, the method comprising administering to the subject a composition comprising nucleotide or modified nucleotide sequences encoding a combination of recombinant hypo-allergenic Ara h 1 and Ara h 2 variants disclosed herein, thereby inducing desensitization to peanuts in the subject. In one embodiment, the above composition comprises bacteria carrying the nucleotide sequences. In one embodiment, the nucleotide sequences are in the form of DNA or RNA.

In one embodiment, the composition in the above methods is administered orally. In another embodiment, the composition is administered by a route selected from sub-cutaneous, intra-muscular, intra-nasal, sub-lingual, topical, rectal or inhalation. In one embodiment, the subject in the above methods is an infant. In one embodiment, the composition in the above methods comprises a milk formula or a baby food.

In one embodiment, the present disclosure provides a method of inducing desensitization to peanuts in a subject allergic to peanuts, the method comprising administering to the subject a composition comprising a nucleic acid molecule encoding a recombinant Ara h 1 polypeptide, thereby inducing desensitization to peanuts in the subject. In some embodiments, a nucleic acid molecule used in a method of inducing desensitization to peanuts in a subject allergic to peanuts, comprises a nucleic acid molecule encoding a WT recombinant Ara h 1 polypeptide. In some embodiments, a nucleic acid molecule used in a method of inducing desensitization to peanuts in a subject allergic to peanuts, comprises a nucleic acid molecule or a modified nucleic acid molecule encoding a variant recombinant Ara h 1 polypeptide comprising one or more amino acid substitution mutations that are located within a single epitope recognized by an anti-Ara h 1 antibody. In some embodiments, a nucleic acid molecule used in a method of inducing desensitization to peanuts in a subject allergic to peanuts, comprises a nucleic acid molecule encoding a WT recombinant Ara h 2 polypeptide. In some embodiments, a nucleic acid molecule used in a method of inducing desensitization to peanuts in a subject allergic to peanuts, comprises a nucleic acid molecule or a modified nucleic acid molecule encoding a variant recombinant Ara h 2 polypeptide comprising one or more amino acid substitution mutations that are located within a single epitope recognized by an anti-Ara h 2 antibody.

In one embodiment, the present disclosure provides a method of inducing desensitization to peanuts in a subject allergic to peanuts, the method comprising administering to the subject a composition comprising a nucleic acid or modified nucleic acid molecule encoding a recombinant hypo-allergenic Ara h 1 variant disclosed herein, thereby inducing desensitization to peanuts in the subject.

In one embodiment, the present disclosure provides a method of inducing desensitization to peanuts in subject allergic to peanuts, the method comprising administering to the subject a composition comprising the nucleic acid or modified nucleic acid molecules encoding the recombinant hypo-allergenic Ara h 2 variants disclosed herein, thereby inducing desensitization to peanuts in the subject.

In one embodiment, the present disclosure provides a method of inducing desensitization to peanuts in subject allergic to peanuts, the method comprising administering to the subject a composition comprising the nucleic acid or modified nucleic acid molecules encoding a combination of recombinant hypo-allergenic Ara h 1 and Ara h 2 variants disclosed herein, thereby inducing desensitization to peanuts in the subject.

In one embodiment, the composition in the above methods comprises bacteria carrying the nucleic acid or modified nucleic acid molecules disclosed herein. In one embodiment, the nucleic acid or modified nucleic acid molecules are DNA or mRNA. Examples of DNA or mRNA have been described above.

In some embodiments of a method of inducing desensitization to peanuts in a subject allergic to peanuts, the nucleic acid molecule encodes a WT Ara h 1 polypeptide. In some embodiments of a method of inducing desensitization to peanuts in a subject allergic to peanuts, the nucleic acid molecule encoding a WT Ara h 1 polypeptide is selected from the sequence set forth in any of SEQ ID NO:171 and 172. In some embodiments of a method of inducing desensitization to peanuts in a subject allergic to peanuts, the nucleic acid molecule encodes a WT Ara h 2 polypeptide. In some embodiments of a method of inducing desensitization to peanuts in a subject allergic to peanuts, the nucleic acid molecule encoding a WT Ara h 2 polypeptide is set forth in any of SEQ ID NO: 164 and 165.

In some embodiments of a method of inducing desensitization to peanuts in a subject allergic to peanuts, the nucleic acid molecule encodes a variant Ara h 1 polypeptide comprising one or more amino acid substitution mutations that are located within a single epitope recognized by an anti-Ara h 1 antibody. In some embodiments, the nucleic acid comprises a modified nucleic acid encoding a variant Ara h 1 polypeptide comprising one or more amino acid substitution mutations that are located within a single epitope recognized by an anti-Ara h 1 antibody. In some embodiments of a method of inducing desensitization to peanuts in a subject allergic to peanuts, the nucleic acid molecule encoding a variant Ara h 1 polypeptide comprises the sequence set forth in any of SEQ ID NOs: 173, 175, 177, 179, 181, and 183. In some embodiments of a method of inducing desensitization to peanuts in a subject allergic to peanuts, the nucleic acid molecule encoding a variant Ara h 1 polypeptide having the amino acid sequence set forth in any of SEQ ID NOs: 68-161, 174, 176, 178, 180, 182, 184, 193, 194, 211-246.

In some embodiments of a method of inducing desensitization to peanuts in a subject allergic to peanuts, the nucleic acid molecule encodes a variant Ara h 2 polypeptide comprising one or more amino acid substitution mutations that are located within a single epitope recognized by an anti-Ara h 2 antibody. In some embodiments, the nucleic acid comprises a modified nucleic acid encoding a variant Ara h 2 polypeptide comprising one or more amino acid substitution mutations that are located within a single epitope recognized by an anti-Ara h 2 antibody. In some embodiments of a method of inducing desensitization to peanuts in a subject allergic to peanuts, the nucleic acid molecule encoding a variant Ara h 2 polypeptide comprises the sequence set forth in any of SEQ ID NOs: 167 and 169. In some embodiments of a method of inducing desensitization to peanuts in a subject allergic to peanuts, the nucleic acid molecule encoding a variant Ara h 2 polypeptide having the amino acid sequence set forth in any of SEQ ID NOs:10-63, 168, 170, 195-201, 204-210, 247-249.

In one embodiment, the composition in the above methods is administered orally. In another embodiment, the composition is administered by a route selected from sub-cutaneous, intra-muscular, intravenous, intra-nasal, sub-lingual, topical, rectal or inhalation. In one embodiment, the subject in the above methods is an infant.

### Sublingual Immunotherapy Using Peanut Allergens

There are two major approaches for the treatment of allergy. One possibility is based on the reduction of allergic inflammation by pharmacotherapy and/or biologics. The second approach for treatment is based on allergen-specific forms of intervention, i.e., allergen-specific immunotherapy (AIT). Major advantages of AIT are that the treatment is relatively inexpensive, it is highly effective if performed with high-quality allergens, treatment effects are long lasting after discontinuation if the treatment was performed for more than 2 years and AIT has disease-modifying effects preventing the progression from mild-to-severe manifestations.

Immunotherapy treats the cause of allergies by giving small doses of what a person is allergic to, which increases “immunity” or tolerance to the allergen and reduces the allergic symptoms. Sublingual immunotherapy, or SLIT, is a form of immunotherapy that involves putting liquid drops or a tablet of allergen extracts under the tongue. Many people refer to this process as “allergy drops,” and it is an alternative to allergy shots. SLIT has been used for years in Europe and has recently attracted increased interest in the United States.

There are only a few allergy drops approved by the Food and Drug Administration (FDA) in the United States. In 2014, three SLIT products in the form of tablets were approved by FDA for treating grass or ragweed allergy. More recently, FDA has approved a SLIT product to treat allergic rhinitis and conjunctivitis caused by house dust mites. SLIT is being studied as a potential treatment for peanut allergies. A key drawback of using peanut extract (PE) in SLIT is that it is not as effective as oral immunotherapy (OIT) in achieving desensitization. The amount of proteins used in OIT is about 100-500 fold higher (300-1000 mg per day) compared to that used in SLIT, (limitation of 2-4 mg per tablet). The dose difference might be the reason of SLIT is not as effective as oral immunotherapy in achieving desensitization for peanut allergies.

There are several forms of molecular allergen-specific immunotherapy (AIT), including (i) the production of wild type recombinant allergens, which resemble all of the properties of the corresponding natural allergens, (ii) the synthesis of peptides containing allergen-derived T cell epitopes without IgE reactivity, (iii) the use of allergen-encoding nucleic acids, and (iv) recombinant and synthetic hypoallergens, which exhibit strongly reduced IgE-binding capacity and allergenic activity but at the same time contain allergen-specific T cell epitopes (e.g. long synthetic peptides, recombinant hypoallergenic allergen derivatives) or instead of allergen specific T cell epitopes, they contain carrier elements providing T cell help (e.g. peptide carrier based B cell epitopes.

As used herein, “allergenicity” or “allergenic” refers to the ability of an antigen or allergen to induce an abnormal immune response, which is an overreaction and different from a normal immune response in that it does not result in a protective/prophylaxis effect but instead causes physiological function disorder or tissue damage.

A key difference between SLIT using peanut extract and the SLIT method disclosed herein is the amount of protein that theoretically can be given to the patient. It is well established that the amount of protein applied in immunotherapy via the sublingual route is significantly lower than that of the oral route (10-100-fold). Peanut extract is composed of lipids, carbohydrates and a variety of proteins, which only account for about 25% of the net weight of the peanut extract. Thus, the amount of a single protein in the peanut extract is low (e.g., Ara h 2 comprises just 6-9% of total protein). Consequently, a SLIT tablet of 2-4 mg of peanut extract would only contain ˜60 ug Ara h 2. In contrast, orally administered peanut extract that is in the range of 300 mg-1000 mg would contain ˜4-12 mg of Ara h 2. As a result, using natural peanut extract would not support a sufficient load of Ara h 2 (˜0.1-1 mg).

The method presented herein bypasses this hurdle by using recombinant pure proteins. In one embodiment, the method described herein can deliver up to 4 mg of peanut allergen (e.g., Ara h 1, Ara h 2 or variants thereof in a QD or BID regiment), thereby significantly increasing the amount of a specific protein in a SLIT tablet and getting much better efficacy with no safety problem due to the unique route of administration. The understanding that elevated amounts of peanut allergen will support better efficacy is not trivial, and it is believed that this is the innovative step that no one has tried before. In one embodiment, the dose for SLIT for Ara h 1 is from about 0.2 mg to about 4 mg. In one embodiment, the dose for SLIT for Ara h 2 is from about 0.1 mg to about 4 mg.

The data presented herein comparing SLIT to OIT (oral immunotherapy) demonstrated that SLIT had a similar clinical allergy desensitization effect as OIT, but with 10-fold less peanut protein. While non-sensitized mice show a strong anaphylactic temperature drop in response to peanut challenge, OIT with 500 ug Ara h 2 or SLIT with 50 ug Ara h 2 prevented this anaphylactic event.

The present disclosure presents experiments using Ara h 2 as an example. One of ordinary skill in the art would readily recognize that the method described herein would be equally applicable to other peanut allergens such as Ara h 1.

In one embodiment, the present disclosure provides a method of inducing desensitization to peanuts in a subject, the method comprising administering to the subject sub-lingually a composition comprising about 0.2 mg to about 4 mg of Ara h 1, thereby inducing desensitization to peanuts in the subject. In one embodiment, the subject is allergic to peanuts. In another embodiment, the subject is at risk of peanut allergy. In one embodiment, the Ara h 1 is purified from peanuts according to methods generally known in the art. In another embodiment, the Ara h 1 is produced by recombinant technology generally known in the art. In one embodiment, the Ara h 1 comprises the amino acid sequence set forth in any of SEQ ID NOs:64-67. In another embodiment, the Ara h 1 variant comprises the amino acid sequence set forth in any of SEQ ID NOs: 68-161, 174, 176, 178, 180, 182, 184, 193, 194, 211-246, or the amino acid sequence having at least 80% identity with the amino acid sequence set forth in any of SEQ ID NOs: 68-161, 174, 176, 178, 180, 182, 184, 193, 194, 211-246. In one embodiment, the composition administered sub-lingually is a tablet. In one embodiment, the tablet comprises about 0.2 mg to about 4 mg of Ara h 1, In one embodiment, the present disclosure provides a method of inducing desensitization to peanuts in a subject, the method comprising administering to the subject sub-lingually a composition comprising about 0.1 mg to about 4 mg of Ara h 2, thereby inducing desensitization to peanuts in the subject. In one embodiment, the subject is allergic to peanuts. In another embodiment, the subject is at risk of peanut allergy. In one embodiment, the Ara h 2 is purified from peanuts according to methods generally known in the art. In another embodiment, the Ara h 2 is produced by recombinant technology generally known in the art. In one embodiment, the Ara h 2 comprises the amino acid sequence set forth in any of SEQ ID NOs: 1-4. In another embodiment, the Ara h 2 variant comprises the amino acid sequence set forth in any of SEQ ID NOs: 10-63, 168, 170, 195-201, 204-210, 247-249, or comprises an amino acid sequence having at least 80% identity with the amino acid sequences set forth in any of SEQ ID NOs: 10-63, 168, 170, 195-201, 204-210, 247-249.

In one embodiment, the composition administered sub-lingually is a tablet. In one embodiment, the tablet comprises about 0.1 mg to about 4 mg of Ara h 2.

In one embodiment, the present disclosure provides a method of inducing desensitization to peanuts in a subject, the method comprising administering to the subject sub-lingually a composition comprising a combination of about 0.2 mg to about 4 mg of Ara h 1 and about 0.1 mg to about 4 mg of Ara h 2, thereby inducing desensitization to peanuts in the subject. In one embodiment, the subject is allergic to peanuts. In another embodiment, the subject is at risk of peanut allergy. In one embodiment, the Ara h 1 and Ara h 2 are purified from peanuts according to methods generally known in the art. In another embodiment, the Ara h 1 and Ara h 2 are produced by recombinant technology generally known in the art. In one embodiment, the Ara h 1 comprises the amino acid sequence set forth in any of SEQ ID NOs: 64-67. In one embodiment, the Ara h 2 comprises the amino acid sequence set forth in any of SEQ ID NOs:1-4. In one embodiment, the composition administered sub-lingually is a tablet. In one embodiment, the tablet comprises about 0.1 mg to about 4 mg of Ara h 2.

In one embodiment, the present disclosure provides a method of reducing allergic reaction to peanuts in a subject, the method comprising administering to the subject sub-lingually a composition comprising about 0.2 mg to about 4 mg of Ara h 1, thereby reducing allergic reaction to peanuts in the subject. In one embodiment, the Ara h 1 is purified from peanuts according to methods generally known in the art. In another embodiment, the Ara h 1 is produced by recombinant technology generally known in the art. In one embodiment, the Ara h 1 comprises the amino acid sequence set forth in any of SEQ ID NOs: 64-67. In one embodiment, the composition administered sub-lingually is a tablet. In one embodiment, the tablet comprises about 0.2 mg to about 4 mg of Ara h 1.

In one embodiment, the present disclosure provides a method of reducing allergic reaction to peanuts in a subject, the method comprising administering to the subject sub-lingually a composition comprising about 0.1 mg to about 4 mg of Ara h 2, thereby reducing allergic reaction to peanuts in the subject. In one embodiment, the Ara h 2 is purified from peanuts according to methods generally known in the art. In another embodiment, the Ara h 2 is produced by recombinant technology generally known in the art. In one embodiment, the Ara h 2 comprises the amino acid sequence set forth in any of SEQ ID NOs:1-4. In one embodiment, the composition administered sub-lingually is a tablet. In one embodiment, the tablet comprises about 0.1 mg to about 4 mg of Ara h 2.

In one embodiment, the present disclosure provides a method of reducing allergic reaction to peanuts in a subject, the method comprising administering to the subject sub-lingually a composition comprising a combination of about 0.2 mg to about 4 mg of Ara h 1 and about 0.1 mg to about 4 mg of Ara h 2, thereby reducing allergic reaction to peanuts in the subject. In one embodiment, the Ara h 1 and Ara h 2 are purified from peanuts according to methods generally known in the art. In another embodiment, the Ara h 1 and Ara h 2 are produced by recombinant technology generally known in the art. In one embodiment, the Ara h 1 comprises the amino acid sequence set forth in any of SEQ ID NOs: 64-67. In one embodiment, the Ara h 2 comprises the amino acid sequence set forth in any of SEQ ID NOs:1-4. In one embodiment, the composition administered sub-lingually is a tablet. In one embodiment, the tablet comprises about 0.1 mg to about 4 mg of Ara h 2.

In another embodiment, the present disclosure provides a tablet for sublingual immunotherapy of peanut allergy, wherein the tablet comprises about 0.2 mg to about 4 mg of Ara h 1. In one embodiment, the Ara h 1 is purified from peanuts according to methods generally known in the art. In another embodiment, the Ara h 1 is produced by recombinant technology generally known in the art. In one embodiment, the Ara h 1 comprises the amino acid sequence set forth in any of SEQ ID NOs: 64-67.

In another embodiment, the present disclosure provides a tablet for sublingual immunotherapy of peanut allergy, wherein the tablet comprises about 0.1 mg to about 4 mg of Ara h 2. In one embodiment, the Ara h 2 is purified from peanuts according to methods generally known in the art. In another embodiment, the Ara h 2 is produced by recombinant technology generally known in the art. In one embodiment, the Ara h 2 comprises the amino acid sequence set forth in any of SEQ ID NOs:1-4.

In another embodiment, the present disclosure provides a tablet for sublingual immunotherapy of peanut allergy, wherein the tablet comprises a combination of about 0.2 mg to about 4 mg of Ara h 1 and about 0.1 mg to about 4 mg of Ara h 2. In one embodiment, the Ara h 1 and Ara h 2 are purified from peanuts according to methods generally known in the art. In another embodiment, the Ara h 1 and Ara h 2 are produced by recombinant technology generally known in the art. In one embodiment, the Ara h 1 comprises the amino acid sequence set forth in any of SEQ ID NOs: 64-67. In one embodiment, the Ara h 2 comprises the amino acid sequence set forth in any of SEQ ID NOs: 1-4.

In one embodiment, the present disclosure provides the tablets described above for inducing desensitization to peanuts in a subject. In one embodiment, the subject is allergic to peanuts. In another embodiment, the subject is at risk of peanut allergy.

In one embodiment, the present disclosure provides the tablets described above for reducing allergic reaction to peanuts in a subject.

In another embodiment, the compositions described herein can be formulated into nucleic acid vaccine composition for inducing desensitization to peanuts in a subject, or reducing allergic reaction to peanuts in a subject.

As used herein, “nucleic acid vaccine” refers to a vaccine or vaccine composition which includes a nucleic acid or nucleic acid molecule (e.g., a polynucleotide) encoding an allergen or derivative thereof (e.g., variants of Ara h 1 and/or Ara h 2 protein or polypeptide). In exemplary embodiments, a nucleic acid vaccine includes a ribonucleic (“RNA”) polynucleotide, ribonucleic acid (“RNA”) or ribonucleic acid (“RNA”) molecule. Such embodiments can be referred to as ribonucleic acid (“RNA”) vaccines. In some embodiments, a nucleic acid vaccine includes a messenger RNA (“mRNA”) polynucleotide, messenger RNA (“mRNA”) or messenger RNA (“mRNA”) molecule as described herein. Such embodiments can be referred to as messenger RNA (“mRNA”) vaccines. Said vaccines may comprise other substances and molecules which are required, or which are advantageous when said vaccine is administered to an individual (e.g., pharmaceutical excipients).

In one embodiment, the RNA vaccine comprises RNA sequence encoding the allergen. This RNA sequence can be the sequence of the allergen or can be adapted with respect to its codon usage. Adaption of codon usage can increase translation efficacy and half-life of the RNA. In one embodiment, a poly A tail comprising at least 30 adenosine residues is attached to the 3′ end of the RNA to increase the half-life of the RNA. In one embodiment, the 5′ end of the RNA is capped with a modified ribonucleotide with the structure m7G(5′)ppp(5′)N(cap 0 structure) or a derivative thereof which can be incorporated during RNA synthesis or can be enzymatically engineered after RNA transcription by using Vaccinia Virus Capping Enzyme (VCE, consisting of mRNA triphosphatase, guanylyl-transferase and guanine-7-methytransferase), which catalyzes the construction of N7-monomethylated cap 0 structures. Cap 0 structure plays a crucial role in maintaining the stability and translational efficacy of the RNA vaccine. The 5′ cap of the RNA vaccine can be further modified by a 2′-O-Methyltransferase which results in the generation of a cap 1 structure (m7Gppp[m2′-O]N), which further increases translation efficacy. The vaccine or vaccine formulation according to the present invention can further include an adjuvant.

### Plants and Products

In one embodiment, the present disclosure provides a genetically modified peanut plant, the peanut plant comprising peanuts expressing the Ara h 1 variants disclosed herein.

In one embodiment, the present disclosure provides a genetically modified peanut plant, the peanut plant comprising peanuts expressing the Ara h 2 variants disclosed herein.

In one embodiment, the present disclosure provides a genetically modified peanut plant, the peanut plant comprising peanuts expressing a combination of hypo-allergenic Ara h 1 and Ara h 2 variants disclosed herein.

In one embodiment, the Ara h 1 variants, or the Ara h 2 variants, or a combination thereof, expressed in the above genetically modified peanut plant are expressed from a heterologous nucleic acid.

In one embodiment, the Ara h 1 variants, or the Ara h 2 variants, or a combination thereof, expressed in the above genetically modified peanut plant are endogenously expressed from a genetically modified chromosome.

In some embodiments of the above genetically modified peanut plant, expression of endogenous wild-type Ara h 1 allergen, or endogenous wild-type Ara h 2 allergen, or a combination thereof, is reduced compared with a non-genetically modified peanut plant.

In some embodiments of the above genetically modified peanut plant, the modified plant further expresses at least one RNA silencing molecule that (i) reduces expression of the endogenous Ara h 1 allergen, the endogenous Ara h 2 allergen, or a combination thereof, and (ii) does not reduce the expression of the Ara h 1 variant, the Ara h 2 variant, or a combination thereof.

In some embodiments of the above genetically modified peanut plant, the modified plant further expresses a DNA editing system directed towards reducing expression of the endogenous Ara h 1 allergen, the endogenous Ara h 2 allergen, or a combination thereof.

In one embodiment, the present disclosure provides a processed food product comprising the Ara h 1 variants disclosed herein.

In one embodiment, the present disclosure provides a processed food product comprising the Ara h 2 variants disclosed herein.

In one embodiment, the present disclosure provides a processed food product comprising a combination of Ara h 1 and Ara h 2 variants disclosed herein.

In one embodiment, the above processed food product comprises a reduced amount of endogenous peanut Ara h 1 allergen, or endogenous Ara h 2 allergen, or a combination thereof.

In one embodiment, the above processed food product comprises a peanut harvested from the genetically modified plant described above.

As used herein, the singular form “a”, “an” and “the” include plural references unless the context clearly dictates otherwise. For example, the term “a compound” or “at least one compound” may include a plurality of compounds, including mixtures thereof.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

Throughout this application, various embodiments of Ara h 1 and Ara h 2 variants, and mutation and/or epitope positions thereof may be presented in a range format. It should be understood that the description in range format is merely for convenience and brevity and should not be construed as an inflexible limitation on the scope of Ara h 1 or Ara h 2 variants and mutation and/or epitope positions thereof. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### EXAMPLES

### Example 1: Materials and Methods

### Peptide Microarray Assay

To determine anti-Ara h 1 and anti-Ara h 2 epitopes, a Celluspot™ peptide microarray-based immunoassay (Intavis, Cologne, Germany) was performed (Winkler, Dirk F H, Peptide microarrays. Humana Press, 2009). The peptides, of 15 amino-acids in length with an offset of 4 amino-acids, derived from the primary sequence of peanut allergens Ara h 1 (uniprot entry P43238 positions 25-626; SEQ ID NO: 64), Ara h 2 (uniprot entry Q6PSU2; SEQ ID NO: 1), Ara h 3 (uniprot entry 082580), Ara h 6 (uniprot entry A5Z1R0) and Ara h 8 (uniprot entry Q6VT83), were synthesized and spotted on the microarray in duplicates. The slides were rinsed with a blocking buffer (150 mM NaCl, 0.05% Tween, 2.5% skim milk, 50 mM Tris pH7.5) for overnight at 4° C. Then, the slides were washed and incubated with 3 ml of 6.2 ug/ml single-chain variable fragment (scFv) in a blocking buffer incubated for 4 hr at 4° C. on a rotator. For detection, the slides were incubated with 3 ml of horseradish peroxidase (HRP)-tagged goat-anti-human IgE (abeam, Cambridge, United Kingdom), diluted 1:10,000 in a blocking buffer for 2 hr at 25° C. on a rotator. After washes, femtogram HRP Substrate kit [Azure Biosystem, Dublin, California] was added and chemiluminescence was read via ChemiDoc [BioRad, Hercules, CA]. Peptide array images were processed by an in-house python script that detects peptide spots, normalizes their intensities, and reports any series of at least two overlapping spots showing across the duplicate a mean signal that is higher than two standard deviations from the slide mean.

Generation of Human scFv Phage Display Library

Whole blood samples of 5-20 ml were taken from clinically diagnosed peanut allergy patients using Heparin or EDTA treated tubes (BD). Peripheral blood mononuclear cells (PBMC) were extracted from blood samples using Sepmate tubes (STEMCELL) according to the manufacturer's instructions. RNA was purified from 5-15×10⁶ PBMC using the RNAeasy extraction kits (Qiagen; Hilden, Germany) and cDNA was prepared from 1-5 μg RNA (depending on the amount of RNA obtained).

The entire cDNA reaction was divided into PCR reactions to amplify the antibodies hyper-variable domain of each patient's variable genes. Light chains were amplified using gene sub-family specific forward primers carrying an unstructured, non-specific overhang followed by a NotI restriction site and reverse primers specific for the IGLK and IGLL isotypes carrying homology to the 5′ portion of an unstructured linker. Heavy chains were amplified using gene sub-family specific forward primers carrying homology to the 3′ portion of an unstructured linker and reverse primers specific for IGHG and IGHE genes carrying an unstructured, non-specific overhang followed by a NcoI restriction site. Primers were adapted from “Phage display: Methods and Protocols” (2018) Hust M and List T eds. Springer Protocols. PCR 50 μl reactions were performed with Phusion hot start Taq Polymerase kit, 200 μM dNPT, 2% DMSO, 1.25M Betaine, 1-5 μg cDNA and 0.5 μM each primer. Reactions were performed using the following PCR program: 3 min at 98° C., 30 cycles of 98° C. 20 sec+60° C. 60 sec+72° C. 45 sec, and a final elongation stage of 72° C. for 10 min.

PCR products of each family (VHγ, VHκ, VLκ and VLλ) were combined, each pool was concentrated by ethanol precipitation, ran on a 1% agarose gel, extracted using gel extraction kit (Qiagen) and cleaned using Amicon ultra 30K centrifugal filters (Sigma-Aldrich Merck, Israel). A DNA mix of amplified V gene segments was prepared at a ratio of 45% Vγ, 5% VF, 25% Vκ, and 25% Vκ. Production of combinatorial light-heavy scFv libraries was performed by PCR reactions using the same reagent as the first PCR, but at 100 μl per reaction, with 100 ng of the V-gene mix, with “pull-through” primers (complementary to the overhangs flanking the restriction site of each product from the first PCR) at a concentration of 250 nM. Multiple recombination reactions (18-24) were prepared without primer and PCR was performed using the following program: 3 min at 98° C., 5 cycles of 98° C. 20 sec+60° C. 60 sec+72° C. 60 sec. Primers were then added and the reaction was performed using the following program: 1 min at 98° C., 30 cycles of 98° C. 20 sec+67° C. 60 sec+72° C. 45 sec and a final elongation stage of 72° C. for 3 min.

PCR products were concentrated by ethanol precipitation, ran on a 1% agarose gel, extracted using gel extraction kit (Qiagen) and cleaned using Amicon ultra 30K centrifugal filters (Sigma-Aldrich Merck). The pLibGD vector (described below) and the purified scFv DNA (at least 4 ug vector and 2 um scFv) were restricted using hi-fidelity NcoI and NotI enzymes (NEB; MA, USA) according to the manufacturer's instructions. The vector was further treated by QuickCIP (NEB) according to the manufacturer's instructions. The restricted vector was cleaned by extraction from a 1% agarose gel and centrifugal filters as in previous steps. Restricted scFv were purified using PCR cleanup columns (Qiagen).

Ligation reactions of 20 μl were set up according to the manufacturer's instructions using 130 ng vector and 70 ng insert (producing a 3:1 ratio) and carried out at 10° C. overnight. A total of at least 3 g DNA was ligated. Ligations were heat inactivated, cleaned by PCR cleanup columns, and concentrated by Amicon 30K centrifugal filters.

Ligated libraries were transformed to SS320 electrocompetent bacteria (Lucigen; WI, USA) according to manufacturer's instructions. Each library was divided into 2 transformations and seeded on three 15 cm 2YT-agar dishes containing 100 g/ml carbenicillin and 2% glucose. Dishes were incubated overnight at 30° c. Serial dilutions of transformations were seeded on separate kanamycin and ampicillin dishes to estimate transformation efficiencies. Libraries of 107<were considered of sufficient quality and used further.

The next day, SS320 were scraped off of the dishes using 6 ml 2YT, diluted to O.D=0.1 in 60 ml 2YT supplemented with 100 g/ml carbenicillin and 2% glucose, grown to OD=0.5, and infected with KO7 helper phage (NEB) diluted 1:1000 for 30 minutes at 37° C. The bacteria were then centrifuged at 3000 g for 10 minutes, resuspended in 200 ml 2YT+100 g/ml carbenicillin+25 g/ml kanamycin and grown at least overnight or up to 24 hours at 30° C. with 250 RPM shaking in baffled flasks to produce scFv-displaying phages.

The next day, bacteria were centrifuged at 18,000 g for 10 minutes at 16,000 g. Supernatant was moved to fresh tubes and phages were precipitated by adding PEG/NaCl stock (PEG-8000 20%, NaCl 2.5 M) to a final concentration of 20% (1:4 ratio of PEG-NaCl stock to supernatant). Samples were incubated on ice for 20 minutes and centrifuged at 18,000 g, 4° C. for 30 minutes. Supernatant was discarded and the pellet was centrifuged again for 2 minutes to remove the remaining supernatant. Pellet was resuspended with 10 ml PBS/100 ml culture and centrifuged for 10 minutes at 18,000 g to remove residual bacteria cell debris. Samples were then subjected to a second identical round of PEG-NaCl precipitation, and resuspended with 4 ml PBS/100 ml culture. Samples were centrifuged for 15 minutes at 20,000 g to remove residual debris and purified phages were supplemented with 50% glycerol and 2 mM EDTA and stored at −80° C. until use.

Screening of Phage Display Libraries for Allergen-Specific scFv

Isolation of allergen-specific scFv was done by panning phage libraries using either the natural purified allergen or recombinant allergen variants with modified suspected epitopes. Maxisorp high-binding 96-well plates (Nunc) were coated with 100 ul of 5 ug/ml allergen solution in PBS or with 2% BSA solution in PBS (8 wells per library). OmniMAX™ bacteria (Thermo Fisher Scientific; MA, USA) were seeded in 2YT+Tetracycline (5 ug/ml) and grown overnight at 37° C. with 250 RPM shaking.

The next day, OmniMAX™ bacteria were diluted in 2YT+Tetracycline to 0.1 O.D, grown to O.D=0.6-0.8 at 37° C. with 250 RPM shaking and kept on ice until use. Phage stock (2-4 ml) were defrosted, purified by PEG-NaCl purification (as above) and resuspended with 1 ml PBST (PBS+0.05% tween). A sample of un-panned phage stock was put aside for input measurement. If negative selection was performed, maxisorp plates were washed with 200 μl/well PBST×3 and then phage solution was incubated in BSA-coated wells to remove non-specific binders at 100 μl/well for 1 hour at 4° C. with gentle shaking. Phage solutions were then moved to allergen-coated wells and incubated for 1 hour at 4° C. with gentle shaking. If no negative selection was performed, phage-PBST solution was added directly to allergen-coated wells. Plates were then washed twice with 200 μl/well PBST to remove unbound phages. Bound phages were eluted by incubation for 5 minutes with 100 μl/well of 100 mM HCl at R.T with gentle shaking. Elution reaction was stopped with 12.5 μl/well of Tris 1M, pH 11.

Eluted samples were added to 5 ml OmniMAX™ at required O.D and incubated for 30 minutes at 37° C. with 250 RPM shaking. Panning output titration was assessed by performing serial 10-fold dilutions with a sample of the infected stocks and seeding in triplicates 5 μl-drops on LB-agar dishes with carbenicillin or kanamycin or tetracycline. Remaining output was propagated by super-infection with 1:100 KO7 helper stock at 1:1000 for 45 minutes at 37° C. with 250 RPM shaking. Super-infected bacteria stocks were completed to 50 ml 2YT supplemented with carbenicillin and kanamycin and grown overnight at 37° C. with 250 RPM shaking to produce phages for the next round of panning. Panning input titration was assessed by performing serial 10-fold dilutions of input samples, infecting OmniMAX™ bacteria for 30 minutes at 37° C. with 250 RPM shaking and seeding triplicate drops on carbenicillin and kanamycin LB-agar dishes.

Subsequent panning rounds were performed by performing a single PEG-NaCl precipitation of the overnight output propagation and using it as input. From one panning round to the next, the number of wash cycles was increased, and the number of panning wells was decreased to increase panning stringency (3-to-4 panning cycles per library).

To isolate individual allergen-specific scFv, output serial dilutions of a chosen round were seeded onto LB-agar-carbanicillin dishes and grown overnight at 37° C. The next day, individual colonies were inoculated into mini-tubes containing 300 μl 2YT+carbanicillin+1:1000 KO7 and grown overnight at 37° C. with 250 RPM shaking. The next day, supernatants from mini-tubes were assayed by ELISA using plates coated with the allergen or BSA. The scFv from supernatants that bound specifically to the allergen and not to BSA were amplified by PCR with primers flanking the scFv region of the pLibGD plasmid. PCR products that were consistent with a full-length scFv were subjected to standard PCR cleaning by ExoI and rSAP restriction enzymes (NEB) and sequenced by standard sanger reactions (Hylabs). Unique, full-length monoclones were used for production of purified scFv.

scFvs Purification

The monoclonal antibodies variable regions were introduced to scFv polypeptide chain that can be easily expressed in a bacterial expression system. For scFv expression, scFvs were cloned into LibG plasmid encoding periplasmic secretion signal and Flag tag at its N′-terminal, His tag was cloned at its C′-terminal (ST2 secretion signal-Flag-scFv-His tag) under the transcriptional control of Tac promoter. The construct was grown at 37° C., induction was carried out overnight, by addition of 1 mM IPTG at 20° C. when cells reached an OD of 0.8-1.0. Cells were harvested (4800 g for 20 min) and cells pellet was resuspended with PBS-lysis buffer (1% v/v Triton X-100, 250 U Benzonase, 0.2 mM PMSF, 1 mg/ml Lysozyme, 10 mM Imidazole). Lysis took place while cells were shaken at 4° C. for 1 hr. Following that, lysates were separated by centrifugation (15000 g for 30 min). The supernatant was loaded on pre-washed (PBS with 10 mM imidazole) Ni-NTA beads and incubated at 4° C. for 1 hr. Beads were washed with PBS with increased imidazole concentration (up to 250 mM). Buffer was exchanged to PBS by overnight dialysis at 4° C., using SnakeSkin dialysis tubing 3.5 kDA (Thermo Fisher Scientific). ScFvs were concentrated by 3 kDa centricones (Amicon, Mercury) and their concentration was measured by absorbance at 280 nm.

### Single Cell Sorting of Allergen Specific B Cells

Peanut allergy patients PBMC were thawed, washed with PBS, and stained for viability (LIVE/DEAD near-IR kit, Thermo-fisher) according to manufacturer's instructions. Cells were then incubated on ice for 1 hour with target allergens at varying concentrations according to allergen type. Allergens used were either natural purified allergens that were fluorescently labeled with alexa-fluor protein labeling kit (Thermo-fisher, a mix of allergens labeled with 2 different fluorophores, according to manufacturer's instructions), OR wt recombinant allergens with HA-tags on either C or N terminus, OR biotin-avidin labeled wt recombinant allergens (a mix of allergens labeled with 2 different fluorophores). Cells were then washed and stained with flourophore-conjugated antibodies for the following markers: CD14, CD16, IgM, IgD, CD3, CD19, IgG1. If using HA-tagged allergens, two anti-HA antibodies with different fluorophore conjugations were also added. Cells were then washed and sorted on an ARIA-III sorting flow cytometer. Single allergen-specific B cells (LIVE/DEADdim CD14− CD16− IgD− IgM− CD3− CD19+IgG1+ allergen fluorophores double positive) were sorted into 96-well plates containing 4 μl/well ice-cold lysis buffer (PBS×0.5, 10 mM DTT, 8 U RNAse inhibitor). Several wells were left empty in each plate as negative controls for PCR.

Isolation of Antibody Genes from Sorted Cells and Antibody Expression

Single sorted allergen-specific B cell lysates were directly subjected to reverse-transcription (SSIV, Invitrogen, according to manufacturer's instructions). Two sequential PCR reactions (2nd PCR nested) were performed to amplify heavy chain genes (Hotstart taq polymerase, NEB) and light chain genes (Kapa hot-start PCRF mix) using a mix of primers that cover the majority of known antibody gene alleles. PCR products were sequenced and aligned to the genome. Where a cell had reliable sequences for both heavy and light chains, sequences were cloned into mammalian expression plasmids (pSF), and expressed in HEK-293t cells.

### Preparation of Yeast Surface Display Mutant Saturation Library and Flow-Cytometric Cell Sorting

A library consisting of Ara h 2 variants with single mutations in each residue was ordered from TWIST Bioscience (CA, USA) and cloned into a YSD vector (pETCON). To display the Ara h 2 library on the surface of the yeast denoted as S1, the library was grown in an SDCAA selective medium (2% dextrose, 0.67% Difco yeast nitrogen base, 0.5% Bacto casamino acids, 0.52% Na2HPO4, and 0.856% NaH2PO4·H2O) and induced for expression with a galactose medium (as for SDCAA, but with galactose 2%, instead of dextrose) according to an established protocol (Chao, G., Lau, W., Hackel, B. et al. Isolating and engineering human antibodies using yeast surface display. Nat Protoc 1, 755-768 (2006)). Ara h 2 expression was detected by an anti-Myc antibody conjugated to FITC (Miltenyi Biotec, Bergisch Gladbach, Germany) and anti-Ara h 2 scFv binding was detected by secondary anti-FLAG antibody conjugated with APC (Miltenyi Biotec, Bergisch Gladbach, Germany). For pairwise selectivity screen, ˜1×10⁶ yeast cells were incubated with different anti-Ara h 2 scFv in a binding buffer (100 mM Tris, pH=8.0, 1 mM CaCl₂), 1% BSA) for 1 h at room temperature. Then, the cells were washed with the binding buffer and incubated for 30 min with anti-Myc-FITC and anti-FLAG-APC antibodies. Then, the cells were washed again with a binding buffer and sorted for the high and low-selective variants by conducting several independent sorts, using FACSAria. Ara h 2 variants that showed a high and low binding affinity toward the anti-Ara h 2 scFv, i.e., top the lowest up to 1% and highest 1% of the entire population were selected and denoted as mAb_S2_low and mAb_S2_high.

### High-Throughput Sequencing Library Preparation

A YSD vector (pETCON) containing the Ara h 2 gene was isolated from the naïve library and from the sorted libraries by using Zymoprep Yeast Plasmid Miniprep II (Zymo research, Irvine, CA) according to the manufacturer's protocol. Using this kit, ˜200 ng of pETCON were isolated from each yeast library. The extracted pETCON were sent to the NGS laboratory of Hy Laboratories (Hylabs, Rehovot, Israel) for a first and secondary PCR of twenty and eight cycles (respectively), using the Fluidigm Access Array primers, to add the adaptors and barcodes. Then, the DNA library samples were purified with AmpureXP beads (Beckman Coulter, Brea, CA) and the concentrations of the samples were determined in a Qubit by using the DNA high sensitivity assay. The samples were pooled and then ran on a TapeStation (Agilent, Santa Clara, CA) to verify the size of the PCR product. As a final quality test, the pools were subjected to qRT-PCR to determine the concentration of the DNA that can be sequenced. The pools were then loaded for sequencing on an Illumina Miseq, using the 600v2 kit.

### Deep Sequencing Reads Analysis

Paired-end reads were analyzed and filtered for quality using the fastp command-line preprocessing tool (Chen, S., Zhou, Y., Chen, Y., & Gu, J. (2018). fastp: an ultra-fast all-in-one FASTQ preprocessor. Bioinformatics (Oxford, England), 34(17), i884-i890.). All sequences where over 10% or 20% of the sequence had a Phred quality score under 20, depending on whole library quality, were discarded from subsequent analysis. Reads were then aligned based on a probabilistic model of their overlapping region, implemented within the pandaseq assembler (Masella, A. P., Bartram, A. K., Truszkowski, J. M. et al. PANDAseq: paired-end assembler for illumina sequences. BMC Bioinformatics 13, 31 (2012).). Translated sequences were filtered for the appearance of expected mutations (single mutation per sequence, i.e., single mutation per variant) and analyzed for sequence enrichment:

$$Enrich Xi = ( \frac{{fS}_{1} {aa}_{i}}{{fS}_{0} {aa}_{i}} )$$

Where aai is a specific amino acid at position i, fS1 is the fraction of reads of the given amino acid at position i in the sorted library and fS0 is the same fraction, in the input library. This calculation provides the enrichment of each specific Ara h 2 point mutant.

For convenience, the following can also be denoted as the increase index of a specific amino acid at position i.

$$IN {aa}_{i} = \frac{{fS}_{1} {aa}_{i}}{{fS}_{0} {aa}_{i}}$$

integration of this information over all mutations at a given position is performed by calculating the Shannon entropy of each position:

$${Ent}_{i} = {\sum }_{z = 1}^{20} \frac{{INaa}_{z}}{{\sum }_{j = 1}^{20} {Inaa}_{j}} ln {\sum }_{z = 1}^{20} \frac{{INaa}_{z}}{{INaa}_{j}}$$

Where i is a given position, INaaz represents the increase index for a given amino acid, normalized by the increase index of all amino acids.

### Ara h 1 and Ara h 2 Purification

For Ara h 2 and Ara h 1 variant purification, Ara h 2 WT (SEQ ID NO: 2) and mutants were cloned into pET28 plasmid, as were Ara h 1 WT (SEQ ID NO: 65) and mutants thereof. Ara h 2 was fused to DNA encoding His-tagged Trx and TEV protease cleavage sequences (Trx-His*6-TEV site-Ara h 2). For the Ara h 1 variant DNA, DNA sequences of Met-TEV-His*6 tag were added at the N-terminus and for some variants Met as a start codon at the N-terminus was added and His*6 at the C-terminus. Additional restriction sites were incorporated as needed for restriction cloning. All variants were expressed under the transcriptional control of T7 promoter. Cells were grown at 37° C. until an OD of 0.5-0.8 was reached, induction was carried out overnight by addition of 1 mM IPTG at 20° C. or 3 h at 37° C. Cells were harvested (4800 g for 30 min) and cells pellet was resuspended with lysis buffer (50 mM Tris pH 8.0, 350 mM NaCl, 10% v/v glycerol, 0.2% Triton X-100, 250 U Benzonase, 0.2 mM PMSF and 1 mg/ml Lysozyme), lysis was done by sonication (35% amplitude, 10 sec on and 30 sec off for 2 min). Lysates were centrifuged (15000 g, 45 min) and supernatant was loaded on pre-washed with binding buffer (50 mM Tris pH 8.0, 350 mM NaCl and 10% v/v glycerol) Ni-NTA beads and incubated at 4° C. for 1 hr. The beads were washed with a binding buffer containing increased imidazole concentration. For Ara h 2 purification TEV protease was added to samples containing the Trx-Ara h 2 protein and the buffer was exchanged to PBS by overnight dialysis at 4° C., using SnakeSkin dialysis tubing 3.5 kDA (Thermo Fisher scientific). Following TEV cleavage, the Trx-His tag portion and the TEV protease (containing His tag also) were removed by loading the solution onto a Ni-NTA column. The flow-through containing Ara h 2 was collected and concentrated by 3 kDa centricones (Amicon, Mercury), protein concentration was measured by the absorbance at 280 nm. For Ara h 1 purification, an additional gel filtration step on Superdex 200 was performed.

### Analysis of Binding to Monoclonal Antibodies by ELISA

The concentrations of Anti-Ara h 1 and Anti-Ara h 2 scFv required to give 50% of maximal binding to WT-Ara h and Ara h variants (EC50) were determined using an ELISA. Briefly, wells of 96-well microtiter plates (Thermo Fisher Scientific, Waltham, MA) were coated overnight at 4° C. with 200 ng of Ara h 2 or Ara h 1. Plates were blocked with 0.5% BSA in PBS (200 μl/well) for 1 hr at RT. Anti-Ara h scFv variants were prepared by a serial dilution in PBS with starting concentrations of 4 μM, added to the Ara h-coated wells and incubated for 1 hr at 37° C. Following washing steps, the amount of bound scFv was detected by incubation with the Goat-anti-FLAG conjugated with HRP polyclonal antibody (Abcam, Cambridge, United Kingdom) and then TMB substrate.

All incubation steps were performed in PBS containing 0.5% BSA and 0.05% Tween 20. The highest concentrations of Anti-Ara h scFv are saturating, and the amount bound to Ara h 1 or Ara h 2 reaches a maximum at these levels.

Computational Design of Variants with Mutations at Multiple Sites

Based on experimental results which identified point mutations that reduce binding to mAbs and/or to patient sera, the Schrodinger Maestro software suite (Schrödinger, L. L. C. “The Maestro suite of programs: A powerful, all-purpose molecular modeling environment.” New York: Schroedinger LLC (2005)) was used to generate variants with combinations of mutations that are predicted to maintain their stability. The solved crystal structure of Ara h 2 (PDB accession 3ob4) was prepared for analysis (residues belonging to the MBP protein that is fused to Ara h 2 were removed, and the protein preparation wizard was used to remove waters, optimize hydrogen bonds, and minimize the protein backbone). Next, the residue scanning tool was used to perform monte-carlo sampling of up to 5 simultaneous mutations, in cases where mutations were combined at the epitope level, or up to 25 simultaneous mutations, in cases where mutations were combined at the protein level, allowing minimization of the backbone upon side chain mutation and generating 250 structures. Mutations were evaluated by the computed AG, the change in the free energy of protein upon mutation. Sequences were ranked by their AG, eliminating any structure with AG>10 and by their sequence diversity, to eliminate experimental testing of near identical protein sequences.

### RBL SX-38 Cell Degranulation Assays

RBL SX-38 cells were received from Prof. Stephen Dreskin in UC Denver, with permission from BIDMC in Boston. Cells were cultured at 37° C., 5% CO₂ in maintenance media containing 80% MEM, 20% RPMI 1640, 5% FCS (not heat-inactivated), supplemented with L-glutamin, Penicillin-Streptomycin and G418 at 1 mg/ml (all from Gibco-Thermo fisher, USA). At least 48 hours before assay, cells were split and expanded in assay media (maintenance media without RPMI and G418). On day of assay, cells were detached using 0.05% Trypsin-EDTA (Gibco), centrifuged at 300 g for 10 minutes, and resuspended in assay media supplemented with 5-10% clinical sample (plasma/serum from peanut allergy patients, dilution varied from sample to sample) to a final concentration of 3×10⁶ cells/ml. If plasma was produced with any anticoagulant other than heparin, the sample was first supplemented with 30 U/ml Heparin (Sodium-Heparin, Sigma) and incubated at room temperature for 10 minutes before adding to cells. Cells were then seeded at 50 μl per well (final 150,000 cells/well) in 96-well flat-bottom tissue culture plates (Greiner bio-one, Austria) and cultured overnight. The next day, activation solutions were prepared by diluting allergens or un-related protein negative controls at varying concentrations in Tyrode's buffer (137 mM NaCl, 2.7 mM KCl, 0.4 mM NaH₂PO₄, 0.5 mM MgCl₂, 1.4 mM CaCl₂, 10 mM Hepes pH 7.3, 5.6 mM glucose, 0.1% BSA, pH adjusted to 7.4, prepared in a water composition of 80% ddw and 20% D20 heavy water, Merck-Sigma Aldrich, Israel). Cells were then washed 3 times with Tyrode's buffer prepared with ddw only, and 100 μl allergen activating solution was added to appropriate wells in duplicates. For each allergen, 5-6 concentrations at 10-fold dilutions were used. Each clinical sample was tested for WT allergen, variant allergens, and an unrelated protein as negative control (KLH, Sigma). Duplicate wells were also prepared with a lysis buffer (Tyrode's buffer with 1% Triton x-100, Fisher Scientific) for measuring total degranulation and with Tyrode's buffer alone for measuring background degranulation. Cells were then incubated for 1 hour at 37° C., 5% CO₂. Immediately after incubation, 30 μl of each well were transferred to a corresponding well in a clear non-binding 96-well plate (Greiner Bio-one) and supplemented with 50 μl PNAG colorimetric substrate (4-Nitrophenyl N-acetyl-β-D-glucosaminide prepared in 0.1M citric acid to final concentration 1.368 mg/ml pH4.5). Reactions were incubated for 1 hour at 37° C. with gentle shaking in the dark and then 100 μl stop solution (0.2M glycine at pH 10.7) was added to halt reaction and develop color. Optical densities were read at 405 nm for signal and at 630 nm for background absorbance using the Synergy LX microplate spectrophotometer reader (Biotek, Vermont). After subtraction of background absorbance, net degranulation was calculated by dividing the OD of each cell by the OD in the corresponding lysis buffer wells (total degranulation) and subtracting the OD of buffer only wells (background degranulation). EC50 values were calculated per allergen and the relative allergenic potency of each allergen variant was calculated by dividing its EC50 by that of the WT allergen. Where EC50 were not derivable, due to low signal, qualitative analysis was performed.

### BAT Assays

Fresh whole blood samples in heparinized tubes (BD biosciences) were divided into 100 ul per tube. Allergens and controls were diluted in RPMI1640 (Biological Industries) to ×2 stocks, added 1:1 to tubes (final volume 200 ul) and incubated for 30 minutes in a 37° C., 5% CO₂ humidified incubator. The dose range used for each allergen was 1-10000 ng/ml. Crude peanut extract (CPE), fMLP and anti-human IgE antibodies were used as positive controls. KLH protein was used as a negative control. The reaction was stopped by incubation on ice for 5 min. A cocktail of fluorophore-conjugated antibodies was added directly to the samples to detect the following markers: CD203c, CD63, HLA-DR, CD45, CD123. Cells are incubated for 30 min on ice. RBC lysis was performed with a kit according to manufacturer's instructions (BD FACS lysing solution), and cells were washed and analyzed by flow cytometry. Cells were gated for basophil detection and activation rate (% CD63-positive basophils) was measured. At least 500 basophils were analyzed per tube. EC50 values were calculated per allergen and the relative allergenic potency of each allergen variant was calculated by dividing its EC50 by that of the WT allergen.

### T Cell Activation Assay

PBMC were isolated from heparinized peanut allergy patient blood samples. Cells were washed with PBS, stained with Celltrace violet (Thermo-fisher) according to the manufacturer's instructions, and seeded in 96-well round bottom plates at 0.2-0.5×10⁶ cells/well (according to available number of cells following purification and staining) in X-vivo15 media supplemented with 5% human AB serum (Biotag) and 1% penicillin-streptomycin solution (Biological industries). Recombinant WT and variant allergens were purified by Rapid Endotoxin Removal Kit (Abeam), tested for residual endotoxin contamination (LAL Chromogenic Endotoxin Quantitation Kit, Pierce), diluted in same media as cells, sterilized by 0.22 M filtration and added to cells to a final concentration of 50 g/ml in 200 μl per well. Unactivated wells (baseline, media only) and each allergen were tested per patient by 3 or more replicate wells. Each assay included healthy donor samples alongside patients as negative controls for assay quality assurance. Final endotoxin levels in wells for all allergens were <0.5 EU. Cells were incubated for 7 days in a 37° C., 5% CO₂ humidified incubator. If media in any of the wells changed to yellow during the incubation period, half of the media was replaced with fresh media for all wells. After 7 days, cells were harvested, stained for viability (LIVE/DEAD stain, Thermo-fisher), stained with anti-CD3 and anti-CD4 fluorophore-conjugated antibodies (Biolegend; USA) and analyzed by flow cytometry. Live T helper cells were gated (LIVE/DEADlowCD4+CD3+) and the percent of proliferating cells (Celltracedim/Total T helper) was measured. A positive result (allergen causes activation of patient T cells) was determined where the mean of allergen-stimulated wells was greater than Mean+3×SD of unstimulated wells.

Circular dichroism spectroscopy is a useful technique for analyzing protein secondary structure and folding properties in solution using very small amounts of protein. It is based on the differential absorbance of left and right circularly polarized light by a chromophore. The CD analysis of proteins is based on the amide chromophore in the far UV region (below 240 nm), as well as information from the aromatic side chains (260-320 nm). For example, α-helical proteins have negative bands at 222 nm and 208 nm and a positive band at 193 nm, whereas proteins with well-defined antiparallel β-pleated sheets (β-sheet) have negative bands at 218 nm and positive bands at 195 nm.

The circular dichroism spectra of the recombinant Ara h proteins were measured on Chirascan CD spectrometer (Applied Photophysics) at Bar Ilan university. Far-UV CD spectra from 200-260 nm were acquired with a 10 mm path-length cuvette. The Ara h recombinant WT and variants were measured in a PBS buffer and concentrations were determined using 280 nm. Spectra were acquired at 25° C. and at elevated temperatures, 20-90° C., to assess the stability.

### Strains, Plasmid and Growth Conditions

Escherichia coli stable (New England Biolabs) were routinely used for all cloning procedures, Escherichia coli OmniMAX™ (Thermo Fisher scientific) were used for phage display libraries screening, Escherichia coli BL21 (DE3) cells were used for scFv purification and Escherichia coli Origami or BL21 De3 (Novagen) were used for Ara h 2 and Ara h 1 purification. All strains were grown on 2YT broth and LB agar plates at 37° C. A phagemid was used for scFvs phage display libraries derived from peanut allergic patients and for scFv purification. tPCR was used to insert a non-specific scFv that was derived from a healthy donor and designed with a non-structured GGGS×4 linker and to add restriction sites at either ends of the scFv segment—NcoI at the 5′ end and NotI at the 3′ end (the modified plasmid was marked internally as pLibGD). Plasmid pET28 (Invitrogen) was used for recombinant purification of Ara h 2 and Ara h 1 and mutants. Transformations for scFv display were performed using SS320 electrocompetent Escherichia coli (Lucigen).

### Example 2: Epitope Mapping and De-Epitoping of Ara h 1 and Ara h 2 Polypeptides

Objective: The overall objective is to develop a basis for defined targeted mutation of allergenic polypeptides that are stable, retain their T cell activation activity, but have reduced binding to IgE allergenic antibodies. For the purpose of immunotherapy, the functionality of these Ara h 1 and Ara h 2 variant polypeptides includes maintaining immunogenicity, e.g., by the ability to activate T-cells. This series of experiments was performed to identify and map conformational and linear epitopes on the peanut allergens Ara h 1 and Ara h 2, based on the binding of specific monoclonal antibodies from peanut allergic patient samples; and to identify amino acid residues within the Ara h 1 and Ara h 2 mAb binding epitopes that contribute to binding, and which when mutated are not predicted to destabilize the protein.

### Results

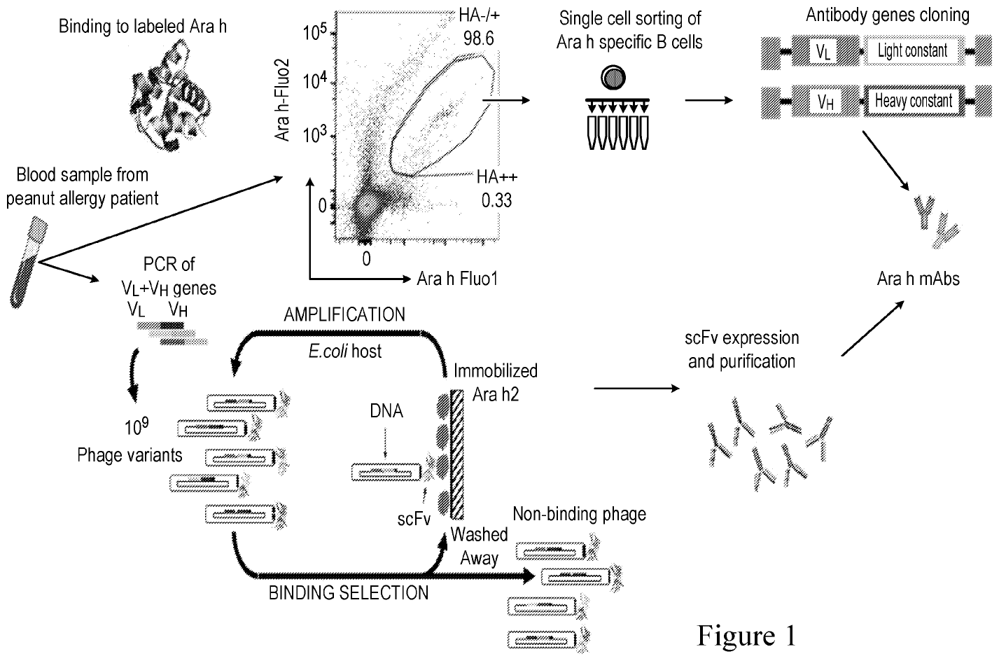

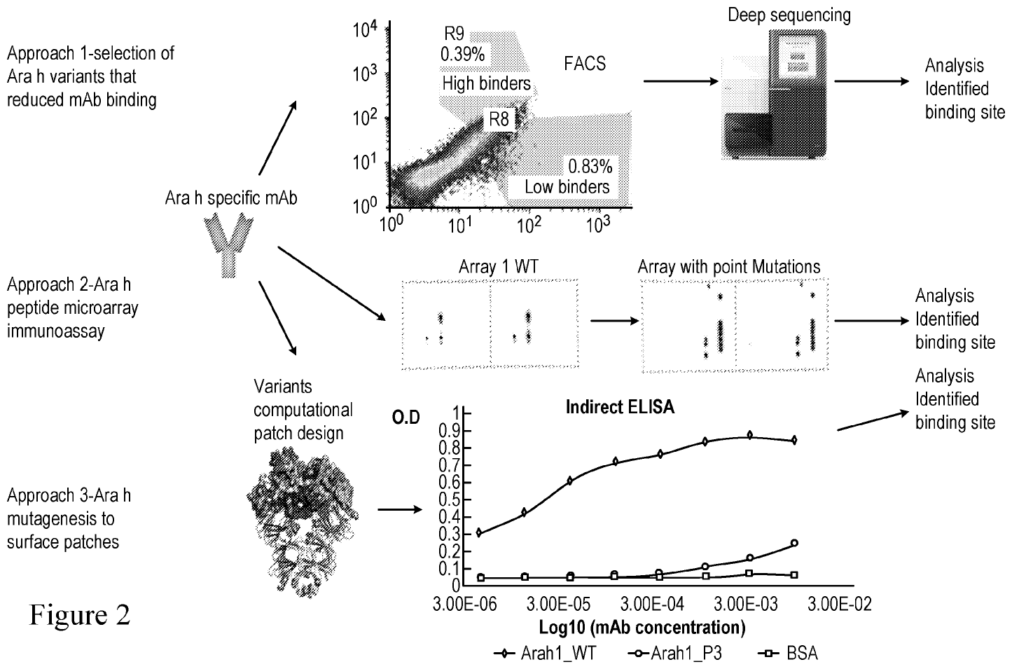

The pipeline for single epitope mapping and de-epitoping of the peanut allergens Ara h 2 and Ara h 1 included two stages—(1) discovery of Ara h 1 and Ara h 2-specific monoclonal antibodies (mAb) from peanut allergic patient samples that exhibit specific IgE binding to Ara h 1 or Ara h 2 (FIG. 1), as measured by ELISA assay and peptide array, and (2) mapping of the epitope that each antibody binds (FIG. 2). Stage 1, mAb discovery, was carried out using scFv phage display libraries by amplification of the variable genes and construction of scFv that are fused to pIII protein and displayed on phages, or by Ara h specific B cells single cell sorting, followed by sequencing of the variable region and production of recombinant mAbs. FIG. 1 presents a schematic description of the process of identifying Ara h 2 antibodies. A similar process was carried out to identify Ara h 1 antibodies.

Briefly, scFv phage display libraries from PBMC of 37 peanut allergic patients were generated as described in Example 1, following a panning process of these libraries, 35 Ara h 1 specific mAbs and 42 Ara h 2 specific mAbs were identified. The scFv mAbs were expressed and purified in E. coli. The epitope mapping procedure, as described below and shown in FIG. 2 was completed for 19 Ara h 1 and 10 Ara h 2 scFV mAbs. From single cell sort analysis of one patient PBMCs, 14 Ara h 2 specific mAbs were identified and expressed in HEK293 cells as IgG and their epitope was mapped in Ara h 2.

In the second stage, the anti-Ara h 1 or anti-Ara h 2 specific purified mAbs were used for epitope mapping in three complementary approaches:

Approach A. Site saturation mutagenesis with yeast surface display (YSD) (Siloto and Weselake (2012) Site saturation mutagenesis: Methods and applications in protein engineering. Biocatalysis and Agricultural Biotechnology, Volume 1(3):181-189) (Cherf G M, Cochran J R. (2015) Applications of Yeast Surface Display for Protein Engineering. Methods Mol Biol. 1319:155-75.)

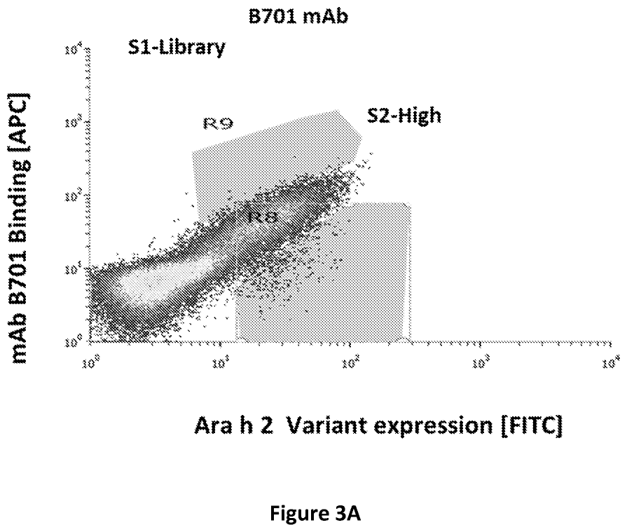

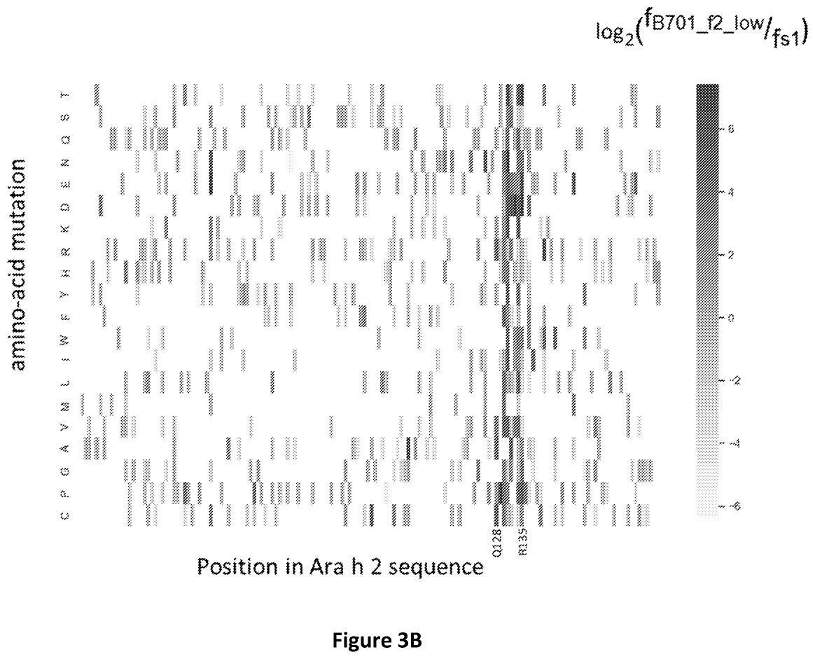

Epitope mapping using Ara h 2 YSD mutagenesis library: For the purpose of epitope mapping, a two-step procedure was performed. First, the Ara h 2 point mutants library was sorted for expression only, collecting those variants that undergo successful YSD, resulting in a sorted library that will be referred to as S1. The threshold for expression was defined as the florescence value that is higher than the unstained cells (background). Each cell that had higher fluorescent signal than the background was collected (S1 lib). Next, S1 library binding to 56 mAbs was assessed. Ara h 2 yeast cell that displayed Ara h 2 variants and exhibited mAb binding signal (APC) in the lower and higher 1% of the population were sorted (Libraries were assigned as S2-mAb-low or S2-mAb-high) See example sort in FIG. 3A, wherein shaded areas R8 and R9 are FACS gates, defining which Ara h 2 expressing yeast cells to collect based on their expression and binding level (R9—Ara h 2 point mutants exhibiting high Ara h 2 binding, R8—mutants exhibiting high expression but low Ara h 2 binding. In some embodiments, the mutants exhibiting lower Ara h 2 binding comprise a technical characteristic of interest.

Deep sequencing was performed to each S2-mAb in order to identify the positions that affect binding to the specific mAb. As the library has undergone a selection for expression and lower mAb binding, sequencing results were analyzed by means of enrichment calculations. Each unique DNA sequence that encodes a point mutant was counted and the fold change in its relative abundance was calculated, to serve as an indirect estimate for the change in mAb binding. Representative results for an example mapping are shown in FIG. 3B.

The population of high affinity Ara h 2 point mutants was compared to the population of low affinity mutants, to allow the identification of mutations that are enriched in the low binding population and not in the high binding population.

From the 56 mAbs that were assessed only 22 mAbs were successfully mapped. A similar approach using YSD is to be carried out for Ara h 1 mutants and mAbs.

Approach B. Structure based in-silico design of surface exposed patches mutagenesis (the patch approach was utilized on Ara h 1, not on Ara h 2). The core domain of Ara h 1 (SEQ ID NO: 66; amino acid 87-503 of SEQ ID NO: 65) has a well-defined trimer structure. Data on surface exposure (calculated by the FreeSASA software (Simon Mitternacht (2016) FreeSASA: An opensource C library for solvent accessible surface area calculation) were combined with evolutionary conservation to mutate surface exposed positions without disrupting the trimeric structure. For each generated variant, a set of 4-7 structurally close surface positions were selected and mutated to alanine, wherever a position exhibited low evolutionary conservation in a multiple sequence alignment, or to an amino-acid identified among its homologs for more conserved amino-acids. Conservation was assessed by collecting homologs of Ara h 1 via BLAST (Altschul, Stephen F., et al. “Gapped BLAST and PSI-BLAST: a new generation of protein database search programs.” Nucleic acids research 25.17 (1997): 3389-3402) with default parameters and generation of a multiple sequence alignment using clustal omega (Sievers, Fabian, et al. “Fast, scalable generation of high-quality protein multiple sequence alignments using Clustal Omega.” Molecular systems biology 7.1 (2011): 539). This surface patches mutagenesis approach was used to map conformational epitopes of Ara h 1. All patches were mutated, and the recombinant variants were tested for binding to Ara h 1 mAbs by ELISA.

### Conformational Epitopes

At least five (5) conformational epitopes were identified in Ara h 1: C4—comprising at least residues 84, 87, 88, 96, 99, 419, and 422 of SEQ ID NO: 65, C3—comprising at least residues 322, 334, 455, and 464 of SEQ ID NO: 65, C1—comprising at least residues 462, 484, 485, 488, 491, and 494, L1 at least comprising residues 194-197 of SEQ ID NO: 65 and L2 at least comprising residues 287-295 of SEQ ID NO: 65.

At least five (5) conformational epitopes were identified in Ara h 2: C3—comprising at least residues 14, 15, 16, 17, 18, 19, 20, 21, 22, 24, 25, 27, 28, 80, 97, 99, 100, 102, 103, 104, 105, 107, 108, 109, 110, 111, 112, and 113 of SEQ ID NO: 3, C1—comprising at least residues 82, 83, 86, 87, 90, and 92 of SEQ ID NO: 3, C2—comprising at least residues 97, 99, 100, 102, 103, 104, 105, 107, 108, 127, 128, 129, 130, 134, 136, 137, 138, 139, 140, 141, 142, and 143 of SEQ ID NO: 3, C4—comprising at least residues 123, 124, 125, 127, 138, 139, 140, 141, 142, 143, and 144 of SEQ ID NO: 3, and L4 comprising at least residues 109-115 of SEQ ID NO: 3.

Approach C. Peptide microarray assay was performed as described in Example 1 with purified mAbs (scFv or IgG) to map some of the consecutive epitopes on the allergens Ara h 1 and Ara h 2 (FIG. 2). This method was also used to validate the data from the YSD saturation or the patch approach for linear epitopes. In Ara h 2, two linear epitopes were identified (L1-residues 12-20 of SEQ ID NO: 3 and L3—residues 44-69 of SEQ ID NO: 3), confirmed with 14 mAbs that were analyzed with the peptide array. In Ara h 1, six linear epitopes were identified (L7—residues 24-30 of SEQ ID NO: 65, L6—residues 209-215 of SEQ ID NO: 65, L3—residues 331-336 of SEQ ID NO: 65, L4—residues 417-422 of SEQ ID NO: 65, L5—residues 480-487 of SEQ ID NO: 65, and L8—residues 260-267 of SEQ ID NO: 65) all confirm with 13 mAbs analyzed using the peptide microarray. An additional Ara h specific point mutation peptide array was used to find hot spots in the 15mer peptide that are crucial for mAb binding.

IgE epitope mapping and de-epitoping of Ara h 1 based on sera from allergic patients (See Example 3). X, Critical positions in 16 epitopes were identified using peptide microarray similar to the process in Approach C. However, instead of mapping isolated monoclonal antibodies, the IgE repertoire from allergic patient sera was used as described in Example 3. Linear epitopes identified include La9—comprising at least residue 12 of SEQ ID NO: 65, La16—comprising at least residue 42 of SEQ ID NO: 65, La23—comprising at least residue 52 of SEQ ID NO: 65, La13—comprising at least residues 57, and 58 of SEQ ID NO: 65, La17—comprising at least residue 73 of SEQ ID NO: 65, La10—comprising at least residues 231, 234, 238, and 249 of SEQ ID NO: 65, La11—comprising at least residue 245 of SEQ ID NO: 65, La21 comprising at least residues 278 and 283 of SEQ ID NO: 65, La12—comprising at least residues 312 and 318 of SEQ ID NO: 65, La22—comprising at least residue 378 of SEQ ID NO: 65, La24 comprising at least residue 441 of SEQ ID NO: 65, La18—comprising at least residue 443 of SEQ ID NO: 65, La14—comprising at least residue 445 of SEQ ID NO: 65, La19—comprising at least residue 463 of SEQ ID NO: 65, La15—comprising at least residue 500 of SEQ ID NO: 65, and La20—comprising at least residue 523 of SEQ ID NO: 65.

### Summary

Table 1 summarizes embodiments of the Ara h 1 variants with mutations at positions with respect to WT Ara h 1, amino acid mutations, and epitopes thereof. Bold letters in the left-hand most column and mutations column designate Primary Hot-Spot; italicized letters designate Secondary Hot-Spots. The mutation/epitope details presented in Table 1 were collated from the results of Example 2 and Example 3.

TABLE 1
Ara h 1 Variants

| WT Ara h 1 | Variant | | |
|---|---|---|---|
| SEQ ID | Positions | | |
| NO: 65 | SEQ ID | | |
| (AA 85-626 | NO: 65 & | | |
| Uniprot | SEQ ID | | |
| P43238) | NO: 67 | Mutations | Epitopes |
| R | 1 | | |
| S | 2 | | |
| P | 3 | | |
| P | 4 | | |
| G | 5 | | |
| E | 6 | | La9 |
| R | 7 | | La9 |
| T | 8 | | La9 |
| R | 9 | | La9 |
| G | 10 | | La9 |
| R | 11 | | La9 |
| Q | 12 | K, A | La9 |
| P | 13 | | La9 |
| G | 14 | | La9 |
| D | 15 | | La9 |
| Y | 16 | | La9 |
| D | 17 | | La9 |
| D | 18 | | |
| D | 19 | | |
| R | 20 | | L7 |
| R | 21 | | L7 |
| Q | 22 | | L7 |
| P | 23 | | L7 |
| R | 24 | V, E | L7 |
| R | 25 | | L7 |
| E | 26 | | L7 |
| E | 27 | A, H | L7 |
| G | 28 | | L7 |
| G | 29 | | L7 |
| R | 30 | E, A | L7 |
| W | 31 | | L7 |
| G | 32 | | L7 |
| P | 33 | | |
| A | 34 | | |
| G | 35 | | |
| P | 36 | | |
| R | 37 | | |
| E | 38 | | |
| R | 39 | | |
| E | 40 | | |
| R | 41 | | La16 |
| E | 42 | L, K | La16 |
| E | 43 | | La16 |
| D | 44 | | La16 |
| W | 45 | | La16 |
| R | 46 | | La16 |
| Q | 47 | | La16 |
| P | 48 | | La16 |
| R | 49 | | La16 |
| E | 50 | | La16 |
| D | 51 | | La16 |
| W | 52 | T, L | La16 |
| R | 53 | | La16 |
| R | 54 | | |
| P | 55 | | La13 |
| S | 56 | | La13 |
| H | 57 | D, L | La13 |
| Q | 58 | S, R | La13 |
| Q | 59 | | La13 |
| P | 60 | | La13 |
| R | 61 | | La13 |
| K | 62 | | |
| I | 63 | | |
| R | 64 | | |
| P | 65 | | |
| E | 66 | | |
| G | 67 | | |
| R | 68 | | |
| E | 69 | | La17 |
| G | 70 | | La17 |
| E | 71 | | La17 |
| Q | 72 | | La17 |
| E | 73 | A, M | La17 |
| W | 74 | | La17 |
| G | 75 | | La17 |
| T | 76 | | La17 |
| P | 77 | | La17 |
| G | 78 | | La17 |
| S | 79 | | |
| H | 80 | | |
| V | 81 | | |
| R | 82 | | |
| E | 83 | | |
| E | 84 | A | C4 |
| T | 85 | | |
| S | 86 | | |
| R | 87 | A | C4 |
| N | 88 | A | C4 |
| N | 89 | | |
| P | 90 | | |
| F | 91 | | |
| Y | 92 | | |
| F | 93 | | |
| P | 94 | | |
| S | 95 | | |
| R | 96 | A | C4 |
| R | 97 | | |
| F | 98 | | |
| S | 99 | A | C4 |
| T | 100 | | |
| R | 101 | | |
| Y | 102 | | |
| G | 103 | | |
| N | 104 | | |
| Q | 105 | | |
| N | 106 | | |
| G | 107 | | |
| R | 108 | | |
| I | 109 | | |
| R | 110 | | |
| V | 111 | | |
| L | 112 | | |
| Q | 113 | | |
| R | 114 | | |
| F | 115 | | |
| D | 116 | | |
| Q | 117 | | |
| R | 118 | | |
| S | 119 | | |
| R | 120 | | |
| Q | 121 | | |
| F | 122 | | |
| Q | 123 | | |
| N | 124 | | |
| L | 125 | | |
| Q | 126 | | |
| N | 127 | | |
| H | 128 | | |
| R | 129 | | |
| I | 130 | | |
| V | 131 | | |
| Q | 132 | | |
| I | 133 | | |
| E | 134 | | |
| A | 135 | | |
| K | 136 | | |
| P | 137 | | |
| N | 138 | | |
| T | 139 | | |
| L | 140 | | |
| V | 141 | | |
| L | 142 | | |
| P | 143 | | |
| K | 144 | | |
| H | 145 | | |
| A | 146 | | |
| D | 147 | | |
| A | 148 | | |
| D | 149 | | |
| N | 150 | | |
| I | 151 | | |
| L | 152 | | |
| V | 153 | | |
| I | 154 | | |
| 0 | 155 | | |
| Q | 156 | | |
| G | 157 | | |
| Q | 158 | | |
| A | 159 | | |
| T | 160 | | |
| V | 161 | | |
| T | 162 | | |
| V | 163 | | |
| A | 164 | | |
| N | 165 | | |
| G | 166 | | |
| N | 167 | R, D | |
| N | 168 | | |
| R | 169 | | |
| K | 170 | | |
| S | 171 | | |
| F | 172 | | |
| N | 173 | | |
| L | 174 | | |
| D | 175 | | |
| E | 176 | | |
| G | 177 | | |
| H | 178 | | |
| A | 179 | | |
| L | 180 | | |
| R | 181 | | |
| I | 182 | | |
| P | 183 | | |
| S | 184 | | |
| G | 185 | | |
| F | 186 | | |
| I | 187 | | |
| S | 188 | | |
| Y | 189 | | |
| I | 190 | | |
| L | 191 | | |
| N | 192 | | |
| R | 193 | | |
| H | 194 | D | L1 |
| D | 195 | A | L1 |
| N | 196 | H | L1 |
| Q | 197 | A | L1 |
| N | 198 | | L1 |
| L | 199 | | |
| R | 200 | V, A, Q | C2 |
| V | 201 | | |
| A | 202 | | |
| K | 203 | | |
| I | 204 | | |
| S | 205 | | |
| M | 206 | | |
| P | 207 | | |
| V | 208 | | |
| N | 209 | S | L6 |
| T | 210 | | L6 |
| P | 211 | | L6 |
| G | 212 | | L6 |
| Q | 213 | H | L6 |
| F | 214 | | L6 |
| E | 215 | R, D, L, I, F, A | L6 |
| D | 216 | | L6 |
| F | 217 | | L6 |
| F | 218 | H, L | L6 |
| P | 219 | | L6 |
| A | 220 | | L6 |
| S | 221 | | L6 |
| S | 222 | | L6 |
| R | 223 | A, D, Q, E | L6 |
| D | 224 | | |
| Q | 225 | | |
| S | 226 | | La10 |
| S | 227 | | La10 |
| Y | 228 | | La10 |
| L | 229 | | La10 |
| Q | 230 | | La10 |
| G | 231 | A | La10 |
| F | 232 | | La10 |
| S | 233 | | La10 |
| R | 234 | E, Q, K | La10 |
| N | 235 | | La10 |
| T | 236 | | La10 |
| L | 237 | | La10 |
| E | 238 | Q | La10 |
| A | 239 | | |
| A | 240 | | |
| F | 241 | | La11 |
| N | 242 | | La11 |
| A | 243 | | La11 |
| E | 244 | | La11 |
| F | 245 | R, Y, A, M | La11 |
| N | 246 | | La11 |
| E | 247 | | La11 |
| I | 248 | | La11 |
| R | 249 | N | La11 |
| R | 250 | | La11 |
| V | 251 | | La11 |
| L | 252 | | La11 |
| L | 253 | | |
| E | 254 | | |
| E | 255 | | |
| N | 256 | | |
| A | 257 | | L8 |
| G | 258 | | L8 |
| G | 259 | | L8 |
| E | 260 | K | L8 |
| Q | 261 | R | L8 |
| E | 262 | | L8 |
| E | 263 | K, L | L8 |
| R | 264 | | L8 |
| G | 265 | S | L8 |
| Q | 266 | R, L | L8 |
| R | 267 | E | L8 |
| R | 268 | | L8 |
| W | 269 | | L8 |
| S | 270 | | |
| T | 271 | | |
| R | 272 | | |
| S | 273 | | |
| S | 274 | | La21 |
| E | 275 | | La21 |
| N | 276 | | La21 |
| N | 277 | | La21 |
| E | 278 | R | La21 |
| G | 279 | | La21 |
| V | 280 | | La21 |
| I | 281 | | La21 |
| V | 282 | | La21 |
| K | 283 | E | La21 |
| V | 284 | | La21 |
| S | 285 | | |
| K | 286 | | L2 |
| E | 287 | D | L2 |
| H | 288 | Q | L2 |
| V | 289 | | L2 |
| E | 290 | R | L2 |
| E | 291 | | L2 |
| L | 292 | | L2 |
| T | 293 | | L2 |
| K | 294 | E | L2 |
| H | 295 | A | L2 |
| A | 296 | | |
| K | 297 | | |
| S | 298 | | |
| V | 299 | | |
| S | 300 | | |
| K | 301 | | |
| K | 302 | | |
| G | 303 | | |
| S | 304 | | |
| E | 305 | | |
| E | 306 | | |
| E | 307 | | |
| G | 308 | | |
| D | 309 | R | La12 |
| I | 310 | | La12 |
| T | 311 | | La12 |
| N | 312 | H, A | La12 |
| P | 313 | | La12 |
| I | 314 | | La12 |
| N | 315 | | La12 |
| L | 316 | | La12 |
| R | 317 | | La12 |
| E | 318 | H | La12 |
| G | 319 | | La12 |
| E | 320 | | La12 |
| P | 321 | | |
| D | 322 | A, K | C3 |
| L | 323 | | |
| S | 324 | | |
| N | 325 | | |
| N | 326 | | |
| F | 327 | | L3 |
| G | 328 | | L3 |
| K | 329 | | L3 |
| L | 330 | | L3 |
| F | 331 | H, W | L3 |
| E | 332 | | L3 |
| V | 333 | | L3 |
| K | 334 | D, N, A | C3 |
| P | 335 | | L3 |
| D | 336 | R, S | L3 |
| K | 337 | | L3 |
| K | 338 | | L3 |
| N | 339 | | L3 |
| P | 340 | | |
| Q | 341 | | |
| L | 342 | | |
| Q | 343 | | |
| D | 344 | | |
| L | 345 | | |
| D | 346 | | |
| M | 347 | | |
| M | 348 | | |
| L | 349 | | |
| T | 350 | | |
| C | 351 | | |
| V | 352 | | |
| E | 353 | | |
| I | 354 | | |
| K | 355 | | |
| E | 356 | | |
| G | 357 | | |
| A | 358 | | |
| L | 359 | | |
| M | 360 | | |
| L | 361 | | |
| P | 362 | | |
| H | 363 | | |
| F | 364 | | |
| N | 365 | | |
| S | 366 | | |
| K | 367 | | |
| A | 368 | | |
| M | 369 | | |
| V | 370 | | |
| I | 371 | | La22 |
| V | 372 | | La22 |
| V | 373 | | La22 |
| V | 374 | | La22 |
| N | 375 | | La22 |
| K | 376 | | La22 |
| G | 377 | | La22 |
| T | 378 | K, E | La22 |
| G | 379 | | La22 |
| N | 380 | | La22 |
| L | 381 | | La22 |
| E | 382 | | La22 |
| L | 383 | | |
| V | 384 | | |
| A | 385 | | |
| V | 386 | | |
| R | 387 | | |
| K | 388 | | |
| E | 389 | | |
| Q | 390 | | |
| Q | 391 | | |
| Q | 392 | | |
| R | 393 | | |
| G | 394 | | |
| R | 395 | | |
| R | 396 | | |
| E | 397 | | |
| E | 398 | | |
| E | 399 | | |
| E | 400 | | |
| D | 401 | | |
| E | 402 | | |
| D | 403 | | |
| E | 404 | | |
| E | 405 | | |
| E | 406 | | |
| E | 407 | | |
| G | 408 | | |
| S | 409 | | |
| N | 410 | | |
| R | 411 | | |
| E | 412 | | |
| V | 413 | | L4 |
| R | 414 | | L4 |
| R | 415 | | L4 |
| Y | 416 | | L4 |
| T | 417 | R | L4 |
| A | 418 | | L4 |
| R | 419 | E, V, A | L4, C4 |
| L | 420 | | L4 |
| K | 421 | S, E | L4 |
| E | 422 | R, A | L4, C4 |
| G | 423 | | L4 |
| D | 424 | | L4 |
| V | 425 | | L4 |
| F | 426 | | L4 |
| I | 427 | | L4 |
| M | 428 | | L4 |
| P | 429 | | |
| A | 430 | | |
| A | 431 | | |
| H | 432 | D, I, L | |
| P | 433 | | |
| V | 434 | | |
| A | 435 | | |
| I | 436 | | |
| N | 437 | | |
| A | 438 | | |
| S | 439 | | |
| S | 440 | | |
| E | 441 | N | La18 |
| L | 442 | | La18 |
| H | 443 | A | La18 |
| L | 444 | | |
| L | 445 | V, I | La14 |
| G | 446 | | La14 |
| F | 447 | | La14 |
| G | 448 | | La14 |
| I | 449 | | |
| N | 450 | | |
| A | 451 | | |
| E | 452 | | |
| N | 453 | | |
| N | 454 | | |
| H | 455 | A, F, Y, D, N, Q | C3, La19 |
| R | 456 | | La19 |
| I | 457 | | La19 |
| F | 458 | | La19 |
| L | 459 | | La19 |
| A | 460 | | La19 |
| G | 461 | | La19 |
| D | 462 | A, K, T, R | C1, La19 |
| K | 463 | S, E | La19 |
| D | 464 | A, S, R, H, F | C3, La19 |
| N | 465 | | La19 |
| V | 466 | | |
| I | 467 | | |
| D | 468 | | |
| Q | 469 | | |
| I | 470 | | |
| E | 471 | | |
| K | 472 | | |
| Q | 473 | | |
| A | 474 | | |
| K | 475 | | |
| D | 476 | | |
| L | 477 | | |
| A | 478 | | L5 |
| F | 479 | | L5 |
| P | 480 | Q, S | L5 |
| G | 481 | A, S | L5 |
| S | 482 | | L5 |
| G | 483 | | L5 |
| E | 484 | R, S, A, M | L5C1 |
| Q | 485 | A | L5, C1 |
| V | 486 | | L5 |
| E | 487 | S, K | L5 |
| K | 488 | A | C1 |
| L | 489 | | L5 |
| I | 490 | | L5 |
| K | 491 | A, E, S | C1 |
| N | 492 | | |
| Q | 493 | | |
| K | 494 | A, E, N, D | C1 |
| E | 495 | | La15 |
| S | 496 | | La15 |
| H | 497 | | La15 |
| F | 498 | | La15 |
| V | 499 | | La15 |
| S | 500 | K, E, I | La15 |
| A | 501 | | La15 |
| R | 502 | | La15 |
| P | 503 | | La15 |
| Q | 504 | | La15 |
| S | 505 | | |
| Q | 506 | | |
| S | 507 | | |
| Q | 508 | | |
| S | 509 | | |
| P | 510 | | |
| S | 511 | | |
| S | 512 | | |
| P | 513 | | |
| E | 514 | | |
| K | 515 | | |
| E | 516 | | |
| S | 517 | | |
| P | 518 | | La20 |
| E | 519 | | La20 |
| K | 520 | | La20 |
| E | 521 | | La20 |
| D | 522 | | La20 |
| Q | 523 | A, K | La20 |
| E | 524 | | La20 |
| E | 525 | | La20 |
| E | 526 | | La20 |
| N | 527 | | La20 |
| Q | 528 | | La20 |
| G | 529 | | La20 |
| G | 530 | | La20 |
| K | 531 | | |
| G | 532 | | |
| P | 533 | | |
| L | 534 | | |
| L | 535 | | |
| S | 536 | | |
| I | 537 | | |
| L | 538 | | |
| K | 539 | | |
| A | 540 | | |
| F | 541 | | |
| N | 542 | | |

Table 2 summarizes embodiments of the Ara h 2 variants with mutations at positions with respect to WT Ara h 2, amino acid mutations, and epitopes thereof. Bold letters in the left-hand most column designate Primary Hot-Spot; italicized letters designate Secondary Hot-Spots. Bold letters in the “Mutations” column designate mutations of the Ara h 2 variant B1001.

TABLE 2
Ara h 2 Variants

| | Variant | SEQ ID | | |
|---|---|---|---|---|
| WT | Positions | NO: 1 | | |
| amino- | SEQ ID | Uniprot | | |
| acid | NO: 3 & | Q6PSU2 | | |
| SEQ ID | SEQ ID | (number- | | Epi- |
| NO: 2 | NO: 4 | ing) | Mutations | topes |
| M | | | | |
| S | 1 | 20 | | |
| A | 2 | 21 | | |
| R | 3 | 22 | | |
| Q | 4 | 23 | | |
| Q | 5 | 24 | | |
| W | 6 | 25 | V | |
| E | 7 | 26 | | |
| L | 8 | 27 | | |
| Q | 9 | 28 | | |
| G | 10 | 29 | | |
| D | 11 | 30 | A, E, F, I, K, | |
| R | 12 | 31 | W, N, Q, E, D, | L1 |
| | | | T, S, G, P, C, | |
| | | | K, H, Y, W, M, | |
| | | | I, L, V, A | |
| R | 13 | 32 | C, P, V, T, S, | L1 |
| | | | D, H, Y, F, W, | |
| | | | L, M | |
| C | 14 | 33 | D, M, N, Y, F, | L1, C3 |
| | | | I, P, S, T, A, | |
| | | | K | |
| Q | 15 | 34 | R, E, K, Y, W, | L1, C3 |
| | | | F, M, I, V, C, | |
| | | | D, G, A | |
| S | 16 | 35 | R, K, D, Q, T, | L1, C3 |
| | | | M, P, C, E, W | |
| Q | 17 | 36 | E, N, P | L1, C3 |
| L | 18 | 37 | R, K, D, E, N, | L1, C3 |
| | | | H, Y, W, I, P, | |
| | | | C, S, V, G | |
| E | 19 | 38 | V, L, M, F, W, | L1, C3 |
| | | | Y, H, Q, N, A, | |
| | | | S, G, P, C, R, | |
| | | | K, D | |
| R | 20 | 39 | A, S, T, N, Q, | L1, C3 |
| | | | E, K, I, L, M, | |
| | | | F, P, C,G | |
| A | 21 | 40 | D, E, K, R, Y, | C3 |
| | | | F, N,P | |
| N | 22 | 41 | F, Y, W, Q, E, | C3 |
| | | | T, S, A, M, I, | |
| | | | L, C, R, H | |
| L | 23 | 42 | V, W, Y, F, I, | |
| | | | P, T | |
| R | 24 | 43 | D, E, H, K, S, | C3 |
| | | | T, N, Q, L, I, | |
| | | | M, W, Y, F, P, | |
| | | | A, G | |
| P | 25 | 44 | T, V, L, M, F, | C3 |
| | | | W, Y, Q, D, E, | |
| | | | K, C,G | |
| C | 26 | 45 | A | |
| E | 27 | 46 | C, G, A, S, T, | C3 |
| | | | N, D, Q, V, I, | |
| | | | M, F, W, Y, K, | |
| | | | R | |
| Q | 28 | 47 | S, T, V, N, A, | C3 |
| | | | P, I, L, F, Y, | |
| | | | H, R, K, E, D | |
| H | 29 | 48 | | |
| L | 30 | 49 | | |
| M | 31 | 50 | | |
| Q | 32 | 51 | R | |
| K | 33 | 52 | | |
| I | 34 | 53 | | |
| Q | 35 | 54 | | |
| R | 36 | 55 | | |
| D | 37 | 56 | | |
| E | 38 | 57 | | |
| D | 39 | 58 | S | |
| S | 40 | 59 | | |
| Y | 41 | 60 | | |
| G | 42 | 61 | | |
| R | 43 | 62 | | |
| D | 44 | 63 | I, A, C, G, H, | L3 |
| | | | L, F, Y, N, P, | |
| | | | Q, K, E, S, T, | |
| | | | V, M, R | |
| P | 45 | 64 | A, Q, Y, N, E, | L3 |
| | | | M, W | |
| Y | 46 | 65 | T, V, E, H, S, | L3 |
| | | | A, G, Q, N, D, | |
| | | | R, P, M, I, L, | |
| | | | C | |
| S | 47 | 66 | T, N, D, K, Y, | L3 |
| | | | W, F, L, P | |
| P | 48 | 67 | V, G, C, E, H, | L3 |
| | | | Q, F, K, L, I, | |
| | | | W, Y, N, R, S, | |
| | | | T, V, A, D | |
| S | 49 | 68 | D, R, A | L3 |
| Q | 50 | 69 | C | L3 |
| D | 51 | 70 | S, G, Y, F, W, | L3 |
| | | | M, N, Q, E, R, | |
| | | | K, H, T, V, D | |
| P | 52 | 71 | S, T, V, A, F, | L3 |
| | | | Y, M, N, D, Q, | |
| | | | R, K, H | |
| Y | 53 | 72 | T, S, Q, V, A, | L3 |
| | | | G, C, P, M, L, | |
| | | | I, E, H, R, K, | |
| | | | N, D | |
| S | 54 | 73 | E, D, G, K, I, | L3 |
| | | | L, V, H, Y, W, | |
| | | | F, P, A, Δ | |
| P | 55 | 74 | G, A, D, E, F, | L3 |
| | | | Y, H, Q, V, I, | |
| | | | L, M, R, K, S, | |
| | | | T, C, W, Δ | |
| S | 56 | 75 | C, E, V, P, Δ | L3 |
| Q | 57 | 76 | Δ | L3 |
| D | 58 | 77 | H, Q, S, T, G, | L3 |
| | | | Δ | |
| P | 59 | 78 | D, I, K, M, L, | L3 |
| | | | Δ | |
| D | 60 | 79 | N, T, S, Y, H, | L3 |
| | | | V, Δ | |
| R | 61 | 80 | Δ | L3 |
| R | 62 | 81 | Δ | L3 |
| D | 63 | 82 | P, C, F, V, I, | L3 |
| | | | L, M, W, Y, N, | |
| | | | S, T, Q, G, H, | |
| | | | K, R, Δ | |
| P | 64 | 83 | W, Y, H, I, N, | L3 |
| | | | K, T, L, M, I, | |
| | | | Q, D, E, A, C, | |
| | | | G, Δ | |
| Y | 65 | 84 | T, A, N, D, Q, | L3 |
| | | | R, K, H, I, L, | |
| | | | M, V, W, P, G, | |
| | | | C, E, Δ | |
| S | 66 | 85 | T, N, D, R, H, | L3 |
| | | | Q, Y, W, I, L, | |
| | | | V, P, G, Δ | |
| P | 67 | 86 | E, Q, N, R, H, | L3 |
| | | | Y, F, W, M, L, | |
| | | | V, T, S, A, G, | |
| | | | P, Δ | |
| S | 68 | 87 | P, G | L3 |
| P | 69 | 88 | A, Q, Y | L3 |
| Y | 70 | 89 | | |
| D | 71 | 90 | | |
| R | 72 | 91 | | |
| R | 73 | 92 | | |
| G | 74 | 93 | | |
| A | 75 | 94 | | |
| G | 76 | 95 | | |
| S | 77 | 96 | | |
| S | 78 | 97 | | |
| Q | 79 | 98 | | |
| H | 80 | 99 | N, S, T, V, A, | C3 |
| | | | I, L, M, F, Y, | |
| | | | W, C, E, K, R, | |
| | | | G | |
| Q | 81 | 100 | R | |
| E | 82 | 101 | C, F, H, I, K, | C1 |
| | | | L, M, N, R, S, | |
| | | | V, W, Y, A | |
| R | 83 | 102 | D, A, C, F, I, | C1 |
| | | | P, T, V, W, Y, | |
| | | | Q | |
| C | 84 | 103 | A | |
| C | 85 | 104 | A | |
| N | 86 | 105 | Y, F, H, R, E, | C1 |
| | | | C, G, I, L, M, | |
| | | | V, T, S, Q | |
| E | 87 | 106 | F, Y, I, L, M, | C1 |
| | | | V, A, S, Q, R, | |
| | | | K, D, P, N, E | |
| L | 88 | 107 | | |
| N | 89 | 108 | R | |
| E | 90 | 109 | S, P, R, Q | C1 |
| F | 91 | 110 | | |
| E | 92 | 111 | M, F, P | C1 |
| N | 93 | 112 | | |
| N | 94 | 113 | | |
| Q | 95 | 114 | | |
| R | 96 | 115 | A | |
| C | 97 | 116 | P, A, N, D, E, | C2, C3 |
| | | | F, H, Y, I, L, | |
| | | | V, Q, S, R | |
| M | 98 | 117 | | |
| C | 99 | 118 | D, E, Y, H, R, | C2, C3 |
| | | | L, M, Q, S, T, | |
| | | | V, A, W | |
| E | 100 | 119 | C, G, H, I, K, | C2, C3 |
| | | | L, M, Q, R, V, | |
| | | | W, Y, P | |
| A | 101 | 120 | | |
| L | 102 | 121 | M, Q, W | C2, C3 |
| Q | 103 | 122 | A, D, E, G, R, | C2, C3 |
| | | | S | |
| Q | 104 | 123 | L, M, K, R, H, | C2, C3 |
| | | | E, D, A, Y, N, | |
| | | | S, W | |
| I | 105 | 124 | A, T | C2, C3 |
| M | 106 | 125 | | |
| E | 107 | 126 | A, C, F, G, H, | C2, C3 |
| | | | I, K, L, M, Q, | |
| | | | P, R, S, T, V, | |
| | | | W, Y | |
| N | 108 | 127 | T, V, D, E, R, | C2, C3 |
| | | | H, Y, W, I, G, | |
| | | | A, Q, K | |
| Q | 109 | 128 | K, C, S, R, G, | L4, C3 |
| | | | P, Y, W, I, L | |
| S | 110 | 129 | R | L4, C3 |
| D | 111 | 130 | C, I, L, M, P, | L4, C3 |
| | | | Q, K, R, E, Y, | |
| | | | S, V, A | |
| R | 112 | 131 | D, E, K, H, F, | L4, C3 |
| | | | I, M, L, W, A, | |
| | | | V, S, T, N, Q, | |
| | | | C | |
| L | 113 | 132 | D, E, G, H, F, | L4, C3 |
| | | | I, K, R, N, P, | |
| | | | S, T, W, Y | |
| Q | 114 | 133 | H, G, K, N, S, | L4 |
| | | | V, A | |
| G | 115 | 134 | V, D, E, I, L, | L4 |
| | | | K, M, N, S, T, | |
| | | | A, I, W, F, Y, | |
| | | | H | |
| R | 116 | 135 | K, E, A, V, F, | |
| | | | G, H, P, S, T, | |
| | | | N, W | |
| Q | 117 | 136 | A | |
| Q | 118 | 137 | N, R, V, Y | |
| E | 119 | 138 | C, N, S, W | |
| Q | 120 | 139 | | |
| Q | 121 | 140 | | |
| F | 122 | 141 | | |
| K | 123 | 142 | I, Q, A | C4 |
| R | 124 | 143 | D, A, C, F, G, | C4 |
| | | | H, I, N, S, T, | |
| | | | V, Y, L, Q, E | |
| E | 125 | 144 | M, I, L, W, Y, | C4 |
| | | | G, K, N, T, V, | |
| | | | A | |
| L | 126 | 145 | | |
| R | 127 | 146 | H, A, D, E, F, | C2, C4 |
| | | | G, L, N, P, S, | |
| | | | T, W, Y, Q, V | |
| N | 128 | 147 | R | C2 |
| L | 129 | 148 | F, Q | C2 |
| P | 130 | 149 | G, I, L, M, W, | C2 |
| | | | R, H, A, N, V | |
| Q | 131 | 150 | A, R | |
| Q | 132 | 151 | | |
| C | 133 | 152 | A | |
| G | 134 | 153 | R | C2 |
| L | 135 | 154 | | |
| R | 136 | 155 | A, C, E, F, W, | C2 |
| | | | Y, G, N, P, V, | |
| | | | Q | |
| A | 137 | 156 | C, F, W, Y, H, | C2 |
| | | | K, R, N, Q, D, | |
| | | | E, G, I, L, M, | |
| | | | T, V | |
| P | 138 | 157 | C, D, F, G, I, | C2, C4 |
| | | | K, M, N, Q, S, | |
| | | | T, V | |
| Q | 139 | 158 | C, D | C2, C4 |
| R | 140 | 159 | G, A, C, E, Y, | C2, C4 |
| | | | F, H, K, L, M, | |
| | | | N, P, Q, S, V | |
| C | 141 | 160 | D, E, F, I, L, | C2, C4 |
| | | | S, T, V, N, A, | |
| | | | W | |
| D | 142 | 161 | M, A, C, E, F, | C2, C4 |
| | | | G, H, I, K, L, | |
| | | | N, P, Q, R, S, | |
| | | | T, V, W, Y | |
| L | 143 | 162 | D, E, F, G, H, | C2, C4 |
| | | | K, P, Q, N, R, | |
| | | | S, T | |
| E | 144 | 163 | G, V, M, Y, R, | C4 |
| | | | P | |
| V | 145 | 164 | | |
| E | 146 | 165 | R | |
| S | 147 | 166 | | |
| G | 148 | 167 | C, D, E, I, N, | |
| | | | S, W, Y | |
| G | 149 | 168 | E, F, M, Q, W, | |
| | | | Y | |
| R | 150 | 169 | A, C, E, G, P | |
| D | 151 | 170 | G, H, I, L, M, | |
| | | | N, P, Q, R, S, | |
| | | | W, Y | |
| R | 152 | 171 | S, Y, K, M, P | |
| Y | 153 | 172 | K, R, P | |

### Example 3: IgE Epitope Mapping and De-Epitoping Based on Allergic Patients' Sera Samples

Objective: Following through with the overall objective of developing a basis for defined targeted mutation of allergenic polypeptides that are stable, retain their functional characteristics, but have reduced binding to IgE allergenic antibodies, the objective of these experiments was to identify the consecutive (linear) IgE epitopes for peanut patients' sera/plasma and analyze mutant variants thereof.

### Results

The same peptide arrays as in the purified mAbs analysis procedure were used to identify all consecutive epitopes on the allergens Ara h 1 and Ara h 2, of polyclonal IgE from allergic patient sera. These arrays were assayed with the sera of 250 peanut allergic patients, testing for sera-derived IgE binding of Ara h 1—and Ara h 2-derived peptides. Of the tested sera, 192 and 168 slides identified IgE binding to at least one peptide from Ara h 1 or Ara h 2, respectively. Analysis and clustering of peptide array results allowed for the mapping of all linear epitopes of the proteins (data not shown).

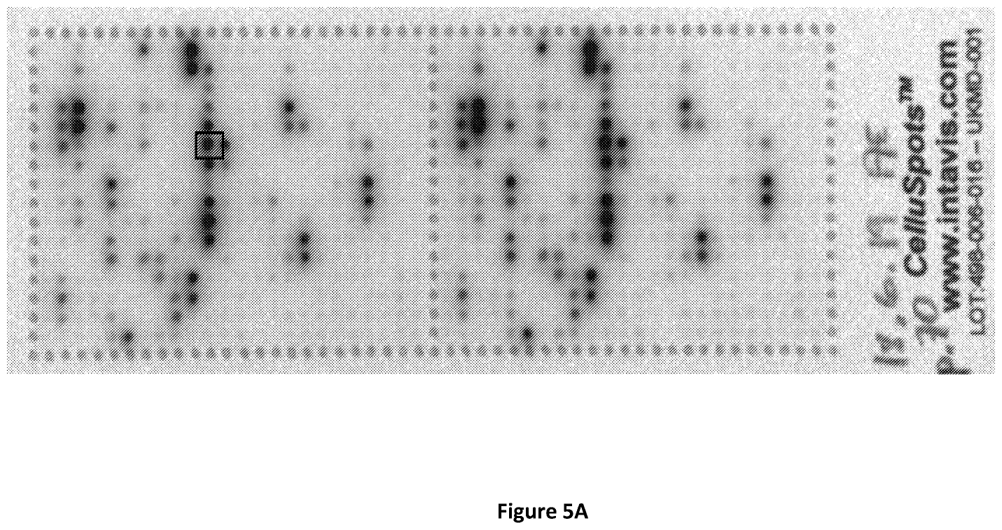

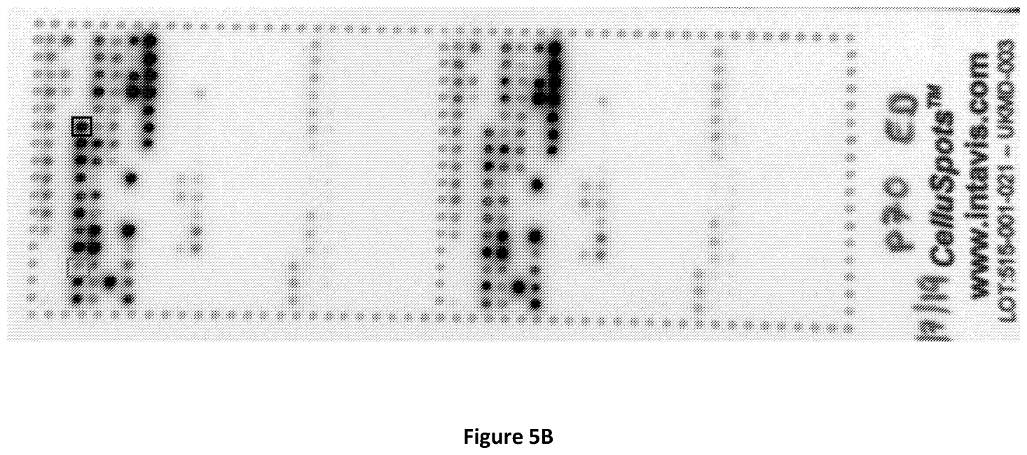

Based on the mapped epitopes, two additional arrays were synthesized, where for each Ara h 1 or Ara h 2 mapped epitope, the WT peptide was spotted along mutated peptides that were computationally designed to diminish IgE binding. Peptides were 15 amino acids long and included either point mutations or double substitution mutations. Next, sera mapped to Ara h 1 or Ara h 2 were assayed with the mutated “de-epitoping” spots containing arrays to screen for those peptides showing the most significant decrease in binding. The results of representative arrays are shown for the linear epitope mapping (FIG. 5A) and de-epitoping (FIG. 5B) of Ara h 2 serum P70. Furthermore, the mutation/epitope details presented in Table 1 of Example 2 were collated from the results of both Example 2 and Example 3.

### Example 4: Mutation of Single or Multiple Epitopes

Objective: Using the data collected in Examples 2 and 3, variants were designed with combinations of mutations.

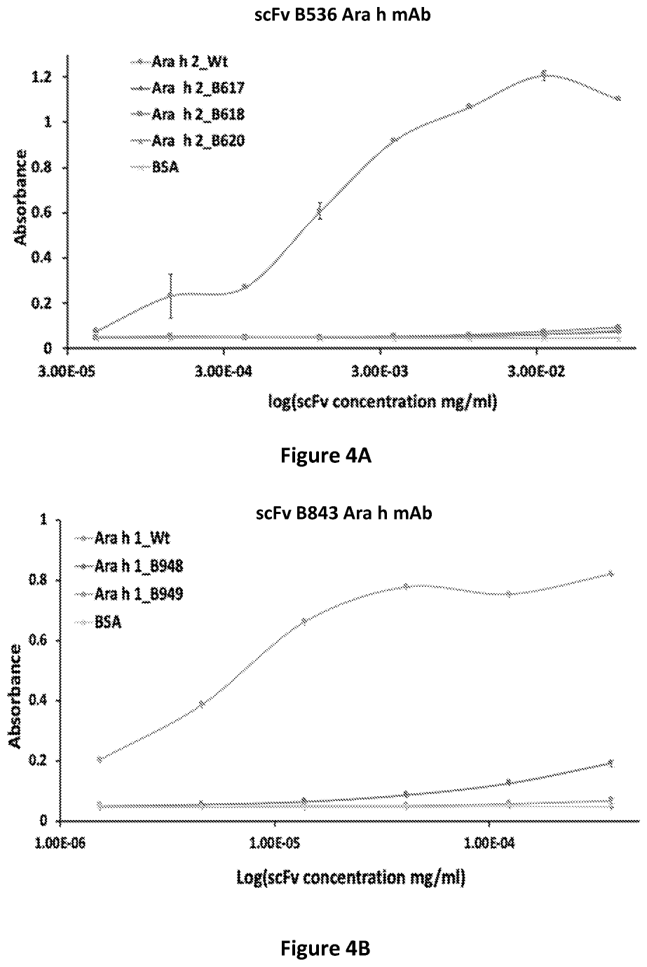

Results: Mutations were combined based on computational prediction of the energetic effect of the mutations on protein stability. Calculations were performed starting from the solved structures of Ara h 2 (PDB accession 3ob4) and Ara h 1 (PDB accession 3s7i). At this stage, each variant is mutated in one epitope. In other embodiments, several epitopes could be mutated within a single variant. In other embodiments, a single variant has multiple epitopes mutated at one time. Mutations included 1-7 substitution mutations within the epitope. The designed variants were produced in E. coli and tested to verify a reduction in binding to Ara h proteins by indirect Enzyme-Linked ImmunoSorbent Assay (ELISA). Representative results are shown for Ara h 1 mAb B843 (FIG. 4B) and Ara h 2 mAb B536 (FIG. 4A) tested against three Ara h 1 or Ara h 2 variants, respectively, mutated at the mapped epitope region.

Tables 3-5 present Ara h 2 variants that were de-epitoped at a single epitope and the effect thereof on binding to specific mAb. Table 6 presents Ara h 2 variants that were de-epitoped at multiple epitopes. Table 7 presents Ara h 1 variants that were de-epitoped at a single epitope (SEQ ID NOs: 68-87) and at multiple epitopes at one time (SEQ ID NOs: 88-161, 174,176, 178, 180, 182, 184, 193, 194, 211-246).

TABLE 3
Ara h 2 Variants with abolished binding to specific mAb

| Epitope | | |
|---|---|---|
| name | Variant name | Variant mutations |
| L1 | Ara h 2_B493 | R12S + R13S + Q15E + S16R + E19D |
| L1 | Ara h 2_B572 | R12N + Q15R + S16R |
| L1 | Ara h 2_B573 | R12Y + R13M + Q15M + S16R |
| L1 | Ara h 2_B574 | R12N + R13H + S16R |
| L1 | Ara h 2_B575 | R12N + Q15R + S16M + R20S |
| L3 | Ara h 2_B549 | D44S + Y46T + P48A + D51S + Y53V + |
| | | P55G + D63T + Y65I + P67A |
| L3 | Ara h 2_B550 | D44I + Y46T + P48V + D51S + Y53T + |
| | | P55G + D63P + Y65T + P67E |
| L3 | Ara h 2_B577 | D44L + P48G + D51W + Y53E + P55W + |
| | | Y65T + P67G |
| L3 | Ara h 2_B987 | Y46A + Y53A + Y65A |
| L4 | Ara h 2_B712 | G115V |
| L4 | Ara h 2_B713 | Q109K + G115S |
| L4 | Ara h 2_B714 | R112H + G115A |
| L4 | Ara h 2_B715 | R112H + G115S |
| L4 | Ara h 2_B716 | R112H + G115V |
| L4 | Ara h 2_B717 | Q109K + G115A |
| L4 | Ara h 2_B718 | Q109K + G115V |
| L4 | Ara h 2_B719 | Q114H + G115A |
| L4 | Ara h 2_B720 | Q114H + G115S |
| L4 | Ara h 2_B721 | Q114H + G115V |
| C1 | Ara h 2_B569 | N86Y |
| C2 | Ara h 2_B622 | E100V + E107K + A137R |
| C2 | Ara h 2_B623 | Q104L + E107A + R127H + R140G |
| C2 | Ara h 2_B624 | E100V + A137F + R140G + D142H |
| C2 | Ara h 2_B625 | Q104L + E107K + R140H + D142A |
| C2 | Ara h 2_B626 | E100V + E107A + R127H + D142Y |
| C3 | Ara h 2_B617 | N22F + Q28S + H80N + N108T |
| C3 | Ara h 2_B618 | Q15R + A21P + R24D + H80R + Q104L |
| C3 | Ara h 2_B620 | N22F + E27R + N108T + S110R |
| C3 | Ara h 2_B621 | R24L + Q28N + E107A + N108K |
| C4 | Ara h 2_B754 | K123I + R124D + E125M + R140E |
| C4 | Ara h 2_B755 | K123I + E125M + N128R + D142M |
| C4 | Ara h 2_B756 | R124Y + E125I + R140V + D142Y + E144P |

TABLE 4
Ara h 2 Variants with reduced binding to specific mAb

| | | |
|---|---|---|
| L3 | Ara h 2_B547 | D44S + Y46T, P48L, D51S, P55L, D63T, Y65K, |
| | | P67E |
| L3 | Ara h 2_B548 | D44M + Y46V + P48V + D51F + Y53V + P55Y + |
| | | D63N + Y65K + P67A |
| C1 | Ara h 2_B556 | E87D + G134R |
| C1 | Ara h 2_B557 | N86Q |
| C1 | Ara h 2_B560 | G134R |
| C1 | Ara h 2_B637 | P25F + D63T + N86Q + E92F |
| C1 | Ara h 2_B638 | P67A + E82A + N86Y + R136Q |
| C1 | Ara h 2_B639 | R83V + N86R + E92P + G134R |
| C1 | Ara h 2_B640 | D63T + R83Y + N86Q + R136Q |
| C1 | Ara h 2_B685 | P67A + E82A + N86Y |
| L4 | Ara h 2_B710 | G115A |
| L4 | Ara h 2_B711 | G115S |
| C3 | Ara h 2_B619 | R24S + Q104L + N108T |
| C4 | Ara h 2_B725 | E125A + D142A |
| C4 | Ara h 2_B726 | R124L + D142V |
| C4 | Ara h 2_B727 | R124Y + D142A |
| C4 | Ara h 2_B728 | R124Y + D142H |
| C4 | Ara h 2_B729 | R124Y + D142V |

TABLE 5
Ara h 2 Variants that do not have reduced binding to specific mAb

| | | |
|---|---|---|
| C1 | Ara h 2_B559 | N86R + L135K |
| C1 | Ara h 2_B570 | E87I |
| C2 | Ara h 2_B627 | E100V + E107K + A137R + D142A |

TABLE 6
Amino Acid Sequences of Ara h 2 Variants

| Ara h 2 Variants | SEQ ID NO: |
|---|---|
| Ara h 2_B1001 | 10 |
| Ara h 2_B764 | 11 |
| Ara h 2_B761 | 12 |
| Ara h 2_B767 | 13 |
| Ara h 2_B768 | 14 |
| Ara h 2_B769 | 15 |
| Ara h 2_B770 | 16 |
| Ara h 2_B771 | 17 |
| Ara h 2_B772 | 18 |
| Ara h 2_B773 | 19 |
| Ara h 2_B774 | 20 |
| Ara h 2_B775 | 21 |
| Ara h 2_B776 | 22 |
| Ara h 2_B777 | 23 |
| Ara h 2_B778 | 24 |
| Ara h 2_B779 | 25 |
| Ara h 2_B780 | 26 |
| Ara h 2_B781 | 27 |
| Ara h 2_B782 | 28 |
| Ara h 2_B783 | 29 |
| Ara h 2_B784 | 30 |
| Ara h 2_B785 | 31 |
| Ara h 2_B786 | 32 |
| Ara h 2_B831 | 33 |
| Ara h 2_B832 | 34 |
| Ara h 2_B833 | 35 |
| Ara h 2_B834 | 36 |
| Ara h 2_B835 | 37 |
| Ara h 2_B836 | 38 |
| Ara h 2_B837 | 39 |
| Ara h 2_B956 | 40 |
| Ara h 2_B957 | 41 |
| Ara h 2_B958 | 42 |
| Ara h 2_B961 | 43 |
| Ara h 2_B962 | 44 |
| Ara h 2_B963 | 45 |
| Ara h 2_B964 | 46 |
| Ara h 2_B967 | 47 |
| Ara h 2_B968 | 48 |
| Ara h 2_B969 | 49 |
| Ara h 2_B970 | 50 |
| Ara h 2_B971 | 51 |
| Ara h 2_B973 | 52 |
| Ara h 2_B974 | 53 |
| Ara h 2_B975 | 54 |
| Ara h 2_B976 | 55 |
| Ara h 2_B977 | 56 |
| Ara h 2_B646 | 57 |
| Ara h 2_B981 | 58 |
| Ara h 2_B982 | 59 |
| Ara h 2_B983 | 60 |
| Ara h 2_B984 | 61 |
| Ara h 2_B985 | 62 |
| Ara h 2_B986 | 63 |
| DE Ara 2 variant 1001 | 168 |
| DE Ara 2 variant 764 | 170 |
| GM Arah2 | 195 |
| GM Arah2-TM | 196 |
| GM Arah2- MITD | 197 |
| Arah2 TM | 198 |
| Arah2 MITD | 199 |
| GM Arah2-IgG1 Fc | 200 |
| GM Arah2-IgG4 Fc | 201 |
| 1001-IgG1 Fc | 204 |
| 1001-IgG4 Fc | 205 |
| GM 1001-IgG1 Fc | 206 |
| GM 1001-IgG4 Fc | 207 |
| 1001 var2 | 208 |
| 1001 var4 | 209 |
| 1001 var6 | 210 |
| Arah2_conbo31 | 247 |
| 1001-TM | 248 |
| GM1001-TM | 249 |

TABLE 7
Amino Acid Sequences of Ara h 1 Variants

| Ara h 1 Variants | SEQ ID NO: |
|---|---|
| Ara h1_B867 | 68 |
| Ara h1_B869 | 69 |
| Ara h1_B871 | 70 |
| Ara h1_B876 | 71 |
| Ara h1_B879 | 72 |
| Ara h1_B923 | 73 |
| Ara h1_B924 | 74 |
| Ara h1_B926 | 75 |
| Ara h1_B946 | 76 |
| Ara h1_B947 | 77 |
| Ara h1_B948 | 78 |
| Ara h1_B949 | 79 |
| Ara h1_B991 | 80 |
| Ara h1_B992 | 81 |
| Ara h1_B996 | 82 |
| Ara h1_B997 | 83 |
| Ara h1_B998 | 84 |
| Ara h1_B1010 | 85 |
| Ara h1_B1011 | 86 |
| Ara h1_B1013 | 87 |
| Ara h1_B1086 | 88 |
| Ara h1_B1087 | 89 |
| Ara h1_B1088 | 90 |
| Ara h1_B1089 | 91 |
| Ara h1_B1090 | 92 |
| Ara h1_B1091 | 93 |
| Ara h1_B1092 | 94 |
| Ara h1_B1093 | 95 |
| Ara h1_B1190 | 96 |
| Ara h1_B1191 | 97 |
| Ara h1_B1192 | 98 |
| Ara h1_B1201 | 99 |
| Ara h1_B1202 | 100 |
| Ara h1_B1203 | 101 |
| Ara h1_B1246 | 102 |
| Ara h1_B1247 | 103 |
| Ara h1_B1248 | 104 |
| Ara h1_B1249 | 105 |
| Ara h1_B1250 | 106 |
| Ara h1_B1251 | 107 |
| Ara h1_B1252 | 108 |
| Ara h1_B1253 | 109 |
| Ara h1_B1256 | 110 |
| Ara h1_B1258 | 111 |
| Ara h1_B1267 | 112 |
| Ara h1_B1268 | 113 |
| Ara h1_B1269 | 114 |
| Ara h1_B1270 | 115 |
| Ara h1_B1271 | 116 |
| Ara h1_B1272 | 117 |
| Ara h1_B1275 | 118 |
| Ara h1_B1281 | 119 |
| Ara h1_B1282 | 120 |
| Ara h1_B1283 | 121 |
| Ara h1_B1284 | 122 |
| Ara h1_B1285 | 123 |
| Ara h1_B1286 | 124 |
| Ara h1_B1287 | 125 |
| Ara h1_B1288 | 126 |
| Ara h1_B1289 | 127 |
| Ara h1_B1290 | 128 |
| Ara h1_B1291 | 129 |
| Ara h1_B1292 | 130 |
| Ara h1_B1293 | 131 |
| Ara h1_B1294 | 132 |
| Ara h1_PLP242 | 133 |
| Ara h1_PLP243 | 134 |
| Ara h1_PLP244 | 135 |
| Arah1_PLP245 | 136 |
| Arah1_PLP246 | 137 |
| Arah1_PLP247 | 138 |
| Arah1_B1298 | 139 |
| Arah1_B1299 | 140 |
| Arah1_B1300 | 141 |
| Arah1_PLP256 | 142 |
| Arah1_PLP257 | 143 |
| Arah1_PLP258 | 144 |
| Arah1_B1305 | 145 |
| Arah1_B1306 | 146 |
| Arah1_B1307 | 147 |
| Arah1_B1308 | 148 |
| Arah1_PLP264 | 149 |
| Arah1_B1309 | 150 |
| Arah1_B1304 | 151 |
| Arah1_PLP317 | 152 |
| Arah1_PLP318 | 153 |
| Arah1_PLP496 | 154 |
| Arah1_PLP499 | 155 |
| Arah1_PLP595 | 156 |
| Arah1_PLP601 | 157 |
| Arah1_PLP607 | 158 |
| Arah1_PLP729 | 159 |
| Arah1_PLP730 | 160 |
| Arah1_PLP731 | 161 |
| DE Arah1 variant 5 | 174 |
| DE Arah1 variant 8 | 176 |
| DE Arah1 variant 25 | 178 |
| DE Arah1 variant 35 | 180 |
| DE Arah1 variant 51 | 182 |
| DE Arah1 variant 52 | 184 |
| combo 68-TM | 193 |
| combo 68-MITD | 194 |
| Arah1_PLP737 | 211 |
| Arah1_PLP738 | 212 |
| Arah1_PLP739 | 213 |
| Arah1_PLP740 | 214 |
| Arah1_PLP741 | 215 |
| Arah1_PLP742 | 216 |
| Arah1_PLP743 | 217 |
| Arah1_PLP744 | 218 |
| Arah1_PLP745 | 219 |
| Arah1_PLP746 | 220 |
| Arah1_PLP747 | 221 |
| Arah1_PLP748 | 222 |
| Arah1_PLP749 | 223 |
| Arah1_PLP750 | 224 |
| Arah1_PLP751 | 225 |
| Arah1_PLP752 | 226 |
| Arah1_PLP753 | 227 |
| Arah1_PLP754 | 228 |
| Arah1_PLP755 | 229 |
| Arah1_PLP756 | 230 |
| Arah1_PLP757 | 231 |
| Arah1_PLP758 | 232 |
| Arah1_PLP759 | 233 |
| Arah1_PLP760 | 234 |
| Arah1_PLP761 | 235 |
| Arah1_PLP762 | 236 |
| Arah1_PLP763 | 237 |
| Arah1_PLP764 | 238 |
| Arah1_PLP765 | 239 |
| Arah1_PLP766 | 240 |
| Arah1_PLP767 | 241 |
| Arah1_PLP768 | 242 |
| Arah1_PLP769 | 243 |
| Arah1_PLP770 | 244 |
| Arah1_PLP771 | 245 |
| Arah1_PLP772 | 246 |

### Summary

Following the above procedures, 7 Ara h 2 epitopes and at least 27 Ara h 1 epitopes were found. Twenty (20) Ara h 1 and 50 Ara h 2 single epitope de-epitope variants were verified by indirect ELISA exhibiting a reduction in the binding EC50 of at least 50% relative to the WT Ara h.

### Example 5: Allergenicity Assessment of Engineered Proteins by Ex-Vivo Basophil Degranulation Assays

Objective: To assess the allergenicity of the engineered Ara h 1 and Ara h 2 variants relative to wild-type proteins.

### Results

Based on the results from the single-site linear and conformational de-epitoping seen in Examples 2-4, mutations that abolish the binding to each epitope were combined to construct Ara h 1 (SEQ ID NOs:68-161, 174, 176, 178, 180, 182, 184, 193, 194, 211-246) and Ara h 2 variants (SEQ ID NOs:10-63, 168, 170, 195-201, 204-210, 247-249; detailed in Table 6) mutated at multiple binding sites. Alternatively, additional sequences have been computationally combined by a Monte-Carlo procedure, starting from residue level data and yielding protein variants mutated at multiple sites. The mutations listed in Tables 1-2 above summarize the individual mutation sites.

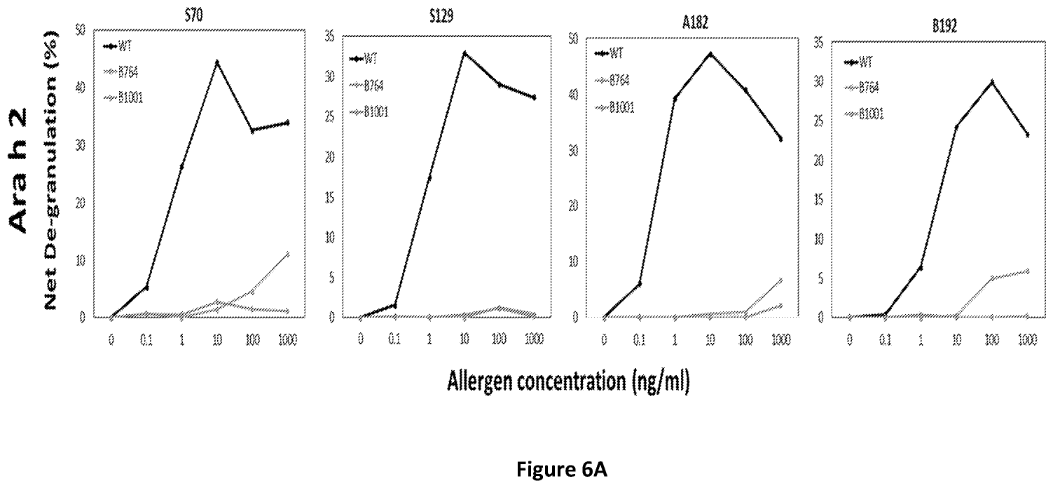

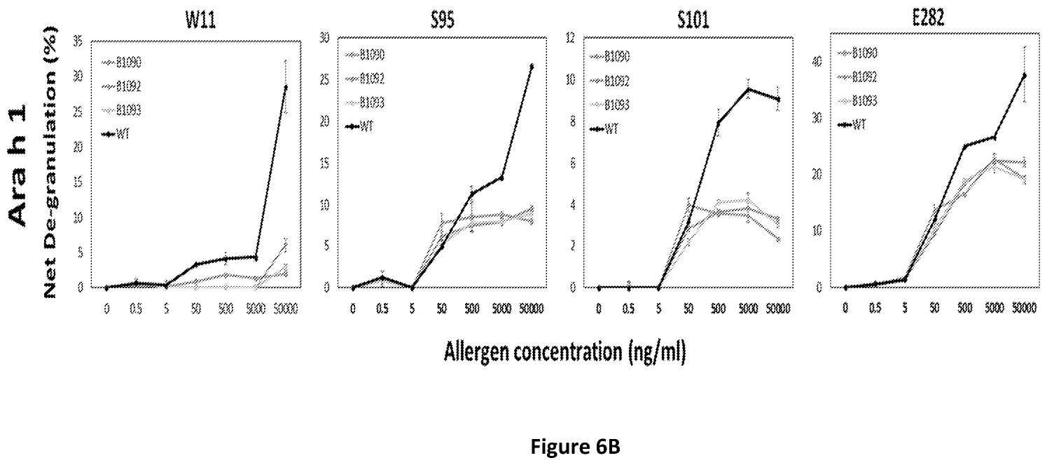

This process yielded variants that showed reduced allergenic potential compared to the WT protein. These engineered recombinant variants were expressed in E. coli, purified and tested for allergenicity. Testing was first performed on a wide ensemble of variants with a cell degranulation assay using a humanized Rat Basophil Leukemia cell line (RBL SX-38) that was sensitized with peanut allergy patient sera. Representative results from RBL assays for Ara h 1 and Ara h 2 are shown in FIG. 6A (Ara h 2) and FIG. 6B (Ara h 1). The variant allergens elicit clearly reduced cellular degranulation compared to the WT allergens, and even a dramatic reduction with the Ara h 2 variants.

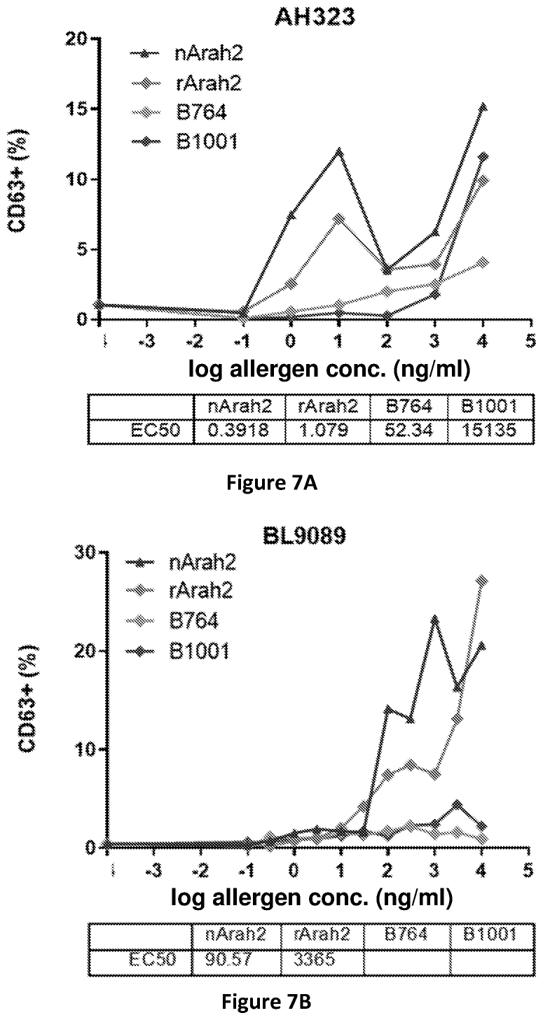

The most promising variants were then evaluated by the Basophil Activation Test (BAT) where they were compared to actual natural allergens that were purified from lightly roasted peanut flour. The BAT is a clinical-grade test that uses fresh allergy patient blood to detect and assess the severity of allergy and is becoming a gold standard for allergy diagnosis. Representative BAT results are shown in FIGS. 7A and 7B for an Israeli and an American patient, respectively, demonstrating reduced activation of basophils by leading Ara h 2 variants B764 and B1001 variants in comparison to the natural allergen.

### Summary

Based on RBL and BAT ex-vivo assays, potential abrogation of allergenicity was observed for multiple Ara h 1 and Ara h 2 mutated variants that harbor combinations of mutations at more than one epitope.

### Example 6: Immunogenicity Assessment by T Cell Activation

Objective: To assess the immunogenicity of representative Ara h 1 and Ara h 2 variants.

### Results

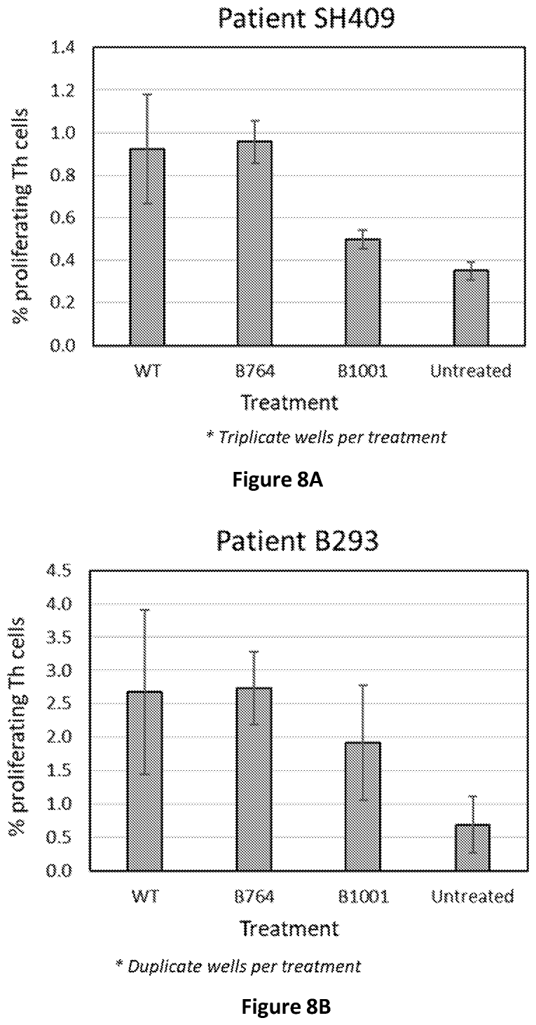

In order to guarantee immunotherapeutic efficacy, the recombinant engineered hypoallergenic variants must retain T-cell immunogenicity that would enable reprogramming of the immune response. To ensure retained immunogenicity, the Ara h 2 variants were tested for their ability to elicit allergen-specific proliferation of T helper cells derived from peanut allergy patient peripheral blood. Similar analysis is underway for Ara h 1 variants. An example of a T-cell proliferation assay performed on PBMCs collected from two peanut allergic patients with two representative variants is presented in FIGS. 8A and 8B (Patients SH409 and B293, respectively). Both WT- and variant-treated cells demonstrate proliferation above the background of the untreated cells, suggesting the variants retained T-cell activation capacity.

### Summary

T cell proliferation assay show that T cell activating properties for two of Ara h 2 mutated variants were conserved, suggesting that successful immunotherapy can be achieved with these variants.

### Example 7: Biophysical Characteristics of the Variants

Objective: It is important to maintain the same oligomerization level of the natural proteins (i.e., trimer for Ara h 1 and monomer for Ara h 2) to ensure the correct 3D folding in the mutated variants. In order to validate the oligomerization state of the proteins, size-exclusion chromatography (SEC) HPLC was performed on each variant and only variants with the correct oligomerization state were considered valid candidates for hypoallergenic variant development (data not shown).

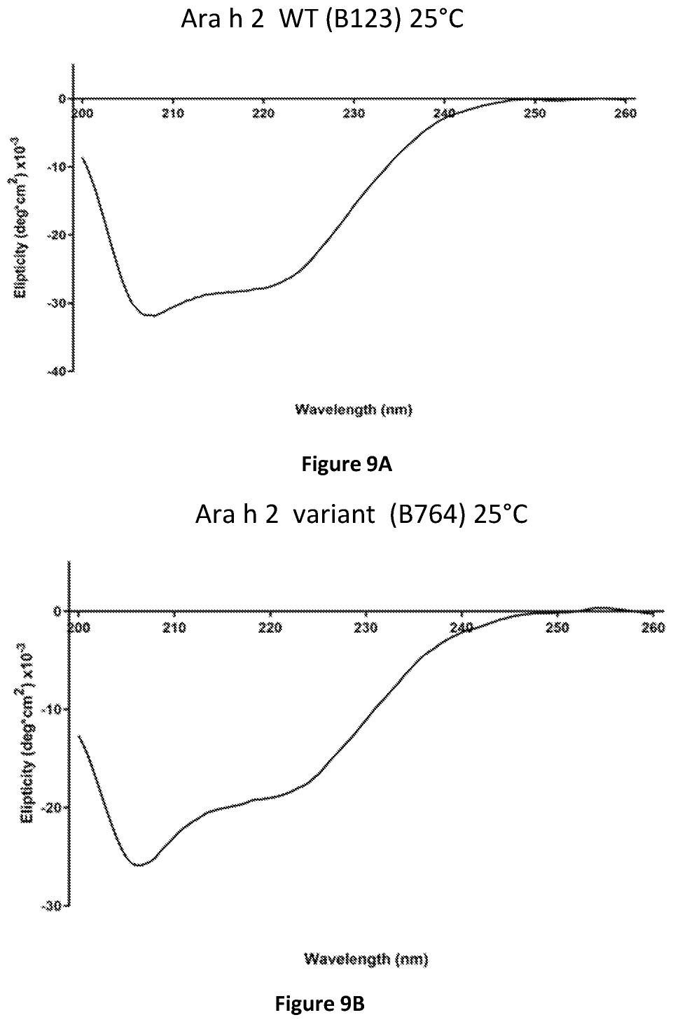

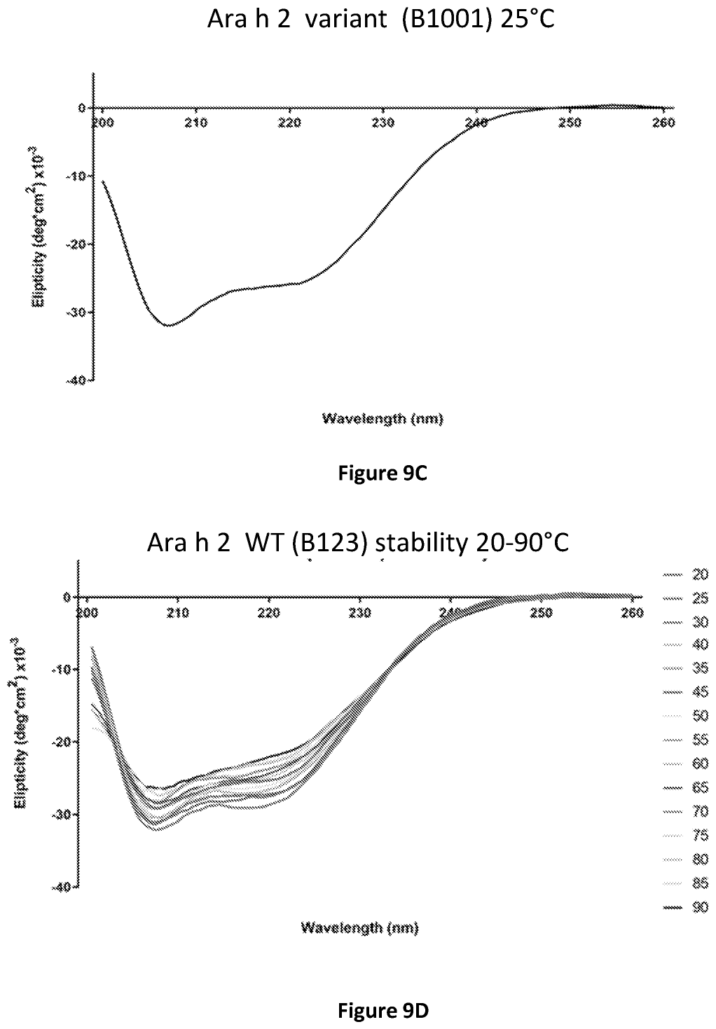

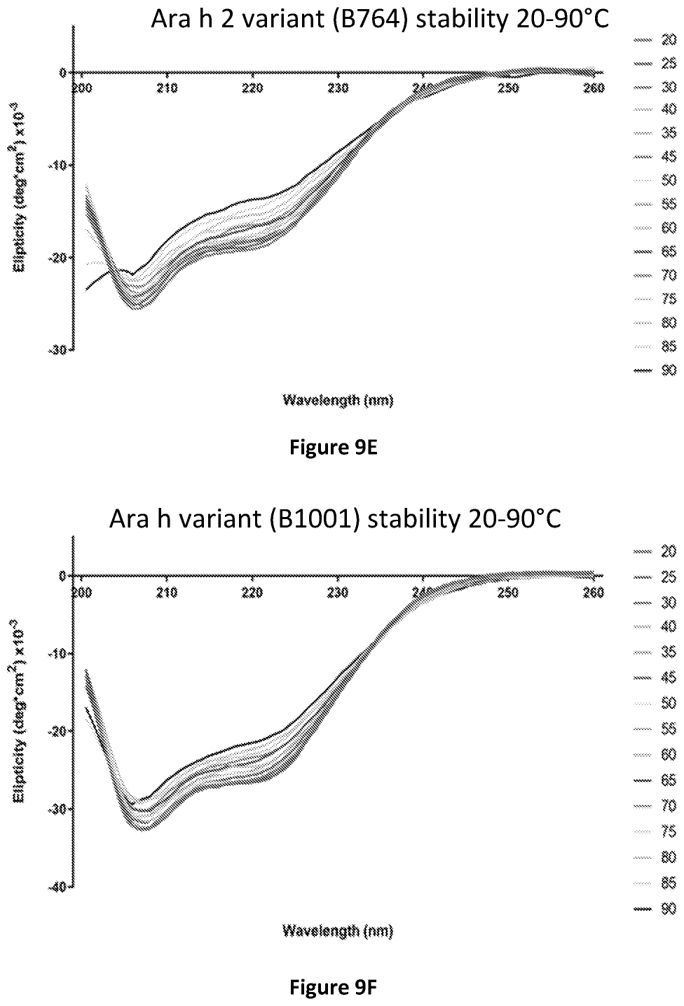

Some of the leading Ara h 2 variants were further analyzed for thermal stability using Circular Dichroism. Both tested variants (B764 and B1001) and the WT exhibited peaks at 208 and 222 nm characteristic of α-helix content. The thermal melting mid-point (TM) of both variants and the WT were >90° C. suggesting high stability and correct fold. (FIGS. 9A-9F).

### Summary

Two of the leading Ara h 2 variants exhibit a high melting point in CD, suggesting thermal stability that is similar to the WT allergen. All the combination variants of both Ara h 2 and Ara h 1 were tested in SEC HPLC and present monomeric mass for Ara h 2 (˜19 kDa) variants and trimeric mass for Ara h 1 variants (˜180 kDa) suggesting correct fold.

While certain features of the variant hypoallergenic peanut allergens Ara h 1 and Ara h 2 have been illustrated and described herein, many modifications, substitutions, changes, and equivalents will now occur to those of ordinary skill in the art. It is, therefore, to be understood that the appended claims are intended to cover all such modifications and changes as fall within the true spirit of these variants and uses thereof.

### Example 8: Expression and Secretion of Allergen Variants from Mammalian Cells

Objective: To demonstrate peanut proteins can be expressed, folded and secreted from mammalian cells, based on DNA vectors. It is also demonstrated that engineered de-epitoped allergen are expressed, folded and secreted from mammalian cells.

### Methods

Cloning—DNA vectors of the wt peanut allergens Ara h 2 and Ara h 1 and de-epitoped (DE) Ara h 2 and Ara h 1 were codon optimized for mammalian cell expression, synthesized and cloned into the pTwist CMV puro plasmid with HMM+38 leader sequence. For mRNA template vectors, sequences were optimized for in vitro transcription and mammalian expression, synthesized and cloned into a proprietary plasmid. The coding sequence of mRNA templates is flanked by an SP6 transcription site for IVT, TEV 5′ leader UTR, Xenopus beta globin 3′ UTR and 120-mer polyA templated in the plasmid. Each sequence was cloned with leader sequences derived from either human IgG kappa light chain, human IgE heavy chain, or human osteonectin (basement-membrane protein 40).

mRNA Production—All mRNA constructs were produced by Vernal Bioscience Inc. The mRNAs used in animal studies were enzymatically cap1 capped and have all uridines substituted with N1-methyl-pseudouridine.

Transient Cell Transfection—Expi293 cells (ThermoFisher Scientific) were transfected according to the manufacturer's protocol. Briefly, cells were split into 125 ml flasks at 2.5×10⁶ cells/ml in 25 ml Expi293 expression medium. Cells were transfected with 25 μg DNA complexed with ExpiFectamine complexed in Opti-MEM. On the day following transfection the growth medium was supplemented with Enhancer1 and Enhancer2 according to the manufacturer's recommended ratios. ExpiCHO cells (ThermoFisher Scientific) were transfected according to the manufacturers protocol. Briefly, cells were split to vented 50 ml tubes, 4×10⁶ cells/ml in 15 ml in ExpiCHO expression medium. Cells were then transfected with 7.5 μg DNA complexed with ExpiFectamineCHO reagent. On the day following transfection the growth medium was supplemented with ExpiCHO enhancer and ExpiCHO Feed according to the manufacturer's recommended ratios.

Protein Purification—The peanut allergens Ara h 2 and Ara h 1 and de-epitoped variants of Ara h 2 and Ara h 1 were expressed in transiently transfected cells as described above for 4-5 days, after which the cells were spun down and the clarified medium dialyzed over night against 20 mM tris pH 8.0, 350 mM NaCl, 5% glycerol. The dialyzed proteins were loaded onto a Ni-NTA resin column equilibrated buffer A—20 mM tris pH 8.0, 350 mM NaCl, 10 mM imidazole, washed with buffer A, and eluted with buffer A with the addition of 240 mM imidazole. The eluted proteins were then concentrated using a centrifugal concentrator and loaded onto an appropriate size exclusion column (Superdex75 or Superdex200 for Arah h 2 and Ara h 1 respectively) equilibrated to PBS. The eluted proteins were analyzed by SDS PAGE and the appropriate fractions pooled and concentrated using a centrifugal concentrator.

Analytical HPLC—Purified recombinant Ara h 1 and Ara h 2 were subjected to analytical size exclusion HPLC to ensure the correct oligomerization and oxidative folding state as compared to a natural peanut allergen standard (INDOOR Biotechnologies). Briefly, roughly 10 μg of protein in 10 μl was injected into a Waters Acquity Arc UHPLC equipped with a BEH 200 Å analytical SEC column equilibrated to PBS and the eluting proteins monitored by UV absorbance. Purity and concentration were calculated from the resulting chromatogram traces and used for later experiments.

Total Mass Analysis—For purified recombinant Ara h 2, the protein was subjected to total mass analysis to determine the correct composition and oxidative state, carried out in the core facility mass spectrometry unit of the Hebrew University. Samples of recombinant Ara h 2 were buffer-exchanged to 20 mM ammonium bicarbonate pH 9.0 and subjected to ESI MS for exact mass determination.

Allergen Antibody Binding Assay—Purified mammalian-expressed recombinant peanut allergens were assayed for their ability to bind panels of either sera from allergic patients or anti-Ara h 1 or anti-Ara h 2 antibodies by ELISA. Briefly, plates were coated with 100 μL of 2 μg/ml antigen in PBS and PBS with 0.5% BSA as a negative control. Plates were sealed and incubated overnight at 4° C. on a shaker. Coating solution was discarded and 200 μl of PBS+0.5% BSA blocking solution was added to each well and incubated shaking for 2 h. To each well, 50 μl of either allergic-patient serum or antibody solution was added and incubated 1 h at RT. Wells were washed 3× with PBST then treated with secondary antibodies, either HRP-conjugated anti-human IgE for samples tested with human sera, or HRP-conjugated anti-FLAG or HRP-conjugated anti-IgG secondary antibodies for samples assayed with ScFv or IgG antibodies respectively. Following 30 minutes incubation with secondary antibodies, wells were washed 3× with PBST, and reacted with TMB solution. The TMB reaction was quenched, and binding quantified by absorbance at 450 nm.

### Results

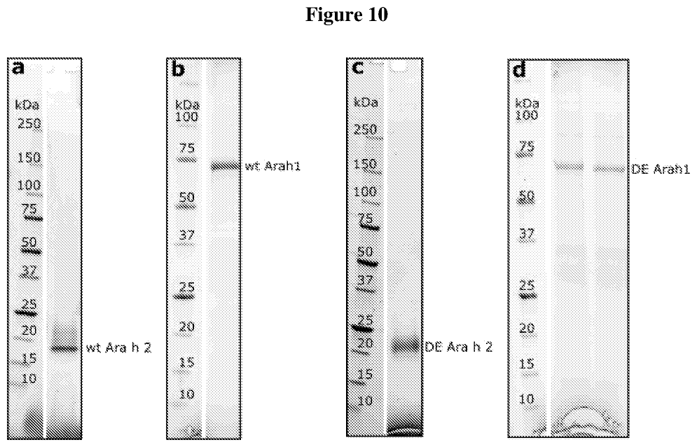

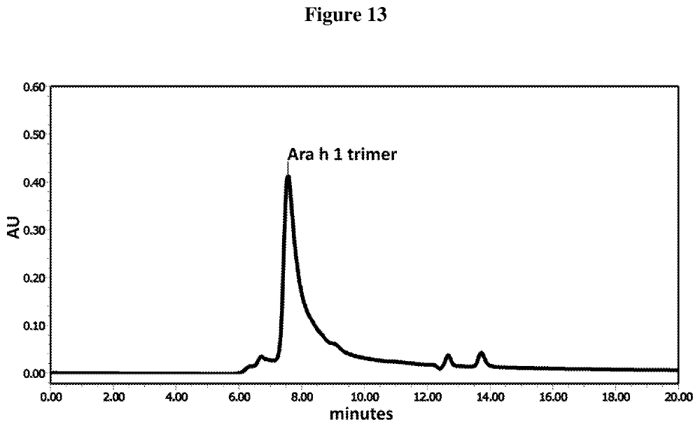

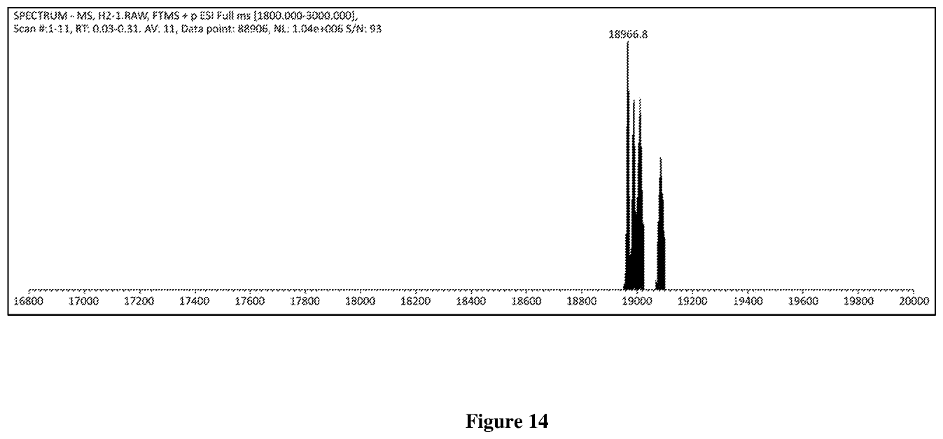

The following results demonstrate that the peanut allergens Arah1, Arah2 and their de-epitoped variants can be expressed at high levels. FIG. 10 shows wild-type or de-epitoped peanut allergens Ara h 2 and Ara h 1 were expressed and secreted from transfected mammalian cells. Purified Ara h 1 from transfected mammalian cells was found to have correct trimeric folding as shown by HPLC analysis (FIG. 13). Total mass measurement as shown in FIG. 14 indicates that Ara h 2 from transfected mammalian cells has the correct mass as expected from the sequence of the transfected Ara h 2.

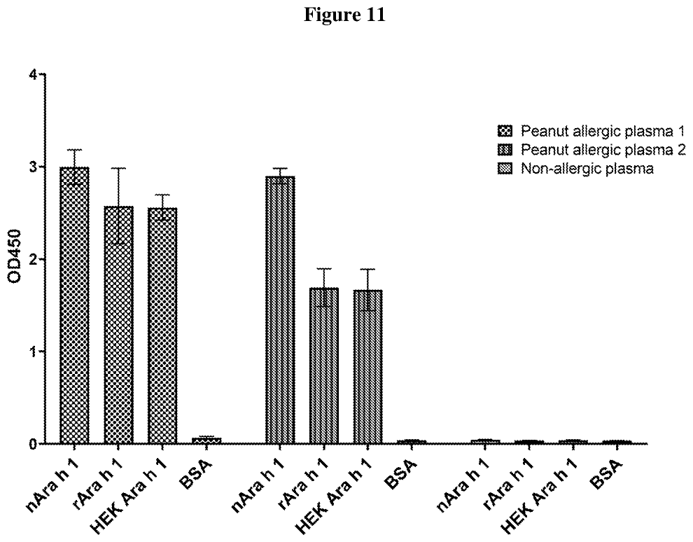

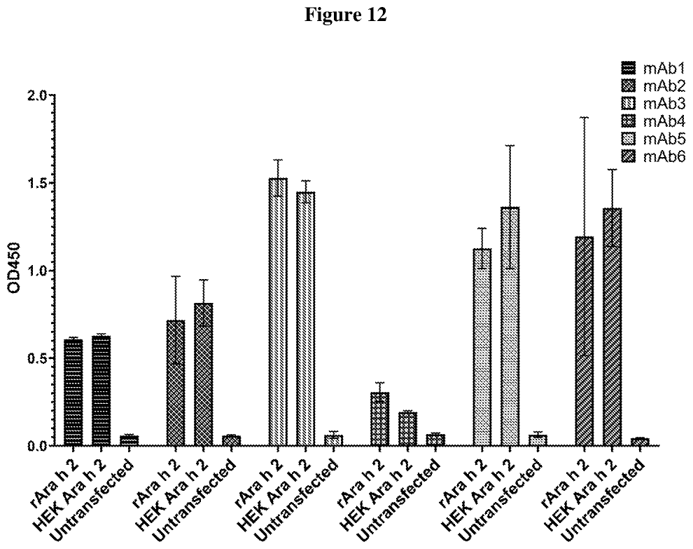

The mammalian cell derived allergens retain their ability to bind anti-allergen antibodies, both as purified monoclonal antibodies, as well as IgE from allergic patient sera. FIG. 11 shows natural Ara h 1, recombinant E. coli-derived wild-type Ara hi, and recombinant HEK cell-derived wild-type Ara h 1 have comparable binding to IgE in allergic patient sera. FIG. 12 shows recombinant Ara h 2 and the HEK-derived wild-type Ara h 2 have comparable binding to a number of well-characterized anti-Ara h 2 monoclonal IgG antibodies.

### Example 9: Preliminary Animal Study

Objective: To determine the feasibility of producing an immune response from peanut allergens delivered by mRNA-gene therapy, and assay leader sequences for increased allergen protein secretion.

### Study Design

Thirty five BALB/c mice were raised exclusively peanut free chow to preclude formation of anti-peanut antibodies. The mice were divided into seven groups of five mice, each group received six weekly i.v injections of 10 μg of a particular mRNA construct (see Table 8) formulated in Trans-IT-mRNA (Mirus Bio) and DMEM according to manufacturer's instructions.

TABLE 8
mRNA Constructs

| Group | Leader sequence | Protein |
|---|---|---|
| 1 | BM-40 | Ara h 1 |
| 2 | IgGk | Ara h 1 |
| 3 | IgE | Ara h 1 |
| 4 | BM-40 | Ara h 2 |
| 5 | IgGk | Ara h 2 |
| 6 | IgE | Ara h 2 |
| 7 | — | Firefly luciferase |

Mice sera were collected at weeks 1, 3 and 5, and sacrificed on week 7. The sera of each group were assayed for formation of anti-peanut allergen antibodies and for the peanut proteins themselves by ELISA.

### Results

Delivery of mRNA encoding for wild-type peanut allergens produced a B-cell response and elicited the production of IgG antibodies for WT Ara h 1, but not for WT Ara h 2 as detected by ELISA assays using natural peanut allergens. Detection of such antibodies indicated a B-cell response towards the secreted allergen proteins, demonstrating mRNA delivery of peanut allergens is a promising strategy for subsequent experiments using de-epitoped allergens for desensitization. The blood serum levels of peanut proteins were compared between the various leader sequences, as well as the levels of anti-allergen IgG, indicating the secretion efficiency of the respective leader sequence. This information was used to determine which leader sequence facilitates the most efficient secretion of each peanut allergen. The BM-40 leader sequence produced the highest antibody titter, corresponding well to the expression level pattern observed in mammalian cells using the same constructs.

### Example 10: Allergy Model Animal Study

Objective: To determine the potential and degree of desensitization of sensitized mice by mRNA delivery of de-epitoped Ara h 2.

### Study Design

Mice (70 female C3H/HeJ) are initially sensitized to peanuts using i.p injections of peanut extract. The mice are then split into 14 cohorts of 5 mice (see Table 9), receiving i.v injections of 30 μg mRNA of either wild-type, or two leading de-epitoped Ara h 2 variants, or control injections, formulated in Trans-IT-mRNA (Mirus Bio) and in DMEM according to manufacturer's instructions, either weekly or every 3 weeks.

TABLE 9

| | Sensitization | | | | Challenge |
|---|---|---|---|---|---|
| | (peanut | | Dose-level | Frequency of | (i.p., i.d. or |
| Group | extract) | Treatment | (μg/mouse) | injection | i.g.) |
| 1 | Yes | Sham (PBS) | 0 | 7 (once a week) | Peanut |
| 2 | No (PBS) | Control (PBS) | 0 | 7 (once a week) | Peanut |
| 3 | Yes | WT Arah2 mRNA | 30 μg/250 μL | 3 (every 3 | Peanut |
| | | | | weeks) | |
| 4 | Yes | WT Arah2 mRNA | 30 μg/250 μL | 3 (every 3 | Natural arh2 |
| | | | | weeks) | |
| 5 | Yes | WT Arah2 mRNA | 30 μg/250 μL | 7 (once a week) | Peanut |
| 6 | Yes | WT Arah2 mRNA | 30 μg/250 μL | 7 (once a week) | Natural arh2 |
| 7 | Yes | DE 1001 Arah2 | 30 μg/250 μL | 3 (every 3 | Peanut |
| | | mRNA | | weeks) | |
| 8 | Yes | DE 1001 Arah2 | 30 μg/250 μL | 3 (every 3 | Natural arh2 |
| | | mRNA | | weeks) | |
| 9 | Yes | DE 1001 Arah2 | 30 μg/250 μL | 7 (once a week) | Peanut |
| | | mRNA | | | |
| 10 | Yes | DE 1001Arah2 | 30 μg/250 μL | 7 (once a week) | Natural arh2 |
| | | mRNA | | | |
| 11 | Yes | DE 764 Arah2 | 30 μg/250 μL | 3 (every 3 | Peanut |
| | | mRNA | | weeks) | |
| 12 | Yes | DE 764 Arah2 | 30 μg/250 μL | 3 (every 3 | Natural arh2 |
| | | mRNA | | weeks) | |
| 13) | Yes | DE 764 Arah2 | 30 μg/250 μL | 7 (once a week) | Peanut |
| | | mRNA | | | |
| 14 | Yes | DE 764 Arah2 | 30 μg/250 μL | 7 (once a week) | Natural arh2 |
| | | mRNA | | | |

At weeks 3, 5, and 7 following the first mRNA injection, sera are collected. Once the mRNA treatment is over (on week 7), the mice are challenged with either peanut extract or purified natural Ara h 2, and the ensuing allergic response monitored and scored (behavioral, physiological and serological measures).

### Anticipated Results

Desensitization will be considered successful if following the administration of de-epitoped Ara h 2, the mice will present statistically significant lower scores of clinical parameters including anaphylaxis, lower levels of mouse mast cell protease, and lower relative levels of anti-Ara h 2 IgE.

While certain features of the nucleic acids encoding variant hypoallergenic peanut allergens Ara h 1 and Ara h 2 have been illustrated and described herein, many modifications, substitutions, changes, and equivalents will now occur to those of ordinary skill in the art. It is, therefore, to be understood that the appended claims are intended to cover all such modifications and changes as fall within the true spirit of these variants and uses thereof.

### Example 11: Characterization of the Biological Activity of Ara h 2 Variant B1001 (SEQ ID NO: 10)

Objective: to demonstrate the biological activity of Ara h 2 variant B1001

### Methods:

### Recombinant Ara h 2 Expression and Purification

Recombinant Ara h 2 sequences (WT or B1001) were cloned into pET28 plasmid and fused to sequences encoding a His-tagged TRX protein and a TEV-protease cleavage site (N-Trx-His X6-TEV site-Ara h 2-C). Plasmid was transformed into ORIGAMI™ cells (New England Labs) and the proteins were expressed under the transcriptional control of a T7 promoter. Cells were grown at 37° C. with shaking at 250 RPM until an OD of 0.5-0.8 was reached, and induction was carried out by addition of 1 mM IPTG for and incubation for further 3 h at 37° C. Cells were pelleted (4800 g for 30 min) and resuspended with ×10 (w/v) lysis buffer (50 mM Tris pH 8.0, 350 mM NaCl, 10% v/v glycerol, 0.2% Triton X-100, 5 U/ml Benzonase (Sigma), 0.2 mM PMSF (thermos-fisher Scientific), 1 mg/ml Lysozyme (Angene). Cells were ruptured by sonication (60% amplitude, 10 sec on, 30 sec off, 2 min). Lysates were centrifuged (15000 g, 45 min) and supernatant was loaded on Ni-NTA columns pre-washed with binding buffer (50 mM Tris pH 8.0, 350 mM NaCl, 10% v/v glycerol). The beads were washed with binding buffer containing gradually increasing imidazole concentrations and the individual fractions were collected and analyzed by SDS-PAGE. Fractions containing desired protein were pooled. The buffer was exchanged to imidazole-free binding buffer by overnight dialysis at RT, using SnakeSkin dialysis tubing 3.5 kDa (Thermo Fisher scientific). On the following day, His-tagged TEV protease (manufactured in-house) was added to the sample at a 1:30 molar ratio (TEV: rAra h 2) and incubated for 3 hours at RT. Trx-His tag portion and the TEV protease were removed by loading the solution onto a Ni-NTA column pre-washed with binding buffer. The flow-through and the Ara h 2-containing 20 mM-imidazole wash fractions were collected, concentrated by 3 kDa Centricones (Amicon, Mercury) to ˜5 mg/ml and loaded onto Superdex 75 μg SEC column pre-washed with PBS buffer (Cytiva). Fractions containing monomeric Ara h 2 were pooled and the concentration was measured and calculated by the absorbance at 280 nm using extension coefficients (0.817 for WT, 0.672 for B1001). Proteins were flash-frozen in liquid Nitrogen and stored at −80° C. until use.

### Circular Dichroism

Purified Arah2 proteins (WT and B1001) were diluted to 0.3 mg/ml in 0.5 ml with PBS buffer and underwent Circular dichroism analysis (CD) (Chirascan™-plus CD Spectrometer) for the 200-260 nm spectra at RT. For secondary structure thermal stability analysis, CD spectra (200-260 nm) were recorded at the following conditions: escalating temperatures from 20-90° C. at a rate of 1° C./minute and a pathlength of 1 mm.

### Western Blot Analysis

Two ug of purified proteins were mix with Leammeli sample buffer (Bio Rad) with beta-mercaproethanol, were ran on stain-free Min-Protean TGX gels (Bio Rad). Prior to transfer, the gel was visualized by Molecular Imager® ChemiDoc™XRS+(Bio Rad), then transferred to Transblot turbo PVDF membrane (Bio Rad). Blocking was done for 1 h at RT using 5% skim milk (Sigma) in PBST. Arah2 detection was done using 1:1000 PAb rabbit anti Arah2 (Indoor) and with 1:10000 secondary antibody anti-rabbit HRP. Femtogram ECL substrate was use for bands visualization.

### SEC- and RP-HPLC

Purified natural Ara h 2 (Indoor), WT Ara h 2 and B1001 Ara h 2 were analyzed by SEC-HPLC at 30° C. (UHPLC Arc System, Waters; Column XBrige Protein BEH SEC 200A, 2.5 um in 0.1M Sodium phosphate buffer as mobile phase). Molecular weights were estimated based on a gel filtration molecular weight standard (Biorad). The proteins were also analyzed by RP-HPLC using a C18 column at 50° C., 0.1% TFA in HPLC-grade water as mobile phase A and 0.1% TFA in Acetonitrile as mobile phase B (UHPLC Arc System, Waters; Column Jupiter 5 um C18 300 A, 250×4.6 mm). For both, analysis detection was done with UV 220 nm.

### Allergy Patient Samples

All samples were obtained from clinically diagnosed peanut allergy patients with recent history of allergic reaction to peanuts. All collaborating medical centers received approval for local institutional review boards for providing samples for this study.

Whole blood was obtained in heparinized tubes from peanut allergy patients in collaborating medical centers in Israel. Plasma was isolated by centrifugation at 800 g for 10 minutes and separation of upper phase. Peripheral blood mononuclear cells (PBMC) were extracted from blood samples using Sepmate tubes (Stemcell, Canada) according to the manufacturer's instructions and cryopreserved using endotoxin-free materials: FBS (Biological industries, ISR), PBS×10 pH7.4 (Gibco, USA), ultra-pure ddw (Bioline, ISR) and Lymphoprep (Stemcell). Fresh whole blood for basophil activation testing was also obtained by Amerimmune from referring clinical in various USA locations.

Additional plasma, sera (isolated by gel-phase lock tubes) and PBMC were obtained using comparable isolation procedures from the following partners and providers: Nadeau lab at Stanford university (CA, USA), AbBaltis (UK), Ebisawa lab at Jeiki university (Tokyo, Japan), Nino Jesus university hospital (Madrid, Spain), Access biologicals (CA, USA), Amerimmune (VA, USA), Mie university hospital (Japan). Expanded clinical samples information can be found in the supplementary data.

### IgE and IgG Level Analysis by ELISA

Maxisorp 96-well plates (Thermofisher scientific) were coated overnight at 4° C. with 1001 of natural Ara h 2 or B1001 at 2 g/ml in PBS. All subsequent steps were carried out at RT with PBST washes (PBS+0.05% Tween 20) between steps. Titration curves were created for each serum or plasma samples by diluting ×10 and then serially ×2.1 (for IgE detection) or ×25 and then serially ×2.5 (for IgG). Plates were blocked with PBST+2% BSA (Sigma) for 2 hours, incubated with titrated samples or without (blanks) for 2 hours, and then incubated with 1:5,000 HRP-Goat Anti-Human IgE (Abcam) or 1:20,000 HRP-Donkey Anti-Human IgG (Jackson labs) for 1 hour. Finally, Plates were incubated with 100 μl 1-Step Ultra TMB (Thermofisher) until color developed and 100 μl H₂SO₄ 0.5M were added to stop reaction. Optical density at 450 nm was recorded using the Synergy LX microplate spectrophotometer (Biotek, Vermont), OD of blank wells (without sample) was subtracted and area under curve was calculated using Prism Graphpad.

### RBL SX-38 Degranulation Assay

RBL SX-38 cells were received from Prof. Stephen Dreskin in UC Denver, with license from BIDMC in Boston. Cells were in cultured breathable flasks (Greiner) at 37° C., 5% CO2 in media containing 80% MEM (Gibco, US), 20% RPMI 1640, 5% FCS (not heat-inactivated), 2 mM L-glutamin, Penicillin-Streptomycin (Biological industries, ISR) and G418 at 1 mg/ml (Formedium, UK). Cells were split and expanded for 48 hours in assay media (without RPMI and G418). On day of assay, cells were detached using 0.05% Trypsin-EDTA (Gibco), centrifuged at 300 g for 10 minutes, and resuspended to 250×10⁵ cells/ml in assay media with 10% clinical sample (plasma or serum from peanut allergy patients). Non-heparinized plasma was first supplemented with 30 U/ml Sodium-Heparin (Sigma) and incubated at RT for 10 minutes before adding to cells to prevent coagulation. Cells were seeded at 50 μl/well (final 125,000 cells) in 96-well flat-bottom tissue culture plates (Greiner bio-one, AUS) and cultured overnight. The next day, activating solutions were prepared by performing serial 10-fold dilutions for natural Ara h 2, B1001 or negative control (KLH, Sigma) in Tyrode's buffer. Buffer composition: 137 mM NaCl, 2.7 mM KCl, 0.4 mM NaH2PO4, 0.5 mM MgCl2, 1.4 mM CaCl₂), 10 mM Hepes pH 7.3, 5.6 mM glucose, 0.1% BSA (Sigma Aldrich, ISR), pH adjusted to 7.4, prepared in a water composition of 80% ddw and 20% D20 heavy water (Sigma Aldrich). Cells were washed ×3 with Tyrode's buffer prepared with ddw only, and 100 μl activating solution was added to appropriate wells in duplicates. Duplicate wells were also prepared with lysis buffer (Tyrode's buffer with 1% Triton x-100, Fisher Scientific) for measuring total degranulation and with Tyrode's buffer alone for measuring baseline degranulation. Cells were then incubated for 1 hour at 37° C., 5% CO2. Immediately after incubation, 30 ul of each well were transferred to a corresponding well in a clear non-binding 96-well plate (Greiner Bio-one) and supplemented with 50 μl colorimetric substrate 4-Nitrophenyl N-acetyl-β-D-glucosaminide (Sigma) prepared in 0.1M citric acid to final concentration 1.368 mg/ml at pH4.5. After 1 hour at 37° C. with gentle shaking, reactions were stopped with 100 μl of 0.2M glycine pH 10.7. Optical densities were recorded at 405 nm for signal and at 630 nm for background absorbance. Net degranulation % was calculated by subtracting background absorbance, subtracting baseline degranulation, and dividing by total degranulation.

### Basophil Activation Test

Fresh whole blood samples in heparinized tubes were divided into 100 μl per reaction (either in individual FACS tubes or in 2 ml deep 96-well plates). Allergens and controls were diluted in RPMI1640 (Biological Industries) to ×2 stocks and added 1:1 to tubes (final volume 200 l). Doses used ranged 0.03-10⁵ ng/ml in 10-fold or ×3 mid-steps (1, 3, 10, 30 etc), depending on available volume, but at no less than 6 10-fold concentrations. Crude peanut extract (CPE), fMLP (Sigma) and goat sera anti human IgE antibodies (a gift from the Dreskin lab) were used as positive controls and KLH (Sigma) was used as a negative control at 10⁵ ng/ml (CPE at 6 concentrations if volume available). Samples were incubated for 30 minutes in a 37° C., 5% CO2 humidified incubator and the reaction was stopped by incubation on ice for 5 minutes. Samples were then stained for 30 min on ice with fluorophore conjugated antibodies for the following markers: CD203c, CD63, HLA-DR, CD45, CD123 (Biolegend). RBC lysis was performed with a lysing solution (BD FACS) according to manufacturer's instructions, cells were washed with PBS×1 and analyzed by flow cytometry. Cells were gated for basophils detection (cells>singlets>CD45-high/SSC-low>CD123-high/HLA-DR-low>CD203c-high) and activation rate (% CD63-positive basophils) was measured. At least 500 basophils were analyzed per tube, baseline was set by gating non-activated wells. Only samples that showed 5% activation or over in at least one of the concentrations of Ara h 2 or CPE were included in the analysis. Averaging of patients and curve fitting was done with Prism Graphpad.

### T Cell Activation Tests: Proliferation Assay and Cytokine ELISA

All materials used were verified endotoxin-free. Peptide pools covering the entire sequence of WT Ara h 2 or B1001 (35-41mer with a 20AA overlap, Peptide 2.0, VA, USA) were prepared in DMSO (Alfa Aesar, MA, USA). Peanut allergy patient PBMC were stained by 10 M Celltrace violet (Thermo-fisher) in PBS+0.5% FBS for 20 minutes at 37° C. with a 5-minute quenching step by RPMI+5% FBS. Cells were washed, resuspended in X-vivo15 media (Lonza, Switzerland) supplemented with 1% penicillin-streptomycin (Biological industries) and seeded in 96-well round bottom plates at 2-2.5×10⁵ cells/well and 4-8 replicates (according to available number of cells). Peptide pools were added to well to a final concentration of 10 g/ml per peptide in 200 ul/well. at equivalent dilution was added to non-stimulated wells, and CPE was used as positive control. Cells were incubated for 7 days in a 37° C., 5% C02 humidified incubator. Cells were then pelleted, and media was removed and retained for Cytokine ELISA. Harvested cells were stained with viability dye (near-IR LIVE/DEAD, Thermo-fisher) and then for CD3 and CD4 with fluorophore-conjugated antibodies (Biolegend, USA) and analyzed by flow cytometry. Cells were gated for live, proliferating T helpers (Singlets>LIVE/DEAD low>CD4 high/CD3 high>Celltrace dim, final proliferation gating was guided by baseline and positive control samples). The % proliferation, Stimulation Index (S.I, average allergen activation/average baseline) and Mann-Whitney U test (MW) significance were calculated (Graphpad Prism). Each sample was regarded as true-activated and included in data only if WT S.I was >2 with a MW p-value 0.1. For final averages, data from each patient was normalized to the value of one of the baseline replicates.

ELISA for detection of IL5, IL13 and IFNγ levels in retained media was performed using unconjugated/biotinylated antibody pairs optimized for sandwich ELISA (Mabtech, Sweden). Maxisorp plates were coated overnight at 4° C. with 50 μl unconjugated capture antibody at 1 g/ml in carbonate bicarbonate buffer (Sigma). The next day, standard curves were prepared with recombinant IL5, IL13 or IFNγ (Peprotech, ISR) in PBST-2% BSA. Plates were blocked with PBST+2% BSA for 2 hours at RT and then incubated overnight at 4° C. with 50 μl assay media or appropriate standards. On the last day, plates were incubated with biotin-conjugated detection antibody at 1 g/ml in PBST+2% BSA for 1 hour and then HRP-conjugated Streptavidin for 1 hour. Finally, plates were incubated with 100 μl 1-Step Ultra TMB until color developed, 100 μl H₂SO₄ 0.5M were added to stop reaction and optical density was recorded at 450 nm. Standard curves were fitted with a non-linear regression model and used to interpolated individual values. WT S.I and MW p-values were calculated and used to include only true-activated samples (S.I>2, p-value 0.1). For final averages, data from each patient was normalized to the value of one of their baseline replicates.

### Murine Allergy Model Studies

All mouse studies were carried out as contracted research by Porsolt SAS (France). Naïve female C3H/HeJ mice (Jackson Laboratory, Bar Harbor, U.S) were raised on peanut-and-soy-free chow to 3 weeks old. Mice were sensitized to peanut by oral gavage with 2 mg peanut de-fatted peanut flour (50% protein) blended in 2501 PBS with 10 μg mucosal adjuvant cholera toxin (List Laboratories, CA, USA), once a week for 4 weeks with the last dose doubled to 4 mg. Safety study: mice were challenged by intraperitoneal (i.p) injection of natural Ara h 2 or B1001 in a final volume of 250 μl. On subsequent days, the B1001-challanged mice were randomized into two sub-groups and re-challenged with a higher dose of Ara h 2 or B1001. Body temperatures were rectally measured at baseline and 10, 20, 30, 45, 60 and 120 minutes after each challenge. Anaphylactic symptoms were evaluated 120 minutes after each challenge using the common clinical scoring system (0—No clinical symptoms. 1—Edema/puffiness around eyes and/or mouth. 2—Decreased activity. 3—Periods of motionless>1 min, lying prone on stomach. 4—No responses to whisker stimuli, reduced or no response to prodding. 5—end point: tremor, convulsion, death).

Oral immunotherapy (OIT) study: Mice were sensitized as with the safety study, with a separate control remaining untreated. Following sensitization, mice were i.p-challenged with 350 μg peanut flour blended in 250 μl PBS and mice that did not show clinical score of 2 or above or a body temperature drop of at least 1.5° C. were excluded from study. Starting 2 weeks after last sensitizing dose, mice were de-sensitized by 5 oral administrations per week for 3 weeks with either PBS (sham OIT), 15 mg peanut flour in 250 μl PBS or 1000 g B1001 in 1000 μl PBS (divided into 2 daily occasions to avoid single administrations of volumes>500 μl). After 12 days from the last de-sensitizing dose, mice were challenged by i.p injection of 35 g natural Ara h 2 in 250 μl PBS and anaphylactic scoring and body temperature were recorded as described above. Surviving mice were sacrificed after 5 days and mesenteric lymph nodes (MLN) were collected and transferred into ice-cold PBS with 100 U/mL penicillin and 100 μg/mL streptomycin (Pen/strp mix). MLN were cut in small pieces, homogenized using the GentleMACS dissociator and cells were isolated by passing homogenate through a 70 μM cell strainer pre-wet with TexMACS medium (Miltenyi Biotec). MLN cells were then seeded in 96-well U-bottom plates (400,000 cells/100 μl) in TexMACS medium containing 10% FBS and pen/strep mix with 200 μg/ml natural Ara h 2 for 72 hours. Culture media was harvested and levels of IL-4, IL-5 and IL-13 in were measured using a Luminex panel assay following manufacturer instructions (ProcartaPlex, ThermoFisher Scientific). Data were analyzed with the Bio-Plex Manager software (Biorad) and concentrations were calculated using the standard curve of the corresponding cytokine (values under detection range were modified to 0). All data was analyzed for significance by Mann-Whitney U test.

### Results

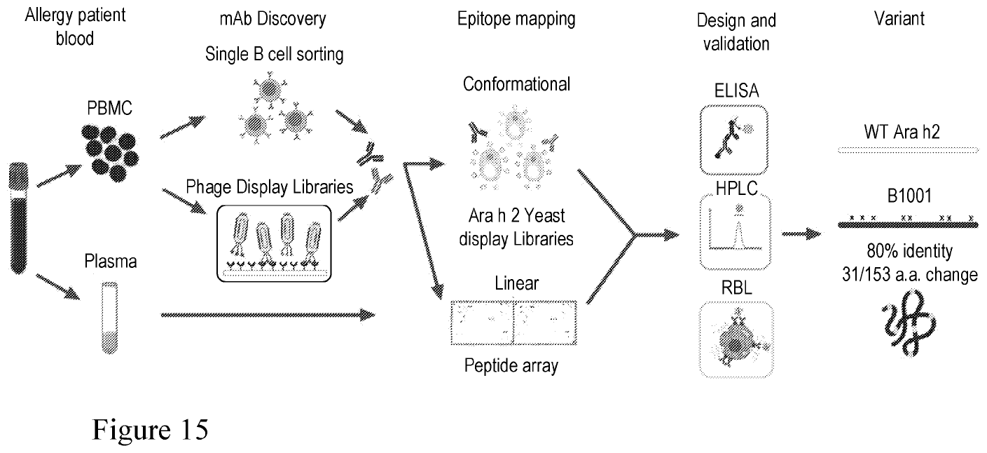

FIG. 15 presents a general outline of a patient sample-based pipeline for allergen de-epitoping. In one embodiment, peripheral blood mononuclear cells (PBMC) and plasma or serum are isolated from blood samples of clinically-verified allergy patients with diverse backgrounds. Fresh blood is provided by collaborating Israeli medical centers and processed or frozen isolates are obtained from various global locations (via collaborations with academic and clinical institutions or purchased from licensed private clinical sample providers). Naturally occurring allergen-specific B cells clones are isolated from patient PBMC either by generating and screening combinatorial scFv antibody phage-display libraries or via single cell sorting flow cytometry. These clones are used to generate and produce patient-derived, allergen-specific monoclonal antibodies (mAbs). Confirmational epitopes are then mapped by generating yeast-display allergen mutant saturation libraries and screening them with allergen-specific mAbs. Linear epitope mapping is carried out by analyzing binding of patient serum/plasma IgE or mAbs to peptide arrays that display a sliding-window coverage or the entire allergen's sequence. The comprehensive mapping process and proprietary bioinformatic process described herein are applied to the careful design allergen variants with minimum possible modifications. These variants are recombinantly expressed and biochemically characterized to validate stability and overall structural similarity to the natural allergen. IgE binding and allergenic potential of well-folded variants and the WT allergen are then compared by ELISA and RBL-SX38 assays using patient sera/plasma. Leading candidate variants are then used as input for repeated iterations of the design-validation process until variants with substantially reduced allergenicity are obtained.

### Biochemical Characterization of the Ara h 2 Variant B1001

Following the mapping and de-epitoping of Ara h 2, the minimal number and identity of mutations required to make it substantially safer while retaining its fundamental identity was determined. a variant that combines these mutations to a final 80% sequence identity to the WT Ara h 2 was designed and expressed. The biochemical properties of this novel variant which named B1001 were characterized.

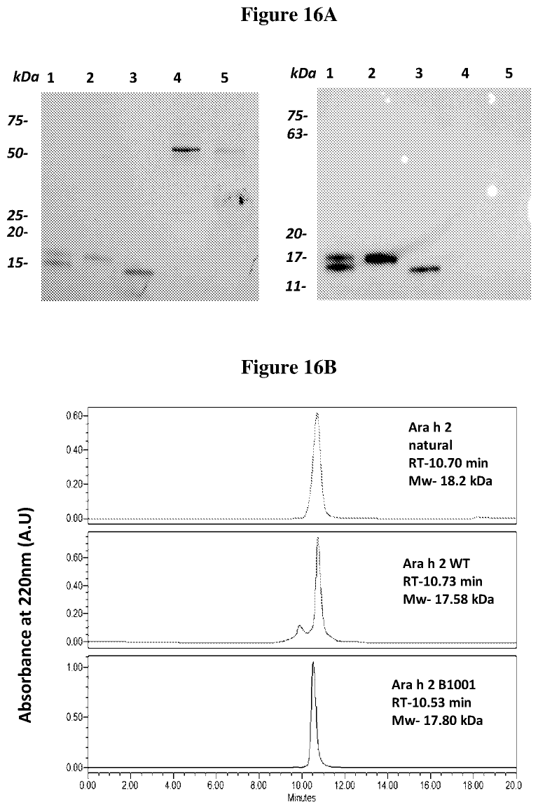

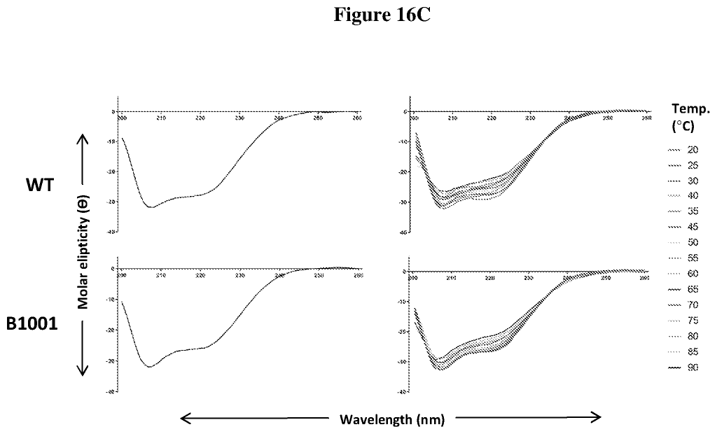

Its basic identity to Ara h 2 was confirmed by using commercial Ara h 2-specific rabbit polyclonal antibodies (pAb) to perform a western blot analysis (FIG. 16A). Indeed, the pAb specifically bound to the natural Ara h 2 (the 2 bands correspond to known isomers), to recombinantly expressed WT Ara h 2 and to B1001 alike (FIG. 16A, right pane). Recombinant WT Ara h 1 was also used as a peanut-related negative control and BSA as a general negative control (lanes 4, 5) and found that the pAbs did not bind either. A loading control of the separated protein prior to the Western blot analysis verifies visible presence of all 5 proteins on the membrane (FIG. 16A, left pane).

Ara h 2 is a monomeric 17 kDa protein, composed mostly of α-helices and containing four disulfide bonds which give it a distinctively high thermo-stability. Size-exclusion HPLC was used to estimate molecular weight and oligomeric state of B1001, who's profile was compared to the recombinant WT and natural Ara h 2. All three proteins had similar retention times and estimated molecular weights of 17-18 kDa (FIG. 16B). Next, the secondary structure of B1001 was examined by Circular Dichroism (CD) and was compared to the WT protein. Both proteins showed a typical α-helix spectral signature (FIG. 16C, left pane), similar to that previously shown for the natural protein. The thermal stability of the secondary structure of B1001 and WT Ara h 2 were also examined by carrying out CD at gradually escalating temperatures (20-90° C.). Both proteins retained their secondary structure even at 90° C. (FIG. 16C, right pane), also previously shown for Ara h 2.

In summary, it was showed that the B1001 variant folds into a stable monomer with molecular weight, secondary structure and thermal stability that are comparable to that of the WT Ara h 2 protein. Moreover, it was show that B1001 has clear immunological cross-reactivity to Ara h 2, demonstrated by binding to Ara h 2-specific pAbs. These results imply that B1001 retains an essential identity of a variant of Ara h 2.

### Differential Decrease in Patient Antibody Binding to Ara h 2 Variant B1001

Engineering an allergen to reduce its allergenicity for immunotherapy is naturally likely to also impair its immunogenicity, and thus requires a compromise between these opposing consequences. At the epitope-antibody interaction level, this means striking a balance between reducing binding of pathogenic IgE and preserving essential identity to the natural allergen, such that IgG binding potential is retained.

ELISA assay was conducted to examine how the modifications altered IgE and IgG binding. Plates coated with natural Ara h 2 or B1001 were incubated with serially diluted plasma or sera from 24 peanut allergy patients. The resulting curves significantly varied in shape, which was to be expected considering the complex interaction between multiple factors that shape each patient's antibody repertoires. This implied that comparing binding at a single dilution or deriving EC50 values might provide a partial and possibly misleading measure. Therefore, the differences in area-under-the-curve (AUC) values were compared, which while not clinically interpretable are nonetheless un-skewed by local bias or regression model fitting.

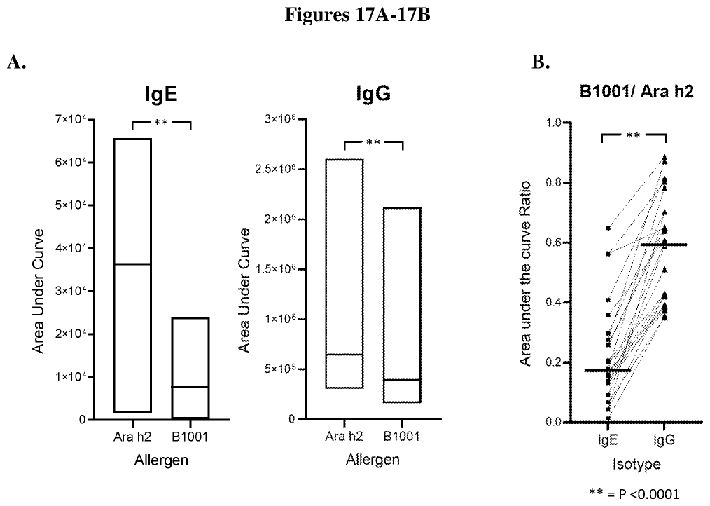

It was found that binding to B1001 was significantly reduced for both the IgE and IgG fractions. However, this reduction was notably more modest for the IgG fraction (FIG. 17A, Wilcoxon matched-pair P value<0.0001). In fact, when observing individual [B1001 AUC/Ara h 2 AUC] ratios it is apparent that the reduction was more pronounced for IgE than for IgG fraction in every single patient, with median ratios being 0.173 for IgE and 0.593 for IgG (FIG. 17B, Wilcoxon P<0.0001). These results demonstrate that the Ara h 2 de-epitoping process was successful in preferentially reducing IgE over IgG binding sites to a significant extent.

### The Allergenic Potential of B1001 is Markedly Reduced Compared to Natural Ara h 2

The overall binding strength of a patient's IgE repertoire to an allergen is shaped by multiple factors such as antigen-specific titer, clonal diversity, individual clone binding strength. However, the allergenic potential of a molecule is a separate trait that may be affected by these factors to different extents that are not easily predictable from straight-forward binding assays. Additional critical factors that influence allergenic potential include among others a patient's allergen-specific IgE relative titers and binding of specific epitopes that are sterically compatible with effector cell activation. Therefore, reduced IgE binding may or may not indicate reduced allergenic potential and warrants separate examination.

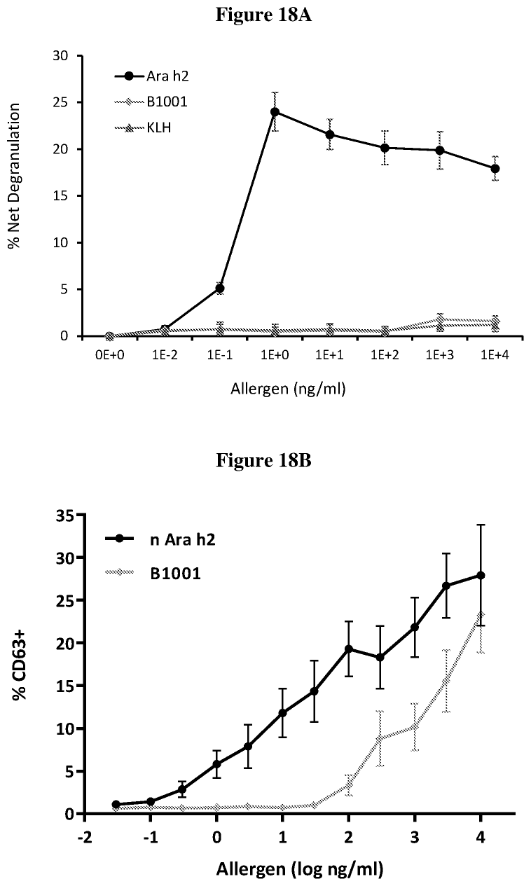

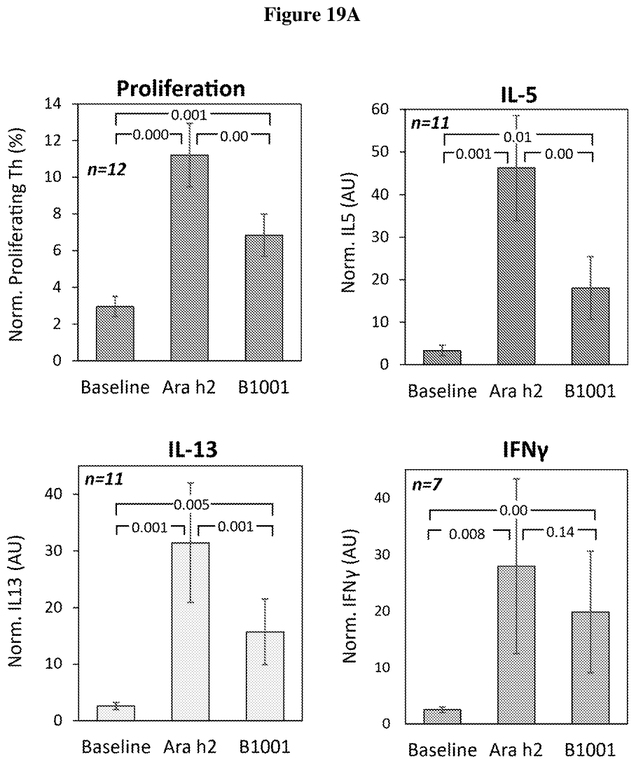

The capability of B1001 to activate the humanized rat basophil-like RBL SX-38 cell line was tested. This widely used cell line can be sensitized with human patient samples to respond to allergen stimulation by cell degranulation. The rate of degranulation is proportionate to the allergenic potency of the stimulating molecule and can be measured by an enzymatic reaction with a colorimetric substrate of the granular enzyme 0-hexosaminidase. RBL SX-38 cells were sensitized overnight with 1:10 plasma or serum from 28 different clinically validated peanut allergy patients from diverse backgrounds and then stimulated the cells with 0.01-10,000 ng/ml of either Ara h 2, B1001 or unrelated negative control protein keyhole limpet hemocyanin (KLH). Strikingly, when plotting the point-by-point averages it was found that B1001 had lost essentially all ability to elicit RBL degranulation within the entire range of concentrations tested, showing unresponsiveness similar to KLH (FIG. 18A). In fact, every single sample tested provided a response to B1001 that was less than 1000-fold decreased compared to natural Ara h 2 (data not shown). These results demonstrate that the de-epitoping process of B1001 dramatically reduced its allergenic potency.

RBL assays allow high throughput comparison of multiple variants using multiple patient samples, making them a powerful tool for engineering and validation of modified allergens. However, sensitivity and accuracy of this assay in predicting patient responses may limited by several factors such as human serum cytotoxicity to rat cells, fluctuating number of surface FcεRI molecules and lack of the human FcεRI β-subunit, lack of human IgG receptors, and lack of individuals immune context. On the other hand, the basophil activation test (BAT) is a well-founded cytometric assay that has been gaining favor with physicians and researchers alike as for its accuracy, sensitivity, and ability to provide clinically predictive data. To test the safety of B1001 compared to Ara h 2, BAT assays were performed with a cohort of 44 Israeli and U.S peanut allergy patients using commonly accepted protocols with allergen concentration ranging 0.03-10,000 ng/ml (range and number of points tested per patient varied according to available blood volume). The relative allergenicity of both proteins was estimated by plotting the point-by-point average, fitting the resulting curves to a 4-parameter logistic regression model and extracting EC50 values for each curve. It was found that Ara h 2 EC50 was 39.3 and B1001 EC50 was 11,986, meaning that on average B1001 was ˜300-folds less allergenic than Ara h 2 (FIG. 18B). These results demonstrate the increased safety of B1001 over Ara h 2 and provide support for its application in immunotherapy.

### T Cell Immunogenicity of B1001

It is well-established that immunotherapy relies on the reprogramming of existing allergen-specific T helper clones from the Th2A towards Th1/iTreg phenotypes. Purportedly, this is achieved by the careful exposure to sub-allergenic doses which causes chronic activation of these clones without the original Th2A-skewing context. Hence, for a modified allergen to be an effective immunotherapeutic drug it must retain at least some immunogenicity towards existing allergen-specific Th clones. No in-vitro T cell activation assay has been calibrated so far to reliably correlate with clinical efficacy to some predictable degree. However, such assays remain a solid approach to assessing if a molecule's immunogenic potential has been critically impaired.

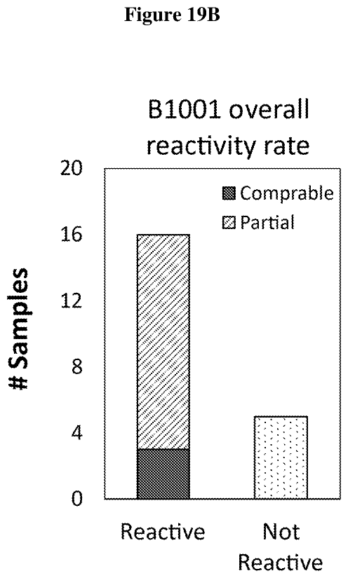

To ensure that the modifications did not abolish B1001 T cell immunogenicity, peanut allergy patient peripheral blood T cells were treated with proliferation detection dye and stimulated with pools of overlapping peptide comprising the entire sequence of either unmodified Ara h 2 or of B1001. Then both cells were retained for cytometric proliferation analysis and media for sandwich ELISA analysis of secretion of Th2 cytokines IL-5 and IL-13 and Th1 cytokine IFNγ (FIG. 19A). Data were collected only from samples that cleared pre-determined thresholds for Ara h 2. It was found that while peanut allergic T cell reactivity to B1001 was reduced compared to WT, activation nonetheless still clearly and significantly retained in all tested parameters (B1001 S.I/WT S.I: 61% for proliferation, 39% for IL-5, 50% for IL-13 and 71% for IFNγ). To assess overall B1001 reactivity, pre-determined thresholds were used to classify each patient sample as non-reactive or has having partial or comparable reactivity compared to Ara h 2. Of total 21 samples tested, 3 were estimated as comparable, 13 as partial and 5 were unreactive (FIG. 19B). These results indicate that de-epitoping for IgE also impaired the allergen's T cell immunogenicity, but only partially. Findings suggest that complete loss of immunogenicity only occurred in a small fraction of patients.

### Safety and Immunotherapeutic Efficacy of B1001 in Mouse Peanut Allergy Models

Current murine food allergy models only provide tentative clinical insight due to several key differences from humans such as prominent IgG-mediated anaphylaxis, different clinical response (e.g systemic hypothermia), differences in epitopes specificities and lack of an IgG4 murine homologue, to name a few. The allergen was de-epitoped specifically in a human-tailored manner, further limiting clinical predictability of studying it with mice. Notwithstanding, numerous peanut allergy and immunotherapy studies have been published using the C3H/HeJ model, which was demonstrated to provide prominent clinical responses. This model was used and established protocols to conduct two studies to provide supporting evidence of B1001 potential safety and efficacy for immunotherapy in humans.

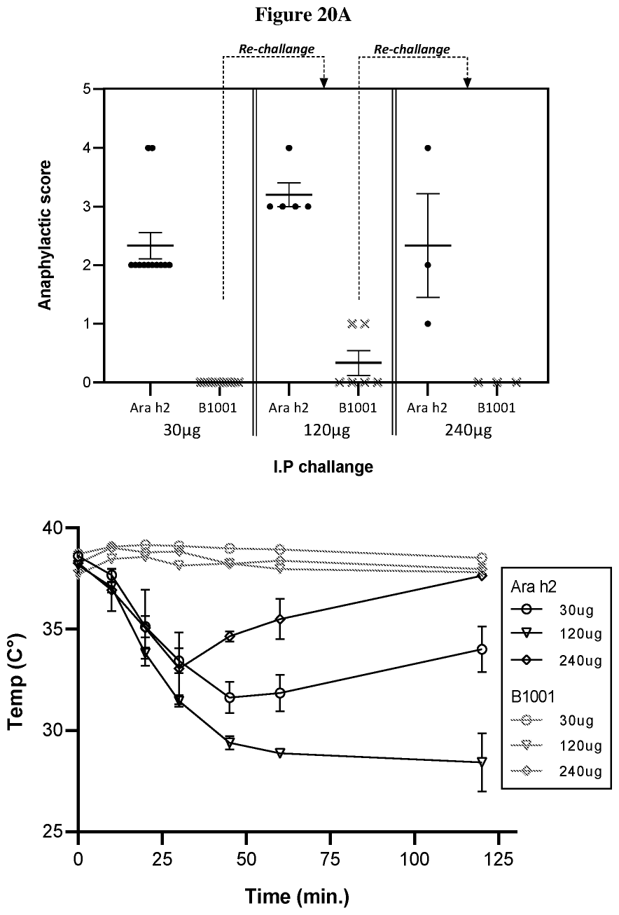

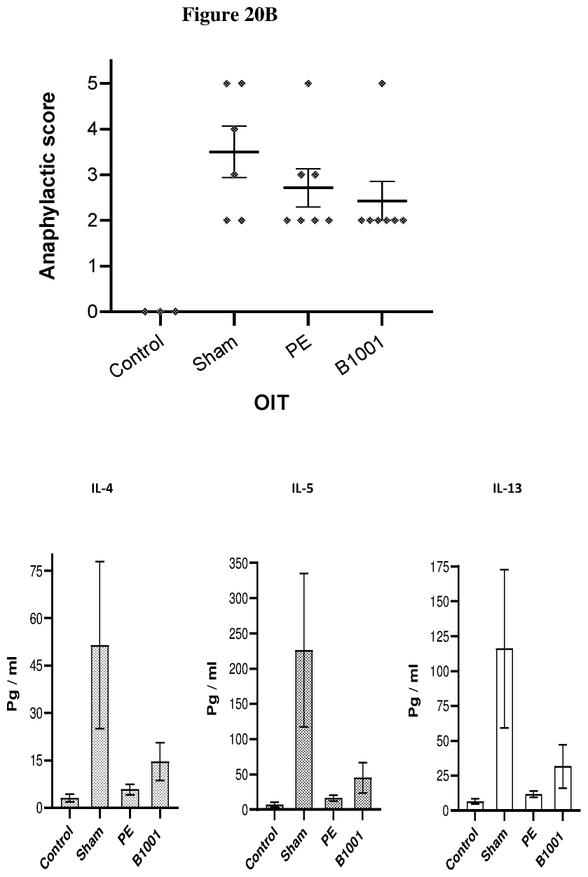

First, C3H/HeJ mice were sensitized by 4 weekly oral gavages with de-fatted peanut flour blended in PBS with the mucosal adjuvant cholera toxin. Then mice were randomized into two groups and sequentially challenged by intraperitoneal (IP) injections with increasing doses of either natural Ara h 2 or B1001. Mice anaphylactic responses were evaluated after 120 minutes by a common clinical scoring index (FIG. 20A, top pane, 0=no response—5=critical response and death) and by rectally recording body temperature over 120 minutes (FIG. 20A, bottom pane). Following the first 30 g challenge, all Ara h 2-challenged mice presented with clear symptoms (n=12, mean clinical score 2.3±0.2, mean maximum temperature drop −7° C.±0.7) while the B1001-challenged mice (n=11) showed no response at all. The next day surviving mice were challenged by 60 g of either protein but this time none of the mice showed any response (data not shown). This is consistent with previously findings suggesting that repeated exposure above a certain dose leads to unresponsiveness, likely due to effector cell exhaustion To avoid this effect on the next day, the B1001-challenged group was further randomized and re-challenged with 120 g Ara h 2 (n=5, score 3.2±0.2, max drop −9.8° C.±1.45) or B1001 (n=6, score 0.3±0.2, no drop in ° C.). This process was repeated on the final day with a similar trend for Ara h 2 (n=3, score 3.2±0.2, max drop −9.8° C.±1.45) and B1001 (n=3, score 0, max drop −0.3° C.±0.1).

Peanut OIT (oral immunotherapy) was performed as a standard alongside B1001 OIT to assess its immunotherapeutic potential. Mice were sensitized by the same protocol as above while retaining an unsensitized group of mice as controls, and then OIT was performed by 5 daily treatments×3 weeks with either peanut flour extract (PE), B1001 or the vehicle PBS. After a 12-day recovery period the mice were IP-challenged with 35 g natural Ara h 2 and anaphylactic scores were recorded (FIG. 20B, top pane). Peanut sensitization was clearly evident (mean scores: control=0, sham=3.5±0.5, MW p-value=0.01). Both PE and B1001 treated groups showed improvement in anaphylactic scores compared to sham with significance (PE mean 2.7±0.4 with p=0.19 vs. sham, B1001 mean 2.4±0.4 with p=0.0.8 vs. sham).

The OIT affected cytokine secretion of mesenteric lymph nodes cells that were stimulated by Ara h 2 were further analyzed. MLNs of surviving mice were harvested and dissociated 5 days after the challenge and then were seeded and stimulated cells for 72 hours. Levels of Th2 cytokines IL-4, IL-5 and IL-13 were then detected in cell culture media using a Luminex panel assay (FIG. 20B, bottom panel). A marked decreased in secretion of all 3 cytokines were found in the PE-treated group compared to sham (p-values: IL-4=0.18, IL-5=0.005, IL-13=0.02). A similar decrease is apparent in the B1001-treated group but to lesser extent and with lower significance (p-values: IL-4=0.26, IL-5=0.087, IL-13=0.095).

In summary, the findings from the murine studies demonstrate that B1001 possesses a decidedly superior in-vivo safety profile compared to Ara h 2 in an allergy model, and provide support for the potential of B1001 as an immunotherapeutic agent.

### Example 12: Characterization of Ara h 1 Variant PLP595 (C159) (SEQ ID NO: 156) and Other Ara h 1 Variants

### Biochemical Characterization of Ara h 1 Variant PLP595

Objective: to characterize Ara h 1 variant PLP595 as compared to WT Ara h 1

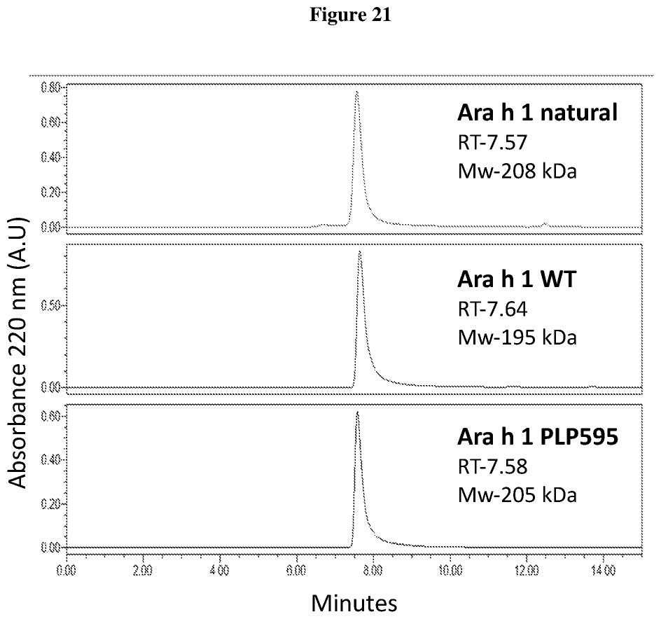

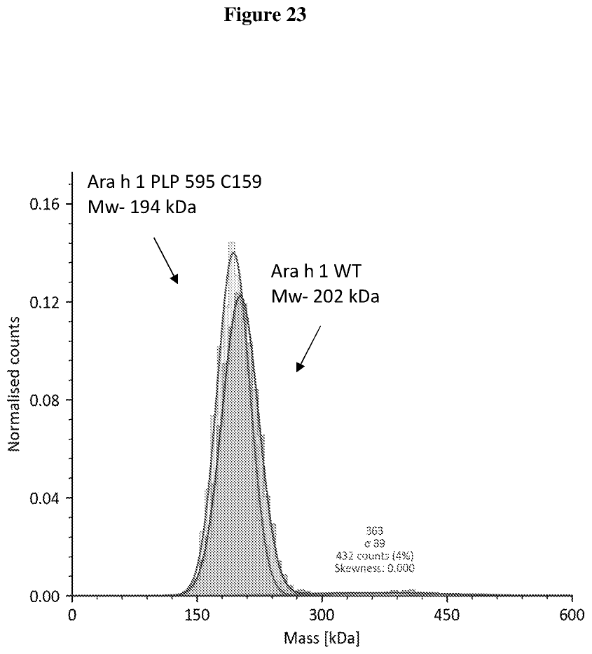

Ara h 1 protein is a trimeric protein, each monomer weight ˜62 kDa, thus the native molecular weight is ˜200 kDa. Size-exclusion HPLC was used to estimate molecular weight and oligomeric state of Ara h 1 variant PLP595, and its profile was compared to the recombinant WT and natural Ara h 1 proteins. All three proteins had similar retention times and estimated molecular weights of ˜200 kDa as shown in FIG. 21. This was further verified by Mass-photometry measurements which resulted in a molecular weight of ˜200 kDa for the WT and Ara h 1 variant PLP595 proteins as shown in FIG. 23. Thus, it was deduced that Ara h 1 variant PLP595 has a similar molecular weight and forms trimers as the WT Ara h 1 protein.

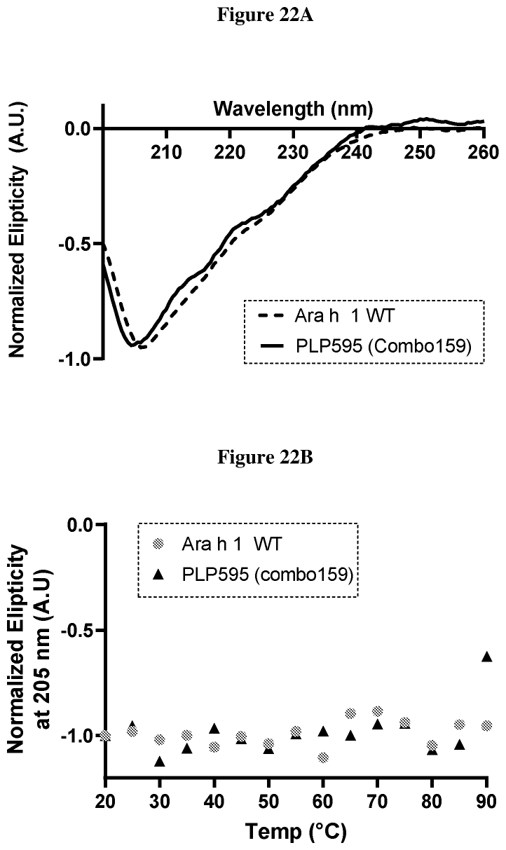

Next, the secondary structure profile of Ara h 1 variant PLP595 was examined by Circular Dichroism (CD) and compared to the WT Ara h 1 protein. Both proteins present a similar CD profile as shown in FIG. 22A. The thermal stability of the secondary structure of Ara h 1 variant PLP595 was also assessed and compared to the WT Ara h 1 protein by carrying out CD at gradually escalating temperatures (20-90° C.). Ara h 1 variant PLP595 was stable up to 85° C. while the WT was stable up to 90° C., as indicating by higher ellipticity at 205 nm (FIG. 22B).

### Differential Decrease in Patient Antibody Binding to Ara h 1 Variant C159

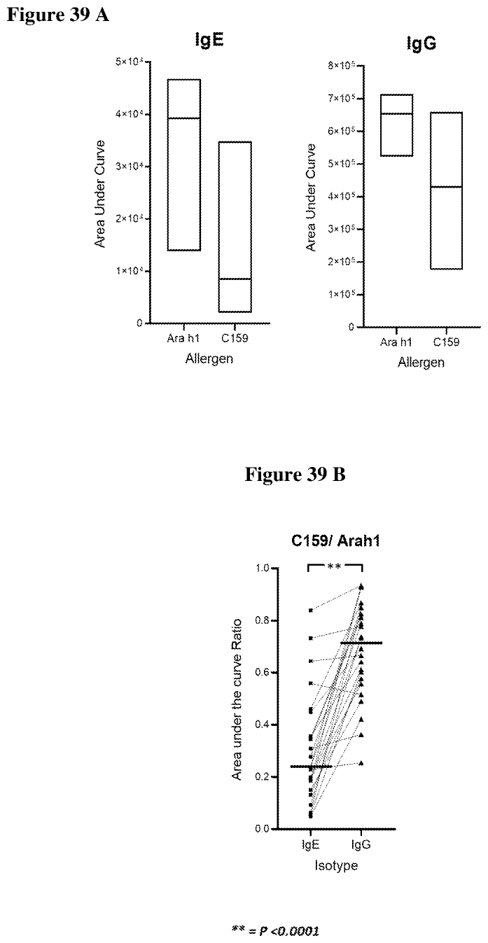

ELISA assay was conducted to examine how the modifications altered IgE and IgG binding. Plates coated with natural Ara h 1 or PLP595 (Combo 159) were incubated with serially diluted plasma or sera from 16 peanut allergy patients. As observed with Ara h 2, the resulting curves significantly varied in shape, therefore, the differences in area-under-the-curve (AUC) values were compared. It was found that binding to C159 was significantly reduced for both the IgE and IgG fractions. However, this reduction was notably more modest for the IgG fraction (FIG. 39A, Wilcoxon matched-pair P value<0.0001). In fact, when observing individual [C159 AUC/Ara h 1 AUC] ratios it is apparent that the reduction was more pronounced for IgE than for IgG fraction in every single patient (FIG. 39B, Wilcoxon P<0.0001). These results demonstrate that the Ara h 1 de-epitoping process was successful in preferentially reducing IgE over IgG binding sites to a significant extent.

### The Allergenic Potential of C159 is Markedly Reduced Compared to Natural Ara h 1

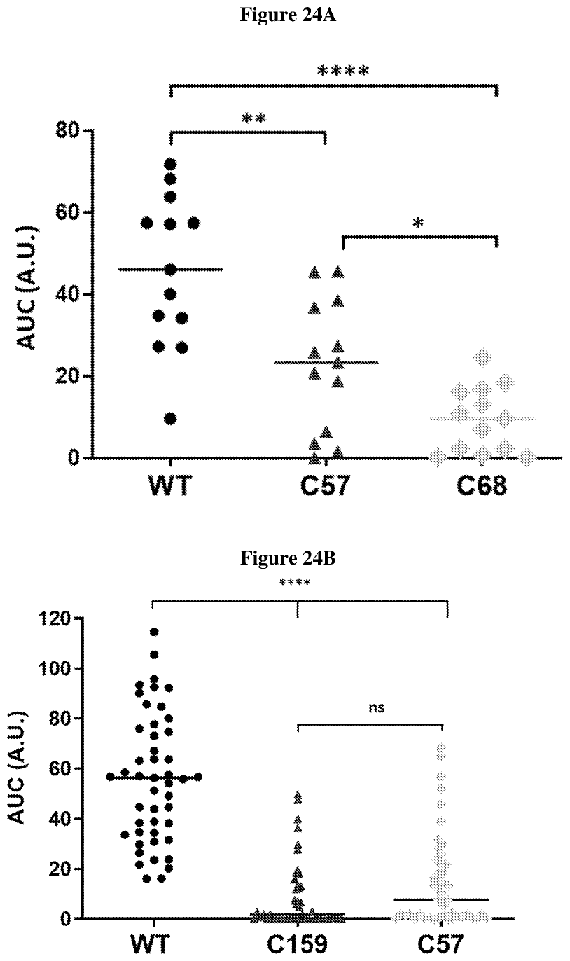

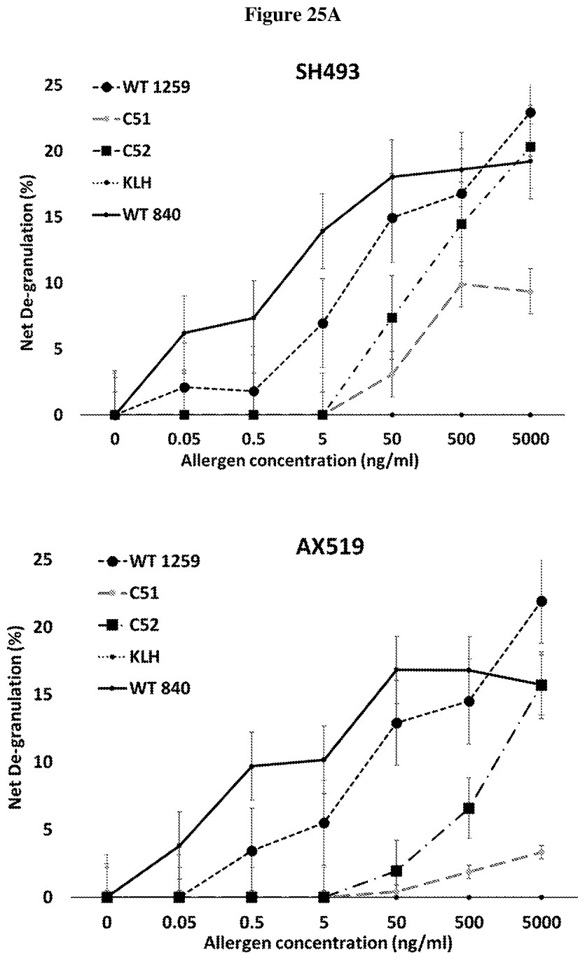

The capability of C57, C68 and C159 to activate the humanized rat basophil-like RBL SX-38 cell line was tested. This widely used cell line can be sensitized with human patient samples to respond to allergen stimulation by cell degranulation. RBL SX-38 cells were sensitized overnight with 1:10 plasma or serum from 13 different clinically validated peanut allergy patients from diverse backgrounds and then stimulated the cells with 0.5-5000 ng/ml of either Ara h 1, C57 or C68. Individual AUC values were calculated in order to compare the responses of each patient to the different allergens. Overall, all patients exhibited reactivity to Ara h 1 (median AUC of 45.7) and reduced reactivity to both variants, with C68 being superior (median AUC of 9.3) compared to C57 (median AUC of 22.5) (FIG. 24A). The same method was utilized for sensitizing RBL SX-38 cells overnight with 1:10 plasma or serum from 47 different clinically validated peanut allergy patients from diverse backgrounds and then stimulated the cells with 0.5-5000 ng/ml of either Ara h 1, C57 or C159. Individual AUC values were calculated to compare the responses of each patient to the different allergens. Overall, all patients exhibited reactivity to Ara h 1 (median AUC 56.3) and reduced reactivity to both variants, with C159 being superior (median AUC of 1.7) compared to C57 (median AUC of 7.5) (FIG. 24A).

These results demonstrate that the de-epitoping process of B1001 dramatically reduced its allergenic potency.

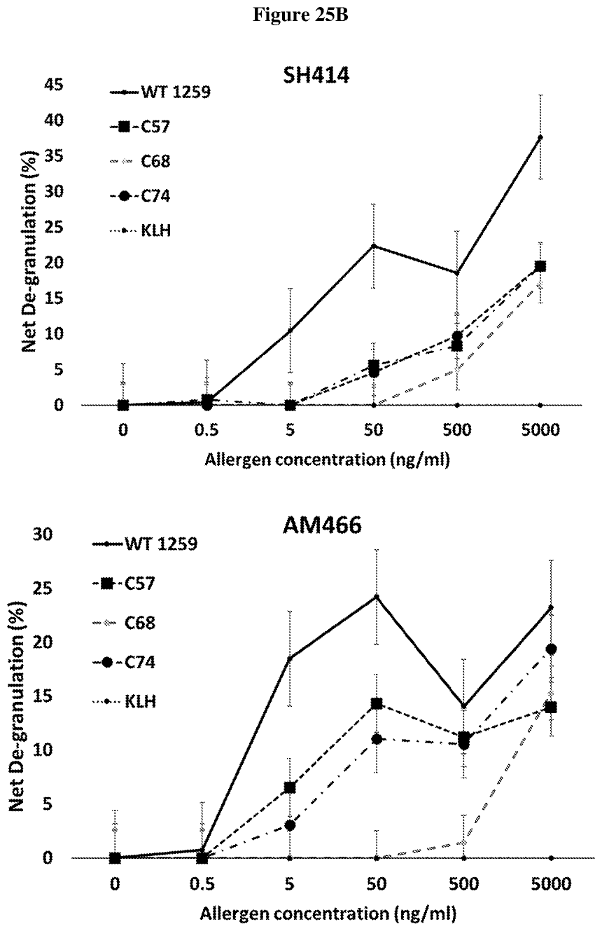

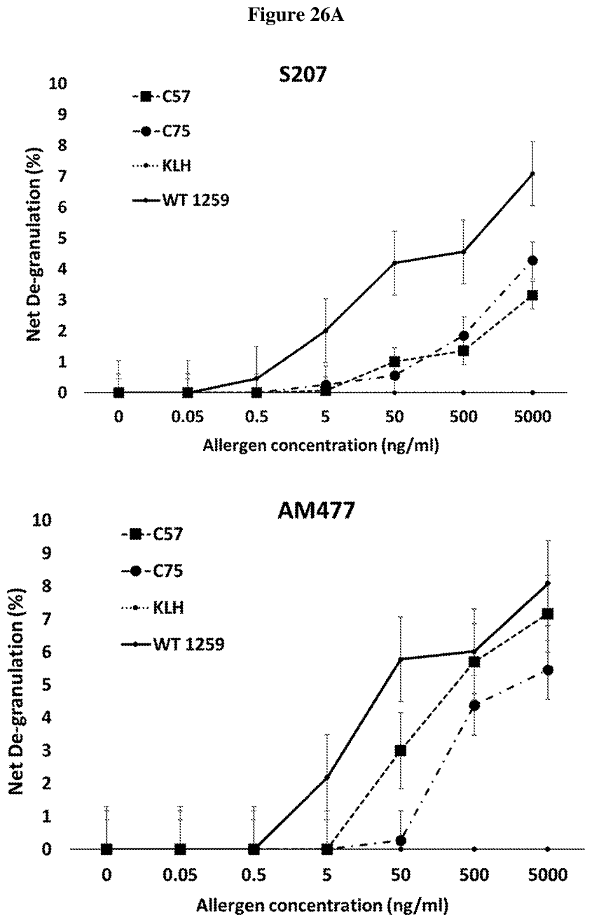

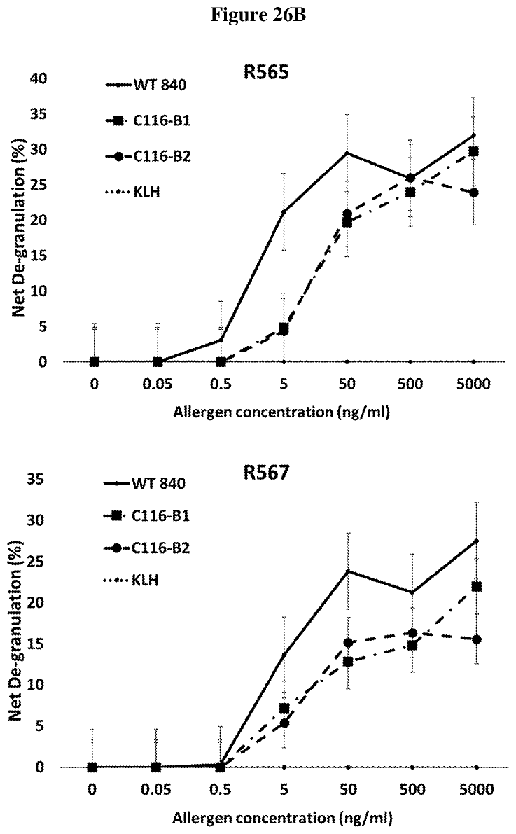

During the Ara h 1 de-epitoping process the performances were tested in SX-38 RBL degranulation assay of additional Ara h 1 variants, including Combo 51 (B1291), 52 (B1292), 74 (B1309), 75 (B1304), or 116 (PLP499). Examples of several tests from individual patients with these variants are shown in FIGS. 25-26.

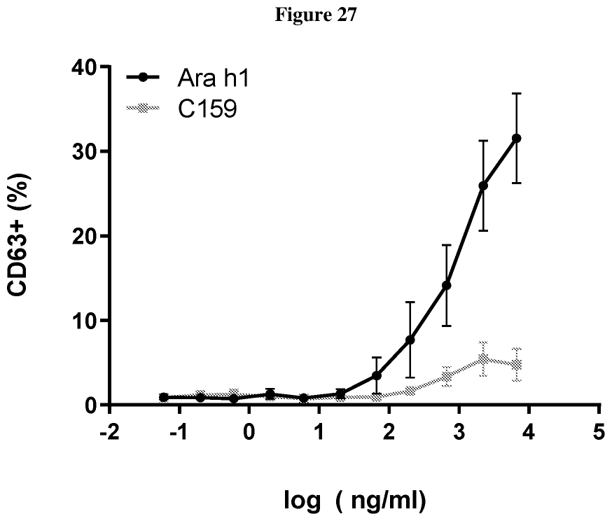

RBL assays allow high throughput comparison of multiple variants using multiple patient samples, making them a powerful tool for engineering and validation of modified allergens. However, sensitivity and accuracy of this assay in predicting patient responses may be limited. To further validate the safety of C159 compared to Ara h 1, BAT assays were performed with a cohort of 19 Israeli and U.S peanut allergy patients using commonly accepted protocols with allergen concentration ranging 0.06-6,600 ng/ml. EC50 values derived from the resulting curves by fitting to a 4-parameter logistic regression model suggest C159 has >1000-fold reduced reactivity at the population level (FIG. 27). These results demonstrate the increased safety of C159 over Ara h 1 and provide support for its application in immunotherapy.

### T Cell Immunogenicity of C159

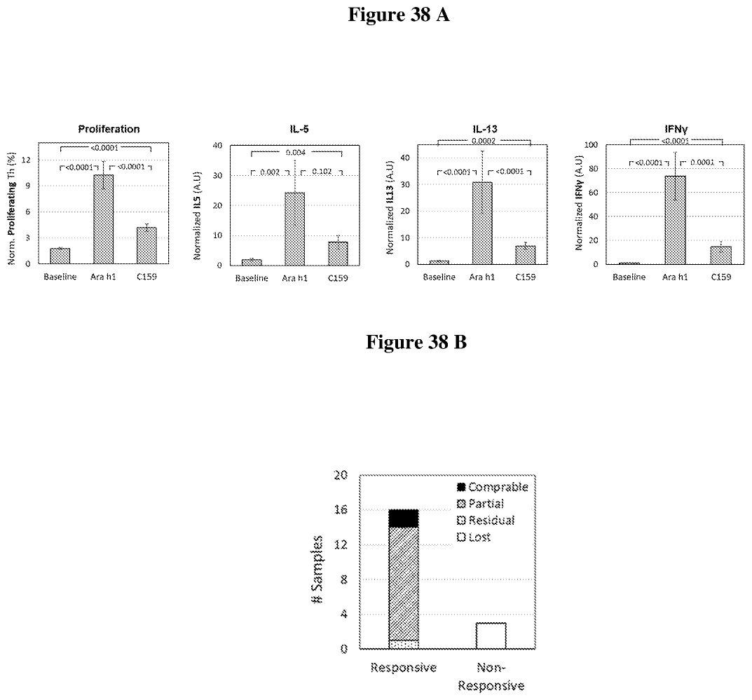

To ensure that the modifications did not abolish C159 T cell immunogenicity, peanut allergy patient peripheral blood T cells were treated with proliferation detection dye and stimulated with either PBS, recombinant WT Ara h 1 or C159. Then both cells were retained for cytometric proliferation analysis and media for sandwich ELISA analysis of secretion of Th2 cytokines IL-5 and IL-13 and Th1 cytokine IFNγ (FIG. 19A). Then cells were retained for cytometric proliferation analysis and media for sandwich ELISA analysis of secretion of Th2 cytokines IL-5 and IL-13 and Th1 cytokine IFNγ (FIG. 38A). Data were collected only from samples that cleared pre-determined thresholds for Ara h 1. It was found that while peanut allergic T cell reactivity to C159 was reduced compared to WT, activation nonetheless still clearly and significantly retained in all tested parameters. To assess overall C159 reactivity, pre-determined thresholds were used to classify each patient sample as non-reactive or has having partial or comparable reactivity compared to Ara h 1. Of total 19 samples tested, 2 were estimated as comparable, 14 as partial and 3 were unreactive (FIG. 38B). These results indicate that de-epitoping for IgE only partially impaired the allergen's T cell immunogenicity.

### Example 13: Increasing the Half-Life of De-Epitoped Ara h 2 for mRNA Therapy

Objective: to increase the half-life of de-epitoped Ara h 2 and improve its therapeutic potential.

De-epitoped Ara h 2 (denoted as 1001) was initially designed for bacterial expression but is poorly expressed in mammalian cells. The inability of this protein to be expressed and secreted from mammalian cells may preclude its use as part of an mRNA therapy. In addition to the poor mammalian cell expression, de-epitoped Ara h 2 is small monomeric protein with a molecular weight<19 kDa and as such, it is expected to be rapidly cleared by the renal pathway. Increasing the half-life of this protein will improve its therapeutic potential by effectively prolonging its exposure to the immune system and so the opportunity to produce the desired immune response.

Ara h 2 and its de-epitoped derivatives were also observed as being spuriously O-glycosylated (validated by ETD mass spectrometry, data not shown) in a manner that interfered with protein expression via the mammalian secretory pathway. Abolishing the glycosylation site had improved the expression levels of wild type Ara h 2 but was not sufficient to increase the expression levels of the de-epitoped derivatives.

Several constructs were designed to address the above issues, tailoring de-epitoped Ara h 2 1001 to function as part of an mRNA therapy.

### Methods

Cell Transfection—Expi293 cells (ThermoFisher) were grown in Expi293 expression medium and transfected according to the manufacturer's protocol. Briefly, prior to transfection cells were grown to viable cell density of 4-5 million cells/ml, diluted to 3 million cells/ml, and transfected with 1 ug DNA per ml medium. DNA was diluted to 6.1% of the expression volume in OptiMEM (ThermoFisher). In a separate tube, ExpiFectamine293 was diluted 1:18.5 in OptiMEM to 6% of the expression volume. Following an incubation for 5 minutes, the diluted Expifectamine293 (Gibco) and DNA were mixed, incubated for 10 minutes, and added to the cell culture.

Protein Expression—Expi293 cells were grown at 37°, 5% CO₂. One day following transfection, cells were supplemented with 1:160 and 1:16 volumes of Expifectamine293 enhancer 1 and 2 respectively. Cells were left to express the protein for a total of 5 days.

Protein Purification—The medium supernatant was clarified by centrifugation at 300×g for 10 minutes and filtered through a 0.45 um PES filter. His tagged constructs were dialyzed overnight against 100 volumes of 20 mM tris pH 8.0, 200 mM NaCl. The dialyzed supernatant was agitated 1 hour with Ni-NTA Superflow resin (ThermoFisher) at 4°. The resin was washed with 20 mM tris pH 8.0, 200 mM NaCl, 10 mM imidazole. The protein was eluted with 20 mM tris pH 8.0, 200 mM NaCl, 250 mM imidazole. For Fc fusion protein, the clarified medium supernatant was incubated 1 hour with protein A-conjugated resin (Toyopearl, HC-650F). The resin was washed with 100 resin volumes of PBS. The protein was eluted with the addition of 0.1 M Na citrate buffer pH 3.0. The eluted fractions were neutralized with the addition of 0.33 elution volumes of 1 M tris pH 9.0. For additional purification, eluted fractions were concentrated by Amicon centrifugal filters (Merk Milipore) of an appropriate MWCO, either 10 or 50 kDa, and loaded onto a Superdex75 PG or Superdex200 PG 16/600 (Cytiva) equilibrated to PBS for size exclusion chromatographic separation.

ELISA Assay—Sera were collected from mice prior to treatment and at day 21 following the first DNA injection. Antigen specific antibodies were detected in mouse sera by an ELISA assay. Briefly, 96 well ELISA plates (MaxiSorp, Nunc) were coated at 4° with 50 μl (1 μg/ml) purified protein in phosphate buffered saline according to the following scheme—Natural Ara h 1 was used for the detection of α-Ara h 1 and α-DE Ara h 1 combo 68 antibodies (both soluble and transmembrane fusions). Natural Ara h 2 was used to detect α-Ara h 2 antibodies, recombinant DE Ara h 2 1001 was used to detect α-DE Ara h 2 1001 antibodies (both Fc fusions and transmembrane fusions). Keyhole limpet hemocyanin (KLH, Sigma Aldrich) at 1 μg/ml was used as a negative control. All conditions were performed in duplicate. After coating, plates were blocked by incubation with PBS 0.1% Tween20 (PBST), 2% BSA for 1 hour at room temperature, then washed once with 200 μl PBST. Sera were diluted 1:200 in PBST 2% BSA and 50 μl/well transferred to the ELISA plate according to the scheme above and incubated 1.5 hours at room temperature. The plates were washed three times with 200 μl/well PBST. All wells were incubated with 50 μl 1:10,000 HRP-conjugated α-mouse IgG (Jackson ImmunoResearch) secondary antibody. Wells were washed three times with 200 μl/well PBST followed by a TMB reaction (Promega) and quenched with the addition of H₂SO₄. The optical density values were subtracted from that of the values of the KLH control.

In Vivo Transfection—Female C3H/HeNHsd mice, 6-8 weeks old were purchased from Envigo (Envigo, Israel), and treated with DNA constructs consisting of a plasmid encoding for the protein of interest flanked by a CMV promoter and SV40 polyadenylation signal (‘pTwist CMV’, Twist Bioscience). Mice were treated by injection of 10 μg DNA in PBS or via PEI transfection. Briefly, for the preparation of 840 μl DNA for PEI transfection, the DNA was diluted in 400 μl at a final concentration of 0.42 mg/ml in 5% glucose. In a second tube 70.4 ul of 1 mg/ml 25 kDa linear PEI (Polyscience) was diluted in 440 μl 5% glucose. The two tubes were mixed by pipetting and incubated for 15 minutes prior to injection. Final DNA n/p ratio=6. Mice were injected I.M, with 50 μl, 0.2 mg/ml into the caudal thigh muscle three times weekly and bled on day 21 following the first administration.

### Results

### Consensus Mutants

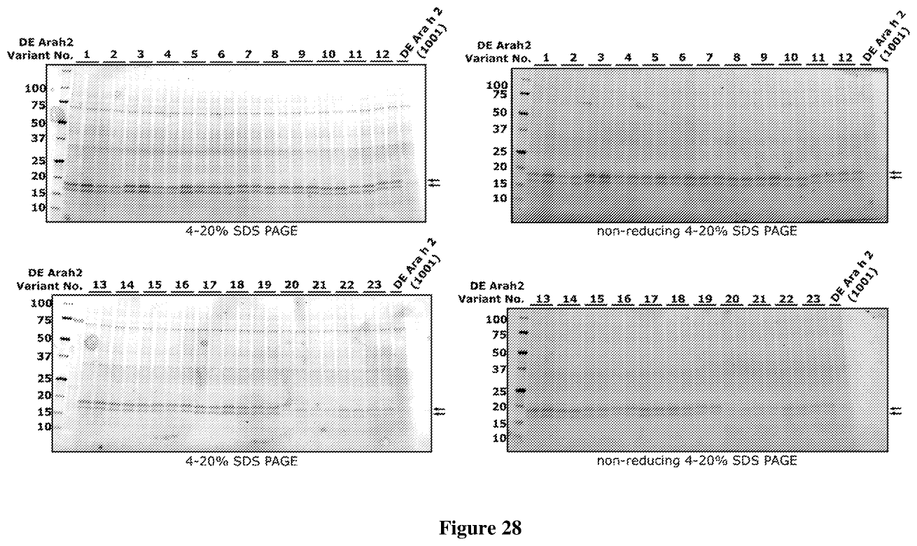

To address the poor expression of de-epitoped Ara h 2 1001 in mammalian cells a set of back-to-consensus mutations were designed with the intention of regaining the stability of the wild type protein, while avoiding re-introducing IgE epitopes. The designs were iteratively tested for both expression levels and RBL activation. After several design iterations, a well-expressing version of de-epitoped Ara h 2 without significantly re-introducing IgE epitopes was attained (Arah2_conbo31). FIG. 28 shows an example of back-to-consensus variants of DE Ara h 2 1001 expressed in HEK293 cells, and demonstrates the increased secretion levels of the subset of the back to consensus mutants when compared to the DE Ara h 2 1001 from which they were derived (right lane). The non-reduced gels demonstrate that the variants are monomeric, and not misfolded, forming intermolecular disulfide bonds, a phenomenon observed with some recombinant versions of Ara h 2.

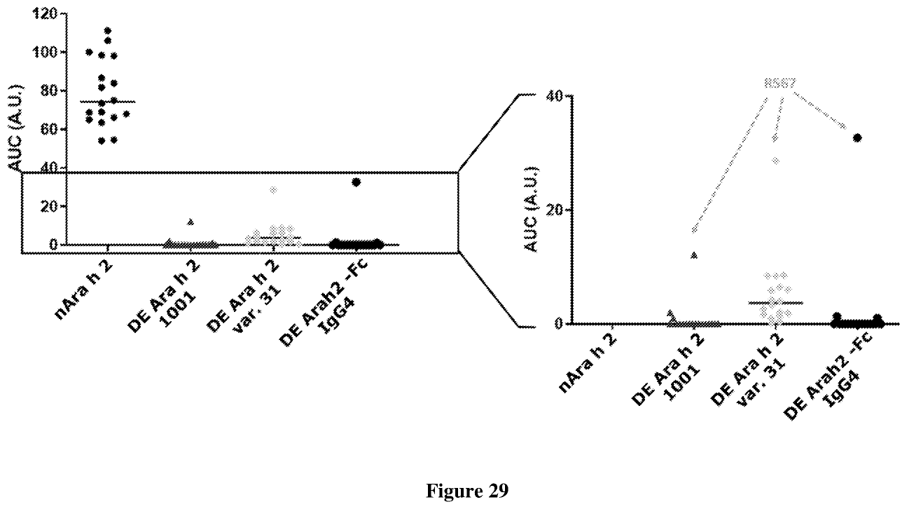

As shown in FIG. 29, the final back-to-consensus mutant (var 31) and the DE Ara h 2 1001-Fc fusion proteins are significantly less allergenic when compared to natural Ara h 2, as measured by RBL assays.

### Fc Fusions

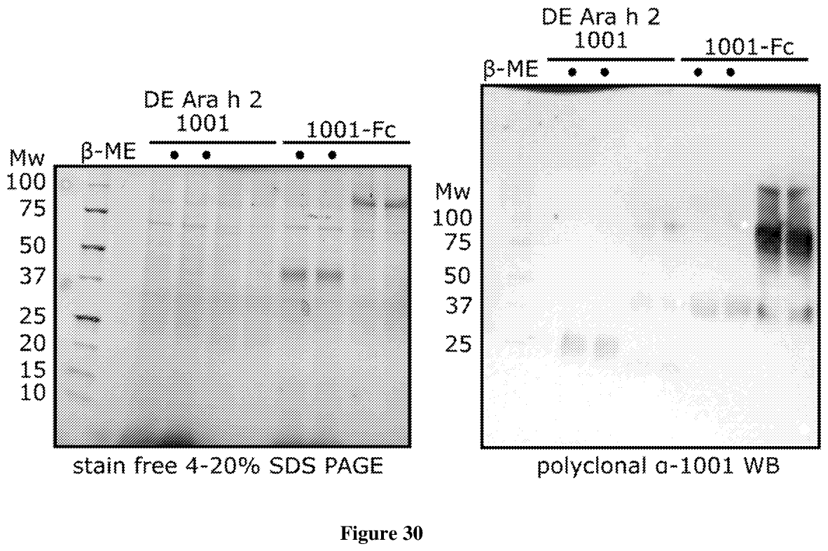

To address the poor expression, short half-life, and fast clearance of de-epitoped Ara h 2, the de-epitoped Ara h 2 was fused to an antibody Fc. The Fc moiety fulfils two functions, acting as both a carrier in the secretory pathway, and increasing the half-life of the fused therapeutic moiety. In addition to the above functions, fusion of the de-epitope allergen to the Fc of IgG4 is expected to inhibit the allergic response by binding to FcγR. FIG. 30 shows that fusion to the Fc dramatically increased the secretion levels of de-epitoped Ara h 2 1001 and demonstrates the markedly increased secretion levels of the de-epitoped Ara h 2 Fc fusion (and assembly of the Fc dimer) when compared the monomeric protein. The monomeric protein can barely be detected by Western blot, where's the FC fusion is clearly overexpressed and secreted to the medium as seen in the SDS PAGE gel. The Western blot confirms the overexpressed protein band contains the de-epitoped Ara h 2 fusion.

### Transmembrane Fusions

To further address the poor expression, short half-life, and fast clearance of de-epitoped Ara h 2, a membrane-anchored version of the de-epitoped Ara h 2 was designed. In addition to facilitating increased expression, the membrane fusion cannot be cleared by the renal system as would occur in a soluble version. Additionally, the membrane fused protein can elicit the production antibodies, but being anchored to a cell membrane and immobilized, cannot likely induce crosslinking of FcεRI and therefore will not likely cause an allergic response.

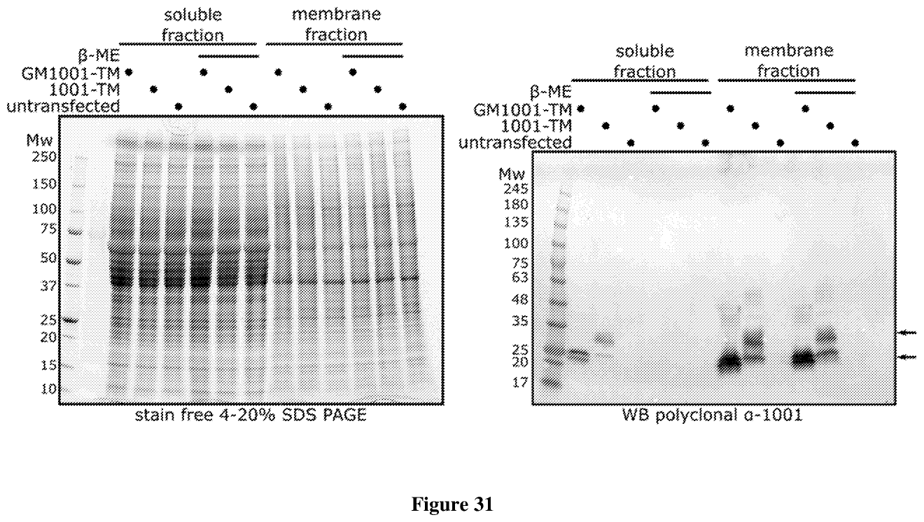

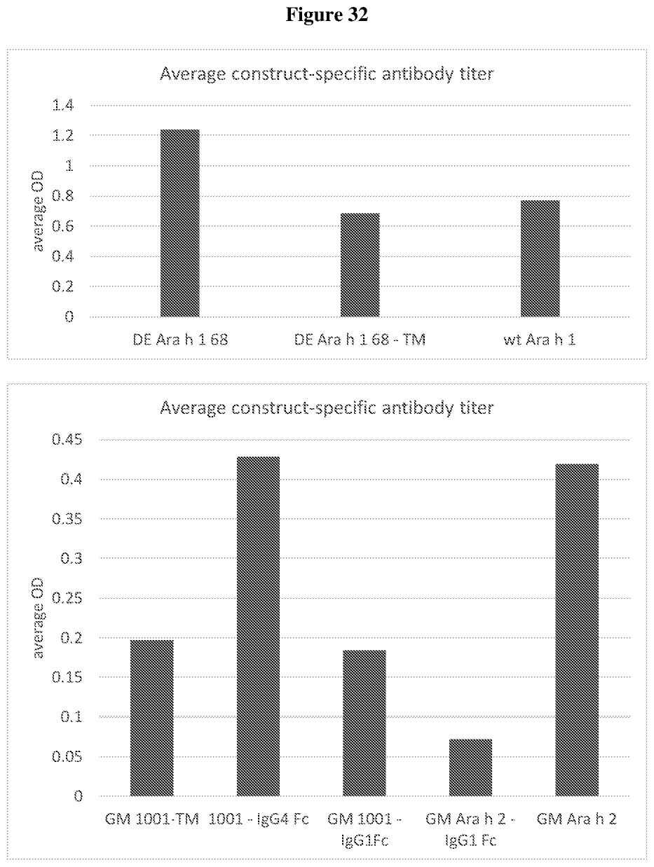

FIG. 31 demonstrates that DE Ara h 2 1001 can be overexpressed anchored to the membrane, with only negligible amounts of the protein found in the soluble fraction. FIG. 32 demonstrates the antibody response to the various constructs when delivered as part of a gene therapy. This response confirms that the constructs are indeed capable of being expressed and secreted in vivo, and that they elicit the immune response that is expected to be integral to the immunotherapy.

### Example 14: Sublingual Immunotherapy for Peanut Allergy

### Peanut Extract Preparation

One hundred grams of defatted peanut flour (Shaked Tavor, ˜48% protein, ˜80% defatted, from lightly roasted peanuts) were mixed with 500 ml extraction buffer (20 mM Tris, pH 8.0), homogenized using hand homogenizer mixer and stirred for 2 hrs at room temperature. The mixture was then centrifuge at 5000 g for 5 min and the supernatant was centrifuged again at 20,000 g for 50 min at 4° C. The obtained supernatant was re-centrifuged 20,000 g for 50 min at 4° C. and filtered through 0.45 μm filter. The filtered peanut extract (PE) was kept at −80° C. till the purification step.

### Isolation of Natural Ara h 2

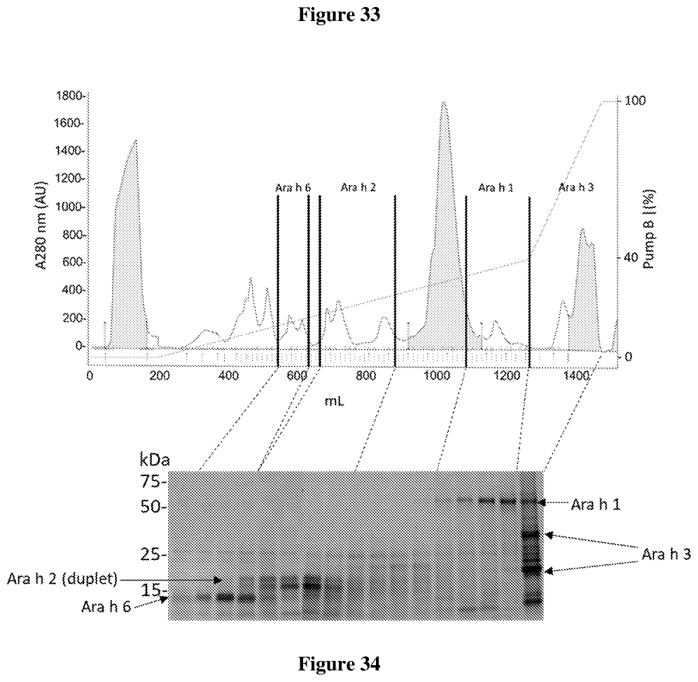

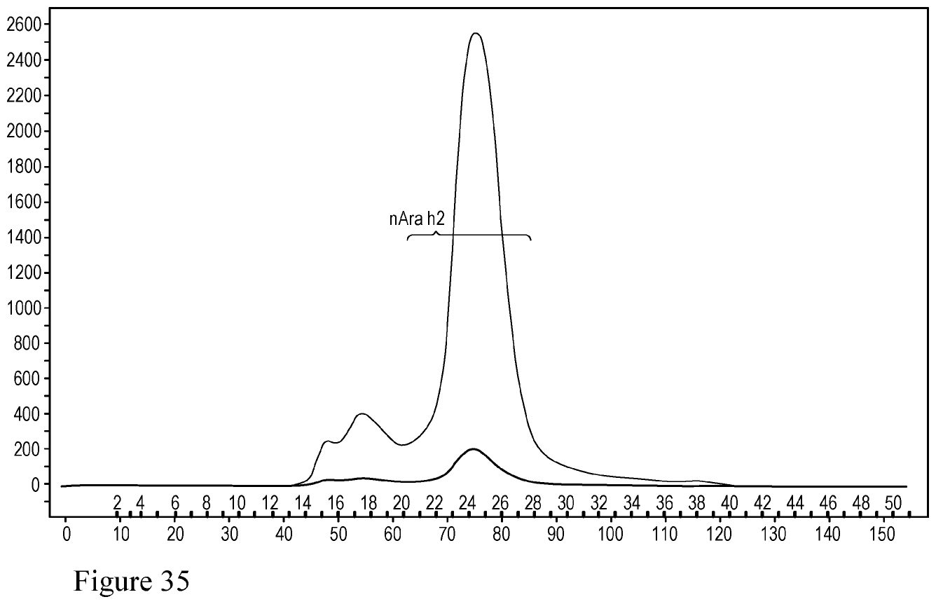

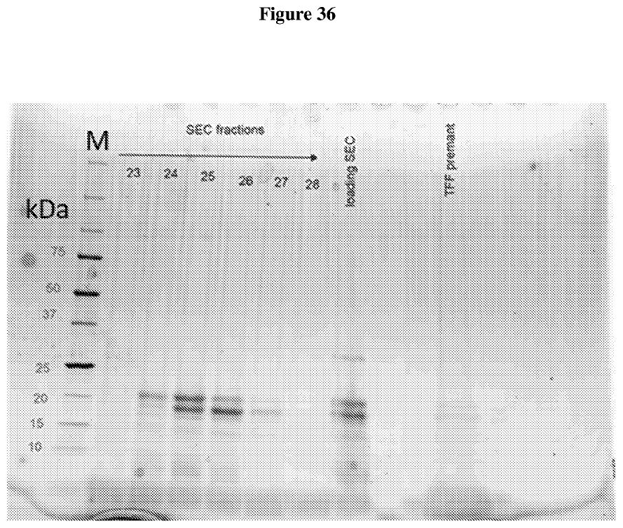

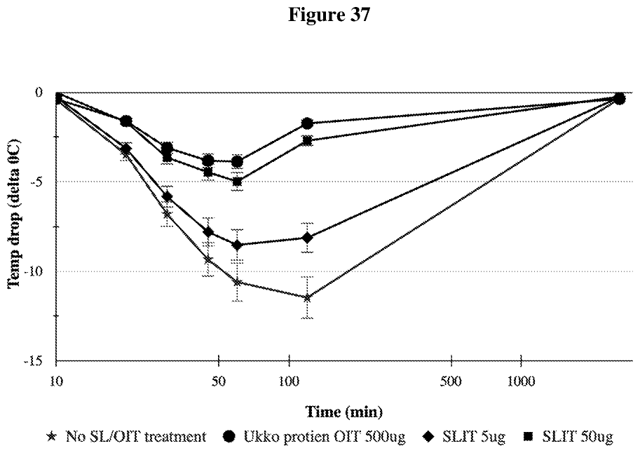

One hundred ml PE were loaded on 70 ml Q Sepharose HP column (Cytiva), pre equilibrated with extraction buffer. Peanut proteins were eluted using 18 column volumes linear gradient of 0-0.4 M NaCl in extraction buffer (FIG. 33A). Natural Ara h 2 (nAra h 2 containing fractions (see FIG. 33B) were pooled, concentrated to 2-5 mg/ml by 3 kDa centricones (Amicon, Mercury), and loaded on Hiload 16/600 Superdex 75PG SEC column (Cytiva) equilibrated with PBS. The pure monomeric nAra h 2-containing fractions were pooled and concentrated to ˜2 mg/ml (see FIGS. 3 and 4). Concentration was determined using absorbance at 280 nm (E=14940). Final concentration of material was determined on SEC-HPLC using BEH SEC 200A column (Waters) and Myoglobin standard curve.

### Sublingual Immunotherapy for Peanut Allergy in A Mouse Model

### Experimental Procedure

Thirty-six naïve female C3H/HeJ mice of 3 weeks old were ordered. On Day 1, the body weight range of the mice was 14-18 g. They were identified using indelible marker on the tail. They were supplied by Jackson Laboratory, Bar Harbor, U.S.

### Sensitization Phase

The 36 mice (including sham animals) were orally sensitized as described below:

Week 1, 2 and 3: once a week 2 mg (50% protein) of peanut extract blended in 0.250 mL of PBS, 10 μg of the mucosal adjuvant cholera toxin (List Laboratories, Campbell, Calif, reference 100B).

Week 4: 4 mg (50% protein) of peanut extract blended in 0.250 mL of PBS, 10 μg of the mucosal adjuvant cholera toxin (List Laboratories, Campbell, Calif).

The mice were deprived of food for 3 hours before each gavage.

On Day 29, all mice were intraperitoneally challenged with 350 μg of peanut extract. Body temperatures were measured with a rectally inserted thermal probe before, 30 and 40 minutes after the i.p. challenge. A drop above 1.5° C. in temperature was considered as positive.

Anaphylactic symptoms were evaluated 40 minutes after the i.p. challenge using the following scoring system:
0: no clinical symptoms;
1: repetitive mouth or ear scratching and ear canal digging with hind legs;
2: decreased activity, edema/puffiness around eyes and/or mouth;
3: periods of motionless for >1 min, lying prone on stomach;
4: no responses to whisker stimuli, reduced or no response to prodding; and
5: end point: tremor, convulsion, death.

Then, a blood sample of approximately 100 μL was collected at the level of the sub-mandibular vein without anesthesia (polypropylene serum tube containing clot activator) for the measurement of total immunoglobulin at Porsolt using an enzyme immunoassay kit. Total blood was mixed with the clotting activation agent by inverting the tube several times. The vial was maintained between 20 and 30 minutes at room temperature (tube standing upright). The blood was then centrifuged at 1000 g for 10 minutes at room temperature. Serum samples (one serum sample of 25 μL+one serum sample of the remaining volume) were transferred in polypropylene tubes and kept frozen at −80° C. until analysis.

Total immunoglobulin quantification (IgA, IgE, IgG1, IgG2b, IgG3, IgM and IgG2c) was performed using clarified plasma samples and an antibody Isotyping 7-Plex Mouse ProcartaPlex™ Panel (reference EPX070-20816-901, ThermoFisher). ProcartaPlex Mouse Basic Kit for IgG2a (reference EPX010-20440-901, ThermoFisher) was used for total IgG2a quantification.

Further to the data obtained on Day 29 (temperature and clinical score) after the i.p. challenge with 350 μg of peanut extract, 28 mice were selected. No additional test was conducted on Day 33.

### Treatment Phase

From Day 36, oral or sublingual immunotherapy was initiated (5 administrations per week for 3 successive weeks). For sublingual administration (i.e., sham and group 3 and 4, see Table 1), the mice were shortly anesthetized by a mixture of ketamine/medetomidine (50/1 mg/kg, 10 mL/kg i.p.) on the first week of treatment. After approximately 10 minutes, the mouse was checked for the depth of narcosis to make sure it was well anesthetized.

Sublingual Administration: The mice were held in a head-up vertical position, and a micropipette was used to apply 10 μL of solution per mouse under the tongue.

Tongue Rolling: After the mice had been dosed, the dorsal surface of the tongue was gently rolled for approximately 1 minute. This was to simulate the normal tongue movements in a conscious animal and can be performed with the tip of micropipette.

Recovery Positioning: Afterwards, the mice were placed in anteflexion (sitting with their head bend over their lower extremities) for approximately 20 minutes after sublingual delivery to minimize the likelihood that the mice swallowed the solution.

After oral administration of the mice in group 2, the mice were shortly anesthetized by a mixture of ketamine/medetomidine (50/1 mg/kg, 10 mL/kg i.p.), as done in the other groups. Therefore, all animals were tested under the same experimental conditions (i.e. with a short anesthesia).

Due to mortality further to anesthesia on the first week of treatment, the protocol of anesthesia was modified: The mice were shortly anesthetized by a mixture of ketamine/medetomidine (25/2 mg/kg, 10 mL/kg i.p.).

After approximately 30 minutes of anesthesia, atipamezole (1 mg/kg, i.p., 10 ml/kg) was used to reverse the anesthetic effects of ketamine/medetomidine.

TABLE 10
Experiment Groups

| | Sensitization | | | Challenge |
|---|---|---|---|---|
| | (peanut | | Dose-level | (i.p., i.d.^{(*)}, |
| Group | extract) | Treatment | (μg/mouse) | i.g.) |
| 1 | Yes | Sham (PBS, | 0 (10 μL of | Natural Ara h 2 |
| (n = 4) | | sublingual) | PBS) | protein |
| 2 | Yes | Natural Ara h 2 | 500 μg/250 μL | Natural Ara h 2 |
| (n = 8) | | protein (Oral | | protein |
| | | treatment) | | |
| 3 | Yes | Natural Ara h 2 | 5 μg/10 μL | Natural Ara h 2 |
| (n = 8) | | protein | | protein |
| | | (Sublingual | | |
| | | treatment) | | |
| 4 | Yes | Natural Ara h 2 | 50 μg/10 μL | Natural Ara h 2 |
| (n = 8) | | protein | | protein |
| | | (Sublingual | | |
| | | treatment) | | |

^{(*)}peanut for the i.d. challenge (right ear only)
Inter-group comparison was performed using an Unpaired Student's t test.

### Challenge (i.p.)

On Day 67, the mice were intraperitoneally challenged with 35 μg natural Ara h 2 protein/250 μL. Core body temperature was measured with a rectally inserted thermal probe before, 10, 20, 30, 45, 60, 120 minutes and 24 hours after i.p. challenge. A 1.5° C. drop in temperature was considered as positive.

Cytokine Secretion from Splenic and Mesenteric Lymph Node Cells

After blood sample collection (Day 72), spleens and mesenteric lymph nodes (MLN) were collected and transferred into 1×PBS containing 100 U/mL penicillin and 100 μg/mL streptomycin in separate Falcon tubes placed on ice. MLN were cut in small pieces using sterile instruments. Spleen was freshly homogenized using the GentleMACS dissociator. Then, they were transferred onto a 70 μM cell strainer pre-wet with TexMACS medium (ref. 130-097-196, Miltenyi Biotec).

Splenocytes were isolated and then centrifugated at 450 g, 8 min. Red blood cells were lysed using Lysing buffer (ref 555899, BD Biosciences). Reaction was stopped using 5 volumes of 2% FBS in PBS and cells were washed once with PBS. MLN cells were isolated by gently pressing the tissues with a syringe plunger with repeated addition of culture medium and then centrifuged at 450 g, 8 min.

Splenocytes and MLN cells were seeded in 96-well U-bottom plates (400,000 cells/100 μL) in TexMACS medium (ref. 130-097-196, Miltenyi Biotec) and 10% FBS containing 100 U/mL penicillin and 100 μg/mL streptomycin and treated with cell culture medium (group 1) or natural Ara h 2 at a final concentration of 200 μg/mL (groups 1-4). Cells from the sham and sensitized mice were also treated with concanavalin A (2.5 μg/mL final concentration) or stimulated with CD3-CD28 beads using mouse T Cell Activation/Expansion Kit (ref. 130-093-627, Miltenyi Biotec) as a control. Supernatants were collected at 24 and 72 hours post-treatment and stored at −80° C. until analysis.

The levels of cytokines (IL-4, IL-5, IL-10, IL-13, INF gamma, IL-12, IL-9 and TGFβ) were measured using a Luminex panel assay following manufacturer instructions (ProcartaPlex 7 plex Assay, ThermoFisher Scientific, reference no. EPX010-20440-901 and TGF beta1 Mouse ProcartaPlex™ Simplex Kit, ThermoFisher Scientific, reference no. EPX01A-20608-901). Data were analyzed with the Bio-Plex Manager software (Biorad) and concentrations were calculated using the standard curve of the corresponding cytokine.

### Results

### Challenge (i.p.): Assessment of Hypersensitivity Reactions

Hypersensitivity reactions as measured by changes in body temperature are shown in FIG. 5. In sham mice, a progressive decrease of temperature was observed over time (maximum −11.5±1.3° C. at 120 minutes after the i.p. challenge).

In mice treated with peanut protein (400 μg/mouse p.o.), the temperature drop was less marked as compared to sham mice (−3.9±1.3C maximum at 60 minutes after the i.p. challenge and −1.7±0.6° C. at 120 minutes). The difference between groups reached statistical significance from 20 to 120 minutes post-challenge.

In mice treated with peanut protein (5 μg/mouse sublingual), the temperature drop was not significantly modified as compared to sham mice.

In mice treated with peanut protein (50 μg/mouse sublingual), the temperature drop was less marked as compared to sham mice (−4.9±1.2° C. maximum at 60 minutes after the i.p. challenge and −2.77±1.3° C. at 120 minutes). The difference between groups reached statistical significance from 20 to 120 minutes post-challenge.

In all mice, the clinical score measured at 30 minutes after the i.p. challenge was 2. No differences were therefore observed between groups.

### Analysis of Cytokine Production

### Positive Controls

Positive controls induced an increase in cytokine secretion for most of the tested cytokines from splenocytes and mesenteric lymph node cells with lower levels for the mesenteric lymph node cells. The spleen and mesenteric lymph nodes were collected in non-responding animals and not in naïve animals which were not available in this study.

### Splenocytes

In the supernatant of splenocytes from sham control mice, the levels of IL-4, IL-5, IL-10, IL-13, INF gamma and IL-12 increased between 24 and 72 hours. The IL-9 level was below the limit of quantification and the TGFβ level remained stable over the time. As negative controls, splenocytes from sham control mice treated with culture medium, the levels of cytokines were very low or below the limit of quantification, except for TGFβ (basal levels of approximately 350 μg/mL).

In the supernatant of splenocytes from orally sensitized mice (400 μg/mouse p.o.), the IL-4, IL-5, IL-10, IL-13, INF gamma and IL-12 levels were not clearly modified as compared to those of sham control mice. The TGFβ level was significantly increased at 24 hours (+81%, p<0.01) and 72 hours (+80%, p<0.05) as compared to those of sham control mice. However, considering the basal levels measured in control conditions, this variation is likely devoid of biological relevance.

In the supernatant of splenocytes from sublingually sensitized mice (5 or 50 μg/mouse), the IL-4, IL-5, IL-10, IL-13, INF gamma, IL-12 and TGFβ levels were not clearly modified as compared to those of sham control mice.

TABLE 11
Cytokine Secretion (pg/ml) By Splenocytes

| | IL-4 | | IL-5 | | IL-10 | | IL-13 | | TNF-B | |
|---|---|---|---|---|---|---|---|---|---|---|
| | 24 h | 72 h | 24 h | 72 h | 24 h | 72 h | 24 h | 72 h | 24 h | 72 h |
| AVG (mean) | | | | | | | | | | |
| PE Sham IT | 33 | 110 | 62 | 938 | 121 | 828 | 45 | 439 | 233 | 212 |
| PE OIT | 46 | 78 | 137 | 699 | 103 | 656 | 59 | 351 | 233 | 212 |
| PE SLIT 5 | 60 | 76 | 158 | 953 | 143 | 859 | 104 | 604 | 330 | 153 |
| PE SLIT 50 | 52 | 49 | 97 | 632 | 111 | 616 | 53 | 309 | 402 | 287 |
| SEM | | | | | | | | | | |
| PE Sham IT | 18 | 9 | 10 | 307 | 5 | 97 | 1 | 96 | 20 | 61 |
| PE OIT | 18 | 9 | 10 | 307 | 5 | 97 | 1 | 96 | 20 | 61 |
| PE SLIT 5 | 15 | 22 | 19 | 111 | 14 | 108 | 18 | 122 | 61 | 28 |
| PE SLIT 50 | 25 | 29 | 37 | 224 | 22 | 199 | 15 | 118 | 37 | 29 |

### Mesenteric Lymph Node Cells

In the supernatant of mesenteric lymph node cells from sham control mice, the levels of IL-4, IL-5, IL-10, IL-13, INF gamma and IL-12 increased between 24 and 72 hours. The IL-9 level was below the limit of quantification. In the two tested wells, the kinetic was different for TGFβ. As negative controls, mesenteric lymph node cells from sham control mice treated with culture medium, the levels of cytokines were very low or below the limit of quantification, except for TGFβ (basal levels of approximately 400 μg/mL).

In the supernatant of mesenteric lymph node cells from orally sensitized mice (400 μg/mouse p.o.), the IL-4, IL-5, IL-10 and IL-13 levels were decreased as compared to those of sham control mice. INF gamma, IL-12 and IL-9 levels were null or below the limit of quantification. The TGFβ level was not clearly modified as compared to those of sham control mice.

In the supernatant of mesenteric lymph node cells from sublingually sensitized mice (5 or 50 μg/mouse), the IL-4, IL-5, IL-10 and IL-13 levels were decreased as compared to those of sham control mice. INF gamma, IL-12 and IL-9 levels were null or below the limit of quantification. The TGFβ level was not clearly modified as compared to that of sham control mice. The effects appeared to be more marked at the highest concentration and at time point 72 h.

TABLE 12
Cytokine Secretion by Mesenteric Lymph Node Cells

| | IL-4 | | IL-5 | | IL-10 | | IL-13 | | TNF-B | |
|---|---|---|---|---|---|---|---|---|---|---|
| | 24 h | 72 h | 24 h | 72 h | 24 h | 72 h | 24 h | 72 h | 24 h | 72 h |
| AVG | | | | | | | | | | |
| PE Sham IT | 7.3 | 12.5 | 42.4 | 188.3 | 25.6 | 87.6 | 17.8 | 62.0 | 561 | 361 |
| PE OIT | 3.8 | 4.3 | 20.6 | 45.2 | 9.5 | 27.5 | 7.3 | 21.3 | 420 | 376 |
| PE SLIT 5 | 3.8 | 7.9 | 19.2 | 79.9 | 15.7 | 39.1 | 7.5 | 33.9 | 501 | 406 |
| | 3.4 | 4.3 | 19.2 | 34.7 | 12.4 | 20.7 | 7.5 | 13.9 | 385 | 400 |
| SEM | | | | | | | | | | |
| PE Sham IT | 1.4 | 4.3 | 10.5 | 51.8 | 0.6 | 22.5 | 4.3 | 2.7 | — | 2 |
| PE OIT | 0.6 | 0.7 | 5.7 | 22.3 | 1.6 | 9.2 | 1.4 | 7.0 | 15 | 21 |
| PE SLIT 5 | 0.2 | 0.7 | 5.4 | 30.9 | 2.7 | 11.9 | 2.1 | 5.8 | 44 | 11 |
| PE SLIT 50 | 0.8 | 3.2 | 6.0 | 26.5 | 2.5 | 6.9 | 1.9 | 7.3 | 20 | 11 |

In conclusion, these results suggest that the oral (400 μg/mouse) or sublingual (50 μg/mouse) treatment with peanut protein decreased the anaphylactic response, reflected by a strong drop in temperature and increased clinical score in female C3H/HeJ mice previously sensitized by peanut extract. The lowest sublingual dose (5 μg/mouse) had no effects on the anaphylactic response. The allergic skin response (ear swelling) was not modified whatever the treatment.

Mice treated with peanut protein demonstrated similar elevation in IgG in all treatments when compared to that of sham control mice. Total IgE, IgA was elevated following treatment with peanut protein (p.o and 5 μg/mouse sublingual) but not elevated following 50 ug/mouse SLIT procedure.

The treatments also modified the increase of cytokines release in the supernatant of splenocytes or mesenteric lymph node cells after ex vivo stimulation with peanut protein, although not statistical a tendency towards a decrease was observed for some cytokines and increase for TNF.

In the supernatant of Natural-Ara h 2 (5 or 50 μg/mouse sublingual)-stimulated (200 μg/mL natural Ara h 2) mesenteric lymph node cells, the IL-4, IL-5, IL-10 and IL-13 levels were decreased as compared to sham-stimulated mesenteric lymph node cells, The TGFβ level was significantly decreased as compared to sham-stimulated mesenteric lymph node cells (−31%, p<0.05) in the group treated with 5 μg/mouse.

1. A recombinant Ara h 2 variant polypeptide comprising one or more substitutions, deletions, insertions, or any combination thereof, that are located at one or more of positions 12, 15, 16, 22, 24, 46, 53, 65, 80, 83, 86, 87, 90, 104, 115, 123, 127, or 140 of SEQ ID NO: 4, as compared with the amino acid residues at those same positions in SEQ ID NO: 3, wherein the variant polypeptide is at least 80% identical to the sequence set forth in SEQ ID NO: 3.

2. The recombinant Ara h 2 variant of claim 1, wherein the substitutions, deletions, insertions, or any combination thereof, comprise one or more of:
N, Q, E, D, T, S, G, P, C, K, H, Y, W, M, I, L, V, or A at position 12;
R, E, K, Y, W, F, M, I, V, C, D, G, or A at position 15;
R, K, D, Q, T, M, P, C, E, or W at position 16;
F, Y, W, Q, E, T, S, A, M, I, L, C, R, or H at position 22;
D, E, H, K, S, T, N, Q, L, I, M, W, Y, F, P, A, or G at position 24;
T, V, E, H, S, A, G, Q, N, D, R, P, M, I, L, or C at position 46;
T, S, Q, V, A, G, C, P, M, L, I, E, H, R, K, N, or D at position 53;
T, A, N, D, Q, R, K, H, I, L, M, V, W, P, G, C, or E at position 65;
N, S, T, V, A, I, L, M, F, Y, W, C, E, K, R, or G at position 80;
D, A, C, F, I, P, T, V, W, Y, or Q at position 83;
Y, F, H, R, E, C, G, I, L, M, V, T, S, or Q at position 86;
F, Y, I, L, M, V, A, S, Q, R, K, D, N, E, or P at position 87;
S, P, Q or R at position 90;
L, M, K, R, H, E, D, A, Y, N, S, or W at position 104;
V, D, E, I, L, K, M, N, S, T, A, I, W, F, Y, or H at position 115;
I, Q, or A at position 123;
H, A, D, E, F, G, L, N, P, S, T, W, Y, Q, or V at position 127; and
G, A, C, E, Y, F, H, K, L, M, N, P, Q, S, or V at position 140.

3. (canceled)

4. The recombinant Ara h 2 variant of claim 1, wherein amino acids at positions 12-16 of SEQ ID NO: 4 comprise the sequence set forth in SEQ ID NO: 5, or amino acids at positions 44-65 of SEQ ID NO: 4 comprise the sequence set forth in SEQ ID NO: 6, or amino acids at positions 44-67 of SEQ ID NO: 4 comprise the sequence set forth in SEQ ID NO: 9, or amino acids at positions 11-90 of SEQ ID NO: 4 comprise the sequence set forth in SEQ ID NO: 7 or SEQ ID NO: 8.

5. The recombinant Ara h 2 variant of claim 1, wherein said variant further comprises additional substitutions, deletions, insertions, or any combination thereof, at one or more of positions 28, 44, 48, 51, 55, 63, 67, 107, 108, 109, 124, 125, or 142 of SEQ ID NO: 4, as compared with the amino acid residues at those same positions in SEQ ID NO: 3, optionally wherein the additional substitutions, deletions, insertions, or any combination thereof, comprise one or more of:
(a) S, T, V, N, A, P, I, L, F, Y, H, R, K, E, or D at position 28;
(b) I, A, C, G, H, L, F, Y, N, P, Q, K, E, S, T, V, M, or R at position 44;
(c) V, G, C, E, H, Q, F, K, L, I, W, Y, N, R, S, T, V, A, or D at position 48;
(d) S, G, Y, F, W, M, N, Q, E, R, K, H, T, D, or V at position 51;
(e) G, A, D, E, F, Y, H, Q, V, I, L, M, R, K, S, T, C, or W at position 55;
(f) P, C, F, V, I, L, M, W, Y, N, S, T, Q, G, H, K, or R at position 63;
(g) E, Q, N, R, H, Y, F, W, M, L, V, T, S, A, P, or G at position 67;
(h) A, C, F, G, H, I, K, L, M, Q, P, R, S, T, V, W, or Y at position 107;
(i) T, V, D, E, R, H, Y, W, I, G, A, Q, or K at position 108;
(j) K, C, S, R, G, P, Y, W, L, or I at position 109;
(k) D, A, C, F, G, H, I, N, S, T, V, Y, L, E, or Q at position 124;
(l) M, I, L, W, Y, G, K, N, T, V, or A at position 125; and
(m) M, A, C, E, F, G, H, I, K, L, N, P, Q, R, S, T, V, W, or Y at position 142.

6. (canceled)

7. (canceled)

8. The recombinant Ara h 2 variant of claim 1, wherein said variant comprises the amino acid sequence as set forth in any of SEQ ID NOs: 10-63, 168, 170, 195-201, 204-210, 247-249, or comprises an amino acid sequence having at least 80% identity with the amino acid sequences set forth in any of SEQ ID NOs: 10-63, 168, 170, 195-201, 204-210, 247-249.

9. An isolated nucleotide or modified nucleotide sequence encoding the recombinant Ara h 2 variant of claim 1, wherein the nucleotide or modified nucleotide sequence is DNA or mRNA.

10-12. (canceled)

13. A composition comprising the recombinant Ara h 2 variant polypeptide of claim 1.

14. (canceled)

15. A method of inducing desensitization to peanuts in a subject allergic to peanuts, said method comprising administering to said subject the composition of claim 13, thereby inducing desensitization to peanuts in said subject.

16. (canceled)

17. A composition comprising the nucleotide or modified nucleotide sequence of claim 9.

18. (canceled)

19. A method of inducing desensitization to peanuts in a subject allergic to peanuts, said method comprising administering to said subject the composition of claim 17, thereby inducing desensitization to peanuts in said subject.

20. (canceled)

21. A recombinant Ara h 1 variant polypeptide comprising one or more amino acid substitutions, deletions, insertions, or any combination thereof, that are located at one or more of positions 194, 195, 213, 215, 231, 234, 245, 267, 287, 294, 312, 331, 419, 422, 443, 455, 462, 463, 464, 480, 494, or 500 of SEQ ID NO: 67, as compared with the amino acid residues at those same positions in SEQ ID NO: 65, wherein the variant polypeptide is at least 80% identical to the sequence set forth in SEQ ID NO: 65.

22. The recombinant Ara h 1 variant of claim 21, wherein the substitutions, deletions, insertions, or any combination thereof, comprise one or more of:
(a) D at position 194;
(b) A at position 195;
(c) H at position 213;
(d) R, D, L, I, F, or A at position 215;
(e) A at position 231;
(f) E at position 234;
(g) R at position 245;
(h) E at position 267;
(i) D at position 287;
(j) E at position 294;
(k) A or H at position 312;
(l) H at position 331;
(m) E, V, or A at position 419;
(n) R or A at position 422;
(o) A at position 443;
(p) A at position 455;
(q) A, K, or T at position 462;
(r) S at position 463;
(s) A or S at position 464;
(t) Q at position 480;
(u) A, E, or N at position 494; and
(v) K at position 500.

23. (canceled)

24. The recombinant Ara h 1 variant of claim 21, wherein said variant further comprises additional substitutions, deletions, insertions, or any combination thereof at one or more of positions 12, 24, 27, 30, 42, 57, 58, 73, or 523 of SEQ ID NO: 67, as compared with the amino acid residues at those same positions in SEQ ID NO: 65, optionally wherein the additional substitutions, deletions, insertions, or any combination thereof, comprise one or more of:
(a) K or A at position 12;
(b) V or E at position 24;
(c) A or H at position 27;
(d) E or A at position 30;
(e) L or K at position 42;
(f) D or L at position 57;
(g) S or R at position 58;
(h) A or M at position 73; and
(i) A or K at position 523.

25. (canceled)

26. The recombinant Ara h 1 variant of claim 24, wherein said variant further comprises additional substitutions, deletions, insertions, or any combination thereof at one or more of positions 87, 88, 96, 99, 196, 197, 200, 209, 238, 249, 260, 261, 263, 265, 266, 278, 283, 288, 290, 295, 318, 322, 334, 336, 378, 417, 421, 441, 443, 481, 484, 485, 487, 488, or 491 of SEQ ID NO: 67, as compared with the amino acid residues at those same positions in SEQ ID NO: 65, optionally wherein the additional substitutions, deletions, insertions, or any combination thereof, comprise one or more of:
(a) A at position 87;
(b) A at position 88;
(c) A at position 96;
(d) A at position 99;
(e) H at position 196;
(f) A at position 197;
(g) V, A or Q at position 200;
(h) S at position 209;
(i) Q at position 238;
(j) N at position 249;
(k) K at position 260;
(l) R at position 261;
(m) K or L at position 263;
(n) S at position 265;
(o) R or L at position 266;
(p) R at position 278;
(q) E at position 283;
(r) Q at position 288;
(s) R at position 290;
(t) A at position 295;
(u) H at position 318;
(v) A or K at position 322;
(w) D, A or N at position 334;
(x) R or S at position 336;
(y) K or E at position 378;
(z) R at position 417;
(aa) E or S at position 421;
(bb) N at position 441;
(cc) A at position 443;
(dd) A or S at position 481;
(ee) R, S, A, or M at position 484;
(ff) A at position 485;
(gg) S or K at position 487;
(hh) A at position 488; and
(ii) A, S or E at position 491.

27. (canceled)

28. The recombinant Ara h 1 variant of claim 26, wherein said variant further comprises a substitution of A at position 84 of SEQ ID NO:67.

29. The recombinant Ara h 1 variant of claim 21, wherein said variant comprises the amino acid sequence set forth in any of SEQ ID NOs: 68-161, 174, 176, 178, 180, 182, 184, 193, 194, 211-246, or comprises an amino acid sequence having at least 80% identity with the amino acid sequences set forth in any of SEQ ID NOs: 68-161, 174, 176, 178, 180, 182, 184, 193, 194, 211-246.

30. An isolated nucleotide or modified nucleotide sequence encoding the recombinant Ara h 1 variant of claim 21, wherein the nucleotide or modified nucleotide sequence is DNA or mRNA.

31-33. (canceled)

34. A composition comprising the recombinant Ara h 1 variant polypeptide of claim 21.

35. (canceled)

36. A method of inducing desensitization to peanuts in a subject allergic to peanuts, said method comprising administering to said subject the composition of claim 34, thereby inducing desensitization to peanuts in said subject.

37. (canceled)

38. A composition comprising the nucleotide or modified nucleotide sequence of claim 30.

39. (canceled)

40. A method of inducing desensitization to peanuts in a subject allergic to peanuts, said method comprising administering to said subject the composition of claim 38, thereby inducing desensitization to peanuts in said subject.

41. (canceled)


SEQUENCE LISTING (249 sequences)

SEQ ID NO: 1 - protein, 172 aa, Arachis hypogaea
  source 1..172: note = Ara h 2 WT- uniprot entry Q6PSU2
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 2 - protein, 154 aa, synthetic construct
  source 1..154: note = Ara h 2 WT - (1, 20-172)
MSARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 3 - protein, 153 aa, Arachis hypogaea
  source 1..153: note = Ara h 2 WT - (AA 20-172)
SARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 4 - protein, 153 aa, synthetic construct
  source 1..153: note = Ara h 2 -e variant poistions -
  VARIANT 6: note = Any amino acid
  VARIANT 11: note = Any amino acid
  VARIANT 12: note = Any amino acid
  VARIANT 13: note = Any amino acid
  VARIANT 14: note = Any amino acid
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 26: note = Any amino acid
  VARIANT 27: note = Any amino acid
  VARIANT 28: note = Any amino acid
  VARIANT 32: note = Any amino acid
  VARIANT 39: note = Any amino acid
  VARIANT 44: note = Any amino acid
  VARIANT 45: note = Any amino acid
  VARIANT 46: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 53: note = Any amino acid
  VARIANT 54: note = Any amino acid
  VARIANT 55: note = Any amino acid
  VARIANT 56: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
  VARIANT 68: note = Any amino acid
  VARIANT 69: note = Any amino acid
  VARIANT 80: note = Any amino acid
  VARIANT 81: note = Any amino acid
  VARIANT 82: note = Any amino acid
  VARIANT 83: note = Any amino acid
  VARIANT 84: note = Any amino acid
  VARIANT 85: note = Any amino acid
  VARIANT 86: note = Any amino acid
  VARIANT 87: note = Any amino acid
  VARIANT 89: note = Any amino acid
  VARIANT 90: note = Any amino acid
  VARIANT 92: note = Any amino acid
  VARIANT 96: note = Any amino acid
  VARIANT 97: note = Any amino acid
  VARIANT 99: note = Any amino acid
  VARIANT 100: note = Any amino acid
  VARIANT 102: note = Any amino acid
  VARIANT 103: note = Any amino acid
  VARIANT 104: note = Any amino acid
  VARIANT 105: note = Any amino acid
  VARIANT 107: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 113: note = Any amino acid
  VARIANT 114: note = Any amino acid
  VARIANT 115: note = Any amino acid
  VARIANT 116: note = Any amino acid
  VARIANT 118: note = Any amino acid
  VARIANT 119: note = Any amino acid
  VARIANT 123: note = Any amino acid
  VARIANT 124: note = Any amino acid
  VARIANT 125: note = Any amino acid
  VARIANT 127: note = Any amino acid
  VARIANT 128: note = Any amino acid
  VARIANT 129: note = Any amino acid
  VARIANT 130: note = Any amino acid
  VARIANT 131: note = Any amino acid
  VARIANT 133: note = Any amino acid
  VARIANT 134: note = Any amino acid
  VARIANT 136: note = Any amino acid
  VARIANT 137: note = Any amino acid
  VARIANT 138: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 146: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
SARQQXELQGXXXXXXXXXXXXXXXXXXHLMXKIQRDEXSYGRXXXXXXXXXXXXXXXXXXXXXXXXXXYDRRGAGSSQXXXXXXXXLXXFXNNQXXMXXAXXXXMXXXXXXXXXXQXXQQFXXXLXXXXXQXXLXXXXXXXXXVXSXXXXXX

SEQ ID NO: 5 - protein, 5 aa, synthetic construct
  source 1..5: note = mutated peptide
NRCRR

SEQ ID NO: 6 - protein, 22 aa, synthetic construct
  source 1..22: note = mutated peptide
DPTSPSQDPTSPSQDPDRRDPT

SEQ ID NO: 7 - protein, 80 aa, synthetic construct
  source 1..80: note = MUTATED AA 30-109
DNRCRRQLERANLRPCEQHLMQKIQRDEDSYGRDPTSPSQDPTSPSQDPDRRDPTSPSPYDRRGAGSSQHQERCCYELNE

SEQ ID NO: 8 - protein, 80 aa, synthetic construct
  source 1..80: note = MUTATED AA 30-90
DNRCRRQLERANLRPCEQHLMQKIQRDEDSYGRIPTSVSQSPTSGSQDPDRRPPTSESPYDRRGAGSSQHQERCCYELNE

SEQ ID NO: 9 - protein, 24 aa, synthetic construct
  source 1..24: note = mutated peptide
IPTSVSQSPTSGSQDPDRRPPTSE

SEQ ID NO: 10 - protein, 153 aa, synthetic construct
  source 1..153: note = Arah2_B1001
SARQQWELQGDNRCRRQLERAFLDPCESHLMQKIQRDEDSYGRIPTSVSQSPTSGSQDPDRRPPTSESPYDRRGAGSSQNQEDCCYFLNSFENNQRCMCEALQLIMATKSDRLQVRQQEQQFIDMLHNLPQQCGLRAPQGCMLEVESGGRDRY

SEQ ID NO: 11 - protein, 153 aa, synthetic construct
  source 1..153: note = Arah2_B764
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 12 - protein, 153 aa, synthetic construct
  source 1..153: note = Arah2_B761
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFKRELHNLPQQCGLRAPQGCDLEVESGGRDRY

SEQ ID NO: 13 - protein, 153 aa, synthetic construct
  source 1..153: note = Arah2_B767
SARQQWELQGDNRCRRQLERANLLPCENHLMQKIQRDEDSYGRIPTSVSQSPTSGSQDPDRRPPTSESPYDRRGAGSSQHQERCCYELNEFENNQRCMCEALQLIMAKQSDRLHARQQEQQFKRELHNLPQQCGLRAPQGCDLEVESGGRDRY

SEQ ID NO: 14 - protein, 153 aa, synthetic construct
  source 1..153: note = Arah2_B768
SARQQWELQGDNRCRRQLERANLLPCENHLMQKIQRDEDSYGRSPTSASQSPVSGSQDPDRRTPISASPYDRRGAGSSQHQERCCYELNEFENNQRCMCVALQQIMAKQSDRLHVRQQEQQFKRELRNLPQQCGLRRPQRCDLEVESGGRDRY

SEQ ID NO: 15 - protein, 153 aa, synthetic construct
  source 1..153: note = Arah2_B769
SARQQWELQGDNRCRRQLERANLLPCENHLMQKIQRDEDSYGRIPTSVSQSPTSGSQDPDRRPPTSESPYDRRGAGSSQHQERCCYELNEFENNQRCMCVALQQIMAKQSDRLHVRQQEQQFKRELRNLPQQCGLRRPQRCDLEVESGGRDRY

SEQ ID NO: 16 - protein, 153 aa, synthetic construct
  source 1..153: note = Arah2_B770
SARQQWELQGDNRCRMQLESAFLRPCESHLMQKIQRDEDSYGRLPYSGSQWPESWSQDPDRRDPTSGSPYDRRGAGSSQNQERCCYELNEFENNQRCMCVALQQIMETQSDRLHVRQQEQQFKRELRNLPQQCGLRFPQGCHLEVESGGRDRY

SEQ ID NO: 17 - protein, 153 aa, synthetic construct
  source 1..153: note = Arah2_B771
SARQQWELQGDNRCRMQLESAFLRPCESHLMQKIQRDEDSYGRIPTSVSQSPTSGSQDPDRRPPTSESPYDRRGAGSSQNQERCCYELNEFENNQRCMCVALQQIMETQSDRLHVRQQEQQFKRELRNLPQQCGLRFPQGCHLEVESGGRDRY

SEQ ID NO: 18 - protein, 153 aa, synthetic construct
  source 1..153: note = Arah2_B772
SARQQWELQGDNRCRMQLESANLLPCENHLMQKIQRDEDSYGRIPTSVSQSPTSGSQDPDRRPPTSESPYDRRGAGSSQHQERCCYELNEFENNQRCMCVALQQIMAKQSDRLHARQQEQQFKRELRNLPQQCGLRRPQRCDLEVESGGRDRY

SEQ ID NO: 19 - protein, 153 aa, synthetic construct
  source 1..153: note = Arah2_B773
SARQQWELQGDNRCRMQLESANLLPCENHLMQKIQRDEDSYGRLPYSGSQWPESWSQDPDRRDPTSGSPYDRRGAGSSQHQERCCYELNEFENNQRCMCVALQQIMAKQSDRLHARQQEQQFKRELRNLPQQCGLRRPQRCDLEVESGGRDRY

SEQ ID NO: 20 - protein, 153 aa, synthetic construct
  source 1..153: note = Arah2_B774
SARQQWELQGDNRCRMQLESANLLPCENHLMQKIQRDEDSYGRSPTSASQSPVSGSQDPDRRTPISASPYDRRGAGSSQHQERCCYELNEFENNQRCMCVALQQIMAKQSDRLHSRQQEQQFKRELRNLPQQCGLRFPQGCHLEVESGGRDRY

SEQ ID NO: 21 - protein, 153 aa, synthetic construct
  source 1..153: note = Arah2_B775
SARQQWELQGDNRCRRQLERAFLRPCESHLMQKIQRDEDSYGRLPYSGSQWPESWSQDPDRRDPTSGSPYDRRGAGSSQNQERCCYELNEFENNQRCMCVALQQIMKTQSDRLHVRQQEQQFKRELRNLPQQCGLRRPQRCDLEVESGGRDRY

SEQ ID NO: 22 - protein, 153 aa, synthetic construct
  source 1..153: note = Arah2_B776
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCVALQQIMKTQSDRLHVRQQEQQFKRELRNLPQQCGLRRPQRCDLEVESGGRDRY

SEQ ID NO: 23 - protein, 153 aa, synthetic construct
  source 1..153: note = Arah2_B777
SARQQWELQGDNRCRMQLESAFLRPCESHLMQKIQRDEDSYGRIPTSVSQSPTSGSQDPDRRPPTSESPYDRRGAGSSQNQERCCYELNEFENNQRCMCEALQLIMKTQSDRLHSRQQEQQFKRELRNLPQQCGLRAPQHCALEVESGGRDRY

SEQ ID NO: 24 - protein, 153 aa, synthetic construct
  source 1..153: note = Arah2_B778
SARQQWELQGDNRCRMQLESAFLRPCESHLMQKIQRDEDSYGRLPYSGSQWPESWSQDPDRRDPTSGSPYDRRGAGSSQNQERCCYELNEFENNQRCMCEALQLIMKTQSDRLHSRQQEQQFKRELRNLPQQCGLRAPQHCALEVESGGRDRY

SEQ ID NO: 25 - protein, 153 aa, synthetic construct
  source 1..153: note = Arah2_B779
SARQQWELQGDNRCRRQLERAFLRPCESHLMQKIQRDEDSYGRSPTSASQSPVSGSQDPDRRTPISASPYDRRGAGSSQNQERCCYELNEFENNQRCMCVALQQIMETQSDRLHARQQEQQFKRELRNLPQQCGLRFPQGCHLEVESGGRDRY

SEQ ID NO: 26 - protein, 153 aa, synthetic construct
  source 1..153: note = Arah2_B780
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCVALQQIMETQSDRLHARQQEQQFKRELRNLPQQCGLRFPQGCHLEVESGGRDRY

SEQ ID NO: 27 - protein, 153 aa, synthetic construct
  source 1..153: note = Arah2_B781
SARQQWELQGDNRCRRQLERANLLPCENHLMQKIQRDEDSYGRLPYSGSQWPESWSQDPDRRDPTSGSPYDRRGAGSSQHQERCCYELNEFENNQRCMCEALQLIMAKQSDRLHSRQQEQQFKRELRNLPQQCGLRAPQHCALEVESGGRDRY

SEQ ID NO: 28 - protein, 153 aa, synthetic construct
  source 1..153: note = Arah2_B782
SARQQWELQGDNRCRRQLERANLLPCENHLMQKIQRDEDSYGRIPTSVSQSPTSGSQDPDRRPPTSESPYDRRGAGSSQHQERCCYELNEFENNQRCMCEALQLIMAKQSDRLHSRQQEQQFKRELRNLPQQCGLRAPQHCALEVESGGRDRY

SEQ ID NO: 29 - protein, 153 aa, synthetic construct
  source 1..153: note = Arah2_B783
SARQQWELQGDNRCRRQLERANLLPCENHLMQKIQRDEDSYGRSPTSASQSPVSGSQDPDRRTPISASPYDRRGAGSSQHQERCCYELNEFENNQRCMCEALQLIMAKQSDRLHARQQEQQFKRELHNLPQQCGLRAPQGCDLEVESGGRDRY

SEQ ID NO: 30 - protein, 153 aa, synthetic construct
  source 1..153: note = Arah2_B784
SARQQWELQGDNRCRRQLESAFLRPCEQHLMQKIQRDEDSYGRIPTSVSQSPTSGSQDPDRRPPTSESPYDRRGAGSSQNQERCCYELNEFENNQRCMCVALQLIMANQRDRLQVRQQEQQFKRELRNLPQQCGLRRPQHCHLEVESGGRDRY

SEQ ID NO: 31 - protein, 153 aa, synthetic construct
  source 1..153: note = Arah2_B785
SARQQWELQGDNRCRRQLESAFLRPCEQHLMQKIQRDEDSYGRSPTSASQSPVSGSQDPDRRTPISASPYDRRGAGSSQNQERCCYELNEFENNQRCMCVALQLIMANQRDRLQSRQQEQQFKRELRNLPQQCGLRRPQHCYLEVESGGRDRY

SEQ ID NO: 32 - protein, 153 aa, synthetic construct
  source 1..153: note = Arah2_B786
SARQQWELQGDNRCRRQLESAFLRPCESHLMQKIQRDEDSYGRLPYSGSQWPESWSQDPDRRDPTSGSPYDRRGAGSSQNQERCCYELNEFENNQRCMCVALQLIMATQRDRLQVRQQEQQFKRELRNLPQQCGLRRPQHCHLEVESGGRDRY

SEQ ID NO: 33 - protein, 153 aa, synthetic construct
  source 1..153: note = Arah2_B831
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFIRMLHRLPQQCGLRAPQGCMLEVESGGRDRY

SEQ ID NO: 34 - protein, 153 aa, synthetic construct
  source 1..153: note = Arah2_B832
SARQQWELQGDNRCRRQLERAFLRPCESHLMQKIQRDEDSYGRSPTSASQSPVSGSQDPDRRTPISASPYDRRGAGSSQNQERCCYELNEFENNQRCMCVALQQIMETQSDRLHARQQEQQFIDMLRNLPQQCGLRFPQECHLEVESGGRDRY

SEQ ID NO: 35 - protein, 153 aa, synthetic construct
  source 1..153: note = Arah2_B833
SARQQWELQGDNRCRRQLERAFLRPCESHLMQKIQRDEDSYGRSPTSASQSPVSGSQDPDRRTPISASPYDRRGAGSSQNQERCCYELNEFENNQRCMCVALQQIMETQSDRLHARQQEQQFIRMLRRLPQQCGLRFPQGCMLEVESGGRDRY

SEQ ID NO: 36 - protein, 153 aa, synthetic construct
  source 1..153: note = Arah2_B834
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCVALQQIMETQSDRLHARQQEQQFIDMLRNLPQQCGLRFPQECHLEVESGGRDRY

SEQ ID NO: 37 - protein, 153 aa, synthetic construct
  source 1..153: note = Arah2_B835
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMCVALQQIMETQSDRLHARQQEQQFIRMLRRLPQQCGLRFPQGCMLEVESGGRDRY

SEQ ID NO: 38 - protein, 153 aa, synthetic construct
  source 1..153: note = Arah2_B836
SARQQWELQGDNRCRRQLERANLLPCENHLMQKIQRDEDSYGRSPTSASQSPVSGSQDPDRRTPISASPYDRRGAGSSQHQERCCYELNEFENNQRCMCEALQLIMAKQSDRLHARQQEQQFIDMLHNLPQQCGLRAPQECDLEVESGGRDRY

SEQ ID NO: 39 - protein, 153 aa, synthetic construct
  source 1..153: note = Arah2_B837
SARQQWELQGDNRCRRQLERANLLPCENHLMQKIQRDEDSYGRSPTSASQSPVSGSQDPDRRTPISASPYDRRGAGSSQHQERCCYELNEFENNQRCMCEALQLIMAKQSDRLHARQQEQQFIRMLHRLPQQCGLRAPQGCMLEVESGGRDRY

SEQ ID NO: 40 - protein, 153 aa, synthetic construct
  source 1..153: note = Arah2_B956
SARQQWELQGDEMCRRQLERAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 41 - protein, 153 aa, synthetic construct
  source 1..153: note = Arah2_B957
SARQQWELQGDSRCQSQLESAHLRPCEQHLMQKIQRDEDSYGRRPNSPSQRPNSPSQDPDRRRPNSPSPYDRRGAGSSQRQERCCHELNEFENNQRCMCEALQQIMEHQSDRLQGRQQEQQFQSELSNLPQQCGLRAPQSCRLEVESGGRDRY

SEQ ID NO: 42 - protein, 153 aa, synthetic construct
  source 1..153: note = Arah2_B958
SARQQWELQGDSRCQSQLERANLRPCEQHLMQKIQRDEDSYGRRPPSPSQRPPSPSQDPDRRRPPSPSPYDRRGAGSSQHQERCCNELNEFENNQRCMCEALQQIMENQSDRLQGRQQEQQFKSELSNLPQQCGLRAPQSCRLEVESGGRDRY

SEQ ID NO: 43 - protein, 153 aa, synthetic construct
  source 1..153: note = Arah2_B961
SARQQWELQGDNRCRRQLERAFLRPCESHLMQKIQRDEDSYGRIPTSNSQSHKSGSQDPDRRPPESESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 44 - protein, 153 aa, synthetic construct
  source 1..153: note = Arah2_B962
SARQQWELQGDQSCQSQLEQANLRPCEQHLMQKIQRDEDSYGRDPYSPSQDPYSPSQDPDRRDPYSPSPYDRRGAGSSQRQERCCNELNEFENNQRCMCEALQQIMENQSDRLQGRQQEQQFKQELQNLPQQCGLRAPQQCDLEVESGGRDRY

SEQ ID NO: 45 - protein, 153 aa, synthetic construct
  source 1..153: note = Arah2_B963
SARQQWELQGDEMCRRQLERAFLRPCESHLMQKIQRDEDSYGRIPTSNSQSHKSPSQDPDRRPPESESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 46 - protein, 153 aa, synthetic construct
  source 1..153: note = Arah2_B964
SARQQWELQGDTMCRRQLVRAFLRPCESHLMQKIQRDEDSYGRIPASNSQSPKSYSQDPDRRMPESESPYDRRGAGSSQNQERCCYFLNSFENNQRCMCEALQLIMATKSDRLQVRQQEQQFIDMLHNLPQQCGLRAPQGCMLEVESGGRDRY

SEQ ID NO: 47 - protein, 153 aa, synthetic construct
  source 1..153: note = Arah2_B967
SARQQWELQGDARCQSQLEAANLRPCEQHLMQKIQRDEDSYGRDPASPSQDPASPSQDPDRRDPASPSPYDRRGAGSSQHQSRCCYELNEFENNQACMCEALQQIMVNQSARLAGRAQEQQFARELRNLPAQCGLAAPQACALEVESGGRDRY

SEQ ID NO: 48 - protein, 153 aa, synthetic construct
  source 1..153: note = Arah2_B968
SARQQWELQGDARCQSQLEAANLAPCEQHLMQKIQRDEDSYGRDPASPSQDPASPSQDPDRRDPASPSPYDRRGAGSSQVQSACCYELNEFENNQACMCVALQQIMVAQSARLAGRQQEQQFARELVNLPAQCGLAAPQACALEVESGGRDRY

SEQ ID NO: 49 - protein, 153 aa, synthetic construct
  source 1..153: note = Arah2_B969
SARQQWELQGDNRARRQLERAFLRPCESHLMQKIQRDEDSYGRIPTSVSQSPTSGSQDPDRRPPTSESPYDRRGAGSSQNQERCCYELNEFENNQRAMCEALQLIMATKSDRLQVRQQEQQFIDMLHNLPQQCGLRAPQGCMLEVESGGRDRY

SEQ ID NO: 50 - protein, 153 aa, synthetic construct
  source 1..153: note = Arah2_B970
SARQQWELQGDNRCRRQLERAFLRPAESHLMQKIQRDEDSYGRIPTSVSQSPTSGSQDPDRRPPTSESPYDRRGAGSSQNQERACYELNEFENNQRCMCEALQLIMATKSDRLQVRQQEQQFIDMLHNLPQQCGLRAPQGCMLEVESGGRDRY

SEQ ID NO: 51 - protein, 153 aa, synthetic construct
  source 1..153: note = Arah2_B971
SARQQWELQGDNRCRRQLERAFLRPCESHLMQKIQRDEDSYGRIPTSVSQSPTSGSQDPDRRPPTSESPYDRRGAGSSQNQERCCYELNEFENNQRCMAEALQLIMATKSDRLQVRQQEQQFIDMLHNLPQQCGLRAPQGAMLEVESGGRDRY

SEQ ID NO: 52 - protein, 153 aa, synthetic construct
  source 1..153: note = Arah2_B973
SARQQWELQGDQRCQSQLERANLQPCEQHLMQKIQRDESSYGRSPQSPSQSPQSPSQDPDRRDPQSPSPYDRRGAGSSQHQEQCCNELNEFENNQRCMCEALQQIMENQSDRLQGRQQEQQFKRELRNLPQQCGLRAPQRCSLEVESGGRDRY

SEQ ID NO: 53 - protein, 153 aa, synthetic construct
  source 1..153: note = Arah2_B974
SARQQWELQGDQRCQSQLERANLRPCERHLMRKIQRDEDSYGRDPYSPSQDPQSPSQDPDRRDPQSPSPYDRRGAGSSQHRERCCNELNEFENNQRCMCEALRQIMENQSDRLQGRQQEQQFKRELRNLPRQCGLRAPQRCDLEVESGGRDRY

SEQ ID NO: 54 - protein, 153 aa, synthetic construct
  source 1..153: note = Arah2_B975
SARQQWELQGDDRCQSQLPRANLDPCEQHLMQKIQRDEDSYGRDPNSPSQDPNSPSQDPDRRDPNSPSPYDRRGAGSSQHQEDCCNPLNPFENNQRCMCPALQQIMPNQSDRLQGRQQEQQFKRELRNLPQQCGLRAPQRCDLPVESGGRDRY

SEQ ID NO: 55 - protein, 153 aa, synthetic construct
  source 1..153: note = Arah2_B976
SARQQWELQGDQRCQSQLERANLQPCEQHLMQKIQRDEDSYGRDPYSQSQDPDSQSQDPDRRDPDSQSPYDRRGAGSSQHQEQCCNELNEFENNQRCMCEALQQIMENQSDRLQGRQQEQQFKRELRNLPQQCGLRAPQRCDLEVESGGRDRY

SEQ ID NO: 56 - protein, 153 aa, synthetic construct
  source 1..153: note = Arah2_B977
SARQQWELQGDQRCQSQLERANLRPCEQHLMQKIQRDESSYGRSPYSPSQSPNSPSQDPDRRDPNSPSPYDRRGAGSSQHQERCCNELNEFENNQRCMCEALQQIMENQSDRLQGRQQEQQFKRELRNLPQQCGLRAPQRCSLEVESGGRDRY

SEQ ID NO: 57 - protein, 153 aa, synthetic construct
  source 1..153: note = Arah2_B646
SARQQWELQGDNRCRMQLESANLRPCEQHLMQKIQRDEDSYGRLPYSGSQWPESWSQDPDRRDPTSGSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 58 - protein, 153 aa, synthetic construct
  source 1..153: note = Arah2_B981
SARQQWELQGDTMCRRQLVRAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 59 - protein, 153 aa, synthetic construct
  source 1..153: note = Arah2_B982
SARQQWELQGDNRCRRQLERAFLRPCESHLMQKIQRDEDSYGRIPASNSQSPKSYSQDPDRRMPESESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 60 - protein, 153 aa, synthetic construct
  source 1..153: note = Arah2_B983
SARQQWELQGDRRCQSQLPRANLRPCEQHLMQKIQRDEDSYGRDPYSPSQDPNSPSQDPDRRDPNSPSPYDRRGAGSSQHQERCCNPLNPFENNQRCMCPALQQIMPNQSDRLQGRQQEQQFKRELRNLPQQCGLRAPQRCDLPVESGGRDRY

SEQ ID NO: 61 - protein, 153 aa, synthetic construct
  source 1..153: note = Arah2_B984
SARQQWELQGDNRCRRQLERAFLRPCESHLMQKIQRDEDSYGRIPTSVSQSPTSGSQDPDRRPPTSESPYDRRGAGSSQNQERCAYELNEFENNQRCMCEALQLIMATKSDRLQVRQQEQQFIDMLHNLPQQAGLRAPQGCMLEVESGGRDRY

SEQ ID NO: 62 - protein, 153 aa, synthetic construct
  source 1..153: note = Arah2_B985
SARQQVELQGKERCASQLKEARLEPCETHLMQKIQRDEDSYGRKPISGSQRPASGSQDPDRRRPTSASPYDRRGAGSSQEQMRCCYKLRRFENNQRCMCHALQQIMKVQSRRNAGRQQEQQFIRELENLPQQCGLRAPQECRLRVRSGGRDRY

SEQ ID NO: 63 - protein, 153 aa, synthetic construct
  source 1..153: note = Arah2_B986
SARQQWELQGKERCASQLEEARLEPCETHLMQKIQRDEDSYGRKPYSGSQRPYSGSQDPDRRDPTSASPYDRRGAGSSQEQMRCCYELREFENNQRCMCHALQQIMKNQSRRLAGRQQEQQFIRELENLPQQCGLRAPQECRLEVRSGGRDRY

SEQ ID NO: 64 - protein, 603 aa, Arachis hypogaea
  source 1..603: note = WT full length Ara h 1 (uniprot P43238)
MAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 65 - protein, 542 aa, synthetic construct
  source 1..542: note = WT Mature Ara h 1 (amino acids 85-626 according to uniprot numbering P43238)
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 66 - protein, 417 aa, synthetic construct
  source 1..417: note = WT core Ara h 1 (amino acids 170-586 according to uniprot numbering P43238)
SRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSAR

SEQ ID NO: 67 - protein, 542 aa, synthetic construct
  source 1..542: note = Ara h 1 All possible variant poistions - any AA
  VARIANT 12: note = Any amino acid
  VARIANT 24: note = Any amino acid
  VARIANT 27: note = Any amino acid
  VARIANT 30: note = Any amino acid
  VARIANT 42: note = Any amino acid
  VARIANT 52: note = Any amino acid
  VARIANT 57: note = Any amino acid
  VARIANT 58: note = Any amino acid
  VARIANT 73: note = Any amino acid
  VARIANT 84: note = Any amino acid
  VARIANT 87: note = Any amino acid
  VARIANT 88: note = Any amino acid
  VARIANT 96: note = Any amino acid
  VARIANT 99: note = Any amino acid
  VARIANT 167: note = Any amino acid
  VARIANT 194: note = Any amino acid
  VARIANT 195: note = Any amino acid
  VARIANT 196: note = Any amino acid
  VARIANT 197: note = Any amino acid
  VARIANT 200: note = Any amino acid
  VARIANT 209: note = Any amino acid
  VARIANT 213: note = Any amino acid
  VARIANT 215: note = Any amino acid
  VARIANT 218: note = Any amino acid
  VARIANT 223: note = Any amino acid
  VARIANT 231: note = Any amino acid
  VARIANT 234: note = Any amino acid
  VARIANT 238: note = Any amino acid
  VARIANT 245: note = Any amino acid
  VARIANT 249: note = Any amino acid
  VARIANT 260: note = Any amino acid
  VARIANT 263: note = Any amino acid
  VARIANT 265: note = Any amino acid
  VARIANT 266: note = Any amino acid
  VARIANT 267: note = Any amino acid
  VARIANT 278: note = Any amino acid
  VARIANT 283: note = Any amino acid
  VARIANT 287: note = Any amino acid
  VARIANT 288: note = Any amino acid
  VARIANT 290: note = Any amino acid
  VARIANT 294: note = Any amino acid
  VARIANT 295: note = Any amino acid
  VARIANT 309: note = Any amino acid
  VARIANT 312: note = Any amino acid
  VARIANT 318: note = Any amino acid
  VARIANT 322: note = Any amino acid
  VARIANT 331: note = Any amino acid
  VARIANT 334: note = Any amino acid
  VARIANT 336: note = Any amino acid
  VARIANT 378: note = Any amino acid
  VARIANT 417: note = Any amino acid
  VARIANT 419: note = Any amino acid
  VARIANT 421: note = Any amino acid
  VARIANT 422: note = Any amino acid
  VARIANT 432: note = Any amino acid
  VARIANT 441: note = Any amino acid
  VARIANT 443: note = Any amino acid
  VARIANT 445: note = Any amino acid
  VARIANT 455: note = Any amino acid
  VARIANT 462: note = Any amino acid
  VARIANT 463: note = Any amino acid
  VARIANT 464: note = Any amino acid
  VARIANT 480: note = Any amino acid
  VARIANT 481: note = Any amino acid
  VARIANT 484: note = Any amino acid
  VARIANT 485: note = Any amino acid
  VARIANT 487: note = Any amino acid
  VARIANT 488: note = Any amino acid
  VARIANT 491: note = Any amino acid
  VARIANT 494: note = Any amino acid
  VARIANT 500: note = Any amino acid
  VARIANT 523: note = Any amino acid
RSPPGERTRGRXPGDYDDDRRQPXREXGGXWGPAGPRERERXEDWRQPREDXRRPSXXQPRKIRPEGREGEQXWGTPGSHVREXTSXXNPFYFPSXRFXTRYGNQNGRIRVLQRFDQRSRQFQNLQNHRIVQIEAKPNTLVLPKHADADNILVIQQGQATVTVANGXNRKSFNLDEGHALRIPSGFISYILNRXXXXNLXVAKISMPVXTPGXFXDFXPASSXDQSSYLQXFSXNTLXAAFNAEXNEIXRVLLEENAGGXQEXRXXXRWSTRSSENNXGVIVXVSKXXVXELTXXAKSVSKKGSEEEGXITXPINLRXGEPXLSNNFGKLXEVXPXKKNPQLQDLDMMLTCVEIKEGALMLPHFNSKAMVIVVVNKGXGNLELVAVRKEQQQRGRREEEEDEDEEEEGSNREVRRYXAXLXXGDVFIMPAAXPVAINASSXLXLXGFGINAENNXRIFLAGXXXNVIDQIEKQAKDLAFXXSGXXVXXLIXNQXESHFVXARPQSQSQSPSSPEKESPEKEDXEEENQGGKGPLLSILKAFN

SEQ ID NO: 68 - protein, 542 aa, synthetic construct
  source 1..542: note = Arah1_B867
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTAALKAGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 69 - protein, 542 aa, synthetic construct
  source 1..542: note = Arah1_B869
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGAKDNVIDQIEKQAKDLAFPGSGAAVEALIANQAESHFVSARPQSQSQSPSSPEKESPEKEDQEEENQGGKGPLLSILKAFN

SEQ ID NO: 70 - protein, 542 aa, synthetic construct
  source 1..542: note = Arah1_B871
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPALSNNFGKLFEVNPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNARIFLAGDKANVIDQIEKQAKDLAFPGSGEQVEKLIKNQKESHFVSARPQSQSQSPSSPEKESPEKEDQEEENQGGKGPLLSILKAFN

SEQ ID NO: 71 - protein, 542 aa, synthetic construct
  source 1..542: note = Arah1_B876
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRDAHANLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 72 - protein, 542 aa, synthetic construct
  source 1..542: note = Arah1_B879
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 73 - protein, 542 aa, synthetic construct
  source 1..542: note = Arah1_B923
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRDDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 74 - protein, 542 aa, synthetic construct
  source 1..542: note = Arah1_B924
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 75 - protein, 542 aa, synthetic construct
  source 1..542: note = Arah1_B926
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHAHQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 76 - protein, 542 aa, synthetic construct
  source 1..542: note = Arah1_B946
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 77 - protein, 542 aa, synthetic construct
  source 1..542: note = Arah1_B947
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPRKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 78 - protein, 542 aa, synthetic construct
  source 1..542: note = Arah1_B948
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPASGEQVEKLIKNQKESHFVSARPQSQSQSPSSPEKESPEKEDQEEENQGGKGPLLSILKAFN

SEQ ID NO: 79 - protein, 542 aa, synthetic construct
  source 1..542: note = Arah1_B949
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPSSGEQVEKLIKNQKESHFVSARPQSQSQSPSSPEKESPEKEDQEEENQGGKGPLLSILKAFN

SEQ ID NO: 80 - protein, 542 aa, synthetic construct
  source 1..542: note = Arah1_B991
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 81 - protein, 542 aa, synthetic construct
  source 1..542: note = Arah1_B992
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFQGSGEQVEKLIKNQKESHFVSARPQSQSQSPSSPEKESPEKEDQEEENQGGKGPLLSILKAFN

SEQ ID NO: 82 - protein, 542 aa, synthetic construct
  source 1..542: note = Arah1_B996
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTAVLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 83 - protein, 542 aa, synthetic construct
  source 1..542: note = Arah1_B997
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTAVLKRGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 84 - protein, 542 aa, synthetic construct
  source 1..542: note = Arah1_B998
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYRAELKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 85 - protein, 542 aa, synthetic construct
  source 1..542: note = Arah1_B1010
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGKKDNVIDQIEKQAKDLAFPGSGEQVEKLIKNQEESHFVSARPQSQSQSPSSPEKESPEKEDQEEENQGGKGPLLSILKAFN

SEQ ID NO: 86 - protein, 542 aa, synthetic construct
  source 1..542: note = Arah1_B1011
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGTKDNVIDQIEKQAKDLAFPGSGEQVEKLIKNQNESHFVSARPQSQSQSPSSPEKESPEKEDQEEENQGGKGPLLSILKAFN

SEQ ID NO: 87 - protein, 542 aa, synthetic construct
  source 1..542: note = Arah1_B1013
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGKKDNVIDQIEKQAKDLAFPGSGMQVEKLIKNQEESHFVSARPQSQSQSPSSPEKESPEKEDQEEENQGGKGPLLSILKAFN

SEQ ID NO: 88 - protein, 542 aa, synthetic construct
  source 1..542: note = Arah1_B1086
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRDDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 89 - protein, 542 aa, synthetic construct
  source 1..542: note = Arah1_B1087
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRDDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 90 - protein, 542 aa, synthetic construct
  source 1..542: note = Arah1_B1088
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRDDNQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 91 - protein, 542 aa, synthetic construct
  source 1..542: note = Arah1_B1089
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRDDNQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 92 - protein, 542 aa, synthetic construct
  source 1..542: note = Arah1_B1090
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 93 - protein, 542 aa, synthetic construct
  source 1..542: note = Arah1_B1091
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 94 - protein, 542 aa, synthetic construct
  source 1..542: note = Arah1_B1092
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 95 - protein, 542 aa, synthetic construct
  source 1..542: note = Arah1_B1093
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 96 - protein, 542 aa, synthetic construct
  source 1..542: note = Arah1_B1190
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPALSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKSNVIDQIEKQAKDLAFQASGEQVEKLIKNQEESHFVSARPQSQSQSPSSPEKESPEKEDQEEENQGGKGPLLSILKAFN

SEQ ID NO: 97 - protein, 542 aa, synthetic construct
  source 1..542: note = Arah1_B1191
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKKSNVIDQIEKQAKDLAFQASGEQVEKLIKNQEESHFVSARPQSQSQSPSSPEKESPEKEDQEEENQGGKGPLLSILKAFN

SEQ ID NO: 98 - protein, 542 aa, synthetic construct
  source 1..542: note = Arah1_B1192
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLAVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPALSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKANVIDQIEKQAKDLAFQASGEQVEKLIKNQEESHFVSARPQSQSQSPSSPEKESPEKEDQEEENQGGKGPLLSILKAFN

SEQ ID NO: 99 - protein, 542 aa, synthetic construct
  source 1..542: note = Arah1_B1201
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 100 - protein, 542 aa, synthetic construct
  source 1..542: note = Arah1_B1202
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFRDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 101 - protein, 542 aa, synthetic construct
  source 1..542: note = Arah1_B1203
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVSTPGHFRDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 102 - protein, 542 aa, synthetic construct
  source 1..542: note = Arah1_B1246
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLVVAKISMPVNTPGHFRDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKKSNVIDQIEKQAKDLAFQASGEQVEKLIKNQEESHFVSARPQSQSQSPSSPEKESPEKEDQEEENQGGKGPLLSILKAFN

SEQ ID NO: 103 - protein, 542 aa, synthetic construct
  source 1..542: note = Arah1_B1247
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLVVAKISMPVNTPGHFRDFFPASSRDQSSYLQGFSRNTLEAAFNAEFNEIRRVLLEENAGGEQEKRGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGKGNLELVAVRKEQQQRGRREEEEDEDEEEEGSNREVRRYTAELKRGDVFIMPAAHPVAINASSELHLLGFGINAENNARIFLAGKKSNVIDQIEKQAKDLAFQASGEQVEKLIENQEESHFVKARPQSQSQSPSSPEKESPEKEDQEEENQGGKGPLLSILKAFN

SEQ ID NO: 104 - protein, 542 aa, synthetic construct
  source 1..542: note = Arah1_B1248
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLVVAKISMPVNTPGHFRDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKKSNVIDQIEKQAKDLAFQASGEQVEKLIKNQEESHFVSARPQSQSQSPSSPEKESPEKEDQEEENQGGKGPLLSILKAFN

SEQ ID NO: 105 - protein, 542 aa, synthetic construct
  source 1..542: note = Arah1_B1249
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLVVAKISMPVNTPGHFRDFFPASSRDQSSYLQGFSRNTLEAAFNAEFNEIRRVLLEENAGGEQEKRGQERWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGKGNLELVAVRKEQQQRGRREEEEDEDEEEEGSNREVRRYTAELKRGDVFIMPAAHPVAINASSELHLLGFGINAENNARIFLAGKKSNVIDQIEKQAKDLAFQASGEQVEKLIENQEESHFVKARPQSQSQSPSSPEKESPEKEDQEEENQGGKGPLLSILKAFN

SEQ ID NO: 106 - protein, 542 aa, synthetic construct
  source 1..542: note = Arah1_B1250
RSPPGERTRGRQPGDYDDDRRQPRREAGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 107 - protein, 542 aa, synthetic construct
  source 1..542: note = Arah1_B1251
RSPPGERTRGRQPGDYDDDRRQPVREAGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 108 - protein, 542 aa, synthetic construct
  source 1..542: note = Arah1_B1252
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 109 - protein, 542 aa, synthetic construct
  source 1..542: note = Arah1_B1253
RSPPGERTRGRQPGDYDDDRRQPVREAGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 110 - protein, 542 aa, synthetic construct
  source 1..542: note = Arah1_B1256
RSPPGERTRGRQPGDYDDDRRQPRREEGGRWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 111 - protein, 542 aa, synthetic construct
  source 1..542: note = Arah1_B1258
RSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 112 - protein, 542 aa, synthetic construct
  source 1..542: note = Arah1_B1267
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGRRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 113 - protein, 542 aa, synthetic construct
  source 1..542: note = Arah1_B1268
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGLRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 114 - protein, 542 aa, synthetic construct
  source 1..542: note = Arah1_B1269
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 115 - protein, 542 aa, synthetic construct
  source 1..542: note = Arah1_B1270
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEREERSQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 116 - protein, 542 aa, synthetic construct
  source 1..542: note = Arah1_B1271
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 117 - protein, 542 aa, synthetic construct
  source 1..542: note = Arah1_B1272
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQAFSENTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 118 - protein, 542 aa, synthetic construct
  source 1..542: note = Arah1_B1275
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 119 - protein, 542 aa, synthetic construct
  source 1..542: note = Arah1_B1281
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 120 - protein, 542 aa, synthetic construct
  source 1..542: note = Arah1_B1282
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAERNEIRRVLLEENAGGEQEERGQRRWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 121 - protein, 542 aa, synthetic construct
  source 1..542: note = Arah1_B1283
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQAFSENTLEAAFNAEFNEIRRVLLEENAGGEQEERGQRRWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 122 - protein, 542 aa, synthetic construct
  source 1..542: note = Arah1_B1284
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 123 - protein, 542 aa, synthetic construct
  source 1..542: note = Arah1_B1285
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQAFSENTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 124 - protein, 542 aa, synthetic construct
  source 1..542: note = Arah1_B1286
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAERNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 125 - protein, 542 aa, synthetic construct
  source 1..542: note = Arah1_B1287
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQRRWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 126 - protein, 542 aa, synthetic construct
  source 1..542: note = Arah1_B1288
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 127 - protein, 542 aa, synthetic construct
  source 1..542: note = Arah1_B1289
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 128 - protein, 542 aa, synthetic construct
  source 1..542: note = Arah1_B1290
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQAFSENTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 129 - protein, 542 aa, synthetic construct
  source 1..542: note = Arah1_B1291
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 130 - protein, 542 aa, synthetic construct
  source 1..542: note = Arah1_B1292
RSPPGERTRGRQPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 131 - protein, 542 aa, synthetic construct
  source 1..542: note = Arah1_B1293
RSPPGERTRGRQPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLVGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 132 - protein, 542 aa, synthetic construct
  source 1..542: note = Arah1_B1294
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 133 - protein, 542 aa, synthetic construct
  source 1..542: note = Arah1_PLP242
RSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 134 - protein, 542 aa, synthetic construct
  source 1..542: note = Arah1_PLP243
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 135 - protein, 542 aa, synthetic construct
  source 1..542: note = Arah1_PLP244
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLVGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 136 - protein, 542 aa, synthetic construct
  source 1..542: note = Arah1_PLP245
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 137 - protein, 542 aa, synthetic construct
  source 1..542: note = Arah1_PLP246
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 138 - protein, 542 aa, synthetic construct
  source 1..542: note = Arah1_PLP247
RSPPGERTRGRKPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLQAAFNAERNEINRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 139 - protein, 542 aa, synthetic construct
  source 1..542: note = Arah1_B1298
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 140 - protein, 542 aa, synthetic construct
  source 1..542: note = Arah1_B1299
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 141 - protein, 542 aa, synthetic construct
  source 1..542: note = Arah1_B1300
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLQAAFNAERNEINRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 142 - protein, 542 aa, synthetic construct
  source 1..542: note = Arah1_PLP256
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSENTLEAAFNAERNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLVGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 143 - protein, 542 aa, synthetic construct
  source 1..542: note = Arah1_PLP257
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLVGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 144 - protein, 542 aa, synthetic construct
  source 1..542: note = Arah1_PLP258
RSPPGERTRGRKPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLQAAFNAERNEINRVLLEENAGGEQEERGQERWSTRSSENNEGVIVKVSKDQVRELTEAAKSVSKKGSEEEGDITHPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLVGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 145 - protein, 542 aa, synthetic construct
  source 1..542: note = Arah1_B1305
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 146 - protein, 542 aa, synthetic construct
  source 1..542: note = Arah1_B1306
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 147 - protein, 542 aa, synthetic construct
  source 1..542: note = Arah1_B1307
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 148 - protein, 542 aa, synthetic construct
  source 1..542: note = Arah1_B1308
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 149 - protein, 542 aa, synthetic construct
  source 1..542: note = Arah1_PLP264
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELALLGFGINAENNHRIFLAGKKDNVIDQIEKQAKDLAFQASGEQVEKLIKNQEESHFVKARPQSQSQSPSSPEKESPEKEDAEEENQGGKGPLLSILKAFN

SEQ ID NO: 150 - protein, 542 aa, synthetic construct
  source 1..542: note = Arah1_B1309
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNEGVIVKVSKDQVRELTEAAKSVSKKGSEEEGDITAPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDAEEENQGGKGPLLSILKAFN

SEQ ID NO: 151 - protein, 542 aa, synthetic construct
  source 1..542: note = Arah1_B1304
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDAEEENQGGKGPLLSILKAFN

SEQ ID NO: 152 - protein, 542 aa, synthetic construct
  source 1..542: note = Arah1_PLP317
RSPPGERTRGRKPGDYDDDRRQPVREAGGEWGPAGPRERERLEDWRQPREDTRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNRGVIVEVSKDQVRELTEAAKSVSKKGSEEEGDITAPINLRHGEPDLSNNFGKLHEVKPRKKNPQLQDLDMMLTCVEIKEGALMLPHFNSKAMVIVVVNKGKGNLELVAVRKEQQQRGRREEEEDEDEEEEGSNREVRRYTAELERGDVFIMPAAHPVAINASSNLALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 153 - protein, 542 aa, synthetic construct
  source 1..542: note = Arah1_PLP318
RSPPGERTRGRKPGDYDDDRRQPVREEGGEWGPAGPREREREEDWRQPREDTRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGEQEERGQERWSTRSSENNRGVIVEVSKDQVRELTEAAKSVSKKGSEEEGDITHPINLREGEPDLSNNFGKLHEVKPRKKNPQLQDLDMMLTCVEIKEGALMLPHFNSKAMVIVVVNKGKGNLELVAVRKEQQQRGRREEEEDEDEEEEGSNREVRRYTAELKRGDVFIMPAAHPVAINASSNLHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 154 - protein, 542 aa, synthetic construct
  source 1..542: note = Arah1_PLP496
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQGFSRNTLEAAFNAEFNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDAEEENQGGKGPLLSILKAFN

SEQ ID NO: 155 - protein, 542 aa, synthetic construct
  source 1..542: note = Arah1_PLP499
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQGFSRNTLEAAFNAEFNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 156 - protein, 542 aa, synthetic construct
  source 1..542: note = Arah1_PLP595
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 157 - protein, 542 aa, synthetic construct
  source 1..542: note = Arah1_PLP601
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSQNTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 158 - protein, 542 aa, synthetic construct
  source 1..542: note = Arah1_PLP607
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSKNTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 159 - protein, 542 aa, synthetic construct
  source 1..542: note = Arah1_PLP729
RSPPGERTRGRKPGDYDDDRRQPVREAGGEWGPAGPRERERLEDWRQPREDTRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 160 - protein, 542 aa, synthetic construct
  source 1..542: note = Arah1_PLP730
RSPPGERTRGRKPGDYDDDRRQPVREAGGEWGPAGPRERERLEDWRQPREDTRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGKGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 161 - protein, 542 aa, synthetic construct
  source 1..542: note = Arah1_PLP731
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFHPTSSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 162 - DNA, 129 bp, synthetic construct
  source 1..129: mol_type = other DNA; note = TEV 5' UTR
tctcaacacaacatatacaaaacaaacgaatctcaagcaatcaagcattctacttctattgcagcaatttaaatcatttcttttaaagcaaaagcaattttctgaaaattttcaccatttacgaacgat

SEQ ID NO: 163 - DNA, 138 bp, synthetic construct
  source 1..138: mol_type = other DNA; note = Xenopus beta globin 3' UTR
ggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 164 - DNA, 453 bp, synthetic construct
  source 1..453: mol_type = other DNA; note = Wt Ara h 2 nucleutide sequence 1
cgccagcagtgggaacttcagggcgacagacggtgtcagagccagctggagagggctaacctccggccctgcgaacagcacctgatgcaaaagatccagcgcgacgaggactcctacggacgcgacccatactcgcctagccaagatccctactccccgtcgcaagaccctgatcggagagatccgtactcaccgtccccgtatgatcggaggggtgccggatccagccagcatcaggaacgctgctgcaacgagctcaacgagttcgaaaacaaccagcgctgtatgtgcgaggcgctgcagcagattatggagaaccagtccgaccgactgcaggggaggcagcaggaacagcagttcaagcgggaactgcggaatctgccccaacaatgcggcttgagagcccctcaaagatgcgacctggaagtggagtcaggaggacgggaccgctac

SEQ ID NO: 165 - DNA, 453 bp, synthetic construct
  source 1..453: mol_type = other DNA; note = Wt Ara h 2 nucleutide sequence 2
cgccaacaatgggaattgcaaggagataggcggtgtcagtcacagctcgagcgagcaaacctgcgtccgtgtgaacagcatttgatgcagaaaatccagagagatgaagattcatacggtcgtgatccttattccccatctcaagatccttattctccaagtcaagatccggacaggcgcgatccctatagcccttccccttacgatcggcgcggtgccggctctagccaacaccaggaaagatgttgtaacgagctgaacgaattcgagaataatcaaaggtgtatgtgtgaagcactgcaacagataatggaaaatcagtcagatcgactgcaaggacggcaacaggaacagcaatttaaacgagagctgaggaatctgcctcaacaatgcggtctccgcgctccacaacggtgtgatctggaagtggagtcaggtggacgagatcgctac

SEQ ID NO: 166 - protein, 151 aa, synthetic construct
  source 1..151: note = Wt Ara h 2 amino acid sequence
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 167 - DNA, 453 bp, synthetic construct
  source 1..453: mol_type = other DNA; note = DE Ara 2 variant 1001 nucleutide sequence
agacaacagtgggaactccagggtgataacaggtgtcgtcgccaattggaaagagccttccttgatccgtgcgagagtcacttgatgcaaaagattcaacgagacgaggacagctatggccgtatccccacgagtgtgtcccaatcacccaccagtggctctcaggacccagatagaaggccccctacctctgaaagtccctatgaccgcagaggcgcgggatcaagtcagaatcaagaggattgctgctatttcctcaattcctttgaaaacaaccagcgctgcatgtgcgaggcgctccagctcatcatggcaaccaagagcgaccgtcttcaggttcgccagcaagagcaacagttcattgatatgctgcacaaccttcctcagcagtgtgggctgcgtgctccccagggctgcatgcttgaggtcgaaagcggcggtcgggacagatat

SEQ ID NO: 168 - protein, 151 aa, synthetic construct
  source 1..151: note = DE Ara 2 variant 1001 amino acid sequence
RQQWELQGDNRCRRQLERAFLDPCESHLMQKIQRDEDSYGRIPTSVSQSPTSGSQDPDRRPPTSESPYDRRGAGSSQNQEDCCYFLNSFENNQRCMCEALQLIMATKSDRLQVRQQEQQFIDMLHNLPQQCGLRAPQGCMLEVESGGRDRY

SEQ ID NO: 169 - DNA, 453 bp, synthetic construct
  source 1..453: mol_type = other DNA; note = DE Ara 2 variant 764 nucleutide sequence
agacaacagtgggaactccagggtgataacaggtgtcgtcgccaattggaaagagccttccttagaccgtgcgagagtcacttgatgcaaaagattcaacgagacgaggacagctatggccgtatccccacgagtgtgtcccaatcacccaccagtggctctcaggacccagatagaaggccccctacctctgaaagtccctatgaccgcagaggcgcgggatcaagtcagaatcaagagagatgctgctatgaactcaatgaatttgaaaacaaccagcgctgcatgtgcgaggcgctccagctcatcatggcaaccaagagcgaccgtcttcaggttcgccagcaagagcaacagttcattgatatgctgcacaaccttcctcagcagtgtgggctgcgtgctccccagggctgcatgcttgaggtcgaaagcggcggtcgggacagatat

SEQ ID NO: 170 - protein, 151 aa, synthetic construct
  source 1..151: note = DE Ara 2 variant 764 amino acid sequence
RQQWELQGDNRCRRQLERAFLRPCESHLMQKIQRDEDSYGRIPTSVSQSPTSGSQDPDRRPPTSESPYDRRGAGSSQNQERCCYELNEFENNQRCMCEALQLIMATKSDRLQVRQQEQQFIDMLHNLPQQCGLRAPQGCMLEVESGGRDRY

SEQ ID NO: 171 - DNA, 1626 bp, synthetic construct
  source 1..1626: mol_type = other DNA; note = Wt Ara h 1 nucleotide sequence 1
cggtcaccccctggagaacgcacccggggtagacagccgggagactacgatgacgataggcgacaacctagacgggaggaaggaggacgctggggtcctgctggaccacgggagagggaaagggaggaggactggcggcaacccagggaggattggcggcgccctagtcaccagcagccgagaaagatcagaccggaaggacgggaaggggaacaggaatggggaacccctgggagccacgtcagagaggaaacttcccgcaacaacccgttctacttcccatcccggcggttctccactcgctacggcaaccagaacggccggattagggtgctgcagcggttcgaccagaggtcgagacagttccagaatctccagaaccatcgcattgtgcaaatcgaggccaagccgaacactctcgtgctgccgaagcacgccgacgcggataacatcctcgtgattcagcagggtcaagccaccgtgacagtggcgaacgggaacaaccggaaatcctttaacctggacgaaggacacgccctgcgcatcccatcggggttcatctcctacatcctgaaccgccacgacaatcagaacctgagggtggccaagatttccatgcccgtgaacacgcccggccagttcgaggactttttccccgcctcatcacgcgaccagtccagctatctgcaaggtttctcgcggaacaccctggaagccgctttcaacgccgaattcaacgaaattcggcgggtgttgcttgaagagaatgcaggaggagagcaggaggaaagaggccaacgccggtggagcaccagatcctccgagaacaatgagggcgtgatcgtgaaagtgtcgaaggagcacgtggaggaactgaccaaacacgcgaagtccgtgtccaagaagggcagcgaggaagaaggagacatcaccaaccccatcaatctgcgcgaaggggagcctgacctgtccaacaacttcggaaagctgttcgaagtcaagccggacaagaagaatccacagctgcaggatctggacatgatgctcacttgcgtggagattaaggaaggcgcactcatgctgccccatttcaactcgaaggccatggtcatcgtggtcgtgaacaagggaactggcaacctcgagcttgtcgccgtgcggaaggaacaacaacagcgcggtcgcagagaagaggaagaggatgaagatgaagaagaggagggctccaaccgcgaggtccggagatacaccgcgcgcctgaaggaaggggacgtgttcatcatgcctgccgcccatcctgtggccattaacgcgtccagcgaactgcatttgctcgggttcggcatcaatgcggagaacaaccacagaatctttctggccggcgacaaggacaacgtgatcgaccaaattgagaagcaggccaaagatctggccttcccgggatccggcgaacaggtcgagaagctgatcaagaaccagaaggaatcgcacttcgtgagcgcacggcctcagagccagtcacagtccccatcttcgcccgaaaaggagtccccggaaaaggaggaccaggaggaagaaaaccagggcggaaagggtcccctgctgagcatcctcaaggccttcaac

SEQ ID NO: 172 - DNA, 1626 bp, synthetic construct
  source 1..1626: mol_type = other DNA; note = Wt Ara h 1 nucleotide sequence 2
agatccccaccaggagagcgaactcgaggacggcagccaggagactatgatgatgatcgccggcagcctcgtagagaagaaggtggacgctggggacccgcagggcctcgcgaacgagagcgggaagaagactggcggcaacctcgtgaagattggcgtcgcccatcccatcagcagccacgcaagatacgaccggaaggccgcgagggagagcaggagtggggtacacctggatctcacgtgcgcgaggagactagcaggaataacccattctatttccccagtcgcagattttccactcggtatggcaatcaaaacggccggatcagagtgctgcaacggttcgatcaaagaagcaggcaattccaaaacttgcaaaatcacaggattgtacagatagaggccaagcctaacactttggtcttgccaaagcatgccgacgccgacaacattctcgtaatccaacaaggacaggccaccgttacagttgctaatggcaataacaggaaatcctttaatctggacgaaggacatgctcttagaatcccatccggattcatttcatacatcctcaacaggcatgacaaccaaaatttgcgggttgctaagatttcaatgccagtaaatacaccgggtcaattcgaagacttcttccccgcctctagcagggaccaatcttcatacctgcaaggcttctcaagaaacaccctcgaagctgcgttcaatgccgaatttaatgaaattcgcagagttctcctcgaggaaaacgctggcggcgagcaagaggaacgcgggcaaagacgttggtcaacacgttccagcgagaataacgagggcgttatcgtaaaggtatcaaaggagcacgtagaagagctcacgaagcatgctaaatctgtgagtaagaagggaagcgaagaagagggagacattaccaaccccataaatttgagggaaggggaacctgacctctctaataattttggcaaattgtttgaggtgaaacccgacaagaagaatccgcaattgcaagacttggacatgatgctgacatgtgtcgagataaaagaaggggcactcatgctccctcacttcaacagcaaagccatggtaattgtcgtcgtaaacaaagggaccgggaatctggagctggtggctgttcgaaaggaacagcagcaacggggcaggcgagaggaagaggaagacgaggacgaggaagaggaaggctctaaccgggaagtgcggcggtacacagctagattgaaagagggagacgtgtttattatgcccgcggcccacccagttgctattaacgcaagttccgagttgcatcttctcggctttgggatcaatgctgaaaacaatcaccgtatattcttggctggggacaaagacaatgttatagatcaaattgagaaacaggcaaaggatctggcattccccggctctggggagcaagttgaaaagctcatcaagaaccaaaaggagtctcacttcgtctccgcaaggccccagagccagtctcaatcacccagcagcccggagaaagaaagcccagaaaaggaagatcaagaggaagagaaccagggcggaaagggccccctgctgtctatcctcaaggctttcaac

SEQ ID NO: 173 - DNA, 1626 bp, synthetic construct
  source 1..1626: mol_type = other DNA; note = DE Arah1 variant 5
cgtagtcctcccggagaaagaacccggggacggcaaccaggagactatgatgatgatcgtcggcagccacgccgggaagaaggtggccggtggggaccagccggtccacgcgaacgcgagagagaggaggactggcgtcagcctcgcgaggattggcgtcgtcccagccatcagcagccaagaaagataagacccgaagggcgcgagggtgagcaggagtggggtacgccgggatcacacgtccgggaggagacttcacgcaataaccccttctatttcccctcgcgtcgttttagcactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgcgaaccaaaatttacgcgtagctaagatctctatgccggtcaatactccaggtcaattcgaagacttcttccctgcgtcctcacgtgaccaaagttcttacttacaaggattctcccgtaacaccttggaagcggctttcaatgcggaatttaatgaaatcagacgtgttctgctggaggaaaacgcgggtggagagcaagaagaacgcgggcaacgccgctggtcgacgcgctcttcagagaataacgagggagtcatcgtaaaggtgtccaaggagcacgtagaagagcttacgaagcatgcgaaatctgtgtctaagaagggcagcgaagaggagggagacattacgaacccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcgtccggagagcaagtcgaaaagctgataaaaaaccaggaagagagccacttcgttagcgcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 174 - protein, 542 aa, synthetic construct; identical to SEQ ID NO: 92
  source 1..542: note = DE Arah1 variant 5

SEQ ID NO: 175 - DNA, 1626 bp, synthetic construct
  source 1..1626: mol_type = other DNA; note = DE Arah1 variant 8
cgtagtcctcccggagaaagaacccggggacggcaaccaggagactatgatgatgatcgtcggcagccacgccgggaagaaggtggccggtggggaccagccggtccacgcgaacgcgagagagaggaggactggcgtcagcctcgcgaggattggcgtcgtcccagccatcagcagccaagaaagataagacccgaagggcgcgagggtgagcaggagtggggtacgccgggatcacacgtccgggaggccacttcagccgcgaaccccttctatttcccctcggcccgttttgccactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgccaaccaaaatttacgcgtagctaagatctctatgccggtcaatactccaggtcaattcgaagacttcttccctgcgtcctcacgtgaccaaagttcttacttacaaggattctcccgtaacaccttggaagcggctttcaatgcggaatttaatgaaatcagacgtgttctgctggaggaaaacgcgggtggagagcaagaagaacgcgggcaacgccgctggtcgacgcgctcttcagagaataacgagggagtcatcgtaaaggtgtccaaggaccaagtaagagagcttacggaagccgcgaaatctgtgtctaagaagggcagcgaagaggagggagacattacgaacccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcctccggagagcaagtcgaaaagctgataaaaaaccaggaagagagccacttcgttagcgcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 176 - protein, 542 aa, synthetic construct; identical to SEQ ID NO: 95
  source 1..542: note = DE Arah1 variant 8

SEQ ID NO: 177 - DNA, 1626 bp, synthetic construct
  source 1..1626: mol_type = other DNA; note = DE Arah1 variant 25
cgtagtcctcccggagaaagaacccggggacggcaaccaggagactatgatgatgatcgtcggcagccagtccgggaagaaggtggcgagtggggaccagccggtccacgcgaacgcgagagagaggaggactggcgtcagcctcgcgaggattggcgtcgtcccagccatcagcagccaagaaagataagacccgaagggcgcgagggtgagcaggagtggggtacgccgggatcacacgtccgggaggccacttcagccgcgaaccccttctatttcccctcggcccgttttgccactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgccaaccaaaatttacgcgtagctaagatctctatgccggtcaatactccaggtcaattcgaagacttcttccctgcgtcctcacgtgaccaaagttcttacttacaaggattctcccgtaacaccttggaagcggctttcaatgcggaatttaatgaaatcagacgtgttctgctggaggaaaacgcgggtggagagcaagaagaacgcgggcaacgccgctggtcgacgcgctcttcagagaataacgagggagtcatcgtaaaggtgtccaaggaccaagtaagagagcttacggaagccgcgaaatctgtgtctaagaagggcagcgaagaggagggagacattacgaacccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcctccggagagcaagtcgaaaagctgataaaaaaccaggaagagagccacttcgttagcgcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 178 - protein, 542 aa, synthetic construct; identical to SEQ ID NO: 108
  source 1..542: note = DE Arah1 variant 25

SEQ ID NO: 179 - DNA, 1626 bp, synthetic construct
  source 1..1626: mol_type = other DNA; note = DE Arah1 variant 35
cgtagtcctcccggagaaagaacccggggacggcaaccaggagactatgatgatgatcgtcggcagccagtccgggaagaaggtggcgagtggggaccagccggtccacgcgaacgcgagagagaggaggactggcgtcagcctcgcgaggattggcgtcgtcccagccatcagcagccaagaaagataagacccgaagggcgcgagggtgagcaggagtggggtacgccgggatcacacgtccgggaggccacttcagccgcgaaccccttctatttcccctcggcccgttttgccactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgccaaccaaaatttacgcgtagctaagatctctatgccggtcaatactccaggtcaattcgaagacttcttccctgcgtcctcacgtgaccaaagttcttacttacaaggattctcccgtaacaccttggaagcggctttcaatgcggaatttaatgaaatcagacgtgttctgctggaggaaaacgcgggtggagagcaagaagaacgcgggcaagagcgctggtcgacgcgctcttcagagaataacgagggagtcatcgtaaaggtgtccaaggaccaagtaagagagcttacggaagccgcgaaatctgtgtctaagaagggcagcgaagaggagggagacattacgaacccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcctccggagagcaagtcgaaaagctgataaaaaaccaggaagagagccacttcgttagcgcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 180 - protein, 543 aa, synthetic construct
  source 1..543: note = DE Arah1 variant 35
GRSPPGERTRGRQPGDYDDDRRQPVREEGGEWGPAGPREREREEDWRQPREDWRRPSHQQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQERWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVSARPQSQSQSPSSPEKESPEKEDQEEENQGGKGPLLSILKAFN

SEQ ID NO: 181 - DNA, 1626 bp, synthetic construct
  source 1..1626: mol_type = other DNA; note = DE Arah1 variant 51
cgtagtcctcccggagaaagaacccggggacggaagccaggagactatgatgatgatcgtcggcagccagtccgggaagaaggtggcgagtggggaccagccggtccacgcgaacgcgagagagaggaggactggcgtcagcctcgcgaggattggcgtcgtcccagccatcagcagccaagaaagataagacccgaagggcgcgagggtgagcaggagtggggtacgccgggatcacacgtccgggaggccacttcagccgcgaaccccttctatttcccctcggcccgttttgccactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgccaaccaaaatttacgcgtagctaagatctctatgccggtcaatactccaggtcatttcgaagacttcttccctgcgtcctcacgtgaccaaagttcttacttacaagcgttctccgaaaacaccttggaagcggctttcaatgcggaacgtaatgaaatcagacgtgttctgctggaggaaaacgcgggtggagagcaagaagaacgcgggcaagaacgctggtcgacgcgctcttcagagaataacgagggagtcatcgtaaaggtgtccaaggaccaagtaagagagcttacggaagccgcgaaatctgtgtctaagaagggcagcgaagaggagggagacattacgcatccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcctccggagagcaagtcgaaaagctgataaaaaaccaggaagagagccacttcgttagcgcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 182 - protein, 542 aa, synthetic construct; identical to SEQ ID NO: 129
  source 1..542: note = DE Arah1 variant 51

SEQ ID NO: 183 - DNA, 1626 bp, synthetic construct
  source 1..1626: mol_type = other DNA; note = DE Arah1 variant 52
cgtagtcctcccggagaaagaacccggggacggcaaccaggagactatgatgatgatcgtcggcagccagtccgggaagaaggtggcgagtggggaccagccggtccacgcgaacgcgagagagaggaggactggcgtcagcctcgcgaggattggcgtcgtcccagcgattcgcagccaagaaagataagacccgaagggcgcgagggtgagcaggagtggggtacgccgggatcacacgtccgggaggccacttcagccgcgaaccccttctatttcccctcggcccgttttgccactcgttatgggaatcaaaacggaagaataagagtacttcaacggttcgatcaacgtagccgccaattccaaaaccttcaaaatcaccgcatagtccagatcgaggccaagcccaacacccttgtcttacctaagcatgcagacgccgacaacattctggttatacaacaaggtcaggccaccgtaacggtcgcaaatgggaataaccgcaaatcatttaatttagacgaaggtcatgcgcttagaataccgtccggattcatttcctacatcttaaaccgtcatgccaaccaaaatttacgcgtagctaagatctctatgccggtcaatactccaggtcaattcgaagacttcttccctgcgtcctcacgtgaccaaagttcttacttacaaggattctcccgtaacaccttggaagcggctttcaatgcggaatttaatgaaatcagacgtgttctgctggaggaaaacgcgggtggagagcaagaagaacgcgggcaacgccgctggtcgacgcgctcttcagagaataacgagggagtcatcgtaaaggtgtccaaggaccaagtaagagagcttacggaagccgcgaaatctgtgtctaagaagggcagcgaagaggagggagacattacgaacccaataaatcttcgtgaaggagaaccggacctttccaataattttggtaaattacacgaggtcaaacccgacaaaaagaatccgcaattgcaagacttagacatgatgttgacttgtgttgagataaaagaaggggcgttgatgctgccacacttcaactctaaagcaatggttattgttgtagtcaacaaagggaccggcaatctggagctggtggctgttagaaaggaacagcagcaaagaggcagacgtgaagaagaggaagacgaggacgaggaagaagaaggttctaaccgcgaggtgcgccggtacacagcagaacttaaaagaggtgacgtatttattatgcccgcggctcacccggttgctattaacgcctcgtccgagttgcatctgttgggttttggaataaatgcggaaaacaatcaccgtatattcttggcagggaagaaagacaatgtaatagatcaaatcgagaaacaggctaaggatctggctttccaagcctccggagagcaagtcgaaaagctgataaaaaaccaggaagagagccacttcgttaaagcccgtccccagtctcagtcgcaatccccatcgagcccggaaaaagaaagccctgagaaggaggatcaggaggaagagaaccagggtggaaagggaccacttttatccatattaaaagccttcaac

SEQ ID NO: 184 - protein, 542 aa, synthetic construct
  source 1..542: note = DE Arah1 variant 52
RSPPGERTRGRQPGDYDDDRRQPVREEGGEWGPAGPREREREEDWRQPREDWRRPSDSQPRKIRPEGREGEQEWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGQFEDFFPASSRDQSSYLQGFSRNTLEAAFNAEFNEIRRVLLEENAGGEQEERGQRRWSTRSSENNEGVIVKVSKDQVRELTEAAKSVSKKGSEEEGDITNPINLREGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKKDNVIDQIEKQAKDLAFQASGEQVEKLIKNQEESHFVKARPQSQSQSPSSPEKESPEKEDQEEENQGGKGPLLSILKAFN

SEQ ID NO: 185 - DNA, 62 bp, synthetic construct
  source 1..62: mol_type = other DNA; note = HMM”leader sequence
atgtggtggcgcctgtggtggctcttgctttgttgcttctcctctggcccatggtgtgggcc

SEQ ID NO: 186 - protein, 21 aa, synthetic construct
  source 1..21: note = HMM”leader sequence
MWWRLWWLLLLLLLLWPMVWA

SEQ ID NO: 187 - DNA, 51 bp, synthetic construct
  source 1..51: mol_type = other DNA; note = BM40 leader sequence
atgcgggcctggatcttcttcctgctctgccttgccgggagagcacttgcc

SEQ ID NO: 188 - protein, 17 aa, synthetic construct
  source 1..17: note = BM40 leader sequence
MRAWIFFLLCLAGRALA

SEQ ID NO: 189 - DNA, 60 bp, synthetic construct
  source 1..60: mol_type = other DNA; note = IgG kappa light chain leader sequence
atgcgcgtgcctgcccaactgcttggactgctcttgctgtggcttcggggagctcgctgt

SEQ ID NO: 190 - protein, 20 aa, synthetic construct
  source 1..20: note = IgG kappa light chain leader sequence
MRVPAQLLGLLLLWLRGARC

SEQ ID NO: 191 - DNA, 54 bp, synthetic construct
  source 1..54: mol_type = other DNA; note = IgE heavy chain leader sequence
atggactggacctggatcctgtttcttgtggcagccgccacccgagtccactcc

SEQ ID NO: 192 - protein, 18 aa, synthetic construct
  source 1..18: note = IgE heavy chain leader sequence
MDWTWILFLVAAATRVHS

SEQ ID NO: 193 - protein, 598 aa, synthetic construct
  source 1..598: note = combo 68-TM
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFNSGSGSGGSGSGSGGSSSSQPTIPIVGIIAGLVLLGAVITGAVVAAVMWRRKSSDRK

SEQ ID NO: 194 - protein, 623 aa, synthetic construct
  source 1..623: note = combo 68-MITD
RSPPGERTRGRKPGDYDDDRRQPVREAGGEWGPAGPRERERLEDWRQPREDWRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGNNRKSFNLDEGHALRIPSGFISYILNRHANQNLRVAKISMPVNTPGHFEDFFPASSRDQSSYLQAFSENTLEAAFNAERNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELERGDVFIMPAAHPVAINASSELALLGFGINAENNHRIFLAGKSDNVIDQIEKQAKDLAFQASGEQVEKLIKNQEESHFVKARPQSQSQSPSSPEKESPEKEDAEEENQGGKGPLLSILKAFNSGSGSGGSGSGSGGSSSSQPTIPIVGIIAGLVLLGAVITGAVVAAVMWRRKSSDRKGGSYTQAASSDSAQGSDVSLTACKV

SEQ ID NO: 195 - protein, 139 aa, synthetic construct
  source 1..139: note = GM Arah2
RQQWELQGDRRCQSQLERANLRPCEQHLMQKIQRDEDSYGRDPYSPAQDPYAPGPYDRRGAGSSQHQERCCNELNEFENNQRCMCEALQQIMENQSDRLQGRQQEQQFKRELRNLPQQCGLRAPQRCDLEVESGGRDRY

SEQ ID NO: 196 - protein, 195 aa, synthetic construct
  source 1..195: note = GM Arah2-TM
RQQWELQGDRRCQSQLERANLRPCEQHLMQKIQRDEDSYGRDPYSPAQDPYAPGPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSSSQPTIPIVGIIAGLVLLGAVITGAVVAAVMWRRKSSDRK

SEQ ID NO: 197 - protein, 220 aa, synthetic construct
  source 1..220: note = GM Arah2- MITD
RQQWELQGDRRCQSQLERANLRPCEQHLMQKIQRDEDSYGRDPYSPAQDPYAPGPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSSSQPTIPIVGIIAGLVLLGAVITGAVVAAVMWRRKSSDRKGGSYTQAASSDSAQGSDVSLTACKV

SEQ ID NO: 198 - protein, 195 aa, synthetic construct; identical to SEQ ID NO: 196
  source 1..195: note = Arah2 TM

SEQ ID NO: 199 - protein, 232 aa, synthetic construct
  source 1..232: note = Arah2 MITD
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSSSQPTIPIVGIIAGLVLLGAVITGAVVAAVMWRRKSSDRKGGSYTQAASSDSAQGSDVSLTACKV

SEQ ID NO: 200 - protein, 382 aa, synthetic construct
  source 1..382: note = GM Arah2-IgG1 Fc
RQQWELQGDRRCQSQLERANLRPCEQHLMQKIQRDEDSYGRDPYSPAQDPYAPGPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 380 aa, synthetic construct
  source 1..380: note = GM Arah2-IgG4 Fc
RQQWELQGDRRCQSQLERANLRPCEQHLMQKIQRDEDSYGRDPYSPAQDPYAPGPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 394 aa, synthetic construct
  source 1..394: note = Arah2-IgG1 Fc
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 392 aa, synthetic construct
  source 1..392: note = Arah2-IgG4 Fc
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRYSGSGSGGSGSGSGGSSGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - protein, 394 aa, synthetic construct
  source 1..394: note = 1001-IgG1 Fc
RQQWELQGDNRCRRQLERAFLDPCESHLMQKIQRDEDSYGRIPTSVSQSPTSGSQDPDRRPPTSESPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 392 aa, synthetic construct
  source 1..392: note = 1001-IgG4 Fc
RQQWELQGDNRCRRQLERAFLDPCESHLMQKIQRDEDSYGRIPTSVSQSPTSGSQDPDRRPPTSESPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 206 - protein, 382 aa, synthetic construct
  source 1..382: note = GM 1001-IgG1 Fc
RQQWELQGDNRCRRQLERAFLDPCESHLMQKIQRDEDSYGRIPTSVSQADYAGGPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 380 aa, synthetic construct
  source 1..380: note = GM 1001-IgG4 Fc
RQQWELQGDNRCRRQLERAFLDPCESHLMQKIQRDEDSYGRIPTSVSQADYAGGPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 208 - protein, 137 aa, synthetic construct
  source 1..137: note = 1001 var2
RQQWELQGDNRCRRQLERANLTPCESHLMQKIQRDEDSYGRIPTSVSQDPYGPYDRRGAGSSQNQEQCCYSLNSFENNQRCMCEALQKIMQTLSDRLQGRQQEQQFIEGLHNLPQQCGLRAPQGCNLEVESGGRDRY

SEQ ID NO: 209 - protein, 137 aa, synthetic construct
  source 1..137: note = 1001 var4
RQQWELQGDNRCRRQLERAHLKPCESHLMQKIQRDEDSYGRIPTSVSQDPYGPYDRRGAGSSQNQEQCCYSLNQFENNQRCMCEALQKIMATKSDRLQGRQQEQQFIEGLHNLPQQCGLRAPQGCNLEVESGGRDRY

SEQ ID NO: 210 - protein, 137 aa, synthetic construct
  source 1..137: note = 1001 var6
RQQWELQGDNRCRRQLERAHLKPCESHLMQKIQRDEDSYGRIPTSVSQDPYGPYDRRGAGSSQNQEQCCYNLNQFENNQRCMCEALQKIMHTLSDRLQGRQQEQQFIRMLHNLPQQCGLRAPQGCNLEVESGGRDRY

SEQ ID NO: 211 - protein, 542 aa, synthetic construct
  source 1..542: note = Arah1_PLP737
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSANVIDQIEKQAKDLAFQASGEQVEKLIKNQEESHFVKARPQSQSQSPSSPEKESPEKEDAEEENQGGKGPLLSILKAFN

SEQ ID NO: 212 - protein, 542 aa, synthetic construct
  source 1..542: note = Arah1_PLP738
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSRNVIDQIEKQAKDLAFQASGEQVEKLIKNQEESHFVKARPQSQSQSPSSPEKESPEKEDAEEENQGGKGPLLSILKAFN

SEQ ID NO: 213 - protein, 542 aa, synthetic construct
  source 1..542: note = Arah1_PLP739
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSHNVIDQIEKQAKDLAFQASGEQVEKLIKNQEESHFVKARPQSQSQSPSSPEKESPEKEDAEEENQGGKGPLLSILKAFN

SEQ ID NO: 214 - protein, 542 aa, synthetic construct
  source 1..542: note = Arah1_PLP740
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNFRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 215 - protein, 542 aa, synthetic construct
  source 1..542: note = Arah1_PLP741
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNARIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 216 - protein, 542 aa, synthetic construct
  source 1..542: note = Arah1_PLP742
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNYRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 217 - protein, 542 aa, synthetic construct
  source 1..542: note = Arah1_PLP743
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNDRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 218 - protein, 542 aa, synthetic construct
  source 1..542: note = Arah1_PLP744
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNNRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 219 - protein, 542 aa, synthetic construct
  source 1..542: note = Arah1_PLP745
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNQRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 220 - protein, 542 aa, synthetic construct
  source 1..542: note = Arah1_PLP746
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNQRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 221 - protein, 542 aa, synthetic construct
  source 1..542: note = Arah1_PLP747
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNNRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 222 - protein, 542 aa, synthetic construct
  source 1..542: note = Arah1_PLP748
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSADQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 223 - protein, 542 aa, synthetic construct
  source 1..542: note = Arah1_PLP749
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSDDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 224 - protein, 542 aa, synthetic construct
  source 1..542: note = Arah1_PLP750
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 225 - protein, 542 aa, synthetic construct
  source 1..542: note = Arah1_PLP751
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSEDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 226 - protein, 542 aa, synthetic construct
  source 1..542: note = Arah1_PLP752
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGDNRKSFNLDEGHALRIPSGFISYILNRHANQNLRVAKISMPVNTPGHFEDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 227 - protein, 542 aa, synthetic construct
  source 1..542: note = Arah1_PLP753
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFRDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAADPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 228 - protein, 542 aa, synthetic construct
  source 1..542: note = Arah1_PLP754
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFDDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 229 - protein, 542 aa, synthetic construct
  source 1..542: note = Arah1_PLP755
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFLDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAIPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 230 - protein, 542 aa, synthetic construct
  source 1..542: note = Arah1_PLP756
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFIDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAALPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 231 - protein, 542 aa, synthetic construct
  source 1..542: note = Arah1_PLP757
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFFDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 232 - protein, 542 aa, synthetic construct
  source 1..542: note = Arah1_PLP758
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFADFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 233 - protein, 542 aa, synthetic construct
  source 1..542: note = Arah1_PLP759
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNDRIFLAGKSANVIDQIEKQAKDLAFQASGEQVEKLIKNQEESHFVKARPQSQSQSPSSPEKESPEKEDAEEENQGGKGPLLSILKAFN

SEQ ID NO: 234 - protein, 542 aa, synthetic construct
  source 1..542: note = Arah1_PLP760
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNNRIFLAGKSRNVIDQIEKQAKDLAFQASGEQVEKLIKNQEESHFVKARPQSQSQSPSSPEKESPEKEDAEEENQGGKGPLLSILKAFN

SEQ ID NO: 235 - protein, 542 aa, synthetic construct
  source 1..542: note = Arah1_PLP761
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGRITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNQRIFLAGKSHNVIDQIEKQAKDLAFQASGEQVEKLIKNQEESHFVKARPQSQSQSPSSPEKESPEKEDAEEENQGGKGPLLSILKAFN

SEQ ID NO: 236 - protein, 542 aa, synthetic construct
  source 1..542: note = Arah1_PLP762
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNQRIFLAGKSANVIDQIEKQAKDLAFQASGEQVEKLIKNQEESHFVKARPQSQSQSPSSPEKESPEKEDAEEENQGGKGPLLSILKAFN

SEQ ID NO: 237 - protein, 542 aa, synthetic construct
  source 1..542: note = Arah1_PLP763
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNNRIFLAGKSHNVIDQIEKQAKDLAFQASGEQVEKLIKNQEESHFVKARPQSQSQSPSSPEKESPEKEDAEEENQGGKGPLLSILKAFN

SEQ ID NO: 238 - protein, 542 aa, synthetic construct
  source 1..542: note = Arah1_PLP764
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFEDFFPASSRDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNFRIFLAGKSFNVIDQIEKQAKDLAFQASGEQVEKLIKNQEESHFVKARPQSQSQSPSSPEKESPEKEDAEEENQGGKGPLLSILKAFN

SEQ ID NO: 239 - protein, 542 aa, synthetic construct
  source 1..542: note = Arah1_PLP765
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFRDFFPASSEDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAADPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 240 - protein, 542 aa, synthetic construct
  source 1..542: note = Arah1_PLP766
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFLDFFPASSEDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAIPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 241 - protein, 542 aa, synthetic construct
  source 1..542: note = Arah1_PLP767
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFIDFFPASSEDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAALPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 242 - protein, 542 aa, synthetic construct
  source 1..542: note = Arah1_PLP768
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFFDFFPASSEDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 243 - protein, 542 aa, synthetic construct
  source 1..542: note = Arah1_PLP769
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFRDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAADPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 244 - protein, 542 aa, synthetic construct
  source 1..542: note = Arah1_PLP770
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFLDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAIPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 245 - protein, 542 aa, synthetic construct
  source 1..542: note = Arah1_PLP771
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFIDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAALPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 246 - protein, 542 aa, synthetic construct
  source 1..542: note = Arah1_PLP772
RSPPGERTRGRKPGDYDDDRRQPVREAGGEWGPAGPRERERLEDWRQPREDLRRPSDRQPRKIRPEGREGEQAWGTPGSHVREATSAANPFYFPSARFATRYGNQNGRIRVLQRFDQRSRQFQNLQNHRIVQIEAKPNTLVLPKHADADNILVIQQGQATVTVANGRNRKSFNLDEGHALRIPSGFISYILNRHANQNLRVAKISMPVNTPGHFFDFFPASSQDQSSYLQAFSENTLEAAFNAEYNEIRRVLLEENAGGKQEKRGQERWSTRSSENNEGVIVKVSKDQVRELTEAAKSVSKKGSEEEGDITAPINLRHGEPDLSNNFGKLHEVKPDKKNPQLQDLDMMLTCVEIKEGALMLPHFNSKAMVIVVVNKGTGNLELVAVRKEQQQRGRREEEEDEDEEEEGSNREVRRYTAELKRGDVFIMPAAHPVAINASSELHLLGFGINAENNHRIFLAGKSDNVIDQIEKQAKDLAFQASGEQVEKLIKNQEESHFVKARPQSQSQSPSSPEKESPEKEDAEEENQGGKGPLLSILKAFN

SEQ ID NO: 247 - protein, 151 aa, synthetic construct
  source 1..151: note = Arah2_conbo31
RQQWELQGDNRCRRQLERAHLKPCESHLMQKIQRDEDSYGRIPTSVSQAPGAGGQDPDRRPPAGEGPYDRRGAGSSQNQEQCCYSLNQFENNQRCMCEALQKIMATKSDRLQDRQQEQQFIEGLHNLPQQCGLRAPQGCPLEVESGGRDRY

SEQ ID NO: 248 - protein, 207 aa, synthetic construct
  source 1..207: note = 1001-TM
RQQWELQGDNRCRRQLERAFLDPCESHLMQKIQRDEDSYGRIPTSVSQSPTSGSQDPDRRPPTSESPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSSSQPTIPIVGIIAGLVLLGAVITGAVVAAVMWRRKSSDRK

SEQ ID NO: 249 - protein, 195 aa, synthetic construct
  source 1..195: note = GM1001-TM
RQQWELQGDNRCRRQLERAFLDPCESHLMQKIQRDEDSYGRIPTSVSQADYAGGPYDRRGAGSSQNQEDCCYFLNSFENNQRCMCEALQLIMATKSDRLQVRQQEQQFIDMLHNLPQQCGLRAPQGCMLEVESGGRDRYSGSGSGGSGSGSGGSSSSQPTIPIVGIIAGLVLLGAVITGAVVAAVMWRRKSSDRK